(19) United States
(12) Patent Application Publication
(10) Pub. No.: US 2016/0122713 A1
(43) Pub. Date: May 5, 2016

(54) GENETICALLY-MODIFIED MICRO-ORGAN SECRETING A THERAPEUTIC PEPTIDE AND METHODS OF USE THEREOF

(71) Applicant: Medgenics Medical Israel Ltd., Misgav (IL)

(72) Inventors: Nir Shapir, Misgav (IL); Garry Neil, New Hope, PA (US); Amos Panet, Mevasseret Zion (IL); Reem Miari, Sakhnin (IL)

(73) Assignee: MEDGENICS MEDICAL ISRAEL LTD., Misgav (IL)

(21) Appl. No.: 14/882,425

(22) Filed: Oct. 13, 2015

Related U.S. Application Data
(60) Provisional application No. 62/063,606, filed on Oct. 14, 2014.
(60) Provisional application No. 62/159,918, filed on May 11, 2015.

(51) Int. Cl. C12N 5/071; C07K 14/605; C07K 14/505; A61K 35/36

(52) U.S. Cl. CPC C12N 5/0625; A61K 35/36; C07K 14/605; C07K 14/505; C12N 2510/00; A61K 38/00

(57) ABSTRACT

Provided herein is a genetically-modified micro-organ that provides a sustained delivery of a therapeutic peptide. The genetically-modified micro-organ may comprise a viral vector or expression cassette comprising at least two nucleic acid sequences encoding the therapeutic peptide separated by a cleavable linker. Further provided herein is a method of treating or preventing a disease or disorder in a human subject that can be treated or prevented by administration of a therapeutic peptide over a sustained time period using the genetically-modified micro-organ described herein.

### REFERENCE TO SEQUENCE LISTING SUBMITTED VIA EFS-WEB

This application is being filed electronically via EFS-Web and includes an electronically submitted sequence listing in .txt format. The .txt file contains a sequence listing entitled “2015-10-13_01118-0005-00US_ST25-v2.txt” created on Oct. 13, 2015 and is 294,344 bytes in size. The sequence listing contained in this .txt file is part of the specification and is hereby incorporated by reference herein in its entirety.

### BACKGROUND

Therapeutic peptides are desirable for administration to humans for the treatment of various diseases and conditions. Peptides typically found in the gastrointestinal tract, or variants of those peptides, are desirable for administration to humans for the treatment of gastrointestinal diseases and/or conditions. Peptide hormones derived from preproglucagon and secreted from the L-cells in the gastrointestinal tract include glucagon, GLP-1, GLP-2, glicentin, and oxyntomodulin. The neuropeptide Y family of peptides is another group of peptides secreted in the gastrointestinal tract and includes neuropeptide Y (NPY), peptide YY (PYY), and pancreatic polypeptide (PP). Other peptides and peptide variants are also desirable for administration to humans for the treatment of other diseases.

Glucagon-like peptide-2 (GLP-2) (SEQ ID NO: 1) is a 33-amino acid peptide derived from preproglucagon and secreted from L-cells of the gut. Administration of GLP-2, including subcutaneous administration, has been found to be responsible for inducing a marked increase in bowel weight and villus growth of the jejunum and ileum. The biological role of GLP-2 includes that of stimulating small bowel epithelial proliferation.

Subcutaneous administration of a GLP-2 variant known as teduglutide (GATTEX®) is indicated for patients with short bowel syndrome (SBS) who are dependent on parenteral support. While teduglutide has been found to reduce SBS patients' parenteral nutrition requirements, subcutaneous administration is inconvenient, painful, and difficult for some patients to perform with the kind of regularity needed to maintain therapeutic levels of proteins in the body.

Oxyntomodulin (SEQ ID NO: 22) is a 37-amino acid peptide secreted from the L-cells of the gut following nutrient ingestion. Administration of oxyntomodulin has been shown to delay gastric emptying and to decrease gastric acid secretion. Oxyntomodulin has also been found to cause significant reduction in weight and appetite, leading to its study for treatment of obesity, diabetes, hypothalmic hyperphagia, binge-eating disorder, Prader-Willi syndrome, overeating, and other gastrointestinal conditions and diseases.

PYY (Peptide YY) is co-secreted from L-cells with oxyntomodulin. PYY is generated from a precursor peptide, which undergoes posttranslational processing to generate two forms of PYY: a 36-amino acid form PYY₁₋₃₆ (SEQ ID NO: 25) and a 34-amino acid form PYY₃₋₃₆ (SEQ ID NO: 31) that is also biologically active. As used herein, unless otherwise designated, “PYY” means either form of this peptide.

PYY, either alone or in combination with oxyntomodulin, has been found to decrease food intake and body weight and is useful for treating obesity, diabetes, hypothalmic hyperphagia, binge-eating disorder, Prader-Willi syndrome, overeating, and other gastrointestinal conditions and diseases.

Attempts have been made to employ a delivery vector for in vivo expression of a fusion protein comprising a peptide and a carrier protein (such as GFP or albumin); however, these constructs require a substantially larger vector. See US 2012/0157513. Larger constructs may also enhance immunogenicity or provide a negative effect on potency, such as by potential reduced affinity to the receptor.

Thus, the art needs a mechanism for delivering therapeutic peptides, such as GLP-2, oxyntomodulin, and/or PYY, in vivo.

### SUMMARY

In accordance with the description, the inventors have achieved sustained expression of a therapeutic peptide from a genetically-modified micro-organ (GMMO). In certain embodiments, the therapeutic peptide is GLP-2, oxyntomodulin, PYY, or a combination of these therapeutic peptides, or variants of these peptides that retain their functional activity.

In one embodiment, the invention comprises a peptide GMMO that is capable of providing a sustained delivery of at least one therapeutic peptide when implanted in a human subject in vivo. In certain embodiments, the peptide GMMO comprises a viral vector comprising at least two nucleic acid sequences encoding at least one therapeutic peptide separated by at least one cleavable linker. In other embodiments, the peptide GMMO comprises an expression cassette comprising at least two nucleic acid sequences encoding at least one therapeutic peptide separated by at least one cleavable linker. In each embodiment, the GMMO may comprise two or more copies of the at least one therapeutic peptide, and the nucleic acid sequence encoding the peptide may be the same or different. For instance, optimized nucleic acid sequences may be utilized wherein one copy of the nucleic acid sequence encoding the peptide is optimized in one way and another copy of the nucleic acid sequence encoding the peptide is optimized in a different way or not optimized. Additionally, the nucleic acid sequences may encode the same or different therapeutic peptides.

When implanted into a human subject, or when maintained in culture in vitro, the peptide GMMO of the invention provides the at least one therapeutic peptide as a polypeptide (e.g., peptide-cleavable linker-peptide), wherein the polypeptide is cleaved to produce two or more peptide monomers by an endogenous protease in vivo. The two or more peptide monomers may be the same or different therapeutic peptides. The peptide monomers are substantially free of linker sequences after cleavage in vivo. In one embodiment the polypeptide produced by the GMMO is cleaved intracellularly in a dermal fibroblast within the GMMO to produce therapeutic peptide monomers, which may be secreted from the GMMO. In another embodiment, when the peptide GMMO is implanted in a human subject, the polypeptide produced by the GMMO may be secreted from the GMMO into the serum and the polypeptide may be cleaved in the serum to produce therapeutic peptide monomers.

In one embodiment the peptide GMMO provides the at least one therapeutic peptide as a monomer for a sustained period of at least three months as measured in vitro or in vivo. In other embodiments the peptide GMMO provides the at least one therapeutic peptide as a monomer for a sustained period of at least three, four, five, or six months as measured in vitro or in vivo.

The peptide GMMO may comprise a helper-dependent adenoviral vector (HdAd) or an adeno-associated viral vector (AAV).

In some instances, the peptide GMMO comprises a nucleic acid encoding at least one therapeutic peptide, wherein the therapeutic peptide is operably-linked to an upstream regulatory sequence. The upstream regulatory sequence may be a MAR sequence, a CAG promoter sequence, an EF1α promoter sequence or a WPRE sequence. In other embodiments, the nucleic acid encoding the therapeutic peptide further encodes a downstream regulatory sequence chosen from a MAR sequence, a CAG promoter sequence, an EF1α promoter sequence and a WPRE sequence.

In some instances, the peptide GMMO comprises a nucleic acid encoding at least one therapeutic peptide, wherein the peptide is downstream of a signaling peptide. The signaling peptide may be a proglucagon signaling peptide, an EPO signaling peptide, a tripsinogen-2 signaling peptide, or a PYY signaling peptide.

In some embodiments, the regulatory and signaling sequences are CpG-free. In other embodiments, the therapeutic peptide sequences are CpG-free.

A peptide GMMO comprising the nucleic acids of SEQ ID NO: 7 or SEQ ID NO: 5, or comprising nucleic acids having 85%, 90%, or 95% identity to SEQ ID NO: 7 or SEQ ID NO: 5 is encompassed.

A peptide GMMO comprising a viral vector comprising the nucleic acids of SEQ ID NO: 21, or comprising nucleic acids having 85%, 90%, or 95% identity to SEQ ID NO: 21 is encompassed.

A peptide GMMO comprising the nucleic acids of SEQ ID NO: 55 or SEQ ID NO: 57, or comprising nucleic acids having 85%, 90%, or 95% identity to SEQ ID NO: 55 or SEQ ID NO: 57 is encompassed.

A peptide GMMO comprising a viral vector comprising the nucleic acids of SEQ ID NO: 58 or SEQ ID NO: 59, or comprising nucleic acids having 85%, 90%, or 95% identity to SEQ ID NO: 58 or SEQ ID NO: 59 is encompassed.

A peptide GMMO comprising the nucleic acids of SEQ ID NO: 35 or SEQ ID NO: 39, or comprising nucleic acids having 85%, 90%, or 95% identity to SEQ ID NO: 35 or SEQ ID NO: 39 is encompassed.

A peptide GMMO comprising a viral vector comprising the nucleic acids of SEQ ID NO: 43 or SEQ ID NO: 47 or SEQ ID NO: 45 or SEQ ID NO: 49, or comprising nucleic acids having 85%, 90%, or 95% identity to SEQ ID NO: 43 or SEQ ID NO: 47 or SEQ ID NO: 45 or SEQ ID NO: 49 is encompassed.

The peptide GMMOs of the invention may be genetically modified dermal micro-organs.

In one embodiment, the peptide GMMO comprises at least one therapeutic peptide comprising SEQ ID NO: 1 or SEQ ID NO: 3; or SEQ ID NO: 22; or SEQ ID NO: 25 or SEQ ID NO: 31. Where the GMMO comprises a peptide comprising the amino acids of SEQ ID NO: 1, the vector or expression cassette may comprise the nucleic acids of SEQ ID NO: 2, or nucleic acids having 85%, 90%, or 95% identity to SEQ ID NO: 2. Where the GMMO comprises a peptide comprising the amino acids of SEQ ID NO: 22, the vector or expression cassette may comprise the nucleic acids of SEQ ID NO: 23 or nucleic acids having 85%, 90%, or 95% identity to SEQ ID NO: 23. Where the GMMO comprises a peptide comprising the amino acids of SEQ ID NO: 25, the vector or expression cassette may comprise the nucleic acids of SEQ ID NO: 26, or nucleic acids having 85%, 90%, or 95% identity to SEQ ID NO: 26. Where the GMMO comprises a peptide comprising the amino acids of SEQ ID NO: 31, the vector or expression cassette may comprise the nucleic acids of SEQ ID NO: 60, or nucleic acids having 85%, 90%, or 95% identity to SEQ ID NO: 60.

In other embodiments, the peptide GMMO comprises at least one therapeutic peptide encoded by nucleic acids comprising SEQ ID NO: 10 and/or 11, or nucleic acids having 85%, 90%, or 95% identity to SEQ ID NO: 10 and/or 11.

In certain embodiments, the peptide GMMO of the invention further comprises one or more cleavage sites. In certain embodiments, the peptide GMMO of the invention further comprises a furin or convertase cleavage site. The furin or convertase cleavage site may be non-native to the linker and signaling peptide sequence and may be immediately upstream and/or downstream of the linker and/or signaling peptide. In certain embodiments, the peptide GMMO of the invention further comprises an IP-1, IP-2, GS, peptidylglycine alpha-amidating monooxygenase (PAM), furin 2A, furin 2B, furin 2A2B, or phosphoenolpyruvate carboxykinase (Pck1) cleavage site. The IP-1, IP-2, GS, PAM, furin 2A, furin 2B, furin 2A2B, or Pck1 cleavage site may be non-native to the linker and signaling peptide sequence and may be immediately upstream and/or downstream of the linker and/or signaling peptide.

In certain embodiments, the peptide GMMO of the invention further comprises a propeptide (PP) linker.

In certain method embodiments of the invention, methods of treating or preventing a disease or disorder in a human subject are encompassed. A peptide GMMO of the invention is provided that is capable of providing a sustained delivery of at least one therapeutic peptide; the method comprising optionally determining the therapeutic peptide secretion levels of the at least one GMMO in vitro; implanting the at least one GMMO in the human subject at an effective dosage; and optionally measuring therapeutic peptide levels in the blood of said subject; wherein implantation of said at least one peptide GMMO increases the in vivo serum peptide levels over basal levels for at least three months.

In one method embodiment, the therapeutic peptide is GLP-2 or a GLP-2 variant that retains at least one GLP-2-like activity. Where the therapeutic peptide is GLP-2 or a GLP-2 variant, the methods to be treated and/or prevented include, but are not limited to, short bowel syndrome (SBS), Crohn's disease, ulcerative colitis, inflammatory bowel disease (IBD), osteoporosis. In another embodiment the therapeutic peptide provides adjuvant therapy during cancer chemotherapy.

In one method embodiment, the therapeutic peptide is oxyntomodulin or an oxyntomodulin variant that retains at least one oxyntomodulin activity. Where the therapeutic peptide is oxyntomodulin or an oxyntomodulin variant, the methods to be treated and/or prevented include, but are not limited to over-eating, obesity, diabetes, hypothalmic hyperphagia, binge-eating disorder, Prader-Willi Syndrome, and conditions or diseases characterized by an oxyntomodulin deficiency.

In one method embodiment, the therapeutic peptide is PYY or a PYY variant, e.g. as PYY₃₋₃₆, that retains at least one PYY activity. Where the therapeutic peptide is PYY or a PYY variant, the methods to be treated and/or prevented include, but are not limited to over-eating, obesity, diabetes, Prader-Willi Syndrome, and conditions or diseases characterized by an PYY deficiency.

In one embodiment any of the compositions described herein can be used as a medicament to treat any of the diseases and disorders described herein.

Unless otherwise specified, a “variant” protein or peptide is one that has at least one substitution, insertion, deletion, and the like.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIGS. 1A-1B show schematics of proglucagon (FIG. 1A) and approaches for generating different expression cassettes (FIG. 1A). FIG. 1A provides a schematic of proglucagon. FIG. 1B provides a schematic of an approach taken to generate five different expression cassettes of GLP-2 or a GLP-2 variant. In FIG. 1B, “GLP-2” can refer to wild type or variant GLP-2. The same approaches may also be used with other therapeutic peptides, GLP-2 being only an example.

FIG. 2 provides a representative calibration curve for a GLP-2 measuring system.

FIGS. 3A-3B show nucleofection results on human dermal fibroblast cells. FIG. 3A provides human dermal fibroblast nucleofection results from two representative nucleofection experiments with GLP-2 variant vs. GLP-2 wild type plasmids. Similar secretion levels were obtained when human dermal fibroblasts were transfected with plasmids containing GLP-2 variant or GLP-2 wild type expression cassettes. FIG. 3B provides nucleofection results on dermal fibroblast cells from additional nucleofection experiments.

FIG. 4 provides in vitro results from GMMOs secreting GLP-2 variant.

FIG. 5 provides an in vitro GLP-2 variant secretion profile. GLP-2 GMMO secretion levels are at the range of μg per day. Stable in vitro secretion was observed at the first month; however, reduction of about 95% from initial peak level was observed in a three-month time frame.

FIG. 6 provides the effect of media exchange frequency on secreted GLP-2 variant. Similar secretion levels were obtained when media was exchanged daily or every 3-4 days. These observations suggest that GMMO secreted GLP-2 variant is stable in DMEM media at 32° C. for several days.

FIG. 7 illustrates the in vitro performance of GLP-2 variant secreting GMMOs in various serum free media. GMMOs maintained in TheraPEAK™ MSCGM-CD™ Mesenchymal Stem Cell Medium (Lonza)(“MSCGM”). MSCGM media showed comparable secretion profile to those maintained in DMEM supplemented with 10% serum.

FIG. 8 shows in vitro GMMO skin-to-skin secretion variability on days 9-15. In vitro GLP-2 variant secretion average of 26 μg/day was measured from GMMOs maintained in serum-containing media.

FIGS. 9A/B/C shows the in vivo SCID mice performance of GLP-2 variant GMMOs. The in vivo GLP-2 secretion profile suggests a 75% reduction from peak level one month post implantation, followed by an additional month of stable secretion.

FIG. 10 shows the secreted levels of GLP-2 variant compared to the intracellular levels of GLP-2 variant in GLP-2 variant GMMOs. Results obtained suggest that more than 90% of the GMMO-produced GLP-2 is secreted out of the GMMOs.

FIG. 11 provides GLP-2 variant western blot analysis confirming the presence of GLP-2 in three skin samples.

FIG. 12 provides GLP-2 variant western blot analysis.

FIG. 13 provides the nucleic acid sequence for the vector HDAd-EF1a-GLP-2 variant ver B.

FIG. 14 shows the results of a test to assess GMMO GLP-2 in-vivo performance by looking at intestinal morphology. Top panels show small intestine morphology of jejunum and ileum in non-transduced samples as compared to small intestine morphology of jejunum and ileum in GMMO GLP-2 implanted mice (bottom panels).

FIG. 15 provides estimations of GLP-2 activity as evidenced by villus and crypt length, and by proliferation as assessed by morphology. Ki67 staining was used as a marker for growth by indicating cell proliferation in crypt cells.

FIGS. 16A and 16B shows in vivo effect of GMMO GLP-2. In FIG. 16A, crypt and villi length in SCID mice provided with GLP-2 at 6.25 microgram per mouse twice a day (first set of bars; positive control); GMMO expressing optimized GLP-2 at 54 microgram per mouse per day (second set of bars); Virus expressing optimized GLP-2 at 5×10¹⁰ viral particles per mouse (third set of bars), or no GLP-2 (fourth set of bars; negative control). Injection of GMMO GLP-2 or Virus expressing GLP-2 exhibits potent bioactivity—villi length is higher than the negative control. FIG. 16B shows plasma GLP-2 in ng/ml for GMMO optimized GLP-2 and Virus expressing optimized GLP-2 at 7 and 14 days.

FIGS. 17A and 17B shows crypt and villi length in SCID rats provided with GMMO GLP-2 (first set of bars), Virus expressing GLP-2 (second set of bars; control), or no GLP-2 (third set of bars; control). Rat GMMO GLP-2 demonstrates the highest bioactivity with the highest villi length. FIG. 17B shows plasma GLP-2 in ng/ml for GMMO optimized GLP-2 and Virus expressing optimized GLP-2 at days zero through 29.

FIG. 18 shows a schematic of a representative segment of intestine, detailing one way in which the segment can be processed for analysis.

FIG. 19 provides GLP-2 variant western blot analysis showing the presence of GLP-2 from GMMO collection media in three skin samples (Skin 1-3). The lanes of the immunoblot are as follows: (1) standard of dimer and monomer GLP-2; (2) Marker (Dual xtra, Bio-Rad); (3) Skin 1 sample (untreated with urea; (4) Skin 1 sample; (5) empty lane; (6) Skin 2 sample; (7) empty lane; (8) Skin 3 sample; (9) Skin 3 sample; (10) spent media collected from erythropoietin (EPO) secreting GMMO (Negative control).

FIGS. 20A-B show the in vitro activity of GLP-2 standard and GLP-2 produced by GMMOs expressing either GLP-2 or GLP-2 variant. GLP-2 was tested for ability to stimulate cAMP production in HEK293 cells transfected with the GLP-2 receptor. FIG. 20A shows dose-response of GLP-2 dimer standard. FIG. 20B shows the titration of collection media from GMMO expressing GLP-2 or GLP-2 variant.

FIGS. 21A-B provide the effect of production media volume on detected oxyntomodulin or EPO levels in GMMO spent media. FIG. 21A shows that GMMOs expressing oxyntomodulin maintained in 3 ml media show higher spent media oxyntomodulin levels than GMMOs maintained in 1 ml media. FIG. 21B shows that media volume did not influence secretion levels for GMMOs expressing EPO. The x-axis shows days from transduction, and the y-axis shows EPO secretion, IU/GMMO/day.

FIG. 22 shows in vitro GMMO oxyntomodulin skin-to-skin secretion variability on days 14-16 after transduction. In vitro oxyntomodulin GMMO secretion average of 40.5 μg/day was measured from GMMOs maintained in 3 ml serum-containing media.

FIG. 23 shows the effect of media exchange frequency on measured oxyntomodulin concentration for three different skin samples. When media was exchanged daily (noted by “1”) instead of every 3 days (noted by “3”), higher oxyntomodulin concentrations were measured in the GMMOs spent media.

FIGS. 24A-D show the effects of time since media exchange on secretion of oxyntomodulin or GLP-2. FIGS. 24A-24B show hourly sampling results of oxyntomodulin from the GMMO-oxyntomodulin spent media. FIGS. 24C-24D show hourly sampling results of GLP-2 from GMMO-GLP-2 Variant spent media.

FIGS. 25A-B shows the results of testing to determine whether various DPP-IV inhibitors protect oxyntomodulin in the GMMO system. As shown in FIG. 25A, no positive effects on oxyntomodulin levels were measured after addition of different DPP-IV inhibitors to the GMMO production media at the concentrations tested. FIG. 25B shows the results of testing to determine whether the DPP-IV inhibitor, Diprotin A, protects oxyntomodulinin in the GMMO system. No positive effect on oxyntomodulin secretion was observed after addition of the DPP-IV inhibitor at different concentrations to the production media.

FIGS. 26A-26B show the effect of a protease inhibitor on GLP-2 secretion from GMMO-GLP-2 Variant and oxyntomodulin secretion from GMMO-oxyntomodulin. GMMOs maintained with protease inhibitor showed higher oxyntomodulin concentrations in the spent media (FIG. 26B), while the same protease inhibitor did not have an effect on GLP-2 concentration in the spent media (FIG. 26A).

FIG. 27 illustrates the in vitro performance of oxyntomodulin secreting GMMOs in various serum free media. GMMOs maintained in MSCGM media showed higher secretion levels than those maintained in DMEM supplemented with 10% serum.

FIG. 28 shows the effect of calcium in production media on oxyntomodulin concentration measured in GMMO spent media. The presence of 20 mM of CaCl₂ in GMMO production media increased measured oxyntomodulin levels in the spent media by 2-fold.

FIG. 29 shows the results of an in vivo experiment in SCID mice testing the performance of oxyntomodulin GMMOs. The results from this experiment suggest that mice provided with oxyntomodulin GMMO processed with Active medium and injected with depomedrol post implantation secrete oxyntomodulin above baseline at day 7 (p-value <0.05 on day 7—Active with depomedrol versus MO with depomedrol.).

FIG. 30 shows the effect of implanted oxyntomodulin GMMOs on SCID mice weight. No trend in SCID mice weight was observed in oxyntomodulin secreting GMMOs (1 ng/mouse) post-implantation.

FIG. 31 shows the results of a second in vivo experiment in SCID mice testing the performance of oxyntomodulin GMMOs (215 ng/mouse). An increase of about 500 pg/ml in serum levels of oxyntomodulin above baseline level was detected in mice serum 7 days post-implantation.

FIG. 32 shows the effect of implanted oxyntomodulin GMMOs on SCID mice weight. No trend in SCID mice weight was observed the first 11 days post-implantation with oxyntomodulin secreting GMMOs (215 ng/mouse) implantation.

FIG. 33 shows the results of an in vivo experiment in nude rats testing the performance of oxyntomodulin GMMOs (80 ng/rat). An increase of about 200 pg/ml in serum oxyntomodulin level above baseline was detected in the serum of rats 7 days post-implantation.

FIG. 34 shows the effect of implanted oxyntomodulin GMMOs on nude rat weight. No trend in nude rat weight was observed the first 16 days post-implantation of oxyntomodulin secreting GMMOs (80 ng/day).

FIG. 35 provides a schematic of approaches taken to generate different expression cassettes. Approach A corresponds to the construct used for expressing of oxyntomodulin by GMMOs in previous Figures. Approach B is similar to the approach shown with GLP-2; IP-2: convertase 1/3 and 2. Approaches C and D use a furin cleavage site. Approaches E and F provide co-expression with protein YY (PYY). Approach G uses a PYY signaling peptide.

FIG. 36 provides human dermal fibroblast nucleofection results with different oxyntomodulin plasmid approaches. When compared to the plasmid encoding the previous selected oxyntomodulin concept (i.e., Approach A), results with the plasmid encoding the oxyntomodulin concept of Approach B (Glucagon signaling peptide-oxyntomodulin-IP2 linker-oxyntomodulin) showed an 8-10 fold increase in oxyntomodulin secretion levels. The signaling peptide, linker, and number of target protein cassettes are of Approach B correspond to the Approach B that was selected as a preferred GMMO-GLP-2 Variant plasmid.

FIG. 37 provides a comparison of two oxyntomodulin constructs, oxyntomodulin-ver B and oxyntomodulin ver-A (as described in FIG. 35). Oxyntomodulin-ver B demonstrates higher in vitro OXM secretion levels compared to oxyntomodulin-ver A.

FIGS. 38A-B show activity of diet induced obesity (“DIO”) mice implanted with either GMMOs transduced with oxyntomodulin-ver B or nontransduced MOs. FIG. 38A shows that DIO mice exhibit weight reduction over 63 days when implanted with GMMO-oxyntomodulin-ver B compared to those implanted with nontransduced MOs. FIG. 38B shows that plasma levels of oxyntomodulin are higher through Day 28 post-implantation in DIO mice implanted with GMMOs transduced with oxyntomodulin-ver B versus mice implanted with nontransduced MOs.

FIG. 39 provides a representative calibration curve for a PYY measurement system.

FIGS. 40A-40B provide a schematic of approaches taken to generate different expression cassettes for PYY expression.

FIG. 41 shows the effect of the PYY construct on secreted PYY levels in nucleofection studies.

FIG. 42 shows the effect of PYY construct on secreted and intracellular PYY levels in nucleofection studies.

FIG. 43 shows a comparison of PYY vectors (1.5*10̂10 vp/ml), transduction of human skin.

FIG. 44 provides a western blot analysis of GMMO secreted monomer and dimer of PYY (PYY signal peptide-PYY-PCSK1-propeptide-PCSK1-PYY) in different media. Lanes: (1) GMMO collection media (Negative control). (2) PYY std. (3) Empty lane. (4) HA369 sample 9 in 0.5% serum (5) HA369 sample 9 in 0.5% serum (6) HA369 sample 6 in 2% serum (7) HA369 sample 8 in 2% serum (8) HA369 sample 13 FGM-2 medium (9) HA369 sample 16 in FGM-2 medium. (10) Empty lane (11) HA369 sample 1 in HyClone 10% RBS medium (12) HA369 sample 4 in HyClone 10% RBS medium.

FIG. 45 shows western blot results confirming the presence of oxyntomodulin in GMMOs generated with HDdelta28E4-MAR-EF1a containing the version B cassette of oxyntomodulin.

FIG. 46 shows a representative calibration curve for the in vitro ELISA conducted throughout to measure oxyntomodulin levels.

FIG. 47 shows a representative calibration curve for the in vivo ELISA conducted throughout to measure oxyntomodulin levels.

### DESCRIPTION OF THE EMBODIMENTS

### I. Micro-Organs Expressing Peptides

In some embodiments, the genetically-modified micro-organ (GMMO) of the invention secretes at least one therapeutic peptide. The expression constructs are designed to overcome difficulties in the filed of expressing physiologically relevant levels of peptide due to short half-lives of peptides. The GMMOs of the invention produce therapeutically acceptable levels of peptide post-implantation, and the GMMOs are capable of maintaining therapeutic levels of peptide in vivo for at least 3 months. The peaks and troughs associated with subcutaneous injection of peptides are negated with this invention, as the peptide is continuously and stably expressed by the GMMOs for extended lengths of time.

In one embodiment, the therapeutic peptide may comprise GLP-2. GLP-2 activities include stimulating intestinal growth and up-regulating villus height in the small intestine, concomitant with increasing crypt cell proliferation and decreased enterocyte apoptosis.

The gastrointestinal tract, from the stomach to the colon is a target for GLP-2 action. GLP-2 plays a key role in nutrient homeostasis, enhancing nutrient assimilation through enhanced gastrointestinal function, as well as increasing nutrient disposal. It stimulates intestinal glucose transport and decreases mucosal permeability.

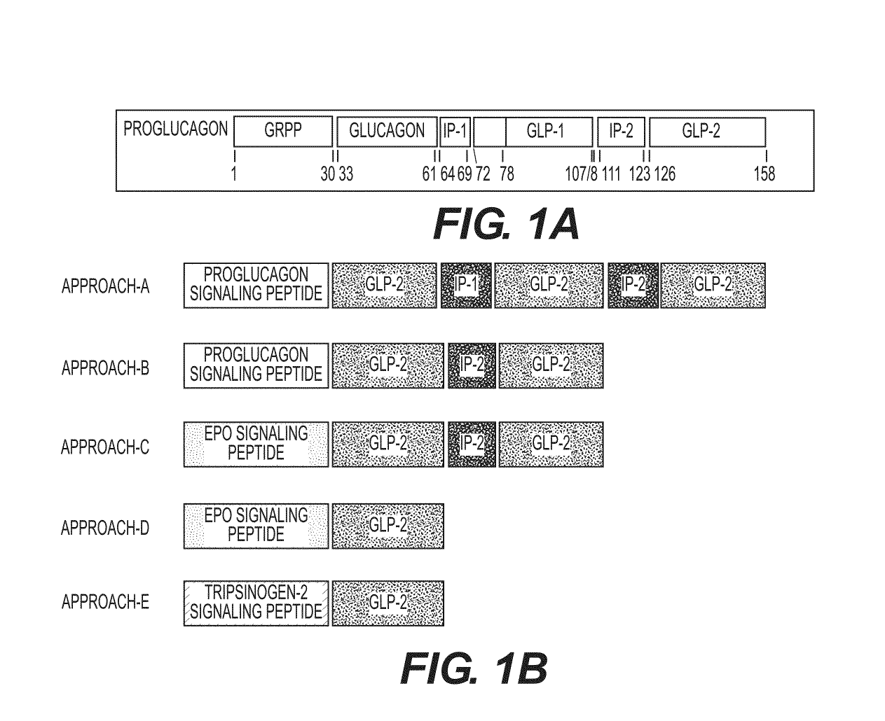

GLP-2 is generated in vivo from the post-translational processing of preproglucagon, a precursor protein that generates several different peptide hormones upon enzymatic cleavage, including glucagon, GLP-1, GLP-2, glicentin, and oxyntomodulin. The open reading frame of preproglucagon includes a 20-amino acid signal peptide or leader sequence, followed by a 158 amino acid proglucagon polypeptide. The GLP-2 sequence is located at amino acids 126 to 158 of proglucagon and is 33 amino acids in length. FIG. 1A.

A plurality of therapeutic peptides may be used herein. One therapeutic peptide is GLP-2, which increases intestinal absorption, stimulates intestinal growth, and reduces bone breakdown. As used herein, the term “GLP-2” or “wild type GLP-2” denotes a human native GLP-2 peptide (e.g. SEQ ID NO: 1). In some embodiments, the therapeutic peptide comprises or consists of the amino acid sequence of SEQ ID NO: 1.

In some embodiments, the therapeutic peptide is a GLP-2 variant. The term “GLP-2 variant” denotes a peptide, which has at least one substitution, insertion, and/or deletion compared to wild type GLP-2 but retains the biological activity of wild type GLP-2. A variant of GLP-2 with a point mutation from Ala to Gly at the second amino acid in the sequence, teduglutide (Gattex®), is currently approved for treatment of small bowel syndrome in patients dependent upon parenteral nutritional support, reducing the need for total parenteral nutrition (TPN). Other variants of GLP-2 for therapeutic use are described in the literature, for example, in United States published patent applications US2009/117104, US2008/249016, US2006/105948, and US2005/282749.

In some embodiments, the GLP-2 variant comprises SEQ ID NO: 1 and has addition mutations at one or more amino acids as compared to the wild type sequence. In some embodiments, the GLP-2 variant comprises or consists of SEQ ID NO: 3. In some embodiments, the GLP-2 variant has one or two amino acid substitutions compared to that of SEQ ID NO: 1 or 3. In some embodiments, the GLP-2 variant has one or two amino acid insertions compared to that of SEQ ID NO: 1 or 3 internally within the sequence. In some embodiments, the GLP-2 variant is 34, 35, or 36 amino acids long. In some embodiments, the GLP-2 variant has one or two amino acid deletions compared to that of SEQ ID NO: 1 or 3, either internally within the sequence or from the N- or C-terminal. In some embodiments, the GLP-2 variant is at least 25, or from 25 to 32 amino acids long, or is at least 30 or from 30-32 amino acids long. In one embodiment, the GLP-2 variant is 33 amino acids long.

The GLP-2 amino acid sequences may be encoded by nucleic acid sequences specifically described herein or they may be encoded by any native or optimized nucleic acid sequences encoding the GLP-2 amino acid sequences due to the degeneracy of the nucleic acid code.

A GLP-2 variant may retain the functional activity of GLP-2. By this, it is meant the ability to increase intestinal absorption, stimulate intestinal growth, and reduce bone breakdown. For instance, in vivo activity may be tested after implantation of a GMMO in a human or animal by evaluating the length of the intestinal villus and cell proliferation. GLP-2 activity may also be evaluated using an in vitro activity assay for GLP-2 such by using the Fluorescent Glucagon-like Peptide 2 Receptor (GLP2R) Internalization Assay Cell Line by Life Sciences B-Bridge (Cupertino, Calif.). A GLP-2 variant is within the scope of the present application if it maintains 100% of the activity of wild type GLP-2, exceeds the activity of wild type GLP-2, or maintains at least 99%, 98%, 97%, 96%, 95%, 90%, 85%, 80%, 75%, 70%, 60%, or 50% of the activity of wild type GLP-2 under any of the assays described herein.

In one embodiment, the therapeutic peptide may comprise oxyntomodulin. Oxyntomodulin activities include acting as an GLP-1 and glucagon agonist and inhibiting gastric acid secretion. Oxyntomodulin also functions to delay gastric emptying, leading to reduced feelings of hunger and reduced food intake. Administration of oxyntomodulin has been shown to result in reduced hunger and food intake in rodents and humans. See Int. J. Ober (London), 2006; 30 (12): 1729-36. It also has been linked with weight loss and increased activity and energy expenditure. See J. Clin. Endocrinol. Metab., 2003, 88 (10); 4696-701. Thus, administration of a therapeutically effective amount of oxyntomodulin may be useful for treating obesity, diabetes, hypothalmic hyperphagia, binge-eating disorder, Prader-Willi syndrome, overeating, and other gastrointestinal conditions and diseases.

Oxyntomodulin, like GLP-2, is generated from the precursor protein preproglucagon. The oxyntomodulin sequence is located at amino acids 33-69 of the proglucagon polypeptide and is 37 amino acids in length. It includes the 29 amino acids of glucagon plus a carboxyterminal extension IP-1. See FIG. 1A.

As used herein, the term “oxyntomodulin” denotes a human native oxyntomodulin peptide (e.g. SEQ ID NO: 22). In some embodiments, the therapeutic peptide comprises or consists of the amino acid sequence of SEQ ID NO: 22. In some embodiments, the therapeutic peptide comprises or consists of an oxyntomodulin variant. The term “oxyntomodulin variant” denotes a peptide, which has at least one substitution, insertion, and/or deletion compared to wild type oxyntomodulin but retains biological activity of wild type oxyntomodulin.

In some embodiments, the oxyntomodulin variant comprises SEQ ID NO: 22 and has addition mutations at one or more amino acids as compared to the wild type sequence. In some embodiments, the oxyntomodulin variant has one or two amino acid substitutions compared to that of SEQ ID NO: 22. In some embodiments, the oxyntomodulin variant has one or two amino acid insertions compared to that of SEQ ID NO: 22 internally within the sequence. In some embodiments, the oxyntomodulin variant is 38, 39, 40, or 41 amino acids long. In some embodiments, the oxyntomodulin variant has one or two amino acid deletions compared to that of SEQ ID NO: 22, either internally within the sequence or from the N- or C-terminal. In some embodiments, the oxyntomodulin variant is at least 30, or from 30 to 36 amino acids long, or is at least 33 or from 33-36 amino acids long. In one embodiment, the oxyntomodulin variant is 37 amino acids long.

The oxyntomodulin amino acid sequences may be encoded by nucleic acid sequences specifically described herein or they may be encoded by any native or optimized nucleic acid sequences encoding the oxyntomodulin amino acid sequences due to the degeneracy of the nucleic acid code.

An oxyntomodulin variant may retain the functional activity of oxyntomodulin. By this, it is meant the ability to agonize GLP-1 or glucagon, inhibit gastric acid secretion, delay gastric emptying. For instance, in vivo activity may be tested after implantation of a GMMO in a human or animal by measuring the gastric acid secretion, food intake, energy levels, or overall body weight. Oxyntomodulin activity may also be evaluated using an in vitro activity assay for oxyntomodulin. Oxyntomodulin variants are within the scope of the present application if they maintain 100% of the activity of wild type oxyntomodulin, exceed the activity of wild type oxyntomodulin, or maintain at least 99%, 98%, 97%, 96%, 95%, 90%, 85%, 80%, 75%, 70%, 60%, or 50% of the activity of wild type oxyntomodulin under any of the assays described herein.

In one embodiment oxyntomodulin activity can be assessed in vitro according to known methods. For example, the in vitro potency of oxyntomodulin can be determined in Chinese hamster ovary cells stably expressing the glucagon-like peptide-1 receptor (GLP1R) or glucagon receptor (GCGR) using standard time-resolved fluorescence energy transfer assays (see Kosinski J R, et al., Obesity (Silver Spring) 20(8):1566-1571 (2012)). As also demonstrated in Kosinski 2012, the ex vivo potency of oxyntomodulin can be determined using perfused mouse livers and measuring glycogen breakdown. Oxyntomodulin variants that retain activity as per these methods are encompassed.

In one embodiment, the therapeutic peptide may comprise peptide YY (PYY). PYY activities include inhibiting gastric, pancreatic and intestinal secretions and stimulating absorption and growth in intestinal epithelium. Administration of PYY has been shown to result in reduced appetite and food intake, leading to it consideration as a weight loss therapy. Thus, administration of a therapeutically effective amount of oxyntomodulin may be useful for treating obesity, diabetes, hypothalmic hyperphagia, binge-eating disorder, Prader-Willi syndrome, overeating, and other gastrointestinal conditions and diseases.

PYY in its main molecular form is 36 amino acids in length (PYY₁₋₃₆), but a 34-amino acid form, PYY₃₋₃₆, is also biologically active. PYY is generated from a precursor peptide, which undergoes posttranslational processing to generate PYY₁₋₃₆ and PYY₃₋₃₆. The enzyme dipeptidyl peptidase-IV (DPP-IV) removes the amino terminal dipeptide of PYY₁₋₃₆ to generate PYY₃₋₃₆.

As used herein, the term “PYY” denotes a human native PYY peptide, e.g. PYY₁₋₃₆ or PYY₃₋₃₆. In some embodiments, the therapeutic peptide comprises or consists of the amino acid sequence of SEQ ID NO: 25 or SEQ ID NO: 31. In some embodiments, the therapeutic peptide comprises or consists of a PYY variant. The term “PYY variant” denotes a peptide, which has at least one substitution, insertion, and/or deletion compared to wild type PYY but retains biological activity of wild type PYY.

In some embodiments, the PYY variant comprises SEQ ID NO: 25 and has addition mutations at one or more amino acids as compared to the wild type sequence. In some embodiments, the PYY variant has one or two amino acid substitutions compared to that of SEQ ID NO: 25. In some embodiments, the PYY variant has one or two amino acid insertions compared to that of SEQ ID NO: 25 internally within the sequence. In some embodiments, the PYY variant is 37, 38, 39, or 40 amino acids long. In some embodiments, the PYY variant has one or two amino acid deletions compared to that of SEQ ID NO: 25, either internally within the sequence or from the N- or C-terminal. In some embodiments, the PYY variant is at least 28, or from 28 to 35 amino acids long, or is at least 32 or from 32-35 amino acids long. In one embodiment, the PYY variant is 36 amino acids long.

In some embodiments, the PYY variant comprises SEQ ID NO: 31 and has addition mutations at one or more amino acids as compared to the wild type sequence. In some embodiments, the PYY variant has one or two amino acid substitutions compared to that of SEQ ID NO: 31. In some embodiments, the PYY variant has one or two amino acid insertions compared to that of SEQ ID NO: 31 internally within the sequence. In some embodiments, the PYY variant is 35, 36, 37, or 38 amino acids long. In some embodiments, the PYY variant has one or two amino acid deletions compared to that of SEQ ID NO: 31, either internally within the sequence or from the N- or C-terminal. In some embodiments, the PYY variant is at least 26, or from 26 to 33 amino acids long, or is at least 30 or from 30-33 amino acids long. In one embodiment, the PYY variant is 34 amino acids long.

The PYY amino acid sequences may be encoded by nucleic acid sequences specifically described herein or they may be encoded by any native or optimized nucleic acid sequences encoding the PYY amino acid sequences due to the degeneracy of the nucleic acid code.

A PYY variant may retain the functional activity of PYY. By this, it is meant the ability to inhibit gastrointestinal and pancreatic secretions and/or stimulate absorption or growth in intestinal tissue. For instance, in vivo activity may be tested after implantation of a GMMO in a human or animal by measuring the gastric acid secretion, food intake, energy levels, or overall body weight. PYY activity may also be evaluated using an in vitro activity assay for PYY. A PYY variant is within the scope of the present application if it maintains 100% of the activity of wild type PYY, exceeds the activity of wild type PYY, or maintains at least 99%, 98%, 97%, 96%, 95%, 90%, 85%, 80%, 75%, 70%, 60%, or 50% of the activity of wild type PYY under any of the assays described herein.

In one embodiment PYY activity can be assessed in vitro according to known methods. For example, the in vitro potency of PYY can be determined in HEK293 cells expressing human NPY receptors (Y receptors) and measuring changes in cAMP levels. It has been shown that a functional cAMP biosensor assay of this type can be run with HEK293 cells expressing the human Y₁, Y₂, Y₄, or Y₅ receptor subtypes (see Albertson L, et al., ACS Med. Chem. Lett. 4:1228-1232 (2013)). PYY variants that retain activity as per these methods are encompassed.

A. Platform for Peptide Expression

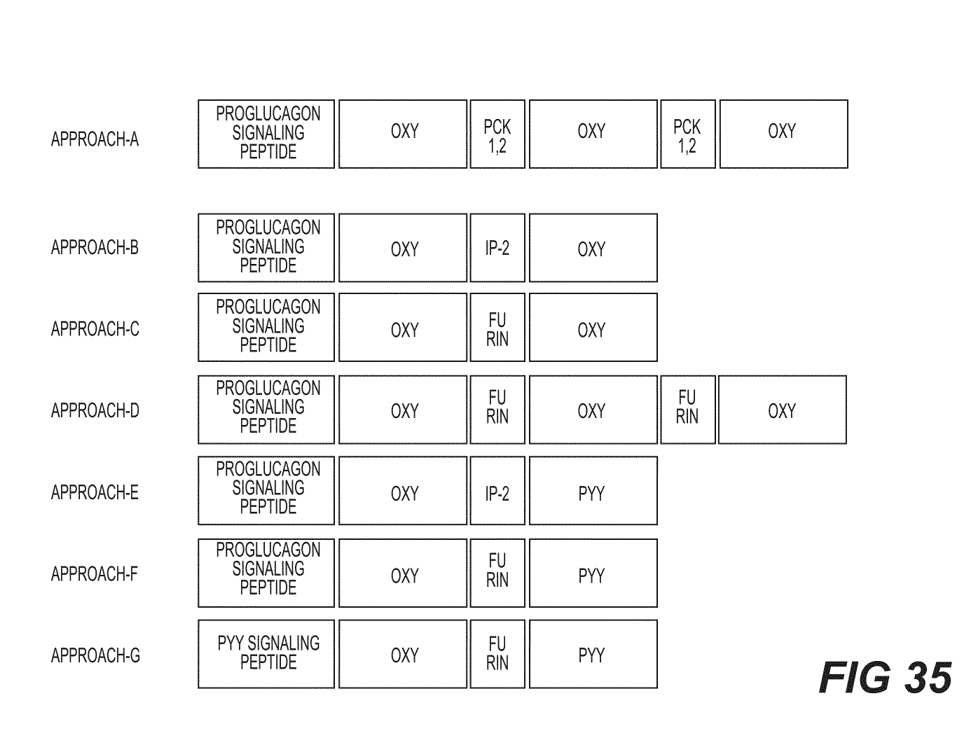

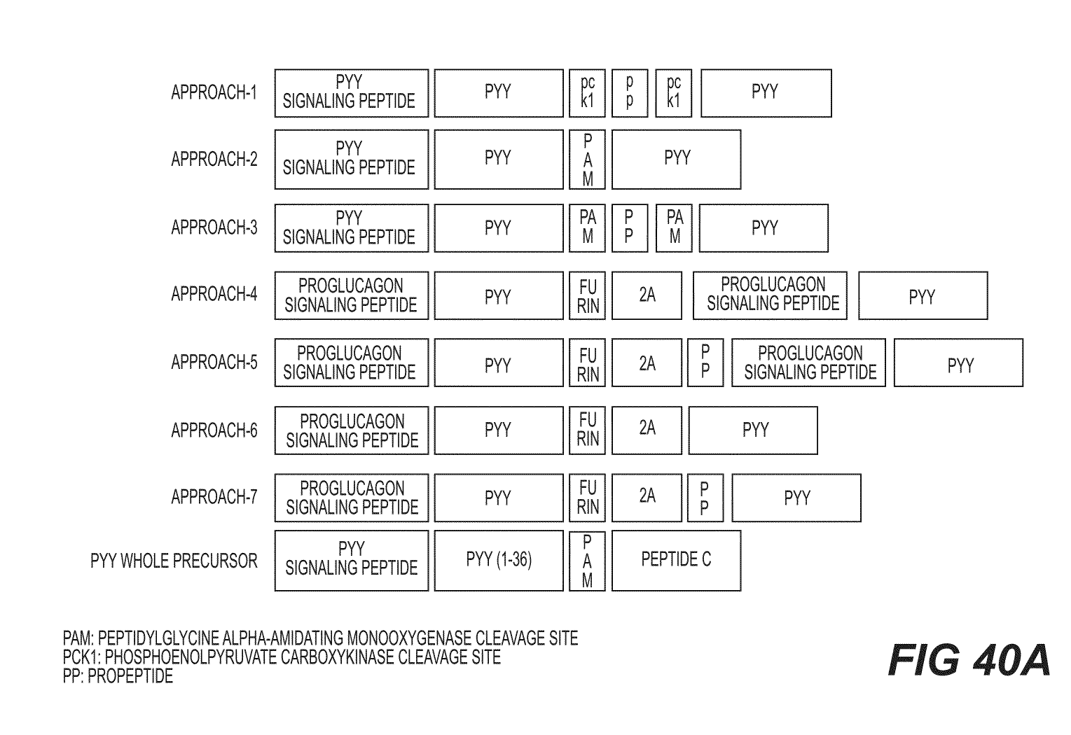

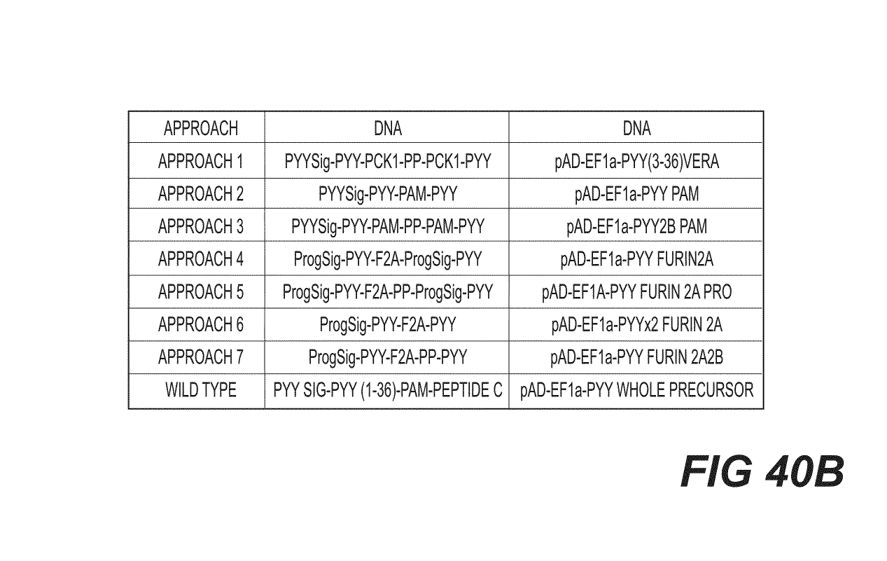

In one embodiment, the therapeutic peptide has about 60, 65, 50, 45, 40, 39, 38, 37, 36, 35, 34, 33, 32, 31, 30, 25, 20, 15, 14, 13, 12, 11, 10, 9, 8, 7 or fewer amino acids. In one embodiment, the signal peptide has about 25, 20, 16, 15 or fewer amino acids. In one embodiment, the linker has about 30, 25, 20, 19, 18, 17, 16, 15, 14, 13, 12, 11, 10 or fewer amino acids. In one embodiment, the amino acid sequence expressed from the vector (for example if the sequence has a signal peptide, two therapeutic peptides, and a linker, the combined length of all of the components) has about 250, 225, 200, 175, 150, 125, 110, 105, 101, 100, 95, 90 or fewer amino acids. Within the vectors herein, DNA sequence cassettes for expression of the therapeutic peptides may have a number of different arrangements. GLP-2 and GLP-2 variants, oxyntomodulin and oxyntomodulin variants, and PYY and PYY variants are only exemplary therapeutic peptides and other therapeutic peptides may be employed according to the same approaches and with the same additional elements. Generally, the cassettes may include one or more regulatory elements such as promoters to control transcription of the gene and coding sequence(s) for the therapeutic peptide operably linked to the one or more regulatory elements. The coding sequences may include a signal peptide (or leader sequence or leader peptide) in order to direct the therapeutic peptide for secretion from the cell. And, because therapeutic peptides are often derived from larger precursor peptides (polypeptides), the coding sequences for the therapeutic peptides may include portions of the larger precursor peptide or other cleavable linker regions that may be cleaved from the therapeutic peptides post-translationally by native enzymes. For example, GLP-2 and oxyntomodulin are derived from proglucagon so the coding sequences for GLP-2 or GLP-2 variant or oxyntomodulin may include portions of the proglucagon coding sequence or other cleavable linker regions that may be cleaved from the GLP-2 or GLP-2 variant or oxyntomodulin peptides post-translationally by native enzymes. See FIG. 1B and FIG. 35. Likewise, PYY is generated from a precursor peptide, which undergoes posttranslational processing to generate PYY₁₋₃₆ and PYY₃₋₃₆ so the coding sequence for PYY may include portions of the precursor peptide or other cleavable linker regions that may be cleaved from the PYY peptide postranslationally by native enzymes (e.g. DPP-IV). See FIG. 40A. In this way it is possible, for example, to include more than one therapeutic peptide coding sequence in the cassette, for example, to further enhance expression. The same approach, however, may also be used for other therapeutic peptides.

In some embodiments, a therapeutic peptide, such as a GLP-2 or GLP-2 variant coding sequence, is placed downstream of a signal peptide sequence. (FIG. 1B, Approaches D and E.) In some embodiments, two therapeutic peptides may be placed downstream of a signal peptide, and separated by at least one linker coding sequence, such as the portion of the native proglucagon coding sequence that encodes the IP-1 peptide (amino acids 64-69 of proglucagon), the IP-2 peptide (amino acids 111-123 of proglucagon), a furin cleavage site, a Pck1, PAM, or furin 2A or furin 2A2B cleavage site. See FIG. 1B, Approaches B and C; FIG. 35, Approaches B and C; FIG. 40A, Approaches 2 and 6. Thus, in some embodiments, with GLP-2 as an example, the cassette may be arranged as follows: signal peptide-GLP-2/GLP-2 variant-linker (e.g. IP-1)-GLP-2/GLP-2 variant. Or, using oxyntomodulin as an example, in some embodiments, the cassette may be arranged as follows: signal peptide-oxyntomodulin-linker (e.g. IP-2)-oxyntomodulin. In some embodiments, two therapeutic peptides may be placed downstream of a signal peptide and separated by two linker coding sequences, and a propeptide. See FIG. 40A, Approaches 1 and 3 and 7. Thus, in some embodiments, with PYY as an example, the cassette may be arranged as follows: signal peptide-PYY-linker (e.g. Pck1 or PAM)-propeptide-linker (e.g. Pck1 or PAM)-PYY). In some embodiments, three or more GLP-2 or GLP-2 variant coding sequences may be placed sequentially as follows: signal peptide-GLP-2/GLP-2 variant/oxyntomodulin-linker-GLP-2/GLP-2 variant/oxyntomodulin-linker-GLP-2/GLP-2 variant/oxyntomodulin. FIG. 1B, Approach A; FIG. 35, Approaches A and D. Thus, the DNA coding sequence for the therapeutic peptide may comprise at least one, at least two, or at least three therapeutic peptide coding sequences (or the same number if using another therapeutic peptide). In such embodiments, the therapeutic peptide encoded by each coding sequence may be the same or different. See FIG. 35, Approaches E, F, and G. In some embodiments, two therapeutic peptides may be placed downstream of a signal peptide, separated by one or more linker or propeptide sequences, and further separated by an additional signal peptide. See FIG. 40A, Approaches 4 and 5.

B. Linker Sequences

In certain embodiments, such as when more than one therapeutic peptide coding sequence is used, a cleavable linker may be employed. In certain instances, the cleavable linker is a synthetic sequence comprising a cleavage site. In some instances, the cleavable linker is a sequence that natively comprises a cleavage site and/or a sequence that is mutated from its native state to add one or more cleavage sites. In one embodiment, there is a single cleavage site in the linker. In one embodiment, there are two cleavage sites in the linker. In some embodiments, the cleavage site may be at the N-terminus of the linker. In some embodiments, the cleavage site may be at the C-terminus of the linker. In some embodiments, there are cleavage sites at both the N-terminus and C-terminus of the linker. By N- or C-terminus, it is meant that the linker is either exactly at the terminus or within 1, 2, or 3 amino acids of the terminus.

In some embodiments the linker may comprise SEQ ID NO: 13 (IP-2 linker), SEQ ID NO: 14 (IP-2 linker with additional RH cleavage site) or SEQ ID NO: 15 (IP-1 linker). Alternatively, the linker may be a glycine-serine linker comprising repeated glycine and serine amino acids. Glycine-serine linkers may, for example, have the following repeating sequences: GS, GGGS (SEQ ID No: 16) or GSGGGS (SEQ ID NO: 17). These may be modified by adding a cleavage site at one or both termini.

In some embodiment, the cleavage site is a convertase cleavage site. In some embodiments, the convertase cleavage site is an RR or RH. In some embodiments, the linker may comprise a furin cleavage site (SEQ ID NO: 24).

In some embodiments, the linker may comprise phosphoenolpyruvate carboxykinase (Pck1) (SEQ ID NO: 14), propeptide (PP) (SEQ ID NO: 32), peptidylglycine alpha-amidating monooxygenase (PAM) (abbreviated sequence GKR), or furin 2A (SEQ ID NO: 24).

In some embodiments, the linker may be from 2 to 20 amino acids long, such as 5-15 amino acids long, 5-10 amino acids long, 10-20 amino acids long, or 2, 3, 4, 5, 6, 7, 8, 9. 10, 11, 12, 13, 14, 15, 16, 17, 18, 19, or 20 amino acids long.

C. Signal Peptide Sequences

In certain embodiments, the vector and/or expression cassette may comprise a nucleic acid sequence encoding a signal peptide. Signal peptides are also called leader sequences or leader peptides in the art. In these embodiments, the signal peptide may facilitate secretion of the therapeutic peptide from the cell in which it is expressed. In some embodiments, the signal peptide comprises or consists of the preproglucagon signal peptide (SEQ ID NO: 9). In some embodiments, a heterologous signal peptide is used, such as the signal peptide from human erythropoietin, human trypsin 1 or trypsin 2 or human erythropoietin (SEQ ID NOs: 18-20). In some embodiments, the signal peptide comprises or consists of the PYY signaling peptide (SEQ ID NO: 29). In some embodiments, the vector and/or expression cassette may comprise a nucleic acid sequence encoding a signal peptide at the N-terminus of the sequence and another (same or different) signal peptide downstream of the first signal peptide. In some embodiments, a second signal peptide is at the N-terminus of the sequence of a second therapeutic peptide.

The signal peptide amino acid sequences may be encoded by nucleic acid sequences specifically described herein or they may be encoded by any native or optimized nucleic acid sequences encoding the signal peptide amino acid sequences due to the degeneracy of the nucleic acid code.

D. Regulatory Elements

In some embodiments, the vector and/or expression cassette of and for use in the methods herein comprises a nucleic acid sequence operably linked to one or more regulatory sequences.

Nucleotide sequences which regulate expression of a gene product (e.g., promoter, stabilizing sequences and enhancer sequences) are selected based upon the type of cell in which the gene product is to be expressed and the desired level of expression of the gene product. For example, a promoter known to confer cell-type specific expression of a gene linked to the promoter can be used. Alternatively, a regulatory element which can direct constitutive expression of a gene in a variety of different cell types, such as a viral regulatory element, can be used. Examples of viral promoters commonly used to drive gene expression include those derived from polyoma virus, Adenovirus 2, cytomegalovirus (CMV) and Simian Virus 40, and retroviral LTRs. Alternatively, a regulatory element which provides inducible expression of a gene linked thereto can be used. The use of an inducible regulatory element (e.g., an inducible promoter) allows for modulation of the production of the gene product in the cell. Examples of potentially useful inducible regulatory systems for use in eukaryotic cells include hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al (1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89:1014-10153). Additional tissue-specific or inducible regulatory systems which may be developed can also be used.

In one embodiment, the term “promoter” refers to a DNA sequence, which, in one embodiment, is operably linked upstream of the coding sequence and is important for basal and/or regulated transcription of a gene. In one embodiment, a promoter is operatively linked to a gene of interest. In another embodiment, the promoter is a mutant of the endogenous promoter, which is normally associated with expression of the gene of interest, under the appropriate conditions.

As used herein, the term “operably linked” refers in one embodiment to a nucleic acid sequence, e.g., a regulatory element or a gene encoding a therapeutic peptide, placed in a functional relationship with another nucleotide sequence, e.g., a regulatory element or a gene encoding a therapeutic peptide. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous.

Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a peptide.

In one embodiment, a promoter of the compositions and for use in the methods is a regulatable promoter. In another embodiment, a regulatable promoter refers to a promoter whereby expression of a gene downstream occurs as a function of the occurrence or provision of specific conditions which stimulate expression from the particular promoter. In some embodiments, such conditions result in directly turning on expression, or in other embodiments, remove impediments to expression. In some embodiments, such conditions result in turning off, or reducing expression.

In one embodiment, such conditions may comprise specific temperatures, nutrients, absence of nutrients, presence of metals, or other stimuli or environmental factors as will be known to one skilled in the art. In one embodiment, a regulatable promoter may be regulated by galactose (e.g. UDP-galactose epimerase (GAL10), galactokinase (GAL1)) or glucose (e.g. alcohol dehydrogenase II (ADH2)), or phosphate (e.g. acid phosphatase (PHO5)). In another embodiment, a regulatable promoter may be activated by heat shock (heat shock promoter) or chemicals such as IPTG or Tetracycline, or others, as will be known to one skilled in the art. It is to be understood that any regulatable promoter and conditions for such regulation is encompassed by the vectors, nucleic acids and methods, and represents an embodiment thereof.

In one embodiment, a regulatory sequence may comprise a constitutive promoter. Known constitutive promoters include SV40, CMV, UBC, EFlalpha, PGK and CAG. Promoters are known to vary considerably from one another in their strength dependent on cell type transduced and growth conditions. Studies indicate that promoter activities might be restricted to specific cell lineages, suggesting the need to carefully select and test promoters for constitutive gene expression.

In one embodiment, a regulatory sequence may comprise a CMV promoter, while in another embodiment; the regulatory sequence may comprise a CAG promoter. In one embodiment, a CAG promoter is a composite promoter that combines the human cytomegalovirus immediate-early enhancer and a modified chicken beta-actin promoter and first intron. In one embodiment, a CAG promoter comprises any CAG promoter known in the art.

In one embodiment, a regulatory sequence comprises an EF1α promoter. The EF1α gene has a housekeeping function in all cells and is expressed to high levels. Due to its indispensable housekeeping function in all cells, EF1α promoter expression is relatively insulated from changes in cell physiology and is cell type independent. In one embodiment, an EF1α promoter comprises any EF1α promoter known in the art.

In one embodiment, a regulatory sequence may comprise a simian virus (SV)-40 polyadenylation sequence, which in one embodiment, is the mechanism by which most messenger RNA molecules are terminated at their 3′ ends in eukaryotes. In one embodiment, the polyadenosine (poly-A) tail protects the mRNA molecule from exonucleases and is important for transcription termination, for export of the mRNA from the nucleus, and for translation. In another embodiment, a formulation may comprise one or more regulatory sequences.

In one embodiment, a regulatory sequence may comprise a scaffold/matrix attachment (S/MAR) sequence, also known as MAR sequences. The terms “S/MAR” and “MAR” are used interchangeably throughout this application, having all the same meanings and qualities. S/MAR sequences are transcription enhancing sequences that have been shown to have a stabilizing effect in vivo on transgene expression (Klehr et al. (1991). Biochemistry 30: 1264-1270). S/MAR-based plasmids can function as stable episomes in primary human fibroblast-like cells, supporting long-term transgene expression. However, S/MAR regulatory elements do not display universal behavior in all cell types. In one embodiment, a vector comprises at least one S/MAR sequence. In another embodiment, a vector comprises at least two S/MAR sequences. S/MAR sequences within a vector may be the same or different. In some embodiments, an S/MAR sequence comprises any S/MAR sequence known in the art.

In one embodiment, a regulatory sequence comprises a woodchuck hepatitis virus post-transcriptional regulation element (WPRE). WPRE have been shown to enhance expression in the context of adenoviral vectors as well other viral vectors (Zanta-Boussif et al. (2009) Gene Therapy 16, 605-619; Kingsman et al., (2005) Gene Therapy 12, 3-4). WPRE sequences were shown to stimulate expression when subcloned in the sense orientation between the transgene and the poly(A) sequence. In another embodiment, a WPRE regulatory sequence is located between a sequence encoding IFN and a poly(A) sequence. In another embodiment, a WPRE sequence comprises any WPRE sequence known in the art.

Each combination of regulatory elements represents another embodiment.

In one embodiment, a GMMO or a therapeutic formulation comprising a GMMO comprises an upstream MAR regulatory sequence and at least one more additional regulatory sequence. In one embodiment, the additional regulatory sequences are selected from the group consisting of a MAR sequence, a CAG sequence, an EF1 alpha sequence, and a WPRE sequence.

In one embodiment, an at least one genetically modified micro-organ comprises a helper-dependent adenoviral vector comprising a nucleic acid sequence encoding one or more copies of a therapeutic peptide operably linked to an upstream MAR regulatory sequence, and wherein said nucleic acid further comprises at least one or more additional regulatory sequences, and wherein the at least one genetically modified micro-organ expresses said therapeutic peptide for a sustained period of at least three months

In one embodiment, regulatory elements comprised in a vector and/or expression cassette include at least an S/MAR sequence, an EFlalpha promoter, and a poly(A) sequence. In another embodiment, regulatory elements comprised in a vector include at least an EFlalpha promoter and a poly(A) sequence. In yet another embodiment, regulatory element includes at least an S/MAR sequence, a EF1α promoter, a WPRE sequence and a poly(A) sequence. In still another embodiment, regulatory element comprised in a vector and expression cassette include at least two S/MAR sequences, a EF1α promoter and a poly(A) sequence. In a further embodiment, regulatory elements comprised in a vector and/or expression cassette include at least two different S/MAR sequences and an EF1α promoter, wherein one of the S/MAR sequences is a B globin s/MAR sequence.

In one embodiment, the vector comprising the peptide nucleic acids is a helper-dependent adenoviral vector (“HDAD”, “HD” or “HDAd” or “HD-Ad”), which in another embodiment, is synonymous with gutless, gutted, mini, fully deleted, high-capacity, A, or pseudo adenovirus, and which in another embodiment are deleted of all viral coding sequences except for sequences supporting DNA replication, which in one embodiment, comprise the adenovirus inverted terminal repeats and packaging sequence (ψ). In another embodiment, HDAd express no viral proteins. In one embodiment, a HDAd comprises only the cis-acting elements of the adenovirus required to replicate and package the vector DNA. In one embodiment, a HDAd comprises approximately 500 bp of wild-type adenovirus sequence. In another embodiment, the adenoviral vector additionally comprises stuffer DNA. In one embodiment, the stuffer sequence is mammalian DNA. In one embodiment, the HDAd vector is a non-replicating vector.

E. Micro-Organs

The term micro-organ “MO” as used herein, refers to an isolated tissue or organ structure derived from or identical to an explant that has been prepared in a manner conducive to cell viability and function. In some embodiments, the explant is an intact tissue explant. In some embodiments, an MO maintains at least some in vivo structures of the tissue or organ from which it was isolated. In some embodiments, an MO maintains cell-to-cell interactions, similar to those of the tissue or organ from which it is obtained. In some embodiments, an MO is an intact, isolated tissue sample. In some embodiments, MO retain the micro-architecture and the three dimensional structure of the tissue or organ from which they were derived and have dimensions selected so as to allow passive diffusion of adequate nutrients and gases to cells within the micro-organ and diffusion of cellular waste out of the cells of the micro-organ so as to minimize cellular toxicity and concomitant cell death due to insufficient nutrition and/or accumulation of waste. In some embodiments, an MO is a sliver of dermal tissue, i.e., a dermal micro-organ (“DMO”). The MO may possess any mixture of the above features.

The MO may be a genetically-modified micro-organ (GMMO) or a genetically-modified dermal micro-organ (GMMDO). Dermal micro-organs (“DMO”) may comprise a plurality of dermis components, where dermis is the portion of the skin located below the epidermis. These components may comprise fibroblast cells, epithelial cells, other cell types, bases of hair follicles, nerve endings, sweat and sebaceous glands, and blood and lymph vessels. In some embodiments, a dermal micro-organ may comprise some fat tissue, wherein in other embodiments, a dermal micro-organ may not comprise fat tissue.

In some embodiments, the dermal micro-organ may contain tissue of a basal epidermal layer and, optionally, other epidermal layers of the skin. In other embodiments, the dermal micro-organ does not include basal layer tissue. In some embodiments, the dermal micro-organ does not include epidermal layers. In yet other embodiments, the dermal micro-organ contains an incomplete epidermal layer. In still other embodiments, the dermal micro-organ may contain a few layers of epidermal tissue. In still other embodiments, the dermal micro-organ may contain invaginations of the epidermis into the dermis. In some embodiments, a dermal micro-organ does not include a complete epidermal layer. In further embodiments, the dermal micro-organ may include additional components such as sweat glands and/or hair follicles.

In some embodiments, the DMO includes the entire cross-section of the dermis. In some embodiments, the dermal micro-organ includes part of the cross-section of the dermis. In further embodiments, the DMO includes most of the cross section of the dermis, namely, most of the layers and components of the dermis including the papillary and reticular dermis. In further embodiments, the DMO includes primarily dermal tissue, but may also include fat tissue. In some embodiments, the DMO does not produce keratin or produces a negligible amount of keratin, thereby preventing the formation of keratin cysts following implantation in a recipient, for example, following subcutaneous or intradermal implantation. Further details regarding dermal micro-organs, including methods of harvesting, maintaining in culture, and implanting said dermal micro-organs, are described in PCT Patent Applications WO2004/099363 and WO 2013/118109.

### II. Methods of Treatment

In general, the invention provides methods of treating or preventing a disease or disorder in a human subject in need over a sustained time period comprising the steps of: providing at least one genetically modified micro-organ that provides a sustained delivery of a peptide, the micro-organ comprising a viral vector comprising a nucleic acid sequence encoding a peptide operably linked to an upstream regulatory sequence, and wherein the nucleic acid optionally further comprises at least one or more additional regulatory sequences; determining peptide secretion levels of the at least one genetically modified micro-organ in vitro; implanting the at least one genetically modified micro-organ in a subject at an effective dosage; and measuring peptide levels in the subject; wherein implantation of the at least one genetically modified micro-organ increases the in vivo serum peptide levels in the subject over basal levels for at least three months, optionally at least 6 month.

Efficacy may be measured by detecting therapeutic peptide (wild type or variant) in the serum. Efficacy may also be evaluated by considering clinical signs. For example, efficacy may be evaluated by measuring if there is an increase at the intestinal villus length and/or cell proliferation, or if there is a change in gastrointestinal secretions, or if there is a change in food intake, body weight, or energy levels.

A. Indications

The present methods may be employed for any condition or disease that can be treated by administration of a therapeutic peptide. The therapeutic peptide may be GLP-2 or GLP-2 variant, oxyntomodulin or oxyntomodulin variant, or PYY or PYY variant.

The disease or condition to be treated may be short bowel syndrome (SBS), including SBS in a patient dependent on parenteral support, colitis, inflammatory bowel disease (IBD), including Crohn's disease and ulcerative colitis, acute pancreatitis, intestinal injury, including intestinal ischemia and reperfusion injury, bowel, colon, or colorectal cancer, intestinal radiation damage, or osteoporosis. In some embodiments, the therapeutic peptide provides adjuvant therapy during cancer chemotherapy.

Short Bowel Syndrome (SBS) is a group of problems related to poor absorption of nutrients that may occur in people who have had a portion of their small intestine removed, such as half or more of their small intestine removed. People with short bowel syndrome often cannot absorb enough water, vitamins, sugars such as glucose, maltose, and fructose, and other nutrients from food to sustain life. Thus, some patients with SBS are placed on parenteral support in order to provide these nutrients intravenously. In newborns, short bowel syndrome may occur following surgery to treat conditions such as necrotizing enterocolitis, a condition that occurs in premature infants and leads to the death of bowel tissue, congenital defects of the bowel, such as midgut volvulus, omphalocele and gastroschisis, jejunoileal atresia, internal hernia, and congenital short bowel meconium ileus, a condition associated with cystic fibrosis. In children and adults, short bowel syndrome may occur following surgery to treat conditions such as intussusception, a condition in which part of the intestine folds into another part of the intestine, Crohn's disease, an inflammatory bowel disease bowel injury from loss of blood flow due to a blocked blood vessel, bowel injury from trauma, cancer and damage to the bowel caused by cancer treatment. Short bowel syndrome can also be caused by disease or injury that prevents the small intestine from functioning as it should despite a normal length. A GLP-2 variant known as teduglutide (Gattex®) is currently approved for treatment of SBS patients who are dependent on parenteral support, and is injected subcutaneously.

In some embodiments, GMMO comprising a therapeutic peptide, e.g. GLP-2 or GLP-2 variant, may be used to treat SBS in a patient in need thereof, including an SBS patient who is dependent on parenteral support. In some embodiments, treatment of SBS with a GMMO expressing may result in a reduction in the parenteral nutrition requirements of the patient (i.e. in the IV fluid requirements), such as at least a 10% reduction, or at least at 20% reduction after three months or after six months of treatment. In some embodiments, GMMO comprising GLP-2 or GLP-2 variant may be used to treat a patient in need of stimulation of intestinal epithelial growth. In some embodiments, GMMO comprising GLP-2 or GLP-2 variant may be used to treat colitis, inflammatory bowel disease (IBD), colon, bowel, or colorectal cancers, or acute pancreatitis. In some embodiments, GMMO comprising GLP-2 or GLP-2 variant may be used for protection of the small intestine from radiation damage, such as during cancer treatment. In some embodiments, GMMO comprising GLP-2 or GLP-2 variant may be used to treat an intestinal injury, such as an intestinal ischemia and reperfusion injury. Treatment of such an injury encompasses providing the GMMO prior to the injury in order to reduce the severity of or prevent the injury.

In some embodiments, GMMO comprising a therapeutic peptide, e.g. GLP-2 or GLP-2 variant, may stimulate intestinal epithelial growth in the patient, may improve absorption of energy, may increase bone mineral density, may reduce fecal wet weight, and/or may reduce mucosal atrophy in the small bowel.

In some embodiments, the GMMO comprising a therapeutic peptide, e.g. GLP-2 or GLP-2 variant, may be used for protection of the small intestine from radiation damage, such as during cancer treatment may be designed to deliver a therapeutically effective amount of the therapeutic peptide. For example, a therapeutically effect amount of GLP-2 may comprise a dose of between 0.01 and 0.2 mg/Kg/day of GLP-2 to the patient, such as between 0.01 and 0.1 mg/Kg/day, or between 0.025 and 0.075 mg/Kg/day, or 0.04 to 0.06 mg/Kg/day, or 0.05 mg/Kg/day. In some embodiments, the GMMO comprising a therapeutic peptide, e.g. GLP-2 or GLP-2 variant, may be designed to deliver a therapeutically effective dose of the therapeutic peptide, e.g. GLP-2, of 0.01, 0.02, 0.03, 0.04, 0.05, 0.06, 0.07, 0.08, 0.09, 0.1, 0.15, or 0.2 mg/Kg/day or any range in between two of those numbers.

In some embodiments, the disease or condition to be treated may be over-eating, obesity, diabetes, hypothalmic hyperphagia, binge-eating disorder, or Prader-Willi syndrome, or another condition or disease characterized by a deficiency of oxyntomodulin or PYY.

In some embodiments, GMMO comprising a therapeutic peptide, e.g. oxyntomodulin or PYY or a variant of oxyntomodulin or PYY, may be used to deliver a therapeutically effective amount of at least one therapeutic peptide to treat over-eating, obesity, diabetes, hypothalmic hyperphagia, binge-eating disorder, or Prader-Willi syndrome. In some embodiments, GMMO comprising oxyntomodulin or an oxyntomodulin variant may be used to treat a patient in need of a GLP-1 or glucagon agonist. In some embodiments, a GMMO comprising oxyntomodulin or an oxyntomodulin variant may be used to treat a patient in need of inhibition of gastric acid secreation or stimulation of gastric emptying. In some embodiments, a GMMO comprising oxyntomodulin or an oxyntomodulin variant may be used to treat a patient in need of weight loss or increased activity or energy expenditure. In some embodiments, a GMMO comprising PYY or a PYY variant may be used to treat a patient in need of inhibition of gastric, pancreatic or intestinal secretions. In some embodiments, a GMMO comprising PYY or a PYY variant may be used to treat a patient in need of stimulation of absorption or growth in intestinal epithelium. In some embodiments, a GMMO comprising PYY or a PYY variant may be used to treat a patient in need of weight loss or increased activity or energy expenditure.

For each of the indications recited herein, a GMMO comprising a combination of oxyntomodulin and PYY may be used.

Because of its implanted nature, the GMMO expressing a therapeutic peptide is expected to provide a more favorable pharmacokinetic profile than peptides administered through other routes, providing physiologic and therapeutically effective levels of continuous therapeutic peptide.

### EXAMPLES

### Example 1

### GLP-2 Measurement System

A commercial sandwich assay ELISA from Millipore was identified and found to be suitable for measuring levels of GLP-2. The reported detection limit by the manufacturer is 0.3 ng/ml and according to the manufacturer it can be used with spent media and human or rat serum or plasma. The commercially-provided instructions for this ELISA assay may be followed to detect levels of GLP-2 in both in vivo and in vitro samples.

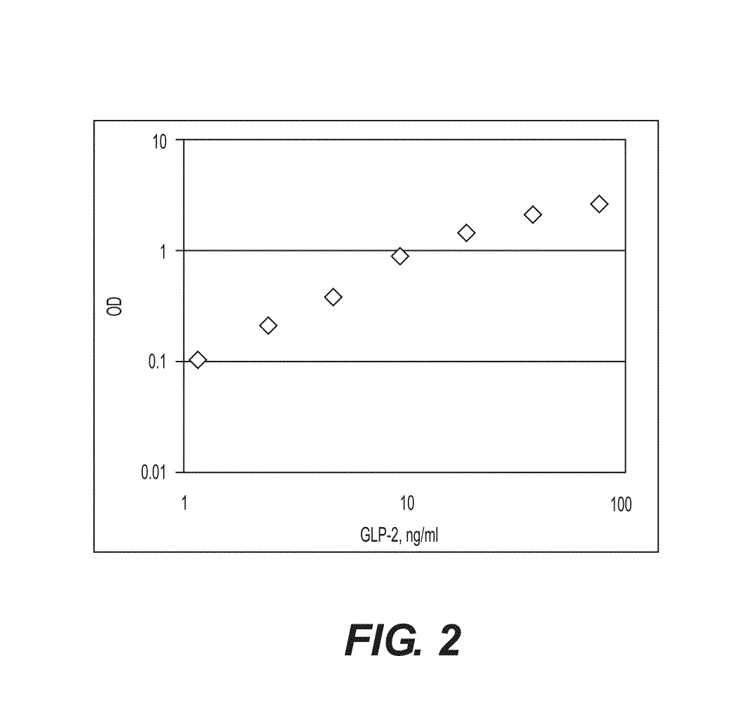

A representative calibration curve is provided in FIG. 2.

### Example 2

### Preparation of Vectors and Other Constructs

A vectors containing the GLP-2 variant therapeutic peptide was prepared as follows:

Proglucagon signaling peptide-GLP-2 variant therapeutic peptide-linker-GLP-2 variant therapeutic peptide (sequence provided in SEQ ID NO: 6).

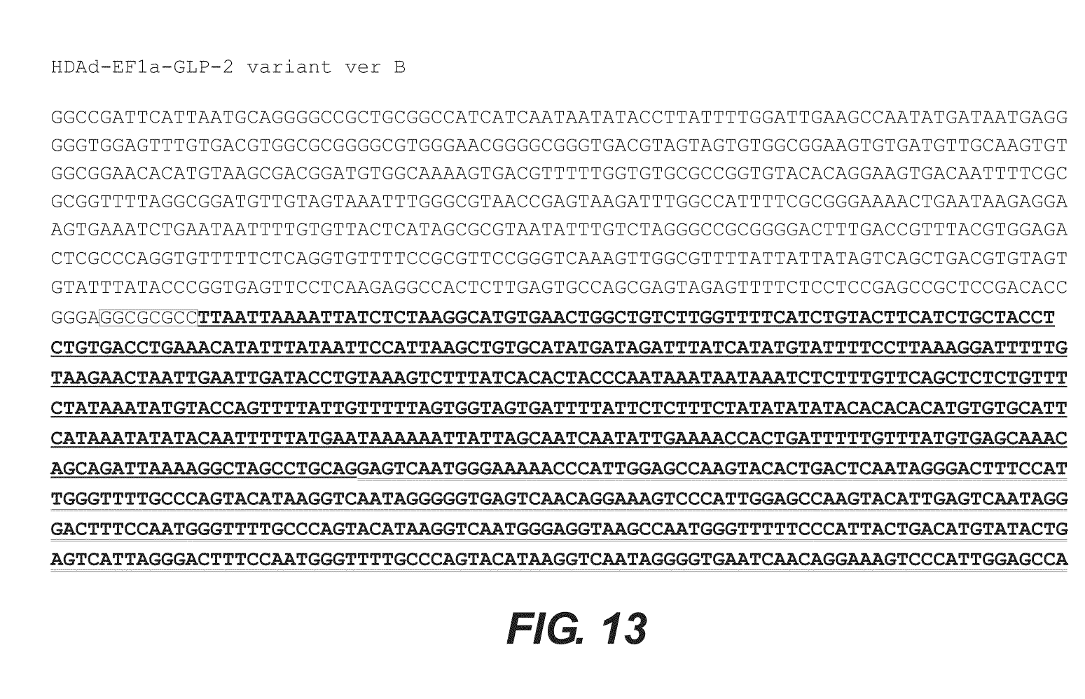

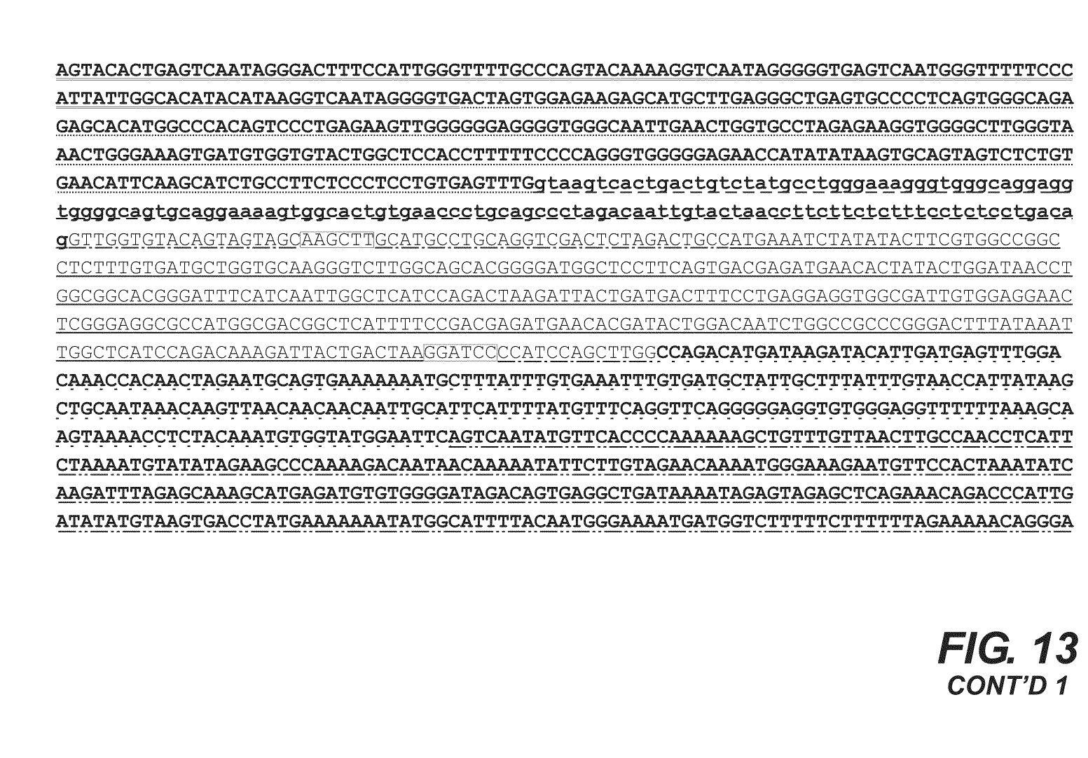

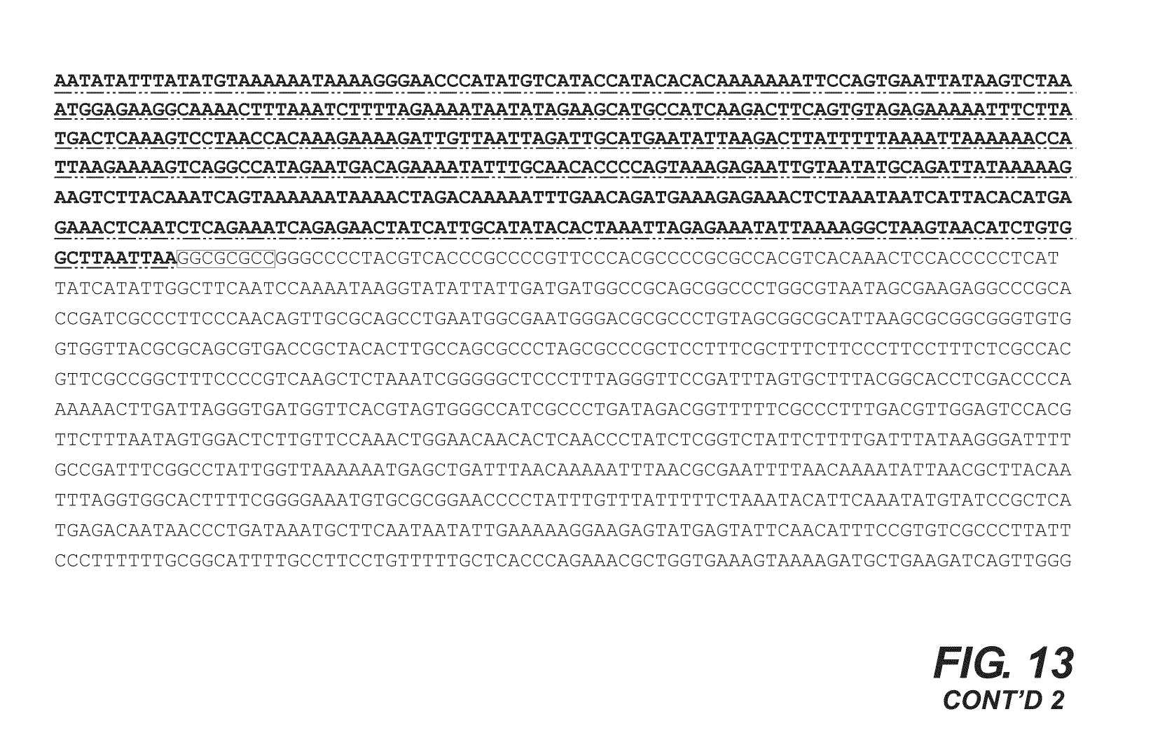

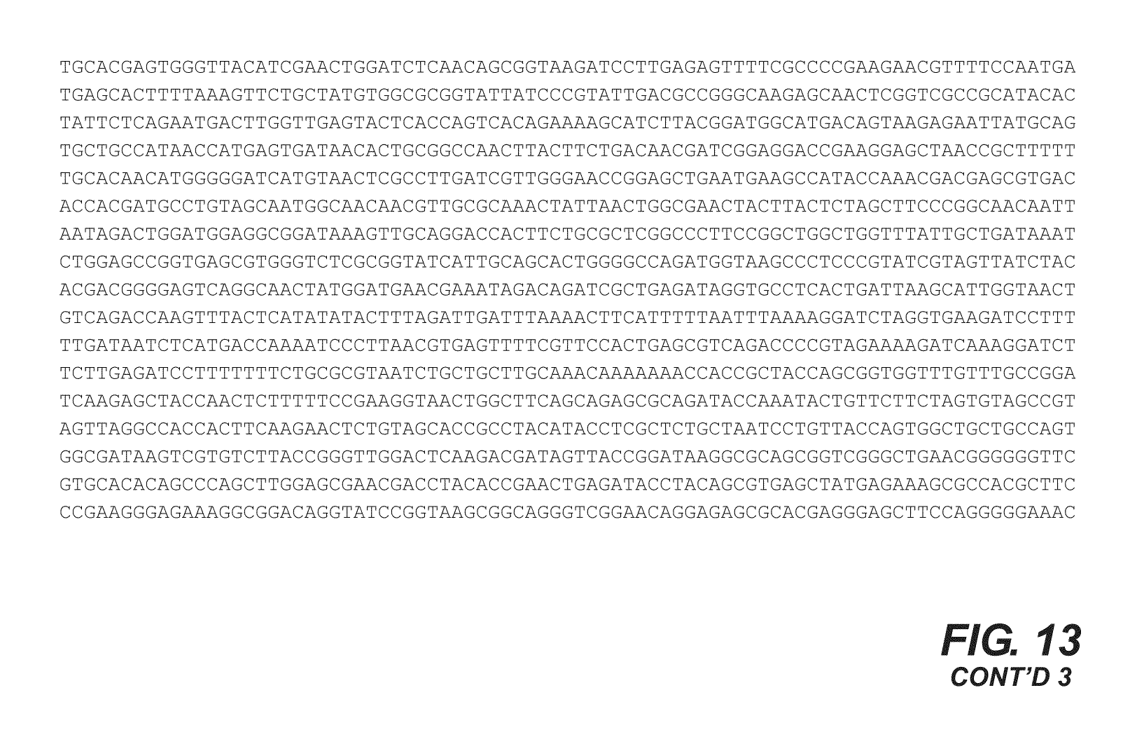

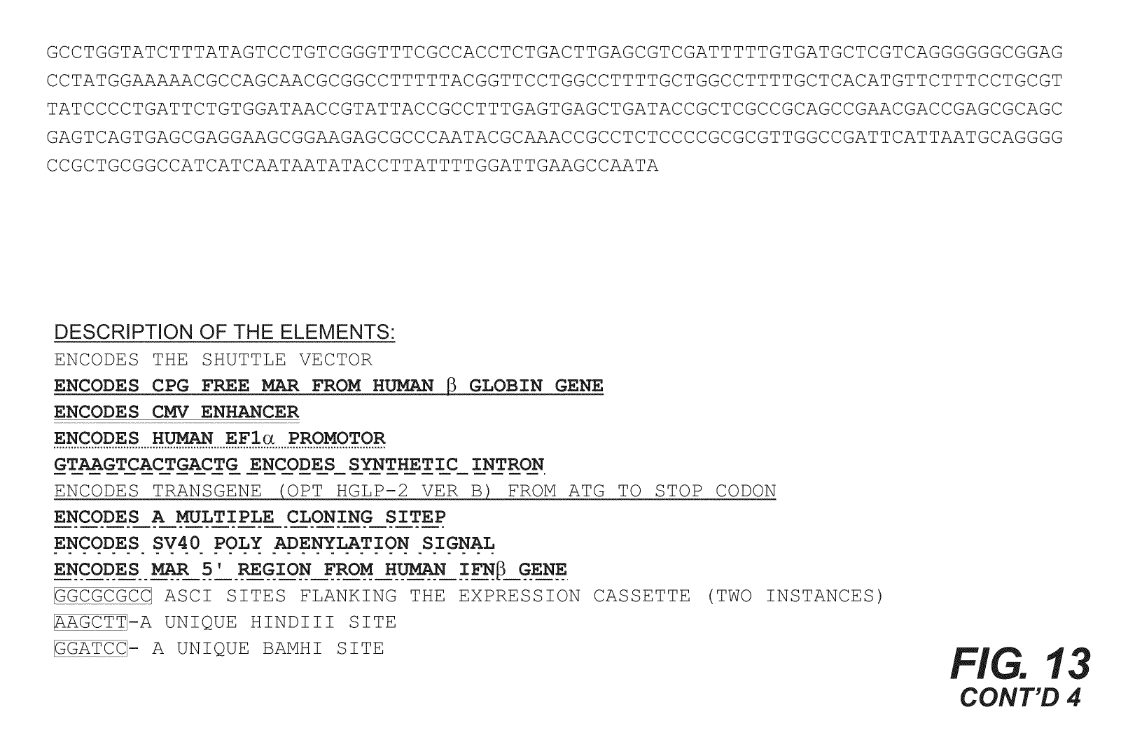

The vector sequence is provided as SEQ ID NO: 21 and shown with annotations in FIG. 13. The vector is designated HDAd-EF1a-GLP-2 variant ver B.

Plasmids were also created using the GLP-2 wild type and the GLP-2 variant encoding nucleic acids.

The GLP-2 wildtype construct (SEQ ID NO: 4) was used in the first nucleofection experiment as compared to the GLP-2 variant construct (SEQ ID NO: 6) (Example 3A). The GLP-2 variant construct (SEQ ID NO: 6) alone was used in all subsequent examples.

### Example 3

### Electroporation of Human Dermal Fibroblasts Using Amaxa® Nucleofector® (Lonza)

A. A First Nucleofection Experiment

A nucleofection experiment was performed according to the following protocol. Human dermal fibroblast cells (HDF) from tummy tuck tissue treated with a collagenase treatment were used after passage 6 or passage 5. The growth medium was DMEM-F-12 (ADCF) with phenol red (Hy Clone). Medium was supplemented with 10% DCS (Defined Calf serum Iron Supplemented HyQ); AmBisome 2.5 μg/ml (Liposomal Amphotericin B 50 mg—GILEAD); Gentamycin sulfate 50 μg/ml (Gentamicin-IKA 80 mg Teva). The trypsin used was trypsin/EDTA (Trypsin/EDTA; Lonza). The Hepes buffered saline (HBS) used was Hepes buffered saline*2 (hepes buffered saline; Lonza). The trypsin neutralizing solution (TNS) used was from Lonza). The growing conditions (pre-nuclefection) were as follows: five days before electroporation cells were seeded in 10 cm² plates; medium was changed every 3 days; cells reached 90% confluency in the experiment day.

The experimental procedure was as follows: Growth medium was removed from four plates of 10 cm. Cells were washed once with 10 ml HBS. Cells were harvested by trypsinization: 3 ml of Trypsin/EDTA solution was added to the plate; the plate was gently swirled to ensure an even distribution of the solution; the plate was incubated at 37° C. for 3 minutes; then the plate was removed from the incubator; TNS was added to inactivate the trypsin; and cells were gently resuspended and removed from flasks by pipetting.

Cells were counted three times—the yield was 7×106 cells. Cells were pelletted in 500 g for 10 min in a 50 ml tubes. Cells were resuspended with 1400 μl of Human Dermal Fibroblast Nucleofector™ Solution (final conc. 5*10⁵ cells/100 μl). 100 μl of cells were mixed with 5 μg DNA. The nucleofection sample was transferred into an amaxa certified cuvette. The electroporation program U-23 was activated. Cells were removed from the cuvette immediately at the end of the program by adding 500 μl of pre-warmed culture medium and transferred into 6 well plate. Reactions containing 5*10⁵ cells/100 μl were seeded into 6 well plate already containing 1.5 ml of growth medium. Cells were transferred from the cuvette to the dish using plastic pipette.

TABLE 1
Nucleofection

| Plate | Cell | | Nucleofector |
|---|---|---|---|
| Designator | number | DNA | program |
| A | 5*10⁵ | PAd-MAR-EF1a-opt-hEPO | U-23 |
| B | 5*10⁵ | pAd-MAR-EF1a-test peptide | U-23 |
| | | whole precursor | |
| C | 5*10⁵ | pAd-MAR-GLP-2 variant verB | U-23 |
| D | 5*10⁵ | pAd-MAR-GLP-2 wt verB | U-23 |
| E | 5*10⁵ | GFP Lonza control | U-23 |
| F | 5*10⁵ | No pulse and no DNA | U-23 |

Media was collected (2.1 ml) and frozen at −80° C. after 24 hours. Medium (2 ml) was collected 24 hrs and 48 hrs, cells were harvest for protein extraction using M-per (Pierce) according to the following protocol: collected growth media from each well to cold Eppendorf tube and centrifuged for 10 min in 5000 rpm; transfer the supernatant to another Eppendorf tube and keep the pellet; on ice—wash each well with 500 μl PBS and transfer all the cells (with PBS) to the tubes with the pellet; centrifuge for 10 min in 5000 rpm; on ice—skim the cells with 200 μl M-per reagent containing protease inhibitor (1:100); centrifuge for 10 min in 13000 rpm; collect supernatant and freeze at −80° C.

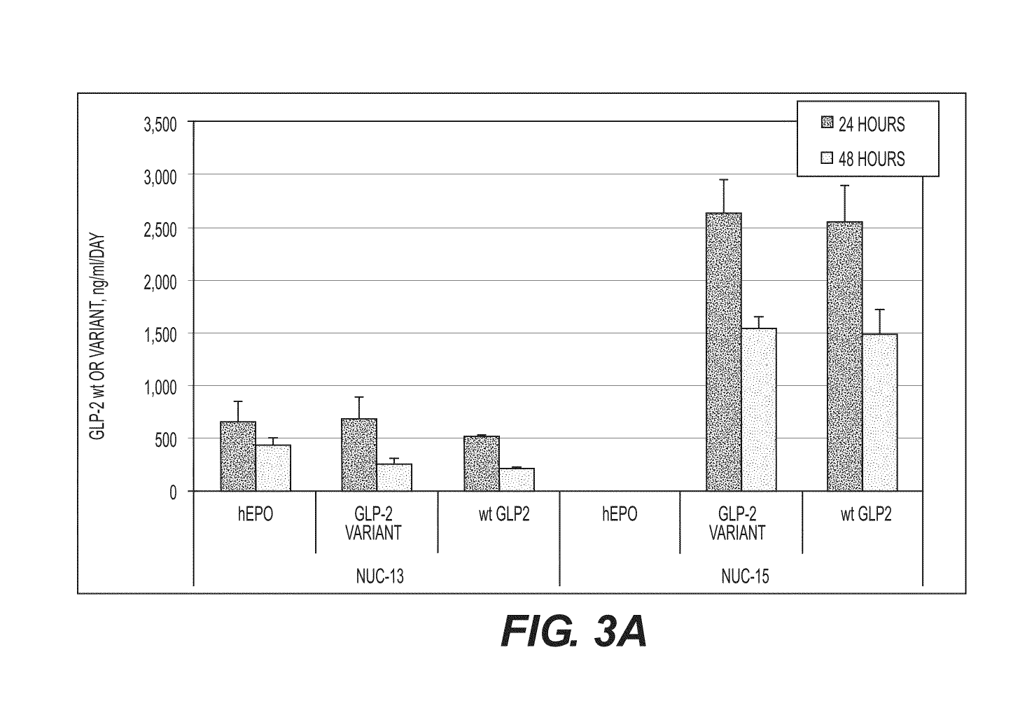

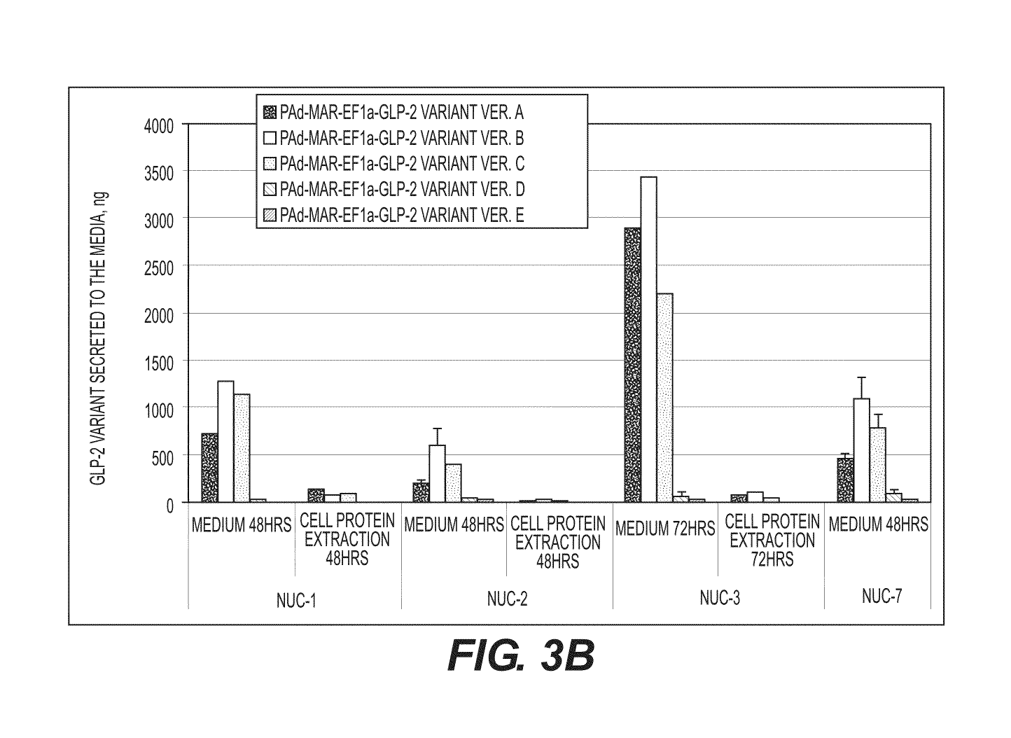

Results are provided in FIG. 3A. This shows that similar secretion levels were obtained when human dermal fibroblasts were transfected with plasmids containing GLP-2 Variant or GLP-2 expression cassettes. Results for Nuc-15 were provided (after passage number 6) and results for Nuc-13 (after passage number 5).

a. A Second Nucleofection Experiment

A second nucleofection experiment was performed under similar conditions, except for passage numbers of the cells: Nuc-1: passage 3, Nuc-2: passage 3, Nuc-3: passage 3, and Nuc-7: passage 12. Results are provided in FIG. 3B.

### Example 4

### Preparation of Genetically-Modified Micro-Organ Comprising GLP-2 Variant Sequence

B. HumanA-280

1. Preparation of GMMO's

This experiment was performed to evaluate the effect of GMMO implantation transduced with HDAd-EF1a-GLP-2 variant ver B (a construct according to approach B and comprising GLP-2 variant sequence). Implantation was performed on day 8 from harvest, with DepoMedrol injections every two weeks.

Materials and Equipment for the experiment was as follows. The experiment used a DME/F-12 medium with 10% DCS (defined calf serum)

HyClone DME/F-12 1:1 (X1)+2.50 mM L-Glutamine+15 mM HEPES Buffer (Thermo scientific). Medium was supplemented with 10% DCS (HyClone Defined Bovine Calf Serum supplemented, Thermo scientific), AmBisome 2.5 μg/ml (Liposomal Amphotericin B 50 mg—Gilead), and Gentamycin sulfate 50 μg/ml (Gentamicin-IKA 80 mg/2 ml-Teva). The viral vector used was HDdelta28E4-MAR-EF1a-optGLP-2verB-1, at 7.608×10¹² vp/ml. The skin used was from tummy tuck tissue.

Dermal core MOs (30 mm) were prepared in a sterile hood using the NOUVAG chuck driller, with the NOUVAG motor set at 7000 rpm chuck driller and double hump Dermavac 3 mm equipment with 14G needles (2.05 mm deep from skin surface) and back vacuum containing 2 ml of saline. The MO's were flushed out from the needles with saline. Needles were replaced every 4-5 harvest. The MO's were incubated for one minute in saline. Then all the MO's were washed 3 times with DMEM F-12 media without serum in a Petri dish (all the MO's were cleaned). Every wash was performed in a new Petri dish.

All the MOs were incubated with 1 ml growth media in 24 well/plate (SARSTEDT for Suspension Cells) at 5% CO₂ incubator 32° C. for 24 hrs.

Certain MO's were transduced with HDAd-EF1a-GLP-2 variant ver B, 7.608*10¹² vp/ml to produce GMMO's and other MO's were not transduced as a negative control. The vector was diluted in Growth media containing 10% DCS serum to final concentration of 1.5×10¹⁰ vp/GMMO (2.0 μl/GMMO). Other MO's were not transduced as a negative control. In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

The viral wash was conducted in an open system (24 well/plate). GMMO's/MO's were washed from the transduction medium, and growth medium was added. The 250 μl of transduction medium was removed from the plate with a pipettor, and 2 ml of fresh growth medium was added (first wash). 3 ml of growth medium was added to wells of a new 6 well plate (“maintenance plate”) and the MO's were transferred into the wells from the plate in which the transduction was done (second wash). The 3 ml of media was removed from each well and fresh 3 ml media was added (third wash). And then another 3 washes were conducted. The GMMO's/MO's were transferred to a new 24 well plate with fresh 1 ml growth media in each well. The plate was incubated at 32° C., 5% CO₂ for 3 days.

During a maintenance phase, the growth media were changed and collected for analyses by ELISA every Sunday and Wednesday.

2. SCID 75 In Vivo Experiment for GLP-2 Variant

The GMMO's/MO's were transferred to the SCID facility in an incubator at 32° C., without CO₂ in 2 ml cryotubes containing 2 ml growth medium (2.5 hrs transport time). All GMMO's/MO's were washed in saline×6 washes prior to implantation.

Two GMMO's/MO's were implanted SQ in each mouse and implantation was done by implantation device with 10 G needle (see Table 2). Depo-Medrol (40 mg/ml, Pfizer) was be injected to groups according to Table 2 on implantation and every two weeks. The injections were as following: 1 mg depomedrol per GMMOs or MOs (25 μl Depomedrol stock +75 μl saline/GMMO or MO).

Mice were bled after one week, and then every 10 days. EPO and GLP-2 (GLP-2 plasma) in the serum were be measured by ELISA.

The duration of the experiment was 2 months. The following table shows the μg/mouse implanted (per 2 GMMOs which were implanted) and μg/day/MO (the level of GLP-2 measured in the spent media before implantation).

TABLE 2
Mice Groups and GMMO's/MO's Implanted

| | | | μg/day/ | Left/ | μg/ |
|---|---|---|---|---|---|
| Group# | Mouse# | MO# | MO | Right | mouse |
| H | H1 | 15 | 2.0 | Left | 4.05 |
| HDAd-EF1a-GLP-2 | | 21 | 2.0 | Right | |
| variant ver B implantation | H2 | 14 | 1.9 | Left | 4.23 |
| Depo-Medrol day 0 & | | 13 | 2.3 | Right | |
| every two weeks from | H3 | 19 | 1.8 | Left | 4.28 |
| implantation | | 20 | 2.4 | Right | |
| | H4 | 24 | 1.3 | Left | 4.28 |
| | | 16 | 3.0 | Right | |
| I | I1 | 25 | 2.4 | Left | 5.21 |
| HDAd-EF1a-opt EPO | | 36 | 2.8 | Right | |
| implantation | I2 | 34 | 2.4 | Left | 5.44 |
| Depo-Medrol day 0 & | | 35 | 3.1 | Right | |
| every two weeks from | I3 | 30 | 2.1 | Left | 5.27 |
| implantation | | 26 | 3.2 | Right | |
| | I4 | 32 | 1.8 | Left | 5.15 |
| | | 31 | 3.3 | Right | |
| J | J1 | 37 | | Left | |
| Non-Transduced | | 38 | | Right | |
| implantation | J2 | 39 | | Left | |
| Depo-Medrol day 0 & | | 40 | | Right | |
| every two weeks from | J3 | 41 | | Left | |
| implantation | | 42 | | Right | |

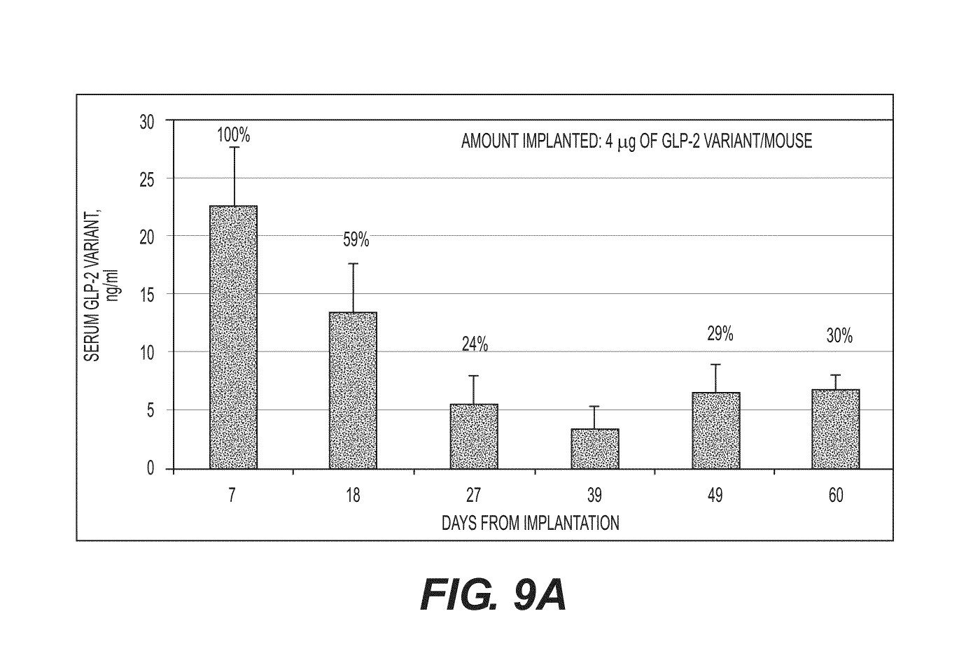

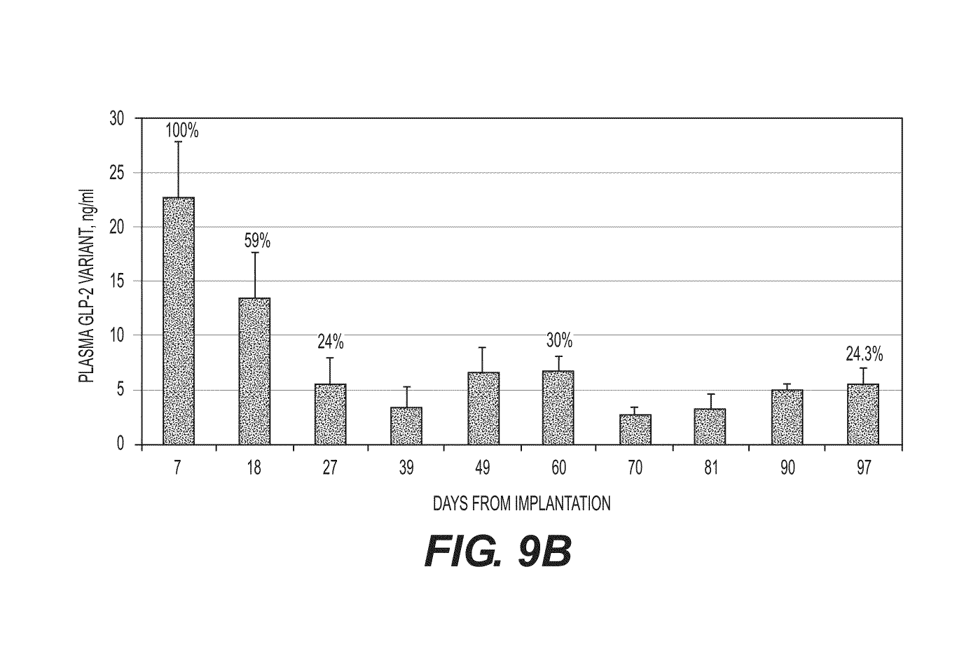

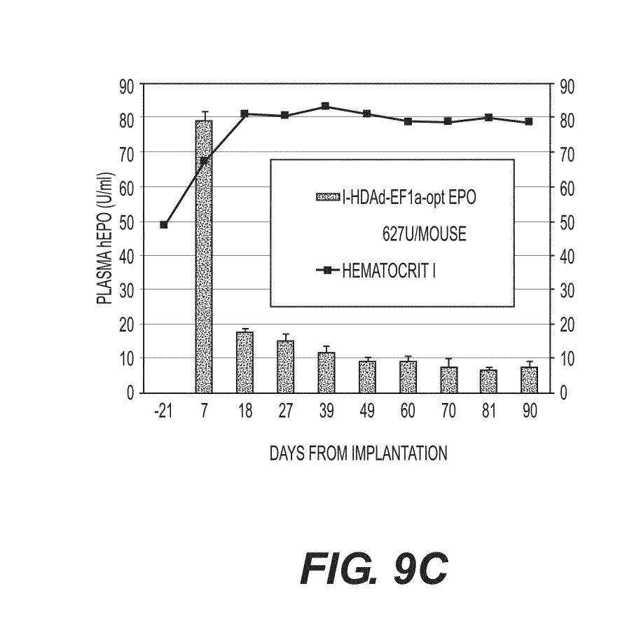

FIG. 9A/B/C shows the in vivo SCID mice performance of GLP-2 variant. The in vivo GLP-2 secretion profile suggests a 75% reduction from peak level one month post implantation, followed by an additional month of stable secretion.

C. HumanA-265

A GLP-2 variant titration experiment was performed. The materials were as follows. The DME/F-12 medium with 10% DCS media included HyClone DME/F-12 1:1 (X1)+2.50 mM L-Glutamine+15 mM HEPES Buffer (Thermo scientific). Medium was supplemented with 10% DCS (HyClone Defined Bovine Calf Serum supplemented, Thermo Scientific); AmBisome 2.5 μg/ml (Liposomal Amphotericin B 50 mg Gilead); Gentamycin sulfate 50 μg/ml (Gentamicin-IKA 80 mg/2 ml-Teva). The viral vector used was HDdelta28E4-EF1a-GLP2 variant verB-1, 7.608×10¹² vp/ml. The skin used was tummy tuck tissue.

A total of 8 dermal core MOs (30 mm) were prepared in a sterile hood using the NOUVAG chuck driller; NOUVAG motor set at 7000 rpm chuck driller and double hump Dermavac 3 mm equipment with 14G needles and back vacuum containing 2 ml of saline. The MO's were flashed out from the needles with saline. Needles were replaced every 4-5 harvest. The MO's were incubated for one minute in saline. Then all the MO's were washed 3 times with DMEM F-12 media without serum in a Petri dish (all the MO's were cleaned). Every wash was performed in a new Petri dish.

All the MOs were incubated with 1 ml growth media with serum, in 24 well/plate (SARSTEDT for Suspension Cells) at 5% CO₂ incubator 32° C. for 24 hrs.

TABLE 3
Experiment plan and samples numbering

| Group | | |
|---|---|---|
| No. | Well No. | Treatment |
| 1 | 1-4 | HDAd-EF1a- GLP-2 variant ver B |
| | | 3.0 × 10¹⁰ vp/GMMO |
| 2 | 5-8 | HDAd-EF1a- GLP-2variant ver B |
| | | 1.5 × 10¹⁰ vp/GMMO |

Viral transduction was performed as follows. MO's 1-4 were transduced with HDAd-EF1a-GLP-2 variant ver B, 7.608*10¹² vp/ml. The vector was diluted in growth media containing 10% DCS serum to final concentration of 3.0×10¹⁰ vp/MO (3.94 μl/MO) (24.4 ul 7.608*10¹² vp/ml+1501 μl growth medium). MO's 5-8 were transduced with HDAd-EF1a-GLP-2 variant ver B, 7.608*10¹² vp/ml. The vector was diluted in growth media containing 10% DCS serum to final concentration of 1.5×10¹⁰ vp/MO. The vector was diluted 1:1 from the 3.0×10¹⁰ vp/MO concentration (510 ul 3.0×10¹⁰ vp/MO+510 μl growth medium). In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

A viral wash was performed as follows. In an open system (24 well/plate), GMMOs were washed from the transduction medium, and growth medium was added. The 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). The GMMOs were transferred to a new 24 well plate with fresh 1 ml growth media in each well. The plate was incubated at 32° C., 5% CO₂ for 3 days.

In a maintenance phase, the growth media was changed and collected for analysis by ELISA every Sunday and Wednesday.

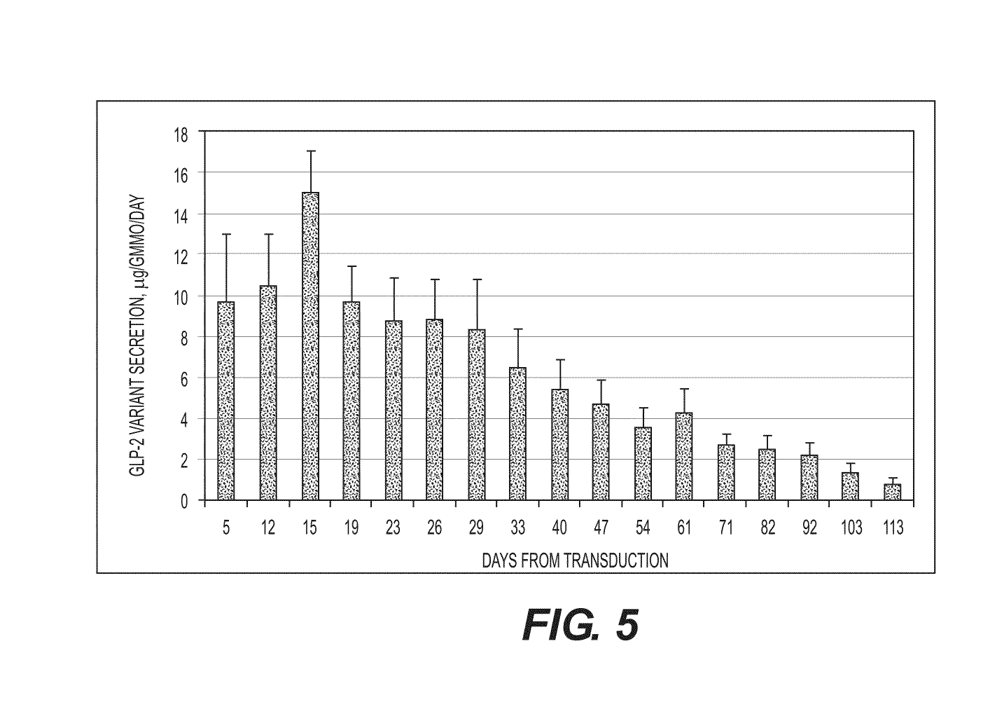

FIG. 5 provides an in vitro GLP-2 variant secretion profile. GLP-2 GMMO secretion levels are at the range of μg per day. Stable in vitro secretion was observed at the first month; however reduction of about 95% from initial peak level was observed in a three-month time frame.

D. HumanA-281

This experiment was performed to examine the effect of different media on GLP-2 variant secretion.

This experiment utilized a variety of candidate media including DME/F-12 medium with 10% DCS, Serum-free ACTive Medium (CellGenix), X-VIVO™ 15, without Phenol Red Serum-free Hematopoietic Cell Medium (Lonza), and TheraPEAK™ MSCGM-CD™ Mesenchymal Stem Cell Medium (Lonza).

A variety of candidate media were prepared as follows. DME/F-12 medium included HyClone DME/F-12 1:1 (X1)+2.50 mM L-Glutamine+15 mM HEPES Buffer (Thermo Scientific). Medium was supplemented with AmBisome 2.5 μg/ml (Amphotericin B Solution 250 ug/ml Biological Industries); and Gentamycin sulfate 50 μg/ml (Gentamicin-IKA 80 mg/2 ml-Teva).

DME/F-12 medium with 10% DCS (defined calf serum) included HyClone DME/F-12 1:1 (X1)+2.50 mM L-Glutamine+15 mM HEPES Buffer (Thermo Scientific). Medium is supplemented with 10% DCS (HyClone Defined Bovine Calf Serum supplemented, Thermo Scientific); AmBisome 2.5 μg/ml (Amphotericin B Solution 250 μg/ml Biological Industries); Gentamycin sulfate 50 μg/ml (Gentamicin-IKA 80 mg/2 ml-Teva).

Serum-free ACTive Medium was Serum-free ACTive Medium for preclinical ex vivo use (CellGro/CellGenix). Medium was supplemented with AmBisome 2.5 μg/ml (Amphotericin B Solution 250 ug/ml Biological Industries) and Gentamycin sulfate 50 μg/ml (Gentamicin-IKA 80 mg/2 ml-Teva).

X-VIVO™ 15 Chemically Defined, Serum-free Hematopoietic Cell Medium is formulated with L-glutamine, without gentamicin and without phenol red (Lonza). Medium was supplemented with AmBisome 2.5 μg/ml (Amphotericin B Solution 250 μg/ml Biological Industries) and Gentamycin sulfate 50 μg/ml (Gentamicin-IKA 80 mg/2 ml-Teva).

TheraPEAK™ MSCGM-CD™ Mesenchymal Stem Cell Medium included MSCBM-CD™ Mesenchymal Basal Medium, Chemically defined (Lonza) with MSCGM-CD™ SingleQuots Kit (Lonza)—with L-glutamine, without phenol red and antibiotics. Medium was supplemented with AmBisome 2.5 μg/ml (Amphotericin B Solution 250 ug/ml Biological Industries) and Gentamycin sulfate 50 μg/ml (Gentamicin-IKA 80 mg/2 ml-Teva).

The viral vectors used in this experiment were HDdelta28E4-EF1a-opt hEPO-1, 1.66×10¹² vp/ml and HDdelta28E4-MAR-EF1a-optGLP-2verB-1, 7.608×10¹² vp/ml. The skin used in this experiment was tummy tuck tissue.

The experimental procedure was as follows: dermal core MO's 30 mm were prepared in a sterile hood following the Clinical Harvesting Procedure Protocol (SOP060023 v2) using the NOUVAG chuck driller; NOUVAG motor set at 7000 rpm chuck driller and double hump Dermavac 3 mm equipment with 14G needles and back vacuum containing 2 ml of saline. The MO's were flushed out from the needles with saline. Needles were replaced every 4-5 harvest. The MO's were incubated for one minute in saline. Then all the MO's were washed 3 times in DME/F-12 medium in a Petri dish (all MO's were cleaned). Every wash was performed in a new Petri dish. All the MOs were incubated with 1 ml their respective medium (see Table 4), in 24 well/plate (SARSTEDT for Suspension Cells) at 5% CO₂ incubator 32° C. for 24 hrs.

TABLE 4
Experimental Plan and Sample Numbering

| Group | Well | | |
|---|---|---|---|
| No. | No. | Treatment | Comments |
| 1 | 1-4 | HDAd-EF1a-opt hEPO | 3/4 days maintenance |
| | | DME/F-12 medium with 10% | |
| | | DCS | |
| 2 | 5-8 | HDAd-EF1a-GLP-2 variant ver B | |
| | | DME/F-12 medium with 10% | |
| | | DCS | |
| 3 | 9-12 | HDAd-EF1a-GLP-2 variant ver B | |
| | | X-VIVO™ 15 Medium without | |
| | | Phenol Red | |
| 4 | 13-16 | HDAd-EF1a-GLP-2 variant ver B | |
| | | MSCGM-CD ™ Medium | |
| 5 | 17-20 | HDAd-EF1a-GLP-2 variant ver B | |
| | | Serum-free ACTive Medium | |

Viral transduction occurred as follows. MO's 1-4 were transduced with HdAd-EF1a-opt hEPO, 1.66*10¹² vp/ml. The vector was diluted in media to final concentration of 1.50×10¹⁰ vp/GMMO (9.0 μl/GMMO). MO's 5-20 were transduced with HDAd-EF1a-GLP-2 variant ver B, 7.608*10¹² vp/ml. The vector was diluted in media to final concentration of 1.5×10¹⁰ vp/GMMO (2.0 μl/GMMO). In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

The viral wash was conducted in an open system (24 well/plate). GMMOs were washed from the transduction medium, and DME/F-12 medium was added. The 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). The GMMOs were transferred to a new 24 well plate with fresh 1 ml growth media (see Table 4) in each well. The plate was incubated at 32° C., 5% CO₂ for 3 days.

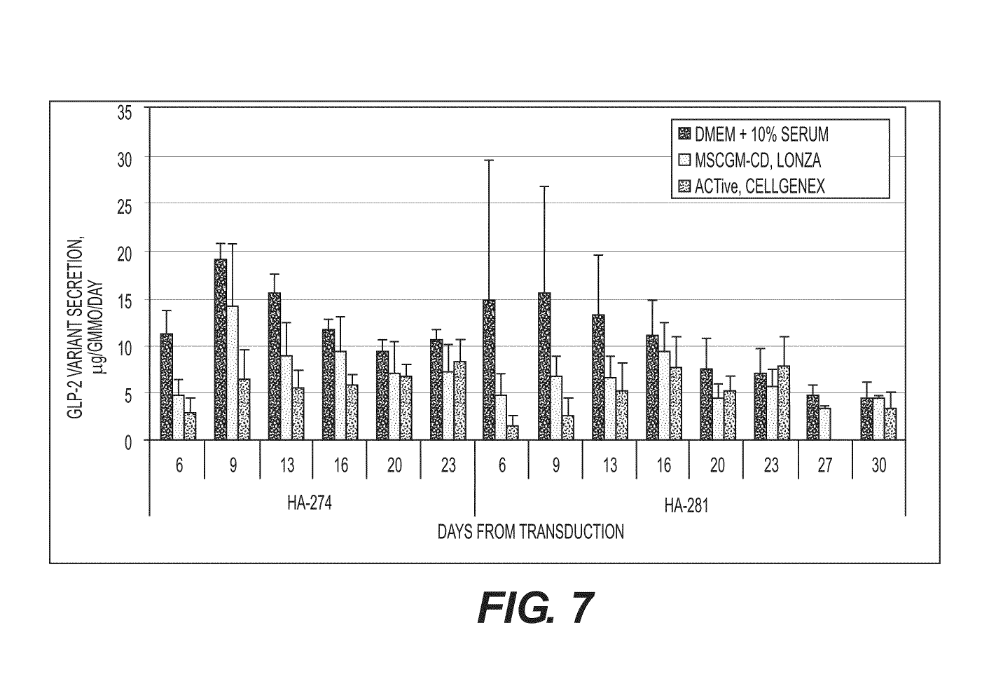

In a maintenance phase, the growth media were changed (see Table 4) and collected for analyses by ELISA every Sunday and Wednesday. Results are shown in the portion of FIG. 7 labeled “HA-281.” FIG. 7 is discussed below.

E. HumanA-274 (Impact of Different Media and Media Exchange Frequency)

As study on the effect of different media and media exchange frequency on GLP-2 secretion was performed with the following media DME/F-12 medium with 10% DCS, Serum-free ACTive Medium (CellGenix), TheraPEAK™ MSCGM-CD™ Mesenchymal Stem Cell Medium (Lonza). The media used were as follows.

DME/F-12 medium was HyClone DME/F-12 1:1 (X1)+2.50 mM L-Glutamine+15 mM HEPES Buffer (Thermo Scientific). Medium is supplemented with AmBisome 2.5 μg/ml (Amphotericin B Solution 250 μg/ml Biological Industries); Gentamycin sulfate 50 μg/ml (Gentamicin-IKA 80 mg/2 ml-Teva). DME/F-12 medium with 10% DCS (defined calf serum) was as follows HyClone DME/F-12 1:1 (X1)+2.50 mM L-Glutamine+15 mM HEPES Buffer (Thermo Scientific). Medium was supplemented with 10% DCS (HyClone Defined Bovine Calf Serum supplemented, Thermo Scientific); AmBisome 2.5 μg/ml (Amphotericin B Solution 250 μg/ml Biological Industries); Gentamycin sulfate 50 μg/ml (Gentamicin-IKA 80 mg/2 ml-Teva).

Serum-free ACTive Medium was serum-free ACTive Medium for preclinical ex vivo use (CellGro/CellGenix). Medium was supplemented with AmBisome 2.5 μg/ml (Amphotericin B Solution 250 μg/ml Biological Industries); Gentamycin sulfate 50 μg/ml (Gentamicin-IKA 80 mg/2 ml-Teva).

TheraPEAK™ MSCGM-CD™ Mesenchymal Stem Cell Medium

MSCBM-CD™ Mesenchymal Basal Medium, Chemically defined (Lonza) with MSCGM-CD™ SingleQuots Kit (Lonza)—with L-glutamine, without phenol red and antibiotics. Medium was supplemented with AmBisome 2.5 μg/ml (Amphotericin B Solution 250 μg/ml Biological Industries); Gentamycin sulfate 50 μg/ml (Gentamicin-IKA 80 mg/2 ml-Teva).

The viral vector used was HDdelta28E4-MAR-EF1a-optGLP-2verB-1, 7.608×10¹² vp/ml. The skin used was tummy tuck tissue. Dermal core MO's 30 mm were prepared in a sterile hood using the NOUVAG chuck driller; NOUVAG motor set at 7000 rpm chuck driller and double hump Dermavac 3 mm equipment with 14G needles and back vacuum containing 2 ml of saline. The MO's were flushed out from the needles with saline. Needles were replaced every 4-5 harvest. The MO's were incubated for one minute in saline. Then all the MO's were washed 3 times in DME/F-12 medium in a Petri dish (all MO's were cleaned). Every wash was performed in a new Petri dish.

All the MOs were incubated with 1 ml their respective medium in 24 well/plate (SARSTEDT for Suspension Cells) (see Table 5), in at 5% CO₂ incubator 32° C. for 24 hrs.

TABLE 5
Experiment plan and samples numbering

| Group No. | Treatment | Comments |
|---|---|---|
| A | HDAd-EF1a-GL2-2 variant ver B | 24 hrs maintenance, |
| | DME/F-12 medium with 10% DCS | 1 ml |
| | 24 hrs maintenance, 1 ml | |
| B | HDAd-EF1a-GLP-2 variant ver B | 3 to 4 days maintenance, |
| | DME/F-12 medium with 10% DCS | 1 ml |
| C | HDAd-EF1a-GLP-2 variant ver B | |
| | MSCGM-CD ™ Medium | |
| D | HDAd-EF1a-GLP-2 variant ver B | |
| | Serum-free ACTive Medium | |

Viral transduction was performed as follows. MO's were transduced with HDAd-EF1a-GLP-2 variant ver B 7.608*10¹² vp/ml. The vector was diluted in media according to Table 5 to final concentration of 1.5×10¹⁰ vp/GMMOs (2.0 μl/GMMOs). In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

The viral wash was performed in an Open system (24 well/plate). GMMOs were washed from the transduction medium, and DME/F-12 medium was added. The 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). The GMMOs were transferred to a new 24 well plate or 6 well plate with fresh growth media (1 ml) in each well. The plate was incubated at 32° C., 5% CO₂ for 3 days.

For maintenance, the growth media were changed and collected for analyses by ELISA every Sunday and Wednesday (except Group A media was collected every day, see table 5).

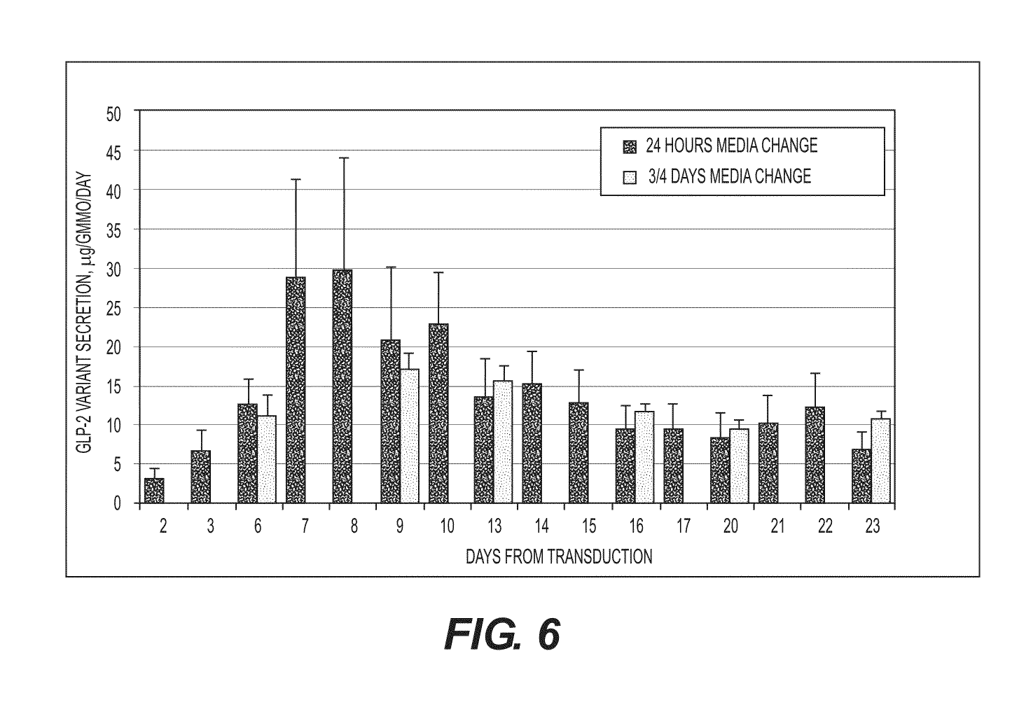

Results from this experiment are shown in FIGS. 6 and 7. FIG. 6 provides the effect of media exchange frequency on secreted GLP-2 variant. Similar secretion levels were obtained when media was exchanged daily or every 3 to 4 days. These observations suggest that GMMO secreted GLP-2 variant is stable in DMEM media at 32° C. for several days.

Data are provided in FIG. 7 in the portion of the graph labeled “HA-274.” FIG. 7 illustrates the in vitro performance of GLP-2 variant secreting GMMOs in various serum free media. GMMOs maintained in MSCGM media showed comparable secretion profile to those maintained in DMEM supplemented with 10% serum.

F. HumanA-283

This experiment was designed to compare intracellular and extracellular distribution of GLP-2 variant (either in the GMMO or outside of it).

The material and equipment used for this experiment included DME/F-12 medium with 10% DCS (defined calf serum), which was HyClone DME/F-12 1:1 (X1)+2.50 mM L-Glutamine+15 mM HEPES Buffer (Thermo Scientific). Medium was supplemented with 10% DCS (HyClone Defined Bovine Calf Serum supplemented, Thermo Scientific); AmBisome 2.5 μg/ml (Liposomal Amphotericin B 50 mg—Gilead); Gentamycin sulfate 50 μg/ml (Gentamicin-IKA 80 mg/2 ml-Teva). T-per (Pierce) and M-PER Mammalian Protein Extraction Reagent (Pierce) were also used.

The following viral vectors were used: HDdelta28E4-MAR-EF1a-optGLP-2verB-1, 7.608×10¹² vp/ml and HDdelta28E4-EF1a-opt hEPO-1, 1.66×10¹² vp/ml. The skin tissue was tummy tuck tissue.

The experimental procedure was as follows. Dermal core MOs (30 mm) were prepared in a sterile hood using the NOUVAG chuck driller; NOUVAG motor set at 7000 rpm chuck driller and double hump Dermavac 3 mm equipment with 14G needles (2.05 mm deep from skin surface) and back vacuum containing 2 ml of saline. The MO's were flushed out from the needles with saline. Needles were replaced every 4-5 harvest. The MO's were incubated for one minute in saline. Then all the MO's were washed 3 times with DMEM F-12 media without serum in a Petri dish (all the MO's were cleaned). Every wash was performed in a new Petri dish.

All the MOs were incubated with 1 ml growth media according to Table 6, in 24 well/plate (SARSTEDT for Suspension Cells) at 5% CO₂ incubator 32° C. for 48 hrs.

TABLE 6
Experimental plan and Sample Numbering

| Group | | |
|---|---|---|
| No. | Treatment | Comments |
| A | HDAd-EF1a-GL2-2 variant ver B | Extraction: Day 1 |
| | Tissue Extraction | |
| B | HDAd-EF1a-GLP-2 variant ver B | Collagenase: Day 1 |
| | Collagenase + Cell Extraction | Extraction: Day 31 |
| C | HDAd-EF1a-opt hEPO | Extraction: Day 1 |
| | Tissue Extraction | |
| D | HDAd-EF1a-opt hEPO | Collagenase: Day 1 |
| | Collagenase + Cell Extraction | Extraction: Day 31 |

Viral transduction was as follows. Certain MO's were transduced with HDAd-EF1a-GLP-2 variant ver B, 7.608*10¹² vp/ml. The vector was diluted in Growth media containing 10% DCS serum to final concentration of 1.5×10¹⁰ vp/GMMO (2.0 μl/GMMO). Another group of MO's were transduced with HdAd-EF1a-opthEPO, 1.66*10¹² vp/ml. The vector was diluted in Growth media containing 10% DCS serum to final concentration of 1.50×10¹⁰ vp/GMMO (9.0 μl/GMMO).

In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

A viral wash was conducted in an open system (24 well/plate). MOs were washed from the transduction medium, and growth medium was added. The 250 μl of transduction medium was removed from the plate with a pipettor, and 2 ml of fresh growth medium was added (first wash). 3 ml of growth medium was added to wells of a new 6 well plate (“maintenance plate”) and the MOs were transferred into the wells from the plate in which the transduction was done (second wash). The 3 ml of media was removed from each well and fresh 3 ml media was added (third wash). And then another 3 washes. The MOs were transferred to a new 24 well plate with fresh 1 ml growth media in each well. The plate was incubated at 32° C., 5% CO₂ for 3 days.

In a maintenance phase, the growth media were changed and collected for analyses by ELISA every Sunday and Wednesday.

Extraction and collagenase treatments were performed on day 11 from transduction. The collagenase treatment (M-per) was performed as follows. GMMOs were transferred into 1 ml of 15 mg/ml collagenase solution (prepared with PBS) in 24 w/p and shaken overnight in 300 rpm. On the next day, collagenase solution was collected with GMMO to Eppendorf and centrifuged at 13000 rpm 10 min 4° C. The supernatant was discarded and the pellet resuspended in 500 μl PBS and centrifuged at 5000 rpm 10 min 4° C. The supernatant was discarded and the pellet resuspended in 200 μl M-per containing protease inhibitor (1:100) kept on ice for 10 min. It was then centrifuged at 5000 rpm 10 min 4° C.; and the supernatant collected and frozen at −80° C.

The tissue protein extraction (T-per) was conducted as follows. On day of experiment termination, medium was collected, GMMO proteins were extracted using T-per (pierce). Growth media was collected from each well to cold Eppendorf tube, then each GMMO was washed with 3 ml saline and all GMMOs transferred to an Eppendorf tube. 200 ul t-per was added to the Eppendorf tube containing protease inhibitor (1:100) and extracted using extraction stick for Eppendorf tubes. Tubes were centrifuged for 10 min in 13000 rpm 4° C. The supernatant was collected and frozen at −80° C.

G. HumanA-273

A study comparing intracellular and extracellular levels of GLP-2 variant was conducted. DME/F-12 medium with 10% DCS was used as follows: HyClone DME/F-12 1:1 (X1)+2.50 mM L-Glutamine+15 mM HEPES Buffer (Thermo Scientific). Medium was supplemented with 10% DCS (HyClone Defined Bovine Calf Serum supplemented, Thermo Scientific); AmBisome 2.5 μg/ml (Liposomal Amphotericin B 50 mg Gilead); Gentamycin sulfate 50 μg/ml (Gentamicin-IKA 80 mg/2 ml-Teva).

The viral vector used was HDdelta28E4-MAR-EF1a-optGLP-2verB-1, 7.608×10¹² vp/ml. The skin used was tummy tuck tissue.

Dermal core MOs (30 mm) were prepared in a sterile hood using the NOUVAG chuck driller; NOUVAG motor set at 7000 rpm chuck driller and double hump Dermavac 3 mm equipment with 14G needles and back vacuum containing 2 ml of saline. The MO's were flashed out from the needles with saline. Needles were replaced every 4-5 harvest. The MO's were incubated for one minute in saline. Then all the MO's were washed 3 times with DMEM F-12 media without serum in a Petri dish (all the MO's were cleaned). Every wash was performed in a new Petri dish.

All the MOs were incubated with 1 ml Growth media with serum, in 24 well/plate (SARSTEDT for Suspension Cells) at 5% CO₂ incubator 32° C. for 24 hrs.

Viral transduction was performed as follows. MO's were transduced with HDAd-EF1a-GLP-2 variant ver B, 7.608*10¹² vp/ml. The vector was diluted in Growth media containing 10% DCS serum to final concentration of 1.5×10¹⁰ vp/GMMO (2.0 μl/GMMO). In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

Viral wash was performed in an open system (24 well/plate). GMMOs were washed from the transduction medium, and growth medium was added. The 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). The GMMOs were transferred to a new 24 well plate with fresh 1 ml growth media in each well. The plate was incubated at 32° C., 5% CO₂ for 3 days.

For maintenance, the growth media was changed and collected for analysis by ELISA every Sunday and Wednesday.

Analyte extraction was performed on day 9 from transduction. On the day of experiment termination, medium was collected and GMMOs were extracted for protein extraction using T-per (Pierce). Growth media was collected from each well to cold Eppendorf tube. Each well was washed with 1000 μl PBS and all GMMOs transferred (with PBS) to the Eppendorf tube (was not washed). 200 μl t-per containing protease inhibitor (1:100) was added and extraction performed using extraction stick for Eppendorf tubes. Tubes were centrifuged for 10 min in 13000 rpm 4° C. The supernatant was collected and frozen at −80° C.

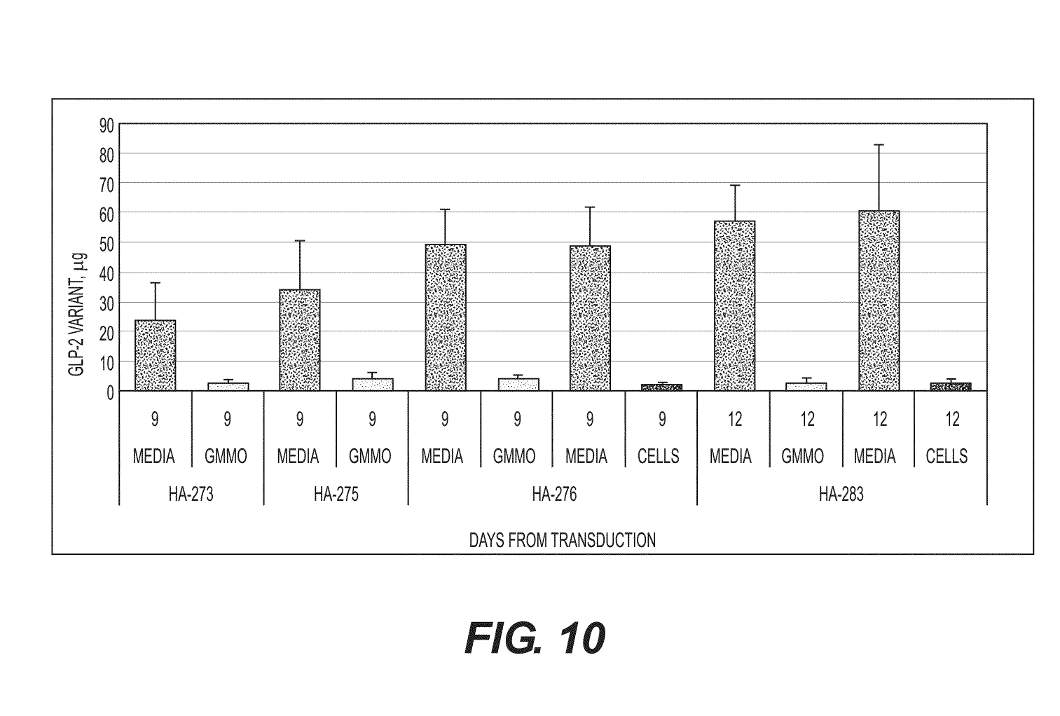

Results are provided in FIG. 10, which is described in detail below.

H. HumanA-275

A study comparing intracellular and extracellular levels of GLP-2 variant was conducted. Materials were as follows. DME/F-12 medium with 10% DCS was HyClone DME/F-12 1:1 (X1)+2.50 mM L-Glutamine+15 mM HEPES Buffer (Thermo Scientific). Medium is supplemented with 10% DCS (HyClone Defined Bovine Calf Serum supplemented, Thermo Scientific); AmBisome 2.5 μg/ml (Liposomal Amphotericin B 50 mg Gilead); Gentamycin sulfate 50 μg/ml (Gentamicin-IKA 80 mg/2 ml-Teva). HDdelta28E4-MAR-EF1a-optGLP-2verB-1, 7.608×10¹² vp/ml was used as the vector. Tummy tuck tissue was employed in this study.

Dermal core MOs (30 mm) were prepared in a sterile hood using the NOUVAG chuck driller; NOUVAG motor set at 7000 rpm chuck driller and double hump Dermavac 3 mm equipment with 14G needles and back vacuum containing 2 ml of saline. The MO's were flushed out from the needles with saline. Needles were replaced every 4-5 harvest. The MO's were incubated for one minute in saline. Then all the MO's were washed 3 times with DMEM F-12 media W/O serum in a Petri dish (all the MO's were cleaned). Every wash was performed in a new Petri dish.

All the MOs were incubated with 1 ml growth media with serum, in 24 well/plate (SARSTEDT for Suspension Cells) at 5% CO₂ incubator 32° C. for 24 hrs.

Viral transduction was performed as follows MO's were transduced with HDAd-EF1a-GLP-2 variant ver B, 7.608*10¹² vp/ml. The vector was diluted in Growth media containing 10% DCS serum to final concentration of 1.5×10¹⁰ vp/GMMO (2.0 μl/GMMO). In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

The viral wash also occurred in an open system (24 well/plate). GMMOs were washed from the transduction medium, and growth medium was added. The 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). The GMMOs were transferred to a new 24 well plate with fresh 1 ml growth media in each well. The plate was incubated at 32° C., 5% CO₂ for 3 days.

For maintenance, the growth media was changed and collected for analysis by ELISA every Sunday and Wednesday.

Analyte extraction occurred on day 9 from transduction. On the day of experiment termination, medium was collected and GMMOs were extracted for protein extraction using T-per (Pierce). Growth media was collected: from each well to cold Eppendorf tube. Each well was washed with 1000 μl PBS and all GMMOs transferred with PBS to the Eppendorf tube (washed 3 times with 3 ml saline). 200 μl t-per was added containing protease inhibitor (1:100) and extract was performed using extraction stick for Eppendorf tubes. Eppendorf tubes were centrifuged for 10 min in 13000 rpm 4° C. The supernatant was collected and frozen at −80° C.

Results are provided in FIG. 10, which is described in detail below.

I. Additional Results

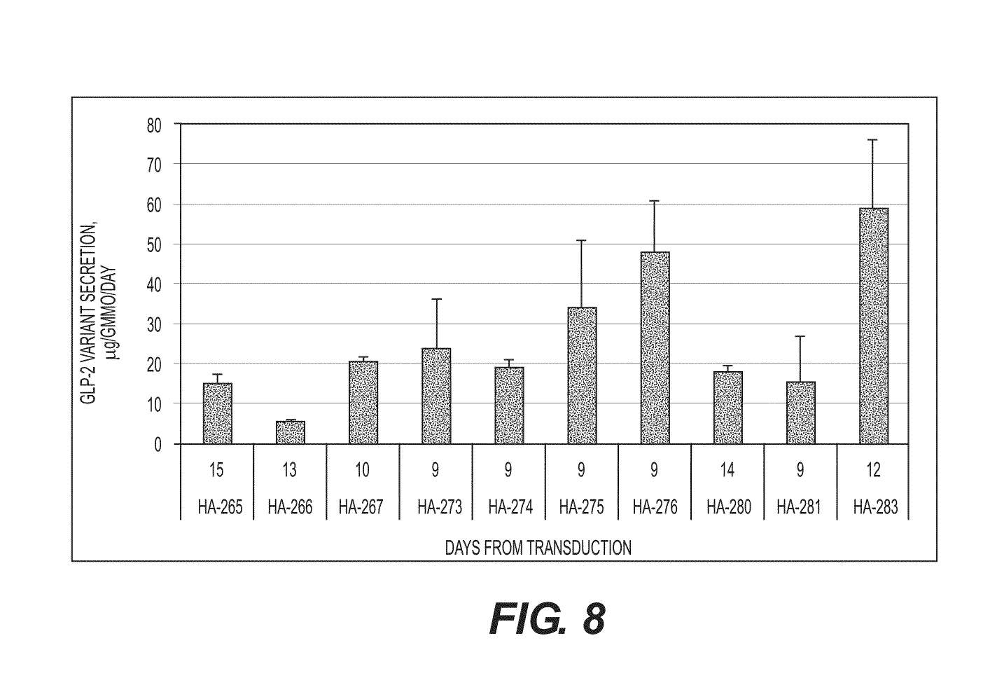

FIG. 8 shows in vitro GMMO skin-to-skin secretion variability on one of days 9-15 (as shown on the X-axis). In vitro GLP-2 variant secretion average of 26 μg/day was measured from GMMOs maintained in serum-containing media. Multiple experiments were conducted to generate the data in this figure, using similar protocols.

FIG. 10 shows the secreted levels of GLP-2 variant compared to the intracellular levels of GLP-2 variant. Results obtained suggest that more than 90% of the GMMO-produced GLP-2 is secreted out of the GMMOs. Multiple experiments were conducted to generate the data in this figure, using similar protocols, as described above.

### Example 5

### Effect of Number of Virus Particles in Transfection

An experiment was performed to evaluate the effect of the number of virus particles in the transduction and its effect on GMMO protein secretion measured in vitro.

In this experiment, DME/F-12 medium with 10% DCS was used as follows: HyClone DME/F-12 1:1 (X1)+2.50 mM L-Glutamine+15 mM HEPES Buffer (Thermo Scientific). Medium was supplemented with 10% DCS (HyClone Defined Bovine Calf Serum supplemented, Thermo Scientific); AmBisome 2.5 μg/ml (Liposomal Amphotericin B 50 mg Gilead); Gentamycin sulfate 50 μg/ml (Gentamicin-IKA 80 mg/2 ml-Teva). The viral vector used was HDdelta28E4-MAR-EF1a-optGLP-2verB-1, 7.608×10¹² vp/ml. The skin used was tummy tuck tissue.

Dermal core MOs (30 mm) were prepared in a sterile hood using the NOUVAG chuck driller; NOUVAG motor set at 7000 rpm chuck driller and double hump Dermavac 3 mm equipment with 14G needles and back vacuum containing 2 ml of saline. The MO's were flushed out from the needles with saline. Needles were replaced every 4-5 harvest. The MO's were incubated for one minute in saline. Then all the MO's were washed 3 times with DMEM F-12 media without serum in a Petri dish (all the MO's were cleaned). Every wash was performed in a new Petri dish.

All the MOs were incubated with 1 ml Growth media with serum, in 24 well/plate (SARSTEDT for Suspension Cells) at 5% CO₂ incubator 32° C. for 24 hrs.

Viral transduction proceeded as follows. MOs were transduced with HDAd-EF1a-GLP-2 variant ver B, 7.608*10¹² vp/ml. The vector was diluted in Growth media containing 10% DCS serum to final concentration of 3.0×10⁹ vp/BP. The vector was diluted 1:4 from the 1.5×10¹⁰ vp/BP concentration (204 ul 1.5×10¹⁰ vp/BP+816 ul growth medium).

In an open system (24 well/plate), 250 μl of transduction medium was added to each well using 1 ml pipettor. The plate was placed on a designated tray and incubated at 32° C., 5% CO₂, for 24 hours, with 150 rpm shaking for the first 4 hours.

The viral wash was conducted in an open system (24 well/plate). GMMOs were washed from the transduction medium, and growth medium was added. The 250 μl of transduction medium was removed from the plate with a pipettor, and 2 ml of fresh growth medium was added (first wash). 3 ml of growth medium was added to wells of a new 6 well plate (“maintenance plate”) and the GMMOs were transferred into the wells from the plate in which the transduction was done (second wash). The 3 ml of media was removed from each well and fresh 3 ml media was added (third wash). The GMMOs were transferred to a new 24 well plate with fresh 1 ml growth media in each well. The plate was incubated at 32° C., 5% CO₂ for 3 days.

For maintenance, the growth media was changed and collected for analysis by ELISA every Sunday and Wednesday.

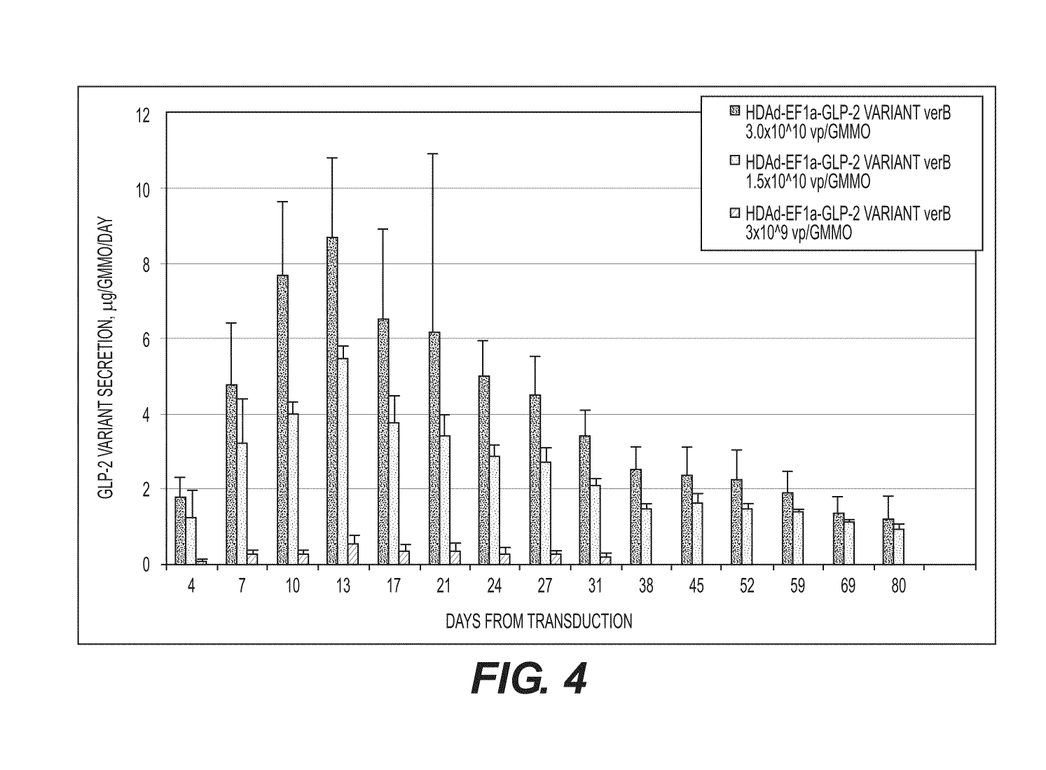

Results are provided in FIG. 4, showing that there is a viral titer effect on GLP-2 amount secreted from GLP-2 transduced GMMOs, where viral titer of 3¹⁰ vp/GMMO gave higher secretion levels mainly at the first 1.5 month.

### Example 6

### Western Blot

GLP-2 samples were analyzed by SDS PAGE (16% precast Tricine gel Product, Life Technologies). Prior to loading on the gel, standard sample, as well as GMMO collection media samples were diluted with reducing sample buffer, and incubated 5 minutes at 95° C. The running conditions were as follows: 100V, 200 minutes in cooled Tricine buffer (Life Technologies). Molecular weight size marker used was: 7 μl of pre-stained Ultra-low Range Molecular Weight Marker (Sigma) or See Blue pre-stained protein standard (Life Technologies). Following electrophoresis, the proteins separated in the gel were electroblotted to a 0.2 μm nitrocellulose membrane (Life Technologies) using wet transfer system (Life Technologies). Blotting conditions used were adequate to small proteins, as follows: 200 mA for 20 minutes. Next, the blotted membrane was blocked with PBS 0.2% Tween (PBST) 10% skim milk solution for overnight at 4° C., followed by incubation with Rabbit anti GLP-2 antibody (Life Span Bioscience) at concentration of 3 μg/ml diluted in PBST 1% milk for 4 hours, at room temperature. After three washes each of 5 minutes with PBST, HRP conugated Goat anti-Rabbit antibody (Jackson Immuno Research) diluted 5000 fold in PBST 1% milk was applied for 1 hour incubation at room temperature. Detection was carried out using chemiluminescent substrate (Thermo Scientific). Imaging was conducted by LAS 500 chemiluminescent imager (General Electric).

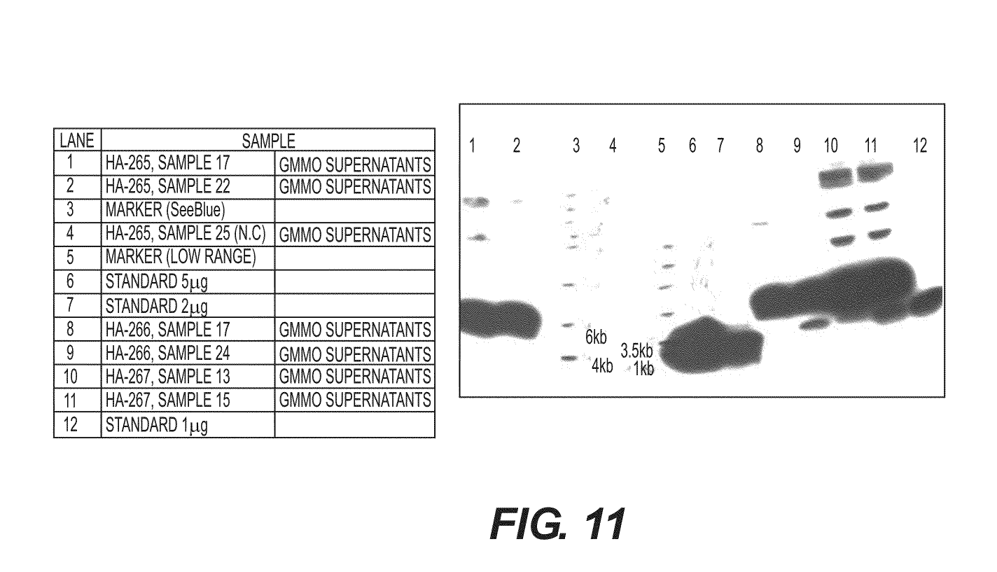

FIG. 11 provides GLP-2 variant western blot analysis confirming the presence of GLP-2 in three skin samples.

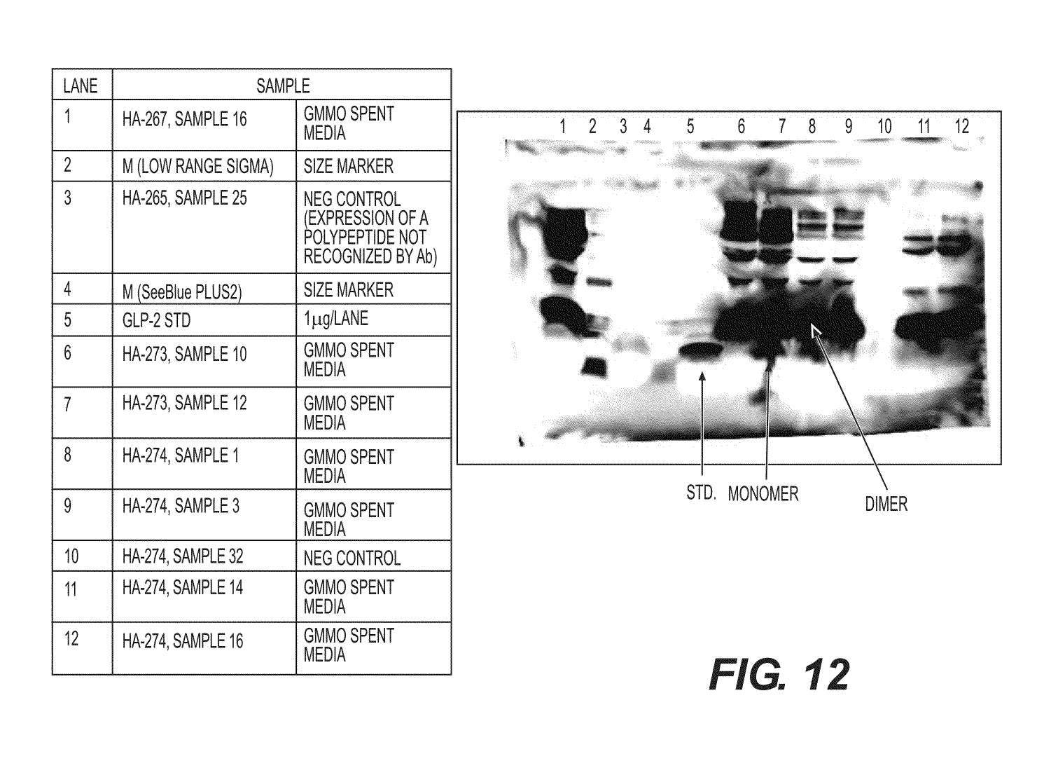

FIG. 12 provides GLP-2 variant western blot analysis.

Both GLP-2 monomer and dimer were recognized according to their corresponding size using western blot analysis with GLP-2 specific antibodies.

### Example 7

### Experimental Conclusions

Several GLP-2 variant expression cassettes were designed, tested, and one was selected for further studies with the GMMO. GLP-2 variant secretion levels in vitro were in the μg per day range. The GMMO-secreted GLP-2 variant was verified by ELISA specific antibodies and by western blot.

Most of the GLP-2 variant produced by the GMMO is secreted out of the GMMO and is stable for several days in the spent media under the experimental conditions. GLP-2 variant GMMOs can also be maintained in serum-free medium.

Initial in vivo SCID mice studies suggest that GMMO-secreted GLP-2 can reach the mice blood stream post-implantation. There is evidence that part of the GLP-2 dimer is digested into its two monomers. Finally, transfection studies suggest that fibroblast cells may secrete similar levels of GLP-2 variant and GLP-2 wild type and that both have similar stability in spent media.

### Example 8

### GMMO GLP-2 Increases Intestinal Villi Length and Intestinal Crypt Cell Proliferation Rate

### Preparation and Implantation of GMMO GLP-2 to SCID Mice and Rats

MOs were harvested from human and rat skin according to standard procedures described herein. The MOs were transduced with HDAd-EF1a-GLP-2 variant ver B vector and processed into GLP-2 secreting GMMOs as described herein. Human GMMO GLP-2 and rat GMMO GLP-2 were implanted in SCID mice and rats, respectively.

Small intestines of mice were weighed without prior wash, taking in consideration that distribution of leftover pieces inside the intestine is similar among the groups. The intestinal segments were cut into 2.5 cm length segments, which were immediately placed in 2 ml formalin for fixation. The morphology of the segments were analyzed as follows:

A. Jejunum

15 cm distal to the end of the stomach were measured (called pylorus) and then 2 segments, 2.5 cm in length each, were cut. The 1^{st} segment was called “proximal segment of the jejunum”, and 2^{nd} segment was called “distal segment of the jejunum.”

Ileum

4 cm distal to the end of the cecum were measured, and then 2 segments, 2.5 cm in length each, were cut. The 1^{st} segment was called “proximal segment of the ileum”, and 2^{nd} segment was called “distal segment of the ileum.”

Each 2.5 cm segment (distal and proximal jejunum, distal and proximal ileum) was cut into five segments of 2.5 mm each, and each of the 5 segments from each of the four sections (distal jejunum, proximal jejunum, distal ileum, proximal ileum) were imbedded in paraffin and the blocks were cut in 5 micrometers thickness to prepare pathology slides. The slides were stained with H&E and were analyzed by a pathologist to determine the length of the villus and crypt. Slides were also stained with Ki67 and were analyzed by a pathologist to determine both villus length (morphology) and crypt cell proliferation (stained with Ki67).

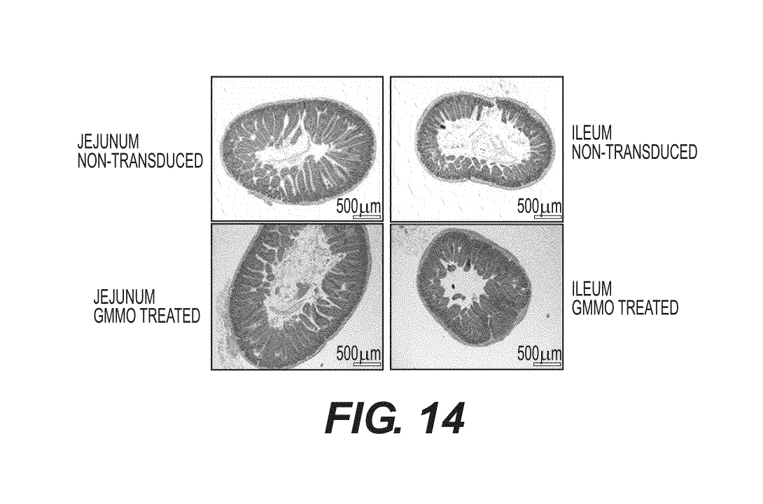

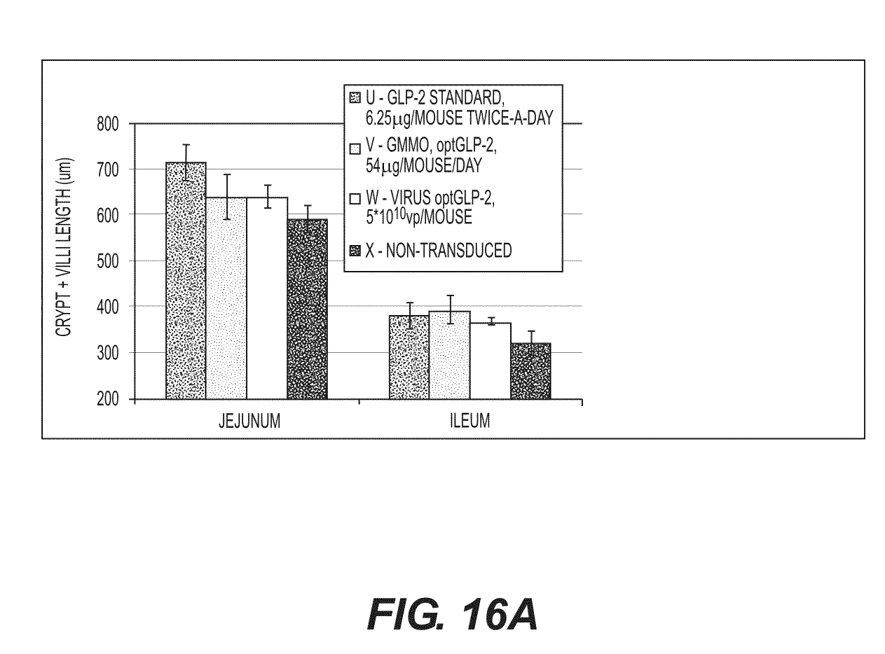

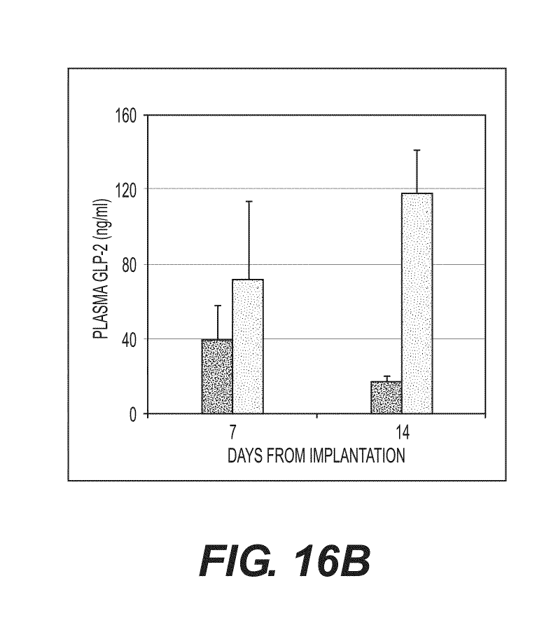

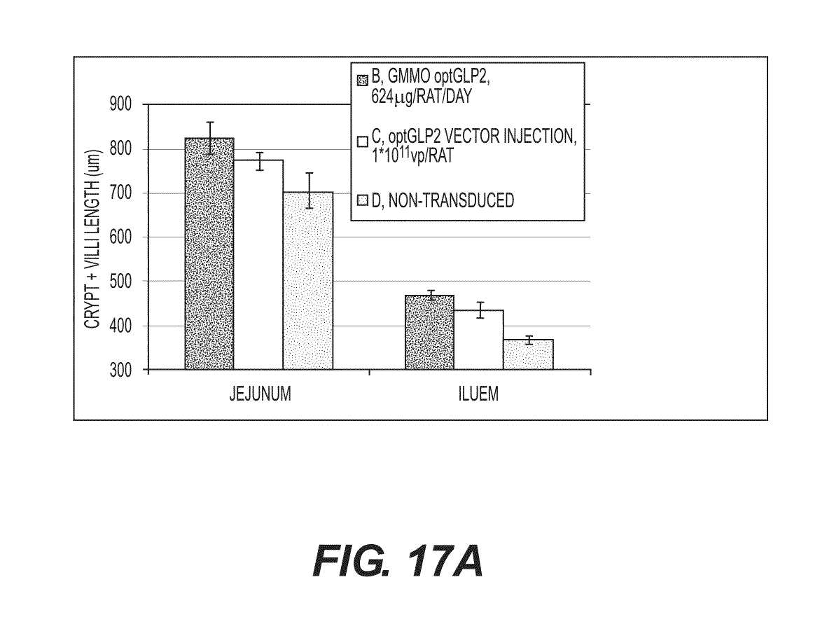

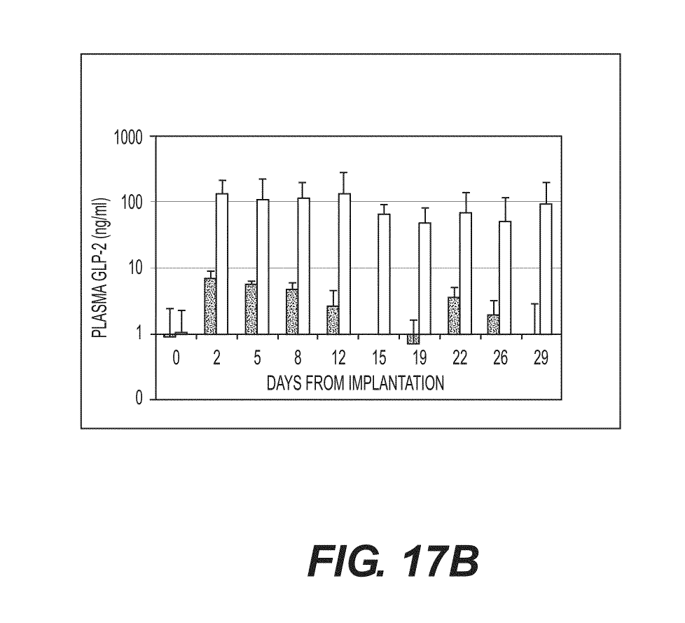

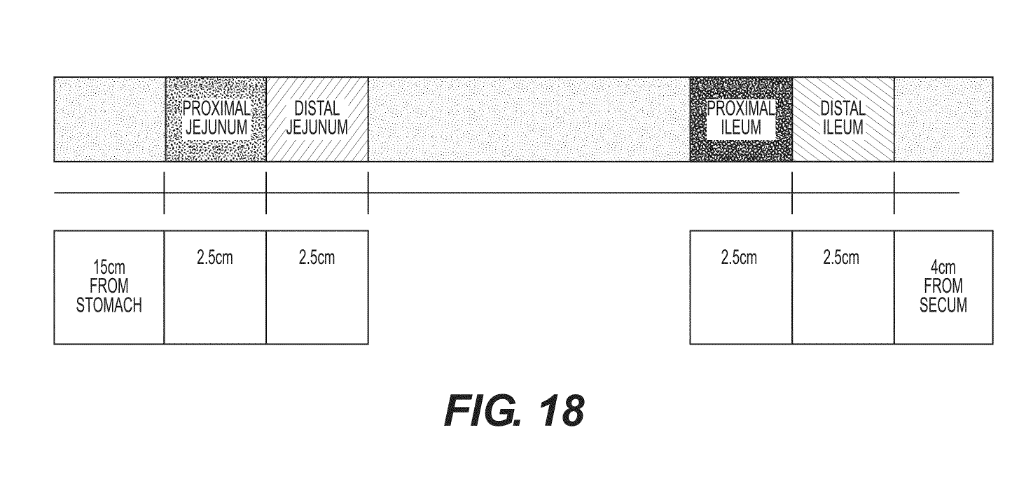

As evidenced in FIGS. 14, 16, and 17, GMMO GLP-2 is capable of increasing villi length in-vivo in jejunum and ileum in SCID mice, as compared to control samples that were not transduced with GMMO GLP-2.

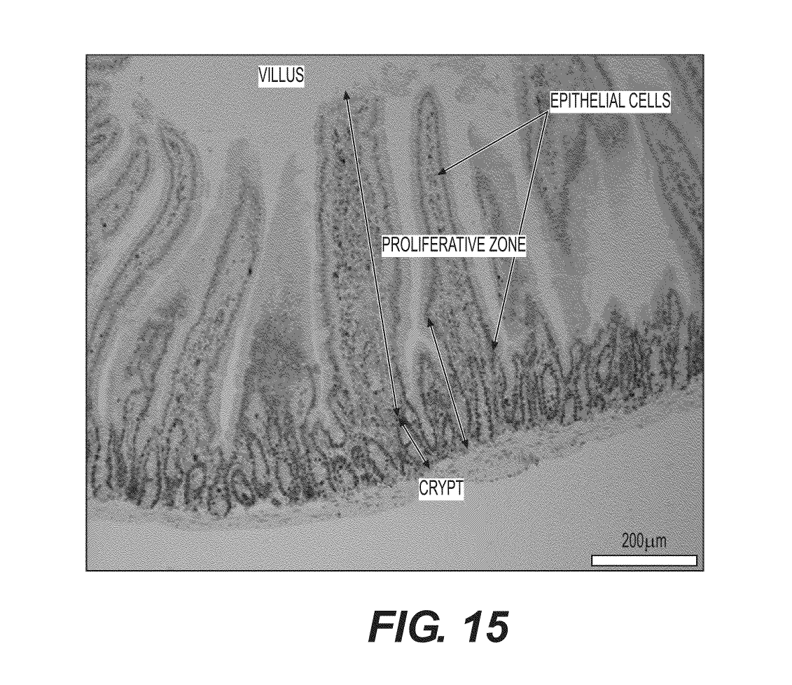

Ki67 is a known marker of proliferating cells. FIG. 15 shows GMMO GLP-2 successfully triggers crypt cell proliferation.

### Example 9

### Western Blot Analysis of GLP-2 from GMMO Collection Media

The presence of GLP-2 from collection media was evaluated by western blot analysis for three GMMOs prepared as described in Example 4. The MOs were transduced with HDAd-EF1a-GLP-2 variant ver B vector and processed into GLP-2 secreting GMMOs as described herein. GMMO collection media samples were lyophilized, reconstituted with urea 8M, and then diluted 3-fold to final urea concentration of 2.66M. Prior to loading on the gel (16% precast Tricine gel Product # EC66952BOX, Life Technologies), standard, negative control, and GMMO samples were diluted with reducing sample buffer and incubated for five minutes at 95° C.

The standard (std) was dimer and monomer GLP-2. The negative control (NC) was DMMO collection media. Samples were taken from three separate GMMOs. Skin 1 is from a first skin donor (HA325), Skin 2 is from a second skin donor (HA334), and Skin 3 is from a third skin donor (HA365).

The running conditions for the SDS PAGE gel were 100V, 200 minutes in cooled Tricine buffer (Product# LC1675, Life Technologies). Molecular weight size was determined using marker (7 μl of pre-stained Ultra-low Range Molecular Weight Marker [Product#1610377, Bio-Rad]). Following electrophoresis, the proteins separated in the gel were electroblotted to a 0.1 um nitrocellulose membrane (Product #10600000, GE) using wet transfer system (Product # EI9051 Life Technologies). Transfer conditions (100 mA for 35 minutes) were optimized for small proteins. Next, the blotted membrane was blocked with PBS 0.2% Tween (PBST)/10% skim milk solution overnight at 4° C., followed by incubation with rabbit anti-GLP-2 antibody (Product # LSC105747, Life Span Bioscience) at a concentration of 3 μg/ml diluted in PBST/1% milk. The blocking period was either 4 hours or overnight at room temperature. After three 5-minute washes with PBST, the blotted membrane was incubated with HRP-conugated goat anti-rabbit antibody (Product #111-035-144, Jackson Immuno Research) at 1:5000 dilution in PBST/1% milk for 1 hour at room temperature. Detection was carried out following standard procedures using chemiluminescent substrate (Product # PIR-34095, Thermo Scientific). Imaging was conducted by Amersham Imager 600 chemiluminescent imager (Product#29-0834-61General Electric).

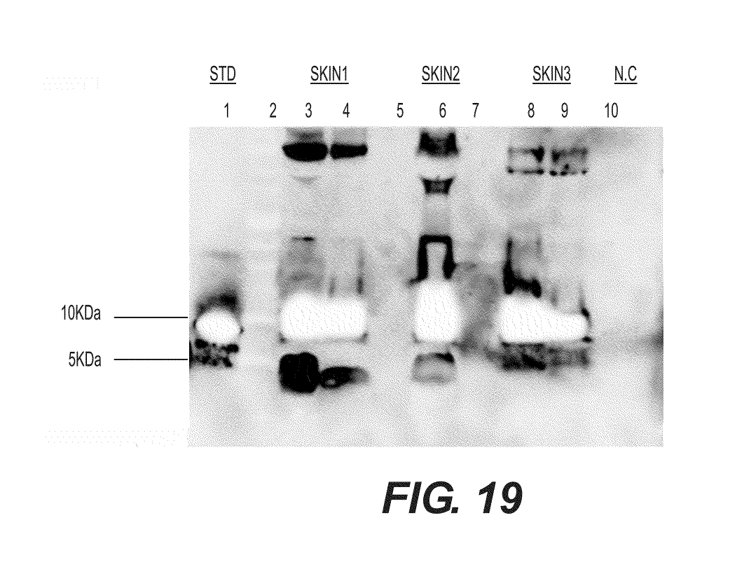

Western blot results are presented in FIG. 19. The lanes of the immunoblot are as follows: (1) standard of dimer and monomer GLP-2; (2) Marker (Dual xtra, Bio-Rad); (3) HA325 sample 1 (untreated with urea); (4) HA325 sample 2; (5) empty lane; (6) HA334 sample 1; (7) empty lane; (8) HA365 sample 1; (9) HA365 sample 2; (10) spent media from DMMO expressing EPO (Negative control). These data indicate that GLP-2 GMMOs can produce GLP-2 dimer, which is further processed intracellularly to active monomer form.

### Example 10

### In Vitro Activity of GLP-2 from GMMO Collection Media

The ability of GMMO secreted GLP-2 from collected from spent media to induce cAMP production was tested. Collection media was tested from GMMO-GLP-2 (transduced with HDAd-EF1α-GLP-2 using a vector comprising SEQ ID No: 5) and GMMO-GLP-2 Variant (transduced with HDAd-EF1α-GLP-2 variant ver B, SEQ ID No: 21).

HEK293 cells transfected with the GLP-2 Receptor (Product#CSC-RG0852, Genescript) were cultured in complete DMEM (Product#01-055-1A, Biological Industries) supplemented with 2 mM L-Glutamine (Product#03-020-1A, Biological Industries), 10% Fetal Bovine serum (Product#04-127-1A, Biological Industries), 2.5 ug/ml Amphoterycin B (Product#03-028-1B, Biological Industries) and 50 μg/ml Gentamycin sulfate (Product# Gentamicin-IKA, Teva).

The activity assay was performed using the cAMP ELISA kit (Product#ADI-901-163, ENZO). Two days prior to activity experiment (Day 0) cells were seeded in 24 well plate at a density of 100,000 cells per well. On Day 2, media was withdrawn, and the cells are incubated for 2 hours in 37° C. with GLP-2 dimer standard or GMMO collection media at volume of 200 μl/well. Following incubation with sample, cells were lysed by addition of Triton 0.1% according to kit protocol. cAMP concentrations were determined using the protocol provided with the kit.

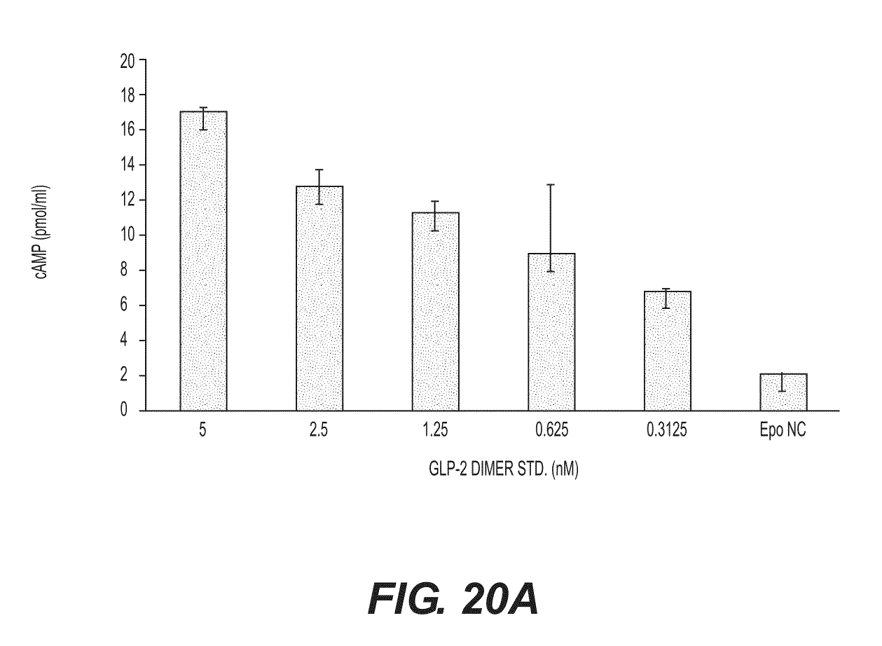

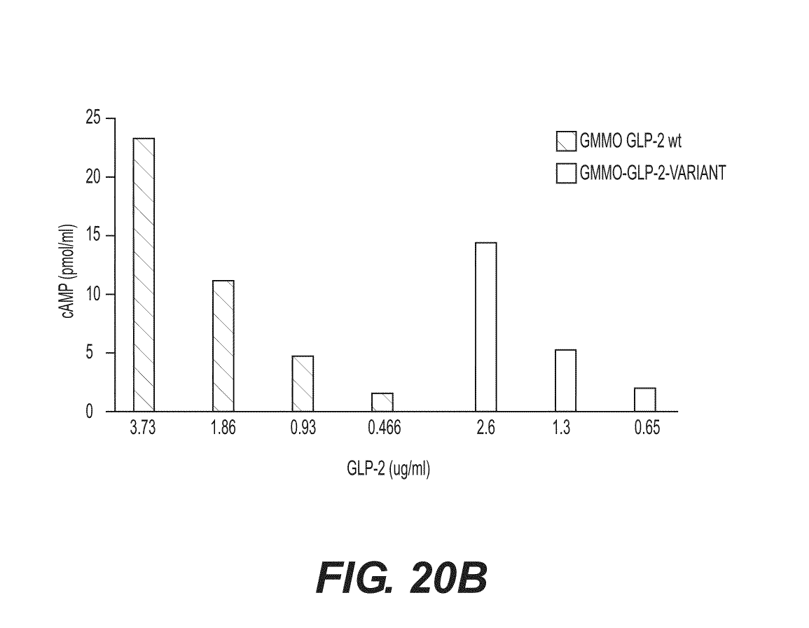

FIG. 20A shows that cAMP production was induced in GLP-2 receptor-expressing HEK293 cells by a range of doses (0.3125-5 nM) of GLP-2 dimer standard. A negative control (NC, collection media from an GMMO transduced to express EPO) stimulated negligible cAMP production. These results indicate that this in vitro assay is a sensitive measure of functional activity of GLP-2.

The in vitro efficacy of collection media from GMMOs expressing either wildtype GLP-2 (GLP-2 wt, SEQ ID No: 1) or GLP-2 Variant (SEQ ID No: 3, i.e., point mutation in second amino acid) were compared. GLP-2 Variant is a variant of GLP-2 with a point mutation from Ala to Gly at the second amino acid in the sequence compared with the wildtype sequence. The sequence of the GLP-2 wild type amino acid sequence in Approach B construct is SEQ ID No: 4. The sequence of the GLP-2 variant amino acid sequence in Approach B construct is SEQ ID No: 6. Nucleofection of human dermal fibroblasts with either construct indicated that produced secretion of similar levels of GLP-2 at 24 and 48 hours (data now shown). These constructs were then used following the methods outlined in Example 4 to generate GMMOs.

Collection media from GMMOs was diluted and tested by GLP-2 ELISA so that a known amount of GLP-2 was present in the media. FIG. 20B shows results of stimulation of GLP-2 receptor-expressing HEK293 cells with collection media from GMMOs expressing GLP-2 wt (GMMO-GLP-2 wt) or GLP-2 Variant (GMMO-GLP-2 Variant). These results indicate that collection media from both GMMO-GLP-2 wt and GMMO-GLP-2 Variant contain functionally active GLP-2.

### Example 11

### In Vitro Activity of GMMO-oxyntomodulins

An expression cassette was designed to assess the ability of GMMOs to express oxyntomodulin. This cassette had a proglucagon signaling peptide (SEQ ID No: 9) and three oxyntomodulin sequences (SEQ ID No: 22) separated by two cleavable linkers corresponding to PCK1/2 (SEQ ID No: 14) (see “Approach-A” of FIG. 35). The PCK1/2 linker is the IP-2 linker with a RH cleavage site added. This cassette was inserted into the HD-Ad-EF1α viral vector (SEQ ID NO: 58). GMMOs were prepared as described in Example 4 using this viral vector for production of oxyntomodulin (i.e., GMMO-oxyntomodulins). In this application, “GMMO-oxyntomodulin” refers to a GMMO generated by the methods of Example 4, wherein the cassette has a glucagon signaling peptide, and three oxyntomodulin sequences separated by two cleavable linkers corresponding to PCK1/2. In some experiments, the viral vector used to produce GMMO-oxyntomodulin is referred to as the HD-Ad-EF1α-Oxy-1 vector.

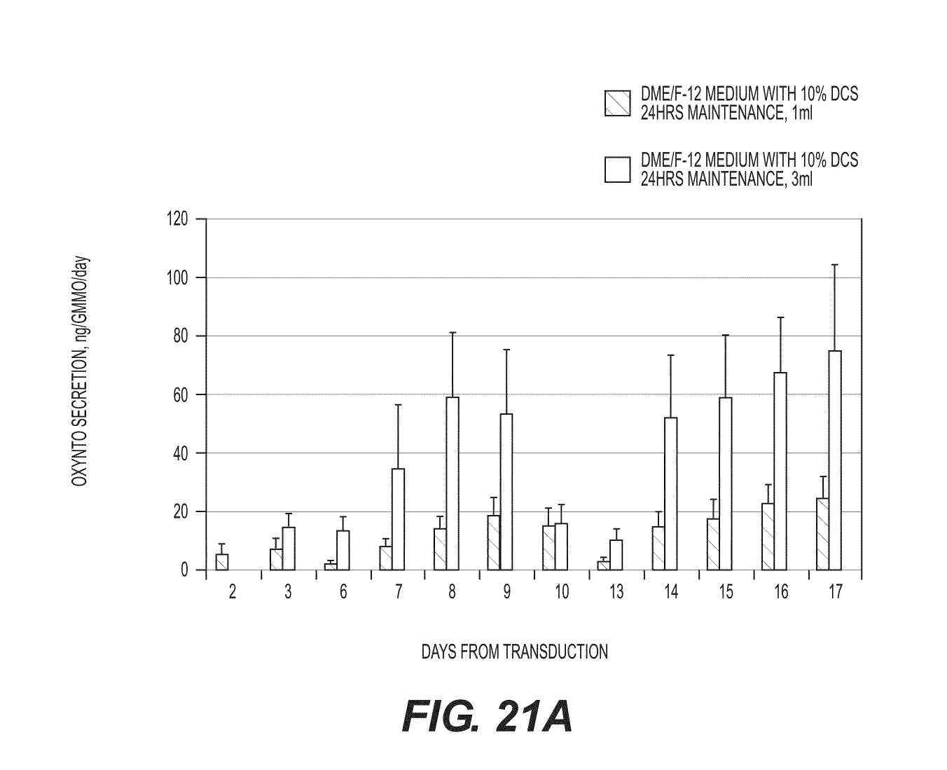

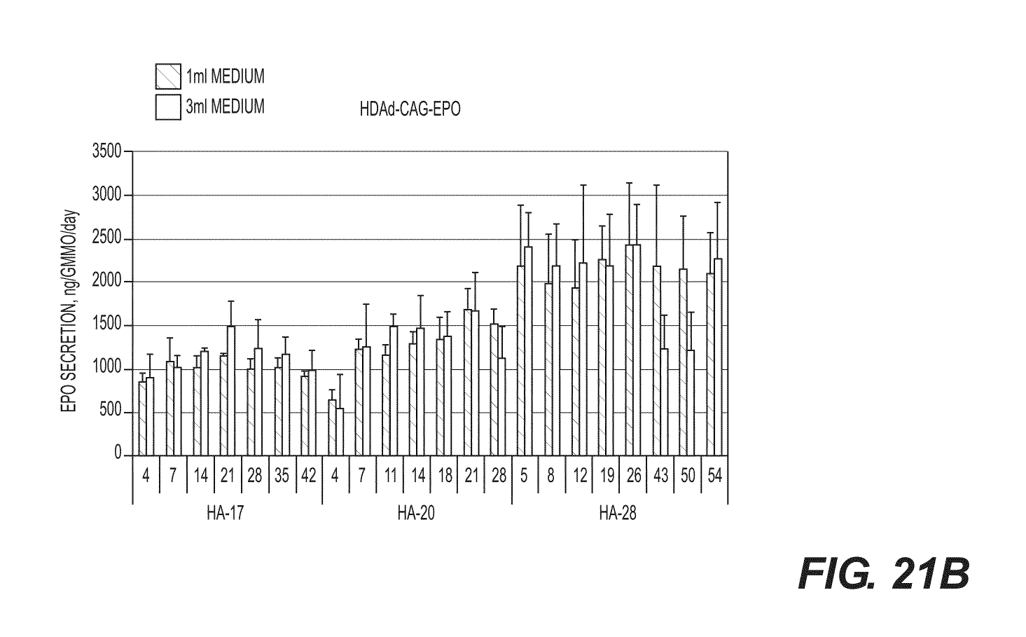

Conditions that produced the greatest secretion of oxyntomodulin were investigated. As shown in FIG. 21A, maintenance of GMMO-oxyntomodulins in 3 ml of DME/F-12 media with 10% dialyzed calf serum (DCS) led to greater secretion of oxyntomodulin compared with maintenance in 1 ml. Oxyntomodulin levels were assessed in all instances by an ELISA described below. As shown in FIG. 21B, control experiments indicated that secretion of EPO from GMMOs was not affected by media volume. Thus, secretion of oxyntomodulin from GMMOs has a novel regulation by media volume.

Oxyntomodulin Measurement System

For In-Vitro Assays:

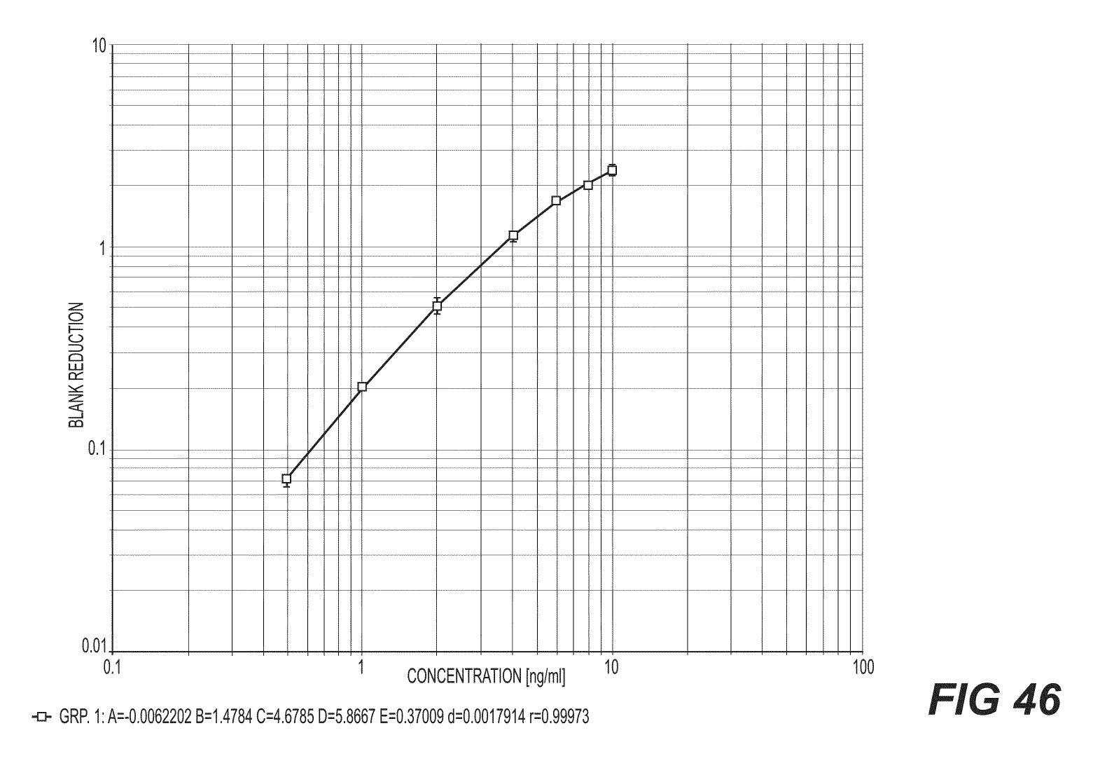

A commercial sandwich assay Glucagon DuoSet ELISA from R&D Systems was identified and found to be suitable for measuring levels of Oxyntomodulin, on the basis of ˜20% crossreactivity with oxyntomodulin, using oxyntomodulin as a reference standard. The detection limit is 0.5 ng/ml and according to the manufacturer it can be used with cell culture supernate samples. The commercially provided instructions for this ELISA assay may be followed to detect levels of Oxyntomodulin in in-vitro samples. See calibration curve at FIG. 46.

For In-Vivo Assays:

A commercial sandwich assay Glucagon Quantikine ELISA from R&D Systems was identified and found to be suitable for measuring levels of Oxyntomodulin, on the basis of ˜30% cross-reactivity with oxyntomodulin. The minimal detectable dose is 6.37 pg glucagon/ml and according to the manufacturer it can be used with cell culture supernatant samples, serum, and plasma. In the absence of specific Oxyntomodulin ELISA assay, this assay was used to estimate in-vivo GMMO secreted Oxyntomodulin. The commercially provided instructions for this ELISA assay may be followed to detect levels of Oxyntomodulin from GMMO source in in-vivo samples.

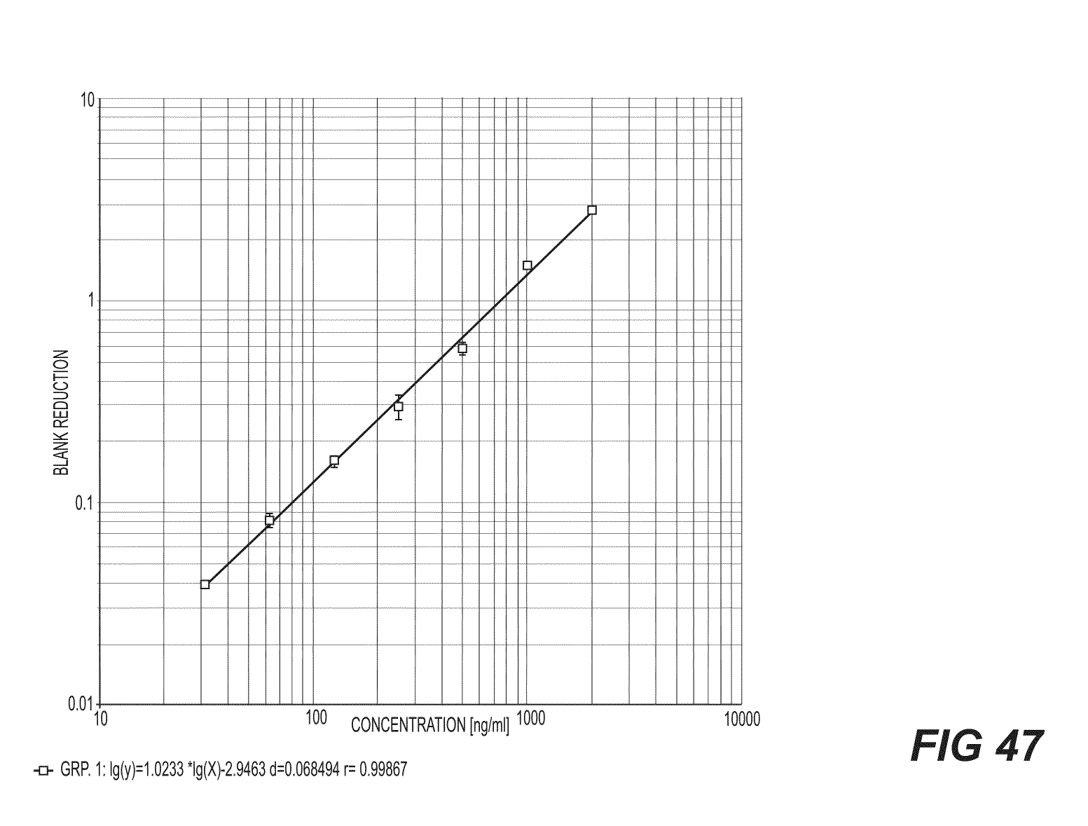

A representative calibration curve is provided in FIG. 47.

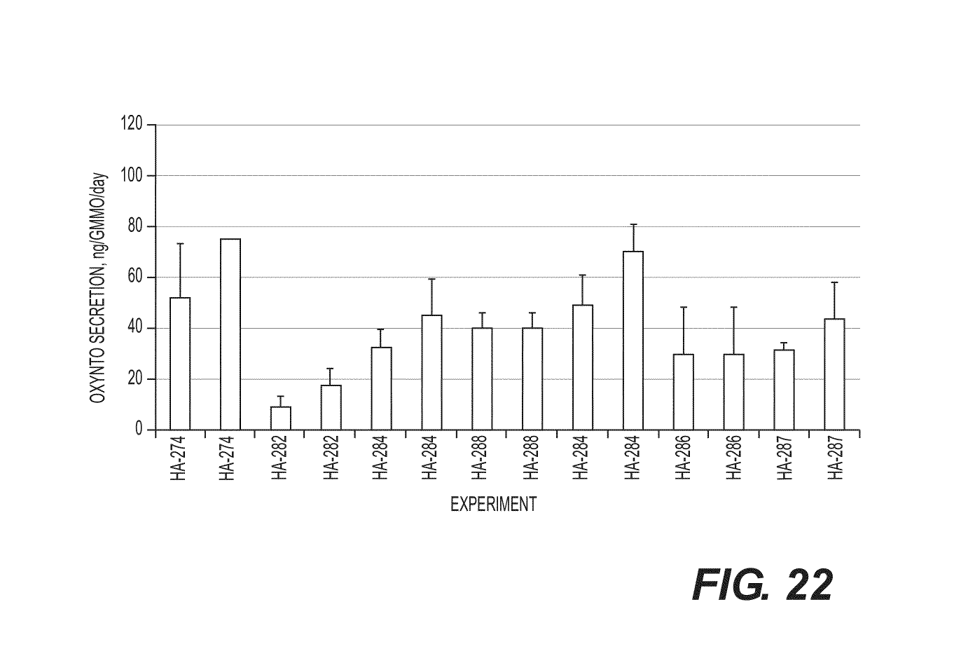

Next, in vitro skin-to-skin variability of GMMO-oxyntomodulins was assessed. Six different GMMO-oxyntomodulins (HA-274, HA-282, HA-284, HA-288, HA-286, and HA-287) were maintained for 14-16 days in 3 ml of serum-containing media, and media was then assessed for secretion of oxyntomodulin. Additionally, multiple samples from the same GMMOs were assessed to measure variability of measurements within an individual sample. The results shown in FIG. 22 indicate that GMMOs secreting oxyntomodulin could be reproducibly generated with an average secretion of about 40.5 ng/day.

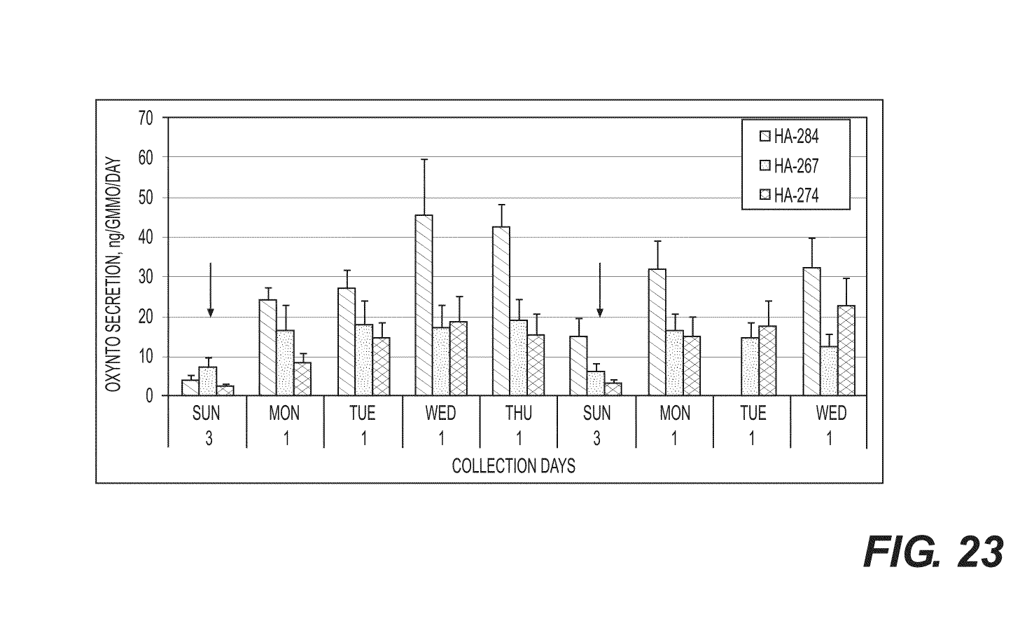

The effect of media exchange frequency was also assessed for oxyntomodulin secretion from GMMO. As shown in FIG. 23, oxyntomodulin concentration was measured in the spent media from three GMMO-oxyntomodulins (HA-284, HA-267, and HA-274). Media was exchanged either daily (indicated with “1”) or every third day (indicated with “3”). Media exchange every three days led to substantially lower secretion of oxyntomodulin from GMMOs.

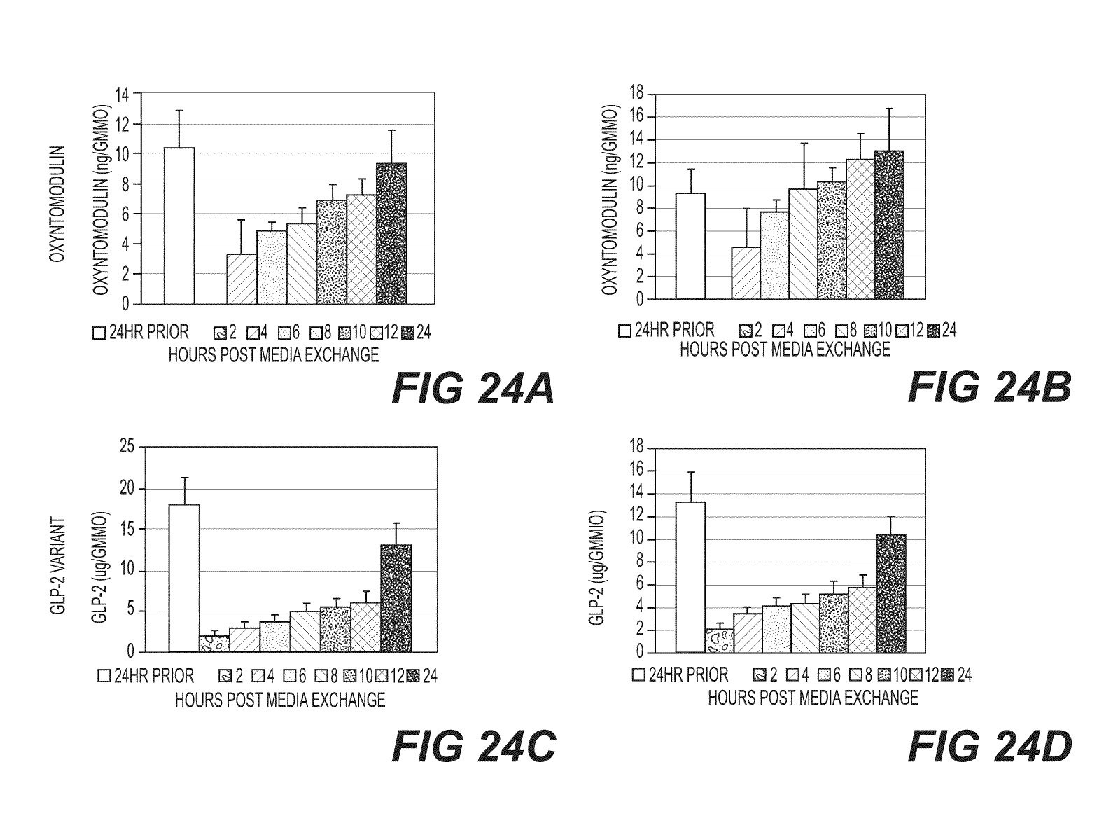

The time course of secretion of either oxyntomodulin or GLP-2 Variant was examined following media exchange (description of GMMO-GLP-2 Variant provided in Example 8). FIGS. 24A and 24B show that the highest levels of secretion of oxyntomodulin were seen in the first 12 hours following media exchange with a relatively lower increase in secretion seen in 24-hour sample as compared with the 12-hour sample. In contrast, FIGS. 24C and 24D indicates that there is significantly more GLP-2 secreted from GMMO-GLP-2 Variant in the 24-hour sample as compared with the 12-hour sample. Thus, the highest levels of oxyntomodulin secretion from GMMO-oxyntomodulin appears to be in the period soon after media exchange, but this is not true for GMMO-GLP-2 Variant.

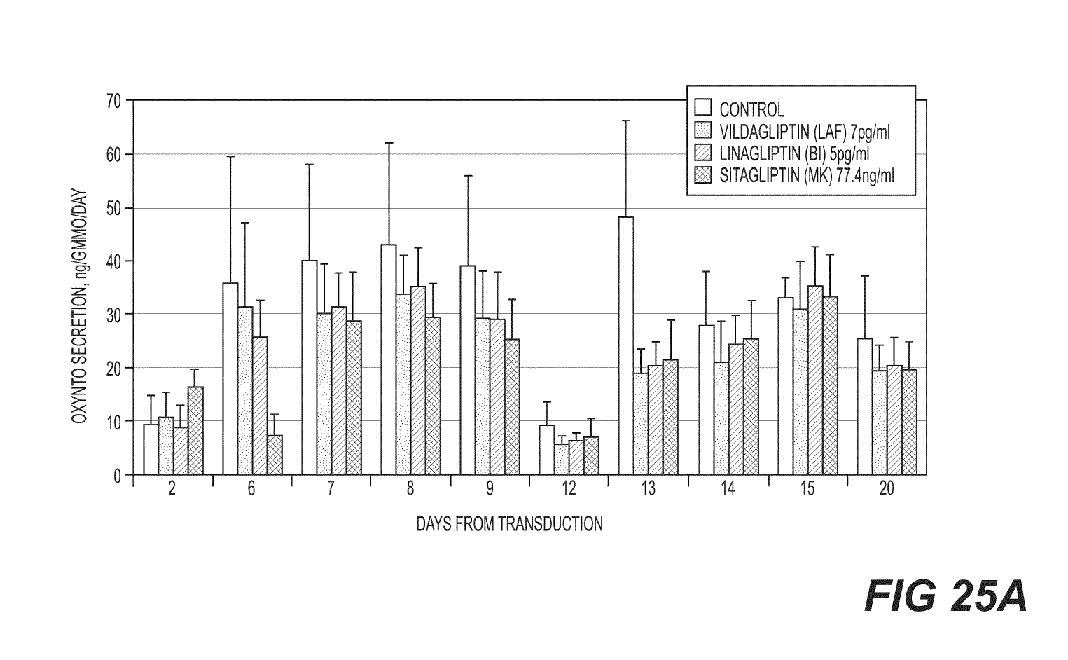

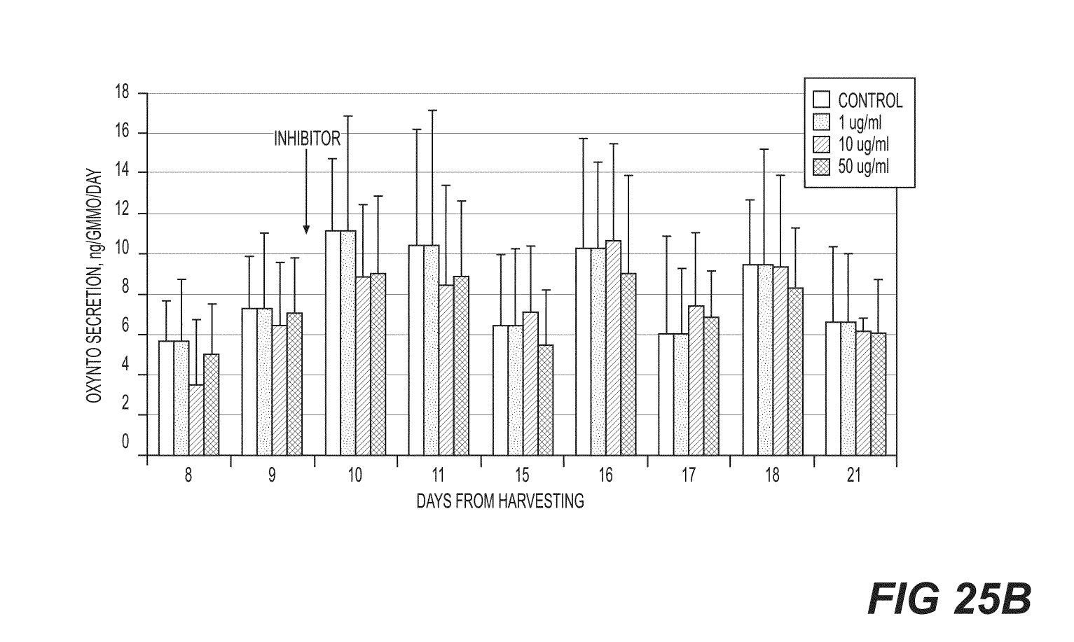

Oxyntomodulin is a substrate for the enzyme dipeptidyl peptidase IV (DPP-IV). Therefore, the effect of DPP-IV inhibitors on levels of oxyntomodulin in spent media from GMMO-oxyntomodulins was assessed. As shown in FIG. 25A, there was no effect of addition of the DPP-IV inhibitors vildagliptin (7 pg/ml), linagliptin (5 pg/ml), or sitagliptin (77.4 ng/ml) to the production media of GMMO-oxyntomodulins over a 20-day period following transduction. In addition, there was no effect of different concentrations of another DPP-IV inhibitor, diprotin A (1 μg/ml-50 g/ml), in the production media on the secretion of oxyntomodulin over a 21-day period following harvesting as shown in FIG. 25B. Vildagliptin (LAF237), 10 mg, Cat#2188-10, BioVision, USA; Linagliptin (BI-2240), 50 mg, Cat#2240-50, BioVision, USA; Sitagliptin Phosphate Monohydrate (MK-0431), 100 mg, Cat#1757-100, BioVision, USA; Diprotin A, 5 mg, Cat#I9759, Sigma, USA.

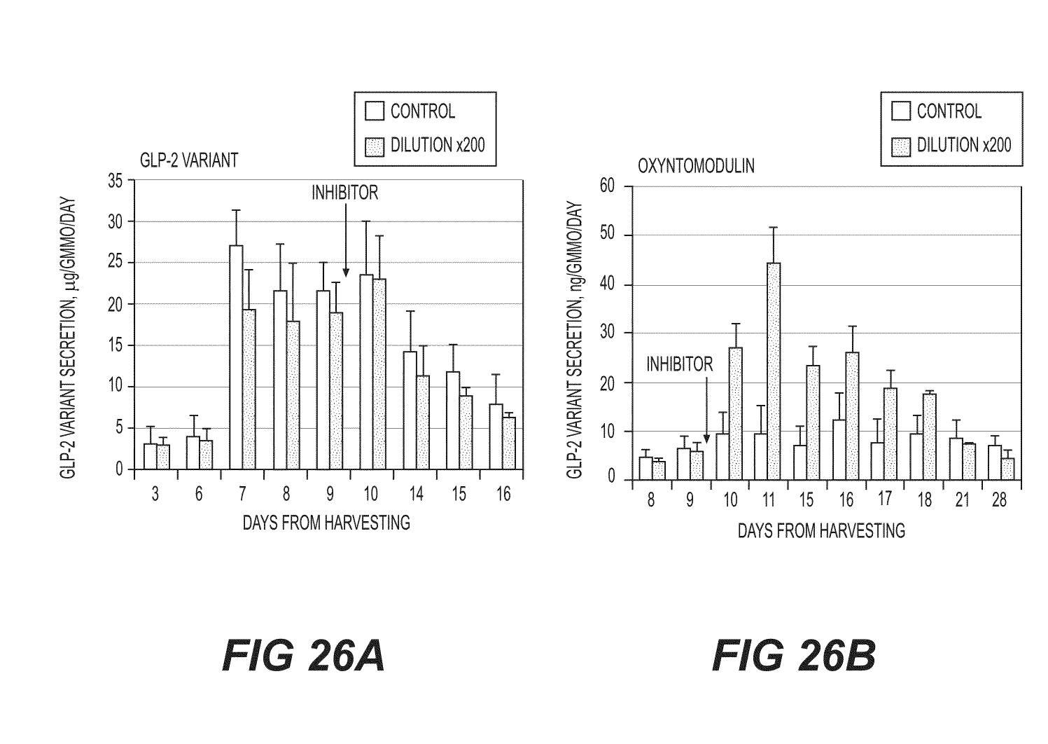

The effect of addition of a protease inhibitor to the production media of GMMOs was then assessed. For this experiment, Protease Inhibitor Cocktail, 1 ml, Cat#P1860, Sigma, USA was used. FIG. 26A shows that addition of protease inhibitor (marked with red arrow) at a 200-fold dilution did not alter the secretion of GLP-2 from GMMO-GLP-2 Variant. In contrast, secretion of oxyntomodulin from GMMO-oxyntomodulins was increased with addition of protease inhibitor versus control as shown in FIG. 26B. These data suggest that the oxyntomodulin secreted may be unstable in spent media.

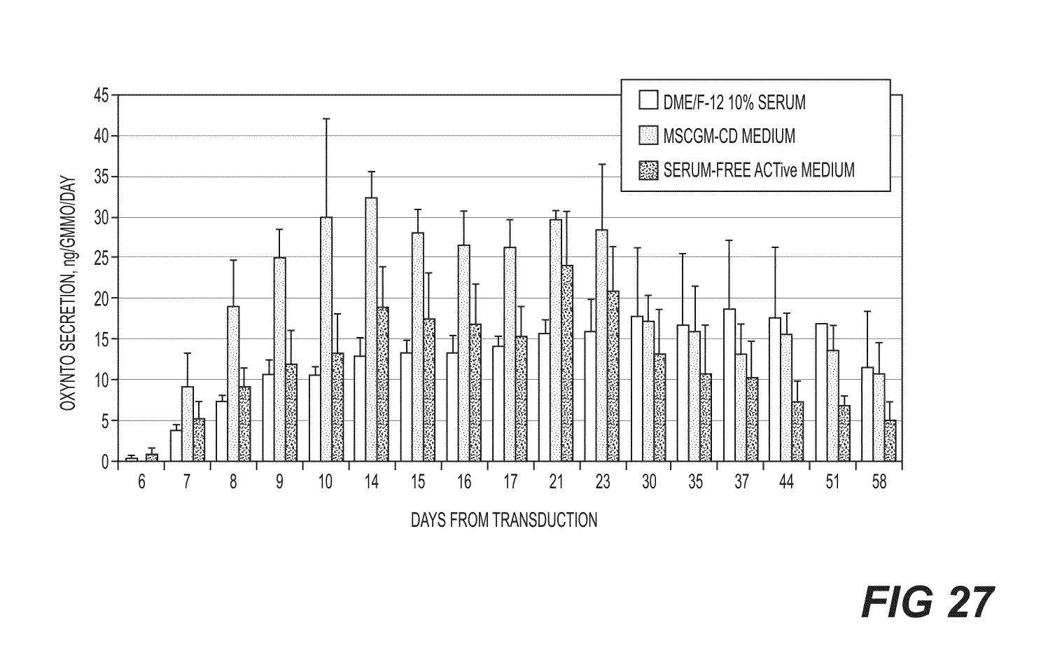

The effect of different media on oxyntomodulin secretion from GMMO-oxyntomodulins was next assessed. GMMO-oxyntomodulins were maintained in DME/F-12 medium with 10% serum, MSCGM-CD medium, or serum-free ACTive medium for 58 days after transduction. TheraPEAK™ MSCGM-CD™ Mesenchymal Stem Cell Medium, Lonza]; Serum-free ACTive Medium, CellGro/CellGenix. Results shown in FIG. 27 indicate that the highest levels of oxyntomodulin secretion was seen for GMMOs maintained in MSCGM-CD medium.

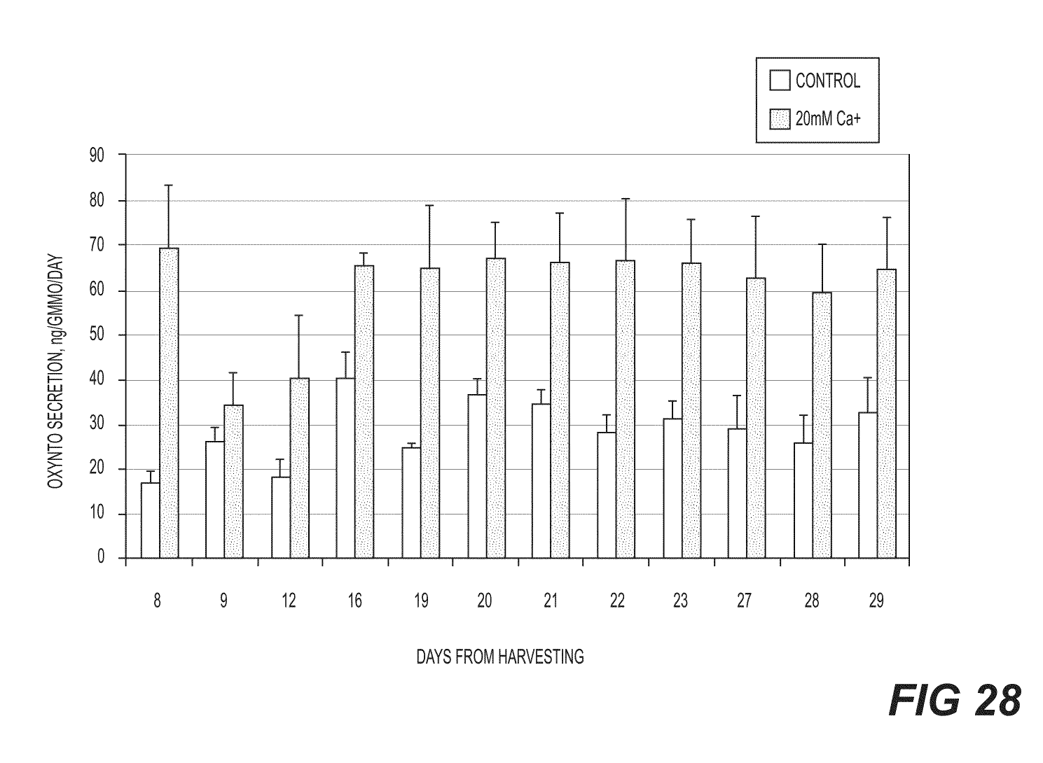

As it is known that calcium levels are a primary regulator of the GLP-1 secretion signaling pathway (see Lim, G. E. and Brubaker, P. L., Diabetes 55:S70-S77 (2006)), the effect of calcium levels on secretion of oxyntomodulin by GMMOs was assessed. As shown in FIG. 28, an increase to 20 mM Ca²⁺ by addition of CaCl₂ to production media increased secretion of oxyntomodulin from GMMOs by approximately 2-fold over a 29-day period after harvesting.

These in vitro data indicate that GMMO-oxyntomodulins are a robust means of expressing oxyntomodulin.

### Example 12

### In Vivo Activity of GMMO-oxyntomodulins

Based on the robust in vitro profile of GMMO-oxyntomodulins, their efficacy in vivo was assessed. A model with SCID mice to assess the performance of GMMO-oxyntomodulins was evaluated following the procedures outlined in Example 4.

The first in vivo experiment of the efficacy of GMMO-oxyntomodulins involved implantation of 1 ng/mouse of GMMO-oxyntomodulin into SCID mice. The experimental groups were as follow:
Active without DepoMedrol (900 ng/day)=Oxynto Active without Depo: GMMO oxyntomodulin, Serum-free ACTive production Medium, mice were not injected with DepoMedrol
Active with DepoMedrol (950 ng/day)=Oxynto Active+Depo: GMMO oxyntomodulin, Serum-free ACTive production Medium, mice were injected with DepoMedrol
F-12 full with DepoMedrol (1280 ng/day)=Oxynto F-12 full+Depo: GMMO EPO, DME/F-12 medium with 10% serum mice were injected with DepoMedrol
MO Active with DepoMedrol=MO Active+Depo: MO non transduced, Serum-free ACTive production Medium, mice were injected with DepoMedrol
MO Active without Depomedrol=MO Active without Depo: MO non transduced, Serum-free ACTive production Medium, mice were not injected with DepoMedrol

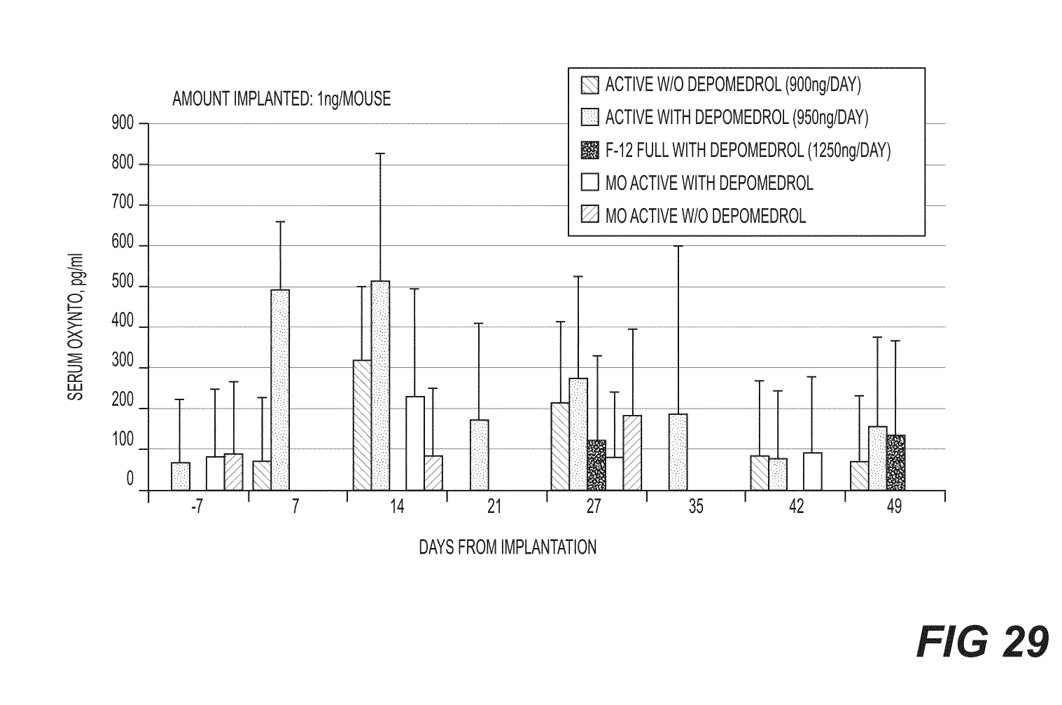

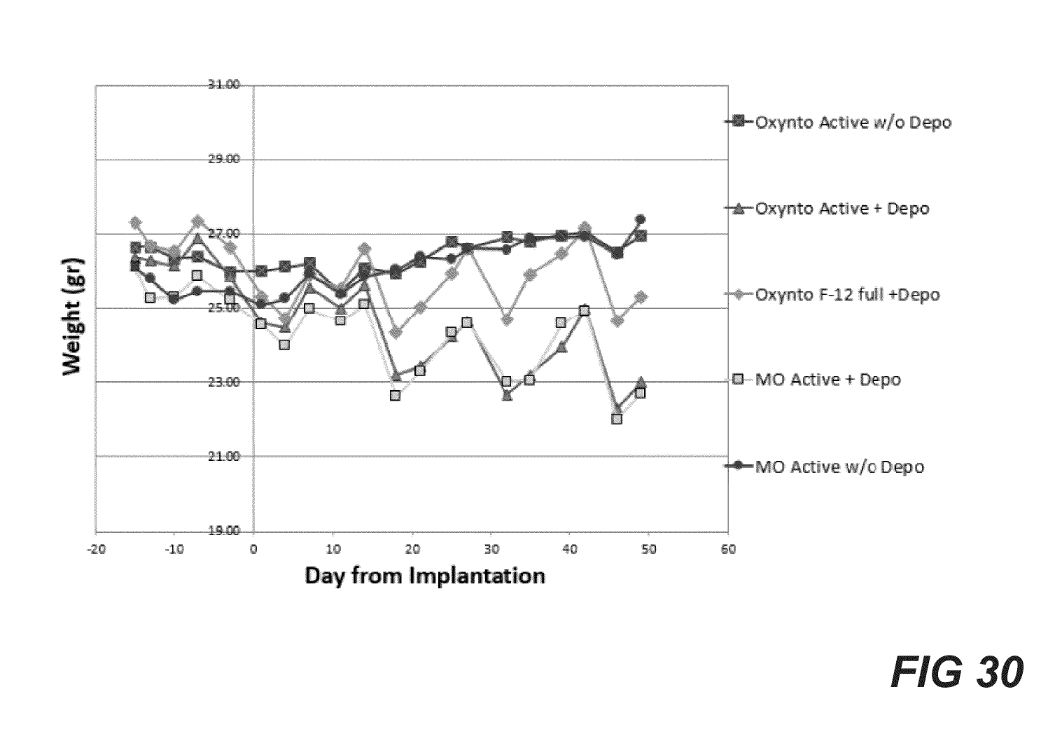

Levels of serum oxyntomodulin were measured over 49 days following implantation. Results shown in FIG. 29 indicate that oxyntomodulin levels were similar to baseline levels for the first two weeks after GMMO implantation. There was also no change in body weight observed for any group following implantation of 1 ng/mouse of GMMO-oxyntomodulins, as shown in FIG. 30.

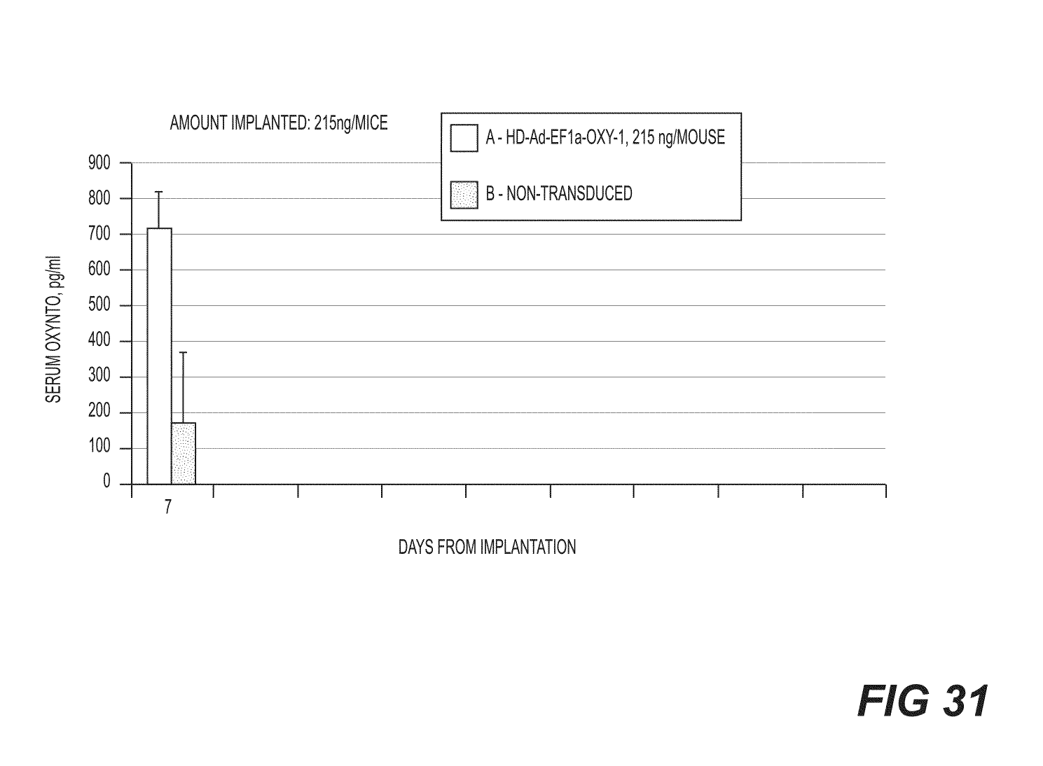

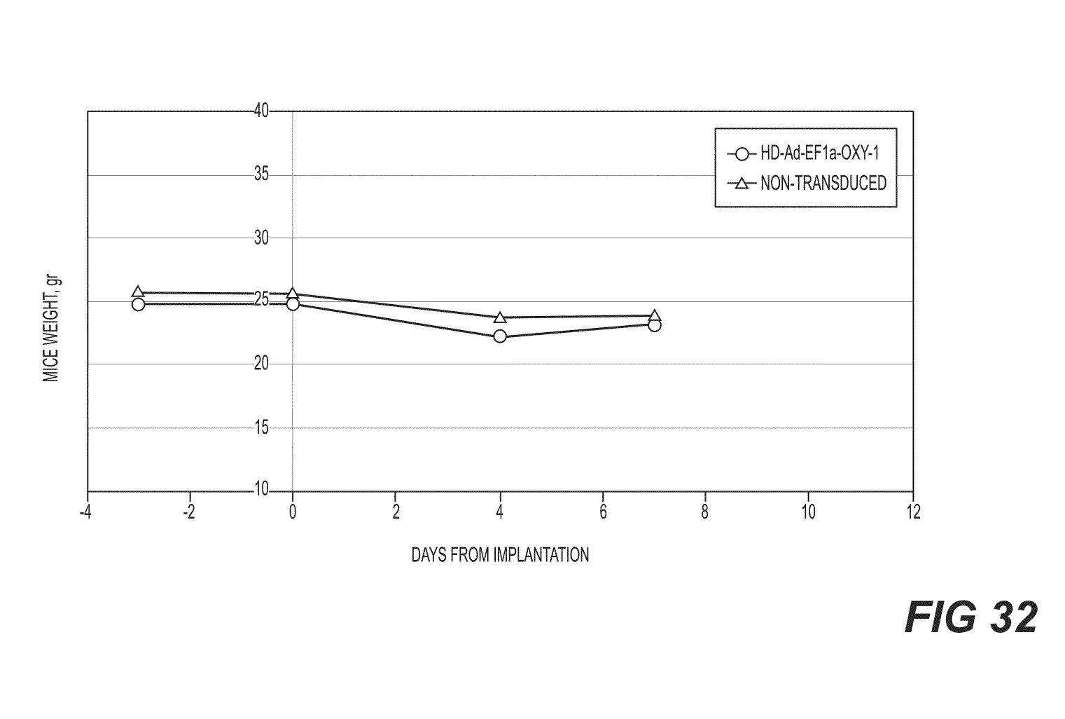

Another experiment evaluated the effect of implantation of a larger amount of GMMO-oxyntomodulin (using transduction with HD-Ad-EF1α-Oxy-1 vector). In this experiment, mice were implanted with 215 ng/mouse of either GMMO-oxyntomodulin or non-transduced MO. Depomedrol was dosed on implantation. DepoMedrol was used at 2 mg DepoMedrol per mouse. Using these conditions, an increase of approximately 500 ng/ml was seen in serum oxyntomodulin levels in mice implanted with GMMO-oxyntomodulins compared with mice implanted with non-transduced MOs, as shown in FIG. 31. There was no change in body weight of mice in this experiment over 11 days after implantation for either group, as shown in FIG. 32.

Next, the performance of GMMO-oxyntomodulins was assessed in nude rats. Rat GMMOs were prepared as described for SCI mice in Example 4, with the only change that the production media is TheraPEAK™ MSCGM-CD™ Mesenchymal Stem Cell Medium, Lonza, volume 3 ml.

The GMMO's/MO's were transferred to the Nude rat facility in an incubator at 32° C., without CO₂ in 2 ml cryotubes containing 2 ml growth medium (a 2.5 hrs transport time). All GMMO's/MO's were washed six times in saline prior to implantation.

Two or four GMMO's/MO's were implanted SQ in each rat and implantation was done by implantation device with 10 G needle. Depo-Medrol (40 mg/ml, Pfizer) was injected to groups according to Table below on implantation and every two weeks. The injections were as following: 1 mg depomedrol/GMMOs and MOs (25 ul Depomedrol stock+75 ul saline per GMMO or MO).

TABLE 7

| | | | | Arranged for implantation | | |
|---|---|---|---|---|---|---|
| Group# | Mouse# | GMMO# | ng/day/GMMO | Left/Right | Comments | ng/rat |

| | | | | | |
|---|---|---|---|---|---|
| C | C1 | 14 | 25.6 | Left Lateral | 95.48 |
| HD-Ad-EF1a-Oxy | | 17 | 15.8 | Left Center | |
| 1 | | 28 | 21.2 | Right Center | |
| MSC Lonza | | 36 | 32.9 | Right Lateral | |
| DepoMedrol on | C2 | 34 | 22.9 | Left Lateral | 87.10 |
| implantation and | | 20 | 14.5 | Left Center | |
| every two weeks | | 13 | 27.4 | Right Center | |
| | | 23 | 22.3 | Right Lateral | |
| | C3 | 27 | 28.7 | Left Lateral | 110.14 |
| | | 25 | 28.3 | Left Center | |
| | | 32 | 27.3 | Right Center | |
| | | 16 | 25.9 | Right Lateral | |
| D | D1 | 33 | 25.5 | Left Lateral | 107.17 |
| HD-Ad-EF1a-Oxy | | 15 | 23.1 | Left Center | |
| 1 | | 22 | 14.3 | Right Center | |
| MSC Lonza | | 31 | 44.2 | Right Lateral | |
| DepoMedrol on | D2 | 26 | 22.8 | Left Lateral | 89.65 |
| implantation and | | 30 | 27.1 | Left Center | |
| every two weeks | | 24 | 12.3 | Right Center | |
| | | 19 | 27.4 | Right Lateral | |
| E | E1 | 37 | | Left Lateral | |
| Non Transduced | | 38 | | Left Center | |
| MSC Lonza | | 39 | | Right Center | |
| DepoMedrol on | | 40 | | Right Lateral | |
| implantation and | E2 | 41 | | Left Lateral | |
| every two weeks | | 42 | | Left Center | |
| | | 43 | | Right Center | |
| | | 44 | | Right Lateral | |
| | E3 | 45 | | Left Lateral | |
| | | 46 | | Left Center | |
| | | 47 | | Right Center | |
| | | 48 | | Right Lateral | |
| F | F1 | 49 | | Left Lateral | |
| Non Transduced | | 50 | | Left Center | |
| MSC Lonza | | 51 | | Right Center | |
| DepoMedrol on | | 52 | | Right Lateral | |
| implantation and | F2 | 53 | | Left Lateral | |
| every two weeks | | 54 | | Left Center | |
| | | 55 | | Right Center | |
| | | 56 | | Right Lateral | |

Rats were bled once a week, and plasma oxyntomodulin levels were measured by ELISA.

The duration of the experiment was 2 months. The above table shows the ng/rat implanted (per 4 GMMOs which were implanted) and ng/day/MO (the level of oxyntomodulin measured in the spent media before implantation).

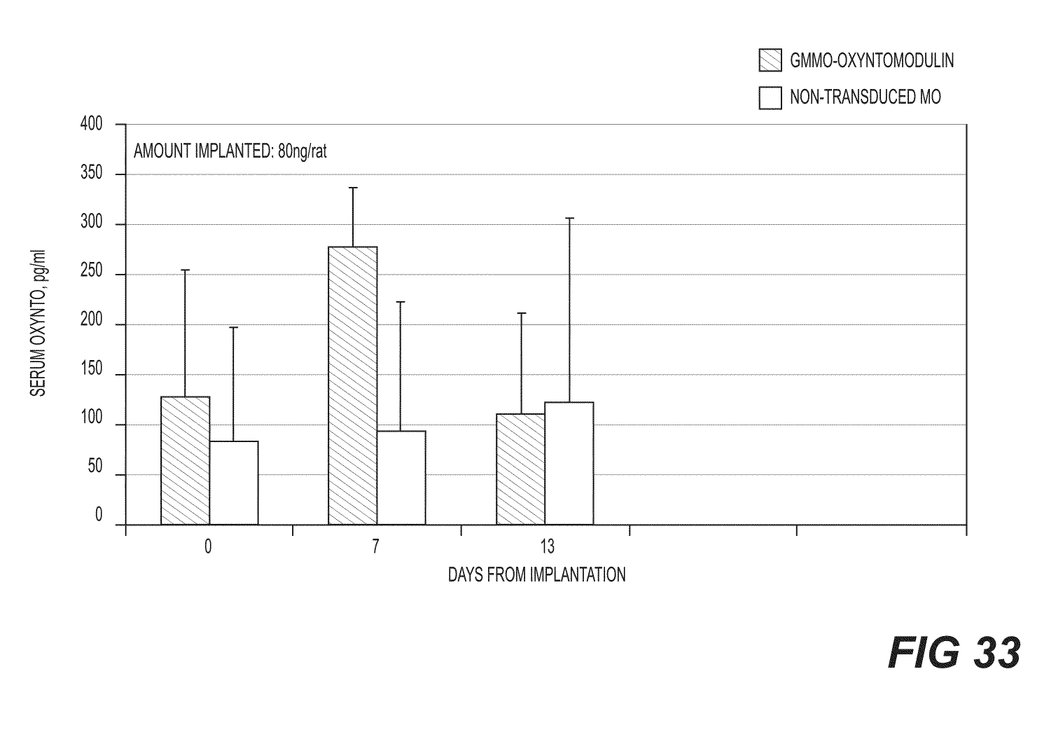

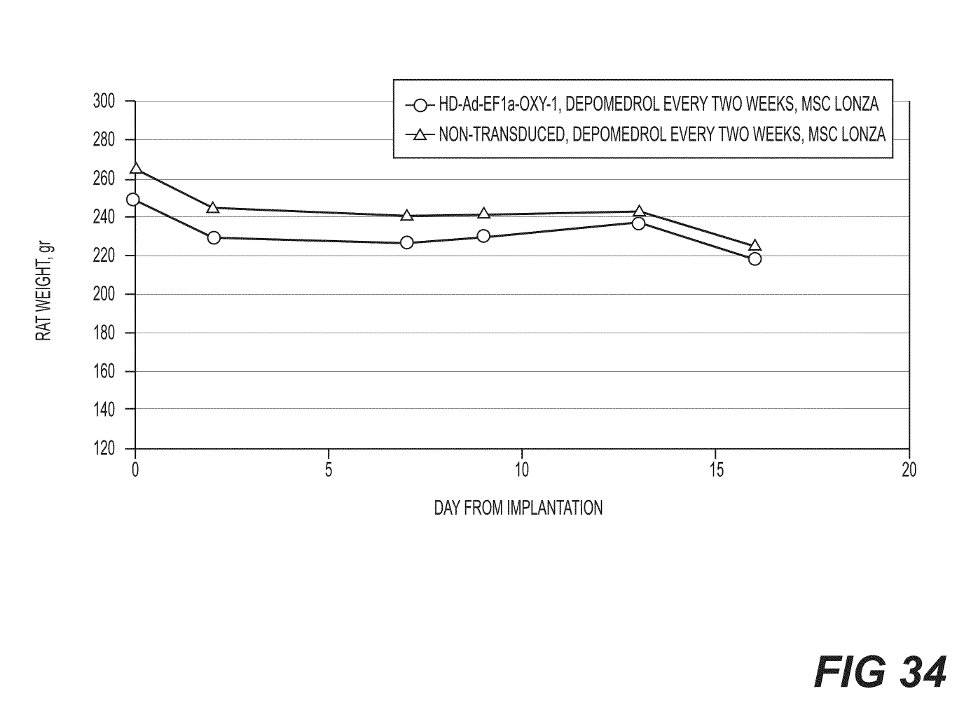

Each rat in this experiment was implanted with 80 ng of either GMMO-oxyntomodulin or nontransduced MO. Depomedrol was dosed at implantation and at every two weeks. As shown in FIG. 33, implantation of GMMO-oxyntomodulin led to an increase in serum oxyntomodulin of approximately 200 pg at 7 days post-implantation compared to implantation of nontransduced MO. FIG. 34 shows that no difference in weight was observed over 16 days post-implantation for rats implanted with GMMO-oxyntomodulin versus nontransduced MOs.

Thus, both data with SCID mice and nude rats indicated the oxyntomodulin secreted from implanted GMMO-oxyntomodulins can reach the animals' bloodstream, as increases in serum oxyntomodulin were seen in both models compared with implanted nontransduced MOs.

### Example 13

### Optimization of Novel Expression Cassettes for GMMO-oxyntomodulin

Based on the initial positive in vitro and in vivo results with GMMO-oxyntomodulins, additional cassettes were designed. Approach-A in FIG. 35 summarizes the expression cassette used for generation of GMMO-oxyntomodulin experiments described in Examples 11 and 12.

Additional cassettes presented in FIG. 35 test a variety of different configurations and components of the cassettes. Nucleic acids corresponding to the amino acids for the components of the cassettes were generated. Some cassettes were designed to express both oxyntomodulin and protein YY (PYY). Approach-B through Approach F investigate the use of the proglucagon signaling peptide (SEQ ID No: 9). Approach-B and Approach-C test the use of two oxyntomodulin peptides (SEQ ID No: 22) separated by either an IP-2 (convertase 1/3 and 2, SEQ ID No: 13) or furin linker (SEQ ID No: 24). Approach-D tests the use of three oxyntomodulin proteins with two furin linkers. Approaches E-G test the use of one oxyntomodulin protein and one peptide YY (PYY) protein (SEQ ID No: 25 or SEQ ID No: 31). Approach-E uses an IP-2 linker, while Approach-F uses a furin linker. Approach G uses a PYY signaling peptide, an oxyntomodulin protein, a furin linker, and a PYY protein.

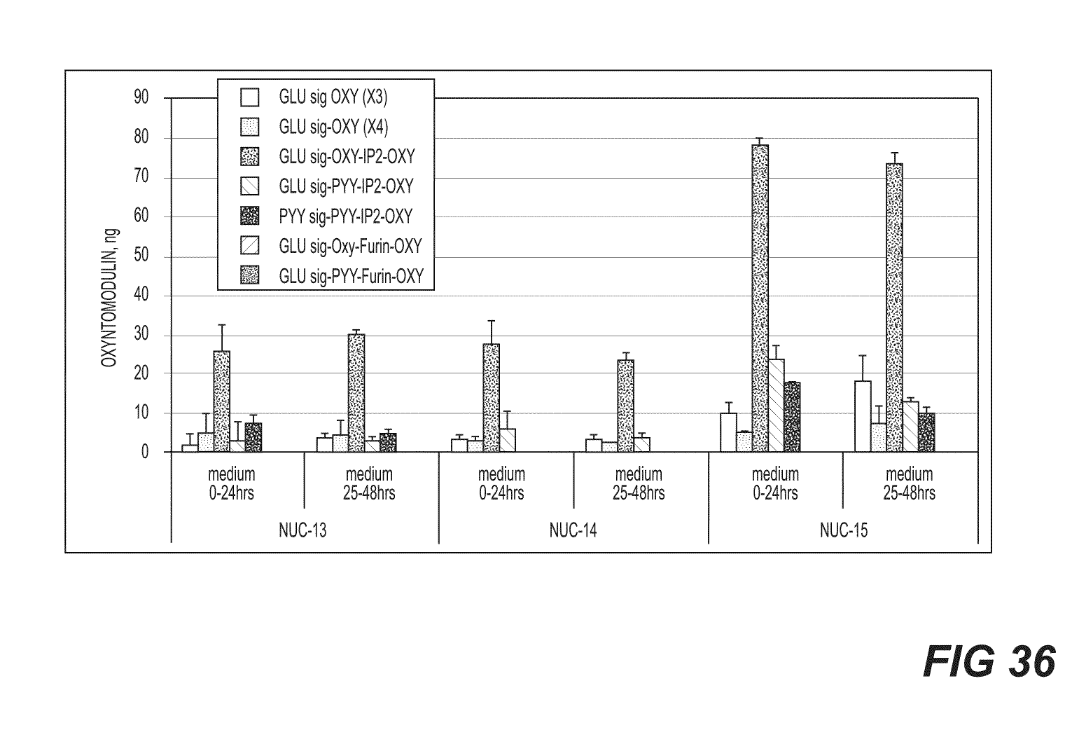

Various oxyntomodulin plasmids were delivered to human dermal fibroblasts via nucleofection following procedures outlined in Example 3. Results are shown in FIG. 36. The plasmids were generated to express the proteins, signaling peptides, and linkers as listed in the legend of FIG. 36. Some constructs were designed to express multiple copies of oxyntomodulin. Other constructs expressed oxyntomodulin and protein YY (PYY).

In FIG. 36, “Glu sig” refers to proglucagon signaling peptide (SEQ ID No: 9). “Oxy” refers to the oxyntomodulin peptide (SEQ ID No: 22). “IP2” corresponds to SEQ ID No: 13. “X3” refers to three copies of the oxyntomodulin peptide separated by the PCK1, 2 linker. “X4” refers to three copies of the oxyntomodulin peptide separated by the PCK1, 2 linker. “Furin” refers to SEQ ID No: 24. “PYY” refers to SEQ ID No: 25 or SEQ ID No: 31. “PYY signaling peptide” refers to SEQ ID No: 29.

The plasmid with the cassette Glu-sig-Oxy (X3), corresponds to Approach-A in FIG. 35. The Glu-sig-Oxy-IP2-Oxy corresponds to Approach B in FIG. 35. The Glu sig-Oxy-Furin-Oxy corresponds to Approach-C in FIG. 35.

Oxyntomodulin released into the media over 0-24 or 25-48 hours after nucleofection of human dermal fibroblasts are presented for three separate nucleofections (NUC-13, NUC-14, and NUC-15). The plasmid used in Examples 11 and 12 is Glu-sig-Oxy (X3) (i.e., Approach-A in FIG. 35). Interestingly, the Glu Sig-Oxy-IP2-Oxy construct (i.e., Approach-B in FIG. 35) caused secretion of 8- to 10-fold more oxyntomodulin into the media compared to the construct of Approach-A.

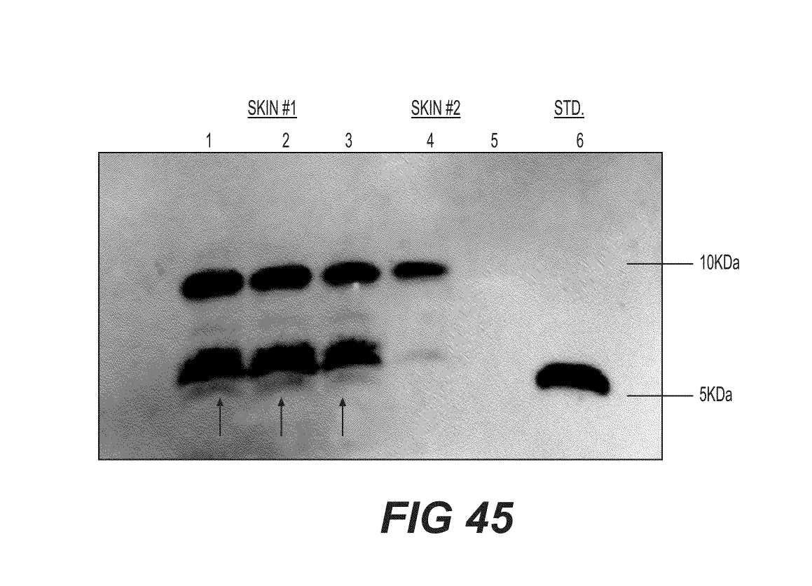

Secreted oxyntomodulin from rat GMMOs transduced with HDAd-EF1α expressing oxyntomodulin Ver B was evaluated. Oxyntomodulin samples were analyzed by SDS PAGE (16% precast Tricine gel Product # EC66952BOX, Life Technologies). For SDS PAGE analysis, GMMO collection media samples were lyophilized followed by reconstitution with Urea 8M followed by 3 fold dilution to final urea concentration of 2.66M. Prior to loading on the gel, standard sample, as well as reconstituted GMMO collection media samples were diluted with reducing sample buffer, and incubated 5 minutes at 95° C. The running conditions were as follows: 100V, 200 minutes in cooled Tricine buffer (Product# LC1675, Life Technologies). Molecular weight size marker used was 7 μl of pre-stained Ultra-low Range Molecular Weight Marker (Product#1610377, Bio-Rad). Following electrophoresis, the proteins separated in the gel were electroblotted to a 0.1 um nitrocellulose membrane (Product #10600000, GE) using wet transfer system (Product # EI9051 Life Technologies). Blotting conditions of 100 mA for 35 minutes were optimized for small proteins. Next, the blotted membrane was blocked with PBS 0.2% Tween (PBST)/10% skim milk solution for overnight at 4° C., followed by incubation with Rabbit anti Oxyntomodulin antibody (Product# OXM11a Alpha Diagnostic International) at a 500-fold dilution from stock in PBST/1% milk for 4 hours or overnight at room temperature. After three 5-minute washes with PBST, HRP conugated Goat anti-Rabbit antibody (Product #111-035-144, Jackson Immuno Research) diluted 5000-fold in PBST/1% milk was applied for 1 hour at room temperature. Detection was carried out using chemiluminescent substrate (Product # PIR-34095, Thermo Scientific). Imaging was conducted by Amersham Imager 600 chemiluminescent imager (Product#29-0834-61General Electric). Lanes 1-4 (Skin 1, 2, 3, and 4) are conditioned media from separate GMMOs. Lane 5 is marker. Lane 6 is an oxyntomodulin standard in media. The arrows in FIG. 45 show the presence of oxyntomodulin monomer in Skin 1, 2, and 3 samples.

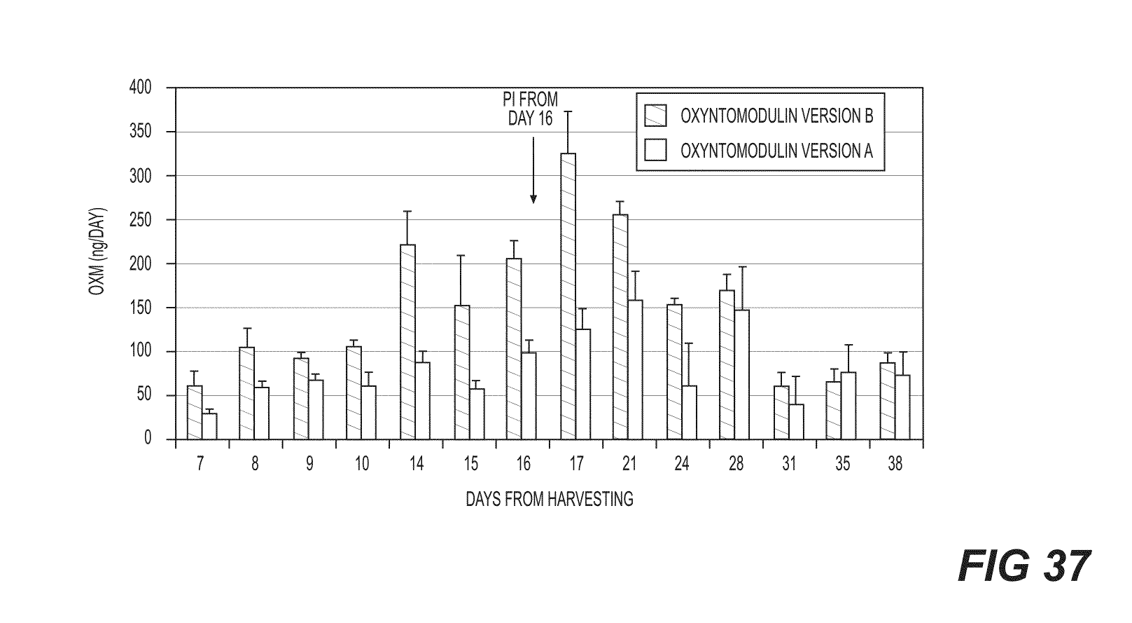

GMMOs were generated following the protocols outlined in Example 4 with the HDAd-EF1α vector comprising the Approach A oxyntomodulin cassette (SEQ ID NO: 58) or the HDAd-EF1α vector comprising the Approach B oxyntomodulin cassette (SEQ ID NO: 59), as specified in FIG. 35. As shown in FIG. 37, in vitro experiments indicated that substantially more oxyntomodulin is secreted as measured over 38 days from harvesting from GMMOs generated with the vector expressing the Approach-B cassette compared to the Approach-A cassette. Protease Inhibitor Cocktail (PI), 1 ml, Cat#P1860, Sigma, USA, was added to the medium starting from Day 16. These data indicate that the oxyntomodulin construct with two copies of oxyntomodulin, an IP-2 linker, and a proglucagon signaling peptide shows evidence of producing greater oxyntomodulin secretion when used to transduce GMMOs. As this construct with improved efficacy parallels the HDAd-EF1α-GLP-2 variant verB (SEQ ID No: 21, a construct according to approach B and comprising GLP-2 variant sequence), these data may indicate that use of the proglucagon signaling peptide and two target protein cassettes separated by an IP-2 linker may be a cassette construction that allows optimal expression and secretion of target peptides.

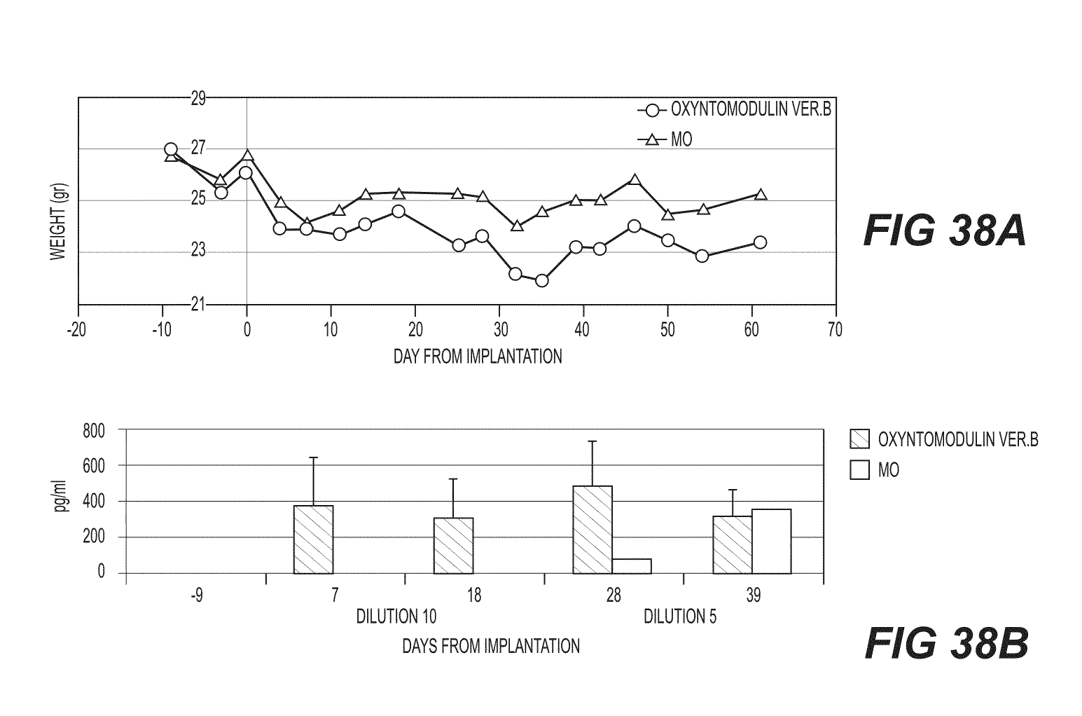

GMMOs generated using HDAd-EF1α vector comprising the Approach B oxyntomodulin cassette (oxyntomodulinVerB, cassette map labeled as Approach B in FIG. 35) were implanted into diet-induced obesity (DIO) mice to evaluate potential changes in body weight due to increased serum levels of oxyntomodulin. The mouse strain for the DOI experiments were Strain B6.129S7-Rag1tm1Mom/J, Jackson. In the DIO model, mice were maintained between ages 6-12 weeks on an 18% calories from fat diet (Cat#2018, Teklad) with a switch after Week 12 to a 60% calories from fat diet, Cat# D12492, Research Diets. The switch in diet occurred two weeks prior to implantation. DIO mice were implanted either with GMMO-oxyntomodulinVerB or nontransduced MO with 2 mg/mouse DepoMedrol, and body weight was evaluated for 63 days. As shown in FIG. 38A, DIO mice implanted with GMMO-oxyntomodulinVerBs had reduced body weight compared with mice implanted with nontransduced MOs starting on Day 18 after implantation. The reduced body weight in mice implanted with GMMO-oxyntomodulinVerBs versus those implanted with nontransduced MOs was still apparent at the end of the experiment at 63 days after implantation. FIG. 38B shows that serum levels of oxyntomodulin were higher in mice implanted with GMMO-oxyntomodulinVerBs versus those implanted with nontransduced MOs through 28 days after implantation. “Dilution 10” and “Dilution 5” refer to the fold dilution used for measurement of serum oxyntomodulin.

These data indicate that both in vitro and in vivo, a construct expressing the proglucagon signaling peptide and two copies of the oxyntomodulin protein separated by an IP-2 linker (i.e. Version B/Approach B as highlighted on FIG. 35) shows substantially greater expression of oxyntomodulin than constructs with different cassette makeups when used to generate GMMOs. These data parallel the efficacy seen with Version B, having a similar makeup of components, when producing GMMOs designed to express GLP-2 or variant GLP-2, as described in Example 4. These data indicate that for some target peptides, use of an expression cassette with components to express the proglucagon signaling peptide and two copies of the target protein separated by an IP-2 linker may be a favorable composition for generating GMMOs capable of producing and secreting relatively large amount of the target protein.

### Example 14

### Preparation of Cassettes, Vectors, and GMMOs for Expression of Protein YY (PYY)

Methodologies herein were used to evaluate the efficacy of GMMOs transduced with vectors comprising cassettes for PYY with a signaling peptide and cleavable linker(s).

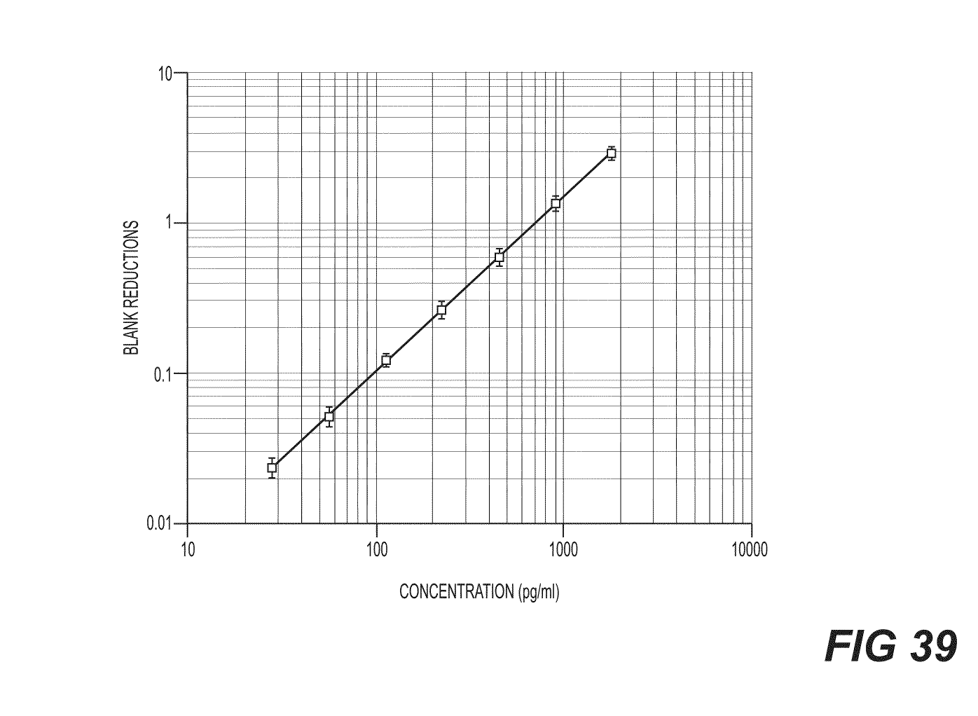

Assays were evaluated to measure PYY in samples. A commercial sandwich assay ELISA from Millipore (Product Number EZHPYYT66K) was identified and found to be suitable for measuring levels of PYY. The reported detection limit by the manufacturer is 6.5 pg/ml and according to the manufacturer it can be used with human serum or plasma. The commercially provided instructions for this ELISA assay may be followed to detect levels of PYY in in vivo samples. A representative calibration curve using reagents provided in the kit is shown in FIG. 39.

A variety of cassettes were designed for expression of signaling peptides, cleavable linkers, and multiple copies of PYY. The components of the different PYY cassettes are shown in FIG. 40A. These include sequences to express a PYY signaling peptide (SEQ ID No: 29), a proglucagon signaling peptide (SEQ ID No: 9), PYY(3-36) (labeled “PYY” on this graphic) (SEQ ID No: 31), a phosphoenolpyruvate carboxykinase (PCK1) linker (SEQ ID No: 14), a peptidylglycine alpha-amidating monooxygenase (PAM) linker (sequence GKR), a furin linker (SEQ ID No: 24), a propeptide linker (SEQ ID No: 32), and a 2A linker (SEQ ID No: 33). These Approaches are labeled 1-7.

FIG. 40A also present a construct expressing the wildtype PYY whole precursor with amino acids 1-36 (“PYY (1-36)”; SEQ ID No: 25). This construct also include a sequence for expression of peptide C (SEQ ID No: 42).

FIG. 40B presents the information of DNA makeup of the cassette (middle column) and DNA makeup of the vector in the right column for Approaches 1-7, as well as a wild-type cassette and corresponding vector that expresses the whole PYY precursor. PYYSig is PYY signaling peptide. ProgSig is proglucagon signaling peptide. F2A is furin-2A.

Nucleofection of various vectors expressing cassettes encoding PYY was performed. Electroporation of human dermal fibroblasts was done using the Amaxa® Nucleofector® (Lonza). Human dermal fibroblast cells (HDF) from tummy tuck tissue treated with a collagenase treatment were used after passage 5. The growth medium was DMEM-F-12 (ADCF) with phenol red (Hy Clone). Medium was supplemented with 10% DCS (Defined Calf serum Iron Supplemented HyQ); AmBisome 2.5 μg/ml (Liposomal Amphotericin B 50 mg—GILEAD); Gentamycin sulfate 50 μg/ml (Gentamicin-IKA 80 mg Teva). The trypsin used was trypsin/EDTA (Trypsin/EDTA; Lonza). The Hepes buffered saline (HBS) used was Hepes buffered saline*2 (hepes buffered saline; Lonza). The trypsin neutralizing solution (TNS) used was from Lonza).

The growing conditions (prenucleofection) for the fibroblasts were that five days before electroporation cells were seeded in 10 cm² plates, medium was changed every 3 days, and cells reached 90% confluency on the day of nucleofection.

For the nucleofection, growth medium was removed from four 10 cm² plates. Cells were washed once with 10 ml HBS. Cells were then harvested by trypsinization: 3 ml of Trypsin/EDTA solution was added to the plate, the plate was gently swirled to ensure an even distribution of the solution, the plate was incubated at 37° C. for 3 minutes, the plate was removed from the incubator, and TNS was added to inactivate the trypsin. Cells were gently resuspended and removed from plates by pipetting. Cells were counted three times with a yield of 13.5×10⁶ cells. Cells were pelleted in 500 g for 10 min in a 50 ml tubes.

For transfection, fibroblasts were resuspended with 1400 μl of Human Dermal Fibroblast Nucleofector™ Solution (final conc. 7.9×10⁵ cells/100 μl). 100 μl of cells were mixed with 5 μg DNA. The nucleofection sample was transferred into an Amaxa certified cuvette. The electroporation program U-23 was activated. Cells were removed from the cuvette immediately at the end of the program by adding 500 μl of pre-warmed culture medium and transferred into 6 well plate. Reactions containing 7.9×10⁵ cells/100 μl were seeded into 6 well plate already containing 1.5 ml of growth medium. Cells were transferred from the cuvette to the dish using plastic pipette.

The following are the samples included in the nucleofection experiment:

TABLE 8

| | Cells | | Cassette | Vector | Nucleofector |
|---|---|---|---|---|---|
| Sample | number | Approach | DNA | DNA | program |
| 1-2 | 7.9 × | Approach | PYYSig- | pAD-EF1a- | U-23 |
| | 10⁵ | 2 | PYY-PAM- | PYY PAM | |
| | | | PYY | | |
| 3-4 | 7.9 × | Approach | PYYSig- | pAD-EF1a- | U-23 |
| | 10⁵ | 3 | PYY-PAM- | PYY2B | |
| | | | PP-PAM- | PAM | |
| | | | PYY | | |
| 5-6 | 7.9 × | Approach | ProgSig- | pAD-EF1a- | U-23 |
| | 10⁵ | 5 | PYY-F2A- | PYYfurin | |
| | | | PP-ProgSig- | 2A Pro | |
| | | | PYY | | |
| 7-8 | 7.9 × | Approach | ProgSig- | pAD-EF1a- | U-23 |
| | 10⁵ | 6 | PYY-F2A- | PYYx2 | |
| | | | PYY | furin 2A | |
| 9-10 | 7.9 × | Approach | ProgSig- | pAD-EF1a- | U-23 |
| | 10⁵ | 7 | PYY-F2A- | PYYfurin | |
| | | | PP-PYY | 2A2B | |
| 11-12 | 7.9 × | Approach | PYYSig- | pAD-EF1a- | U-23 |
| | 10⁵ | 1 | PYY-PCK1- | PYY(3-36) | |
| | | | PP-PCK1- | verA | |
| | | | PYY | | |

Medium (2 ml) was collected at 24 hrs and 48 hrs, and cells were harvested for protein extraction using M-per (Pierce). Collected growth media from each well was added to cold Eppendorf tubes and centrifuged for 10 min at 5000 rpm. The supernatant was transferred to another Eppendorf tube, and the pellet was retained. Each well was washed with cold 500 μl PBS, and the cells were transferred (with PBS) to the tubes with the pellet and centrifuged for 10 min in 5000 rpm. Cells were treated with protease inhibitor (1:100) and centrifuged for 10 min in 13000 rpm. Supernatants were collected and frozen at −80° C.

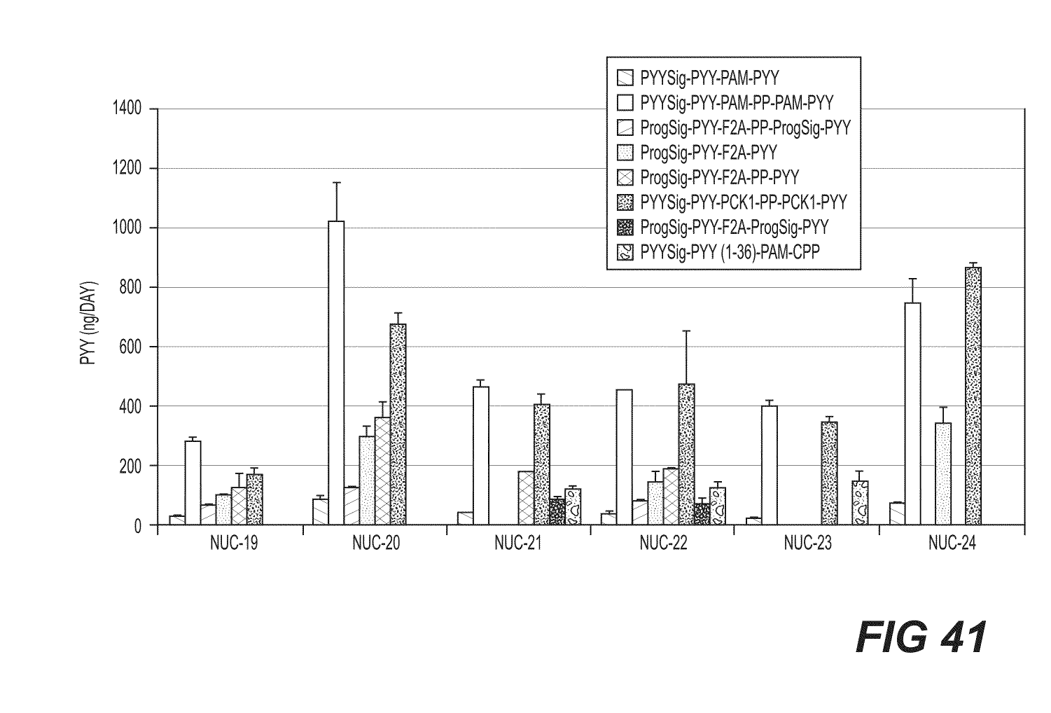

FIG. 41 shows results on levels of secreted PYY from five separate experiments using fibroblasts nucleofected with vectors comprising the constructs listed in FIG. 40. Different NUC numbers indicate separate nucleofections. Secretion was seen for all vectors expressing PYY-containing cassettes. The vectors comprising the PYYSig-PYY-PAM-PP-PAM-PYY (Approach 3) and PYYSig-PYY-PCK1-PP-PCK1-PYY (Approach 1) constructs produced especially robust secretion of PYY following nucleofection of fibroblasts.

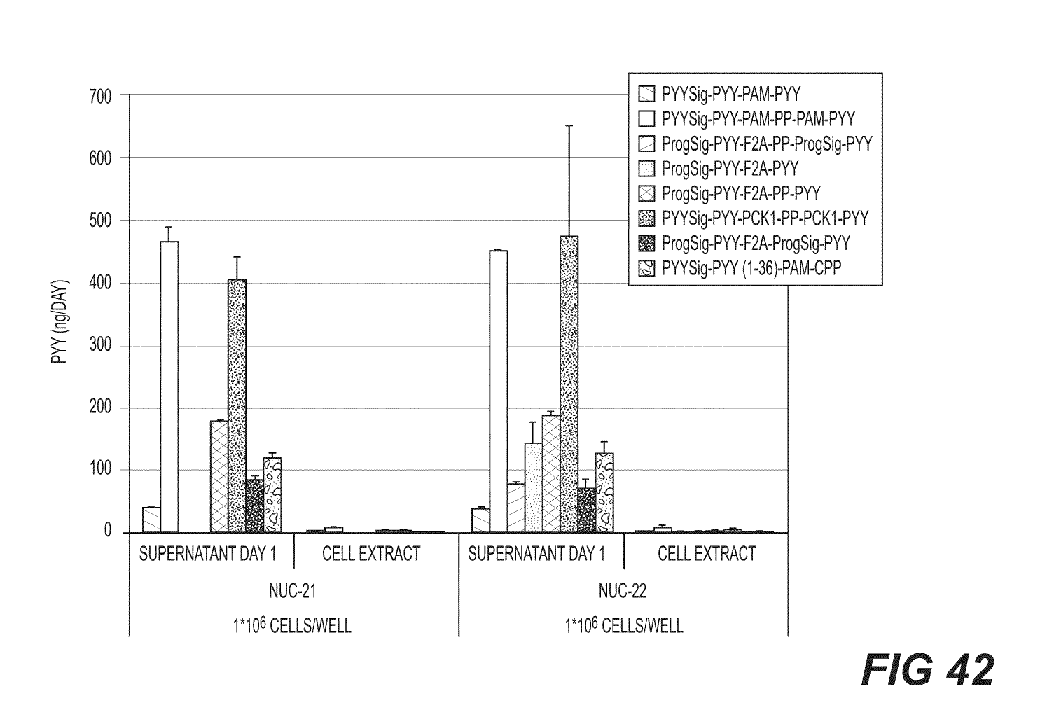

The amount of PYY (ng/day) was measured in the supernatant from collected media or within the cell extract following nucleofection. Different NUC numbers indicate separate nucleofections. As shown in FIG. 42, substantially more PYY is present in the supernatant of fibroblasts following nucleofection compared with the amount present in the cell extract. These data indicate that the PYY produced using vectors expressing the cassettes of Approaches 1-7 is successfully secreted, as most PYY is present in the supernatant and not within the cells.

Experiments were then performed to assess the production of PYY by GMMOs transduced with HDAd-EF1α-PYY. Specific procedures are highlighted here, and the methodology parallels that presented in Example 4. The experiment used DME/F-12 medium with 10% DCS (defined calf serum) HyClone DME/F-12 1:1 (X1)+2.50 mM L-Glutamine+15 mM HEPES Buffer (Thermo scientific). Medium was supplemented with 10% DCS (HyClone Defined Bovine Calf Serum supplemented, Thermo scientific), AmBisome 2.5 μg/ml (Liposomal Amphotericin B 50 mg—Gilead), and Gentamycin sulfate 50 μg/ml (Gentamicin-IKA 80 mg/2 ml-Teva). The viral vector used was HDdelta28E4-MAR-EF1α-Approaches 1, 2, 3, or 6 at 5.92×10¹² vp/ml, 7.46×10¹² vp/ml, 6.95×10¹² vp/ml, 6.61×10¹² vp/ml, respectively. The skin used was from tummy tuck tissue.

Dermal core MOs (30 mm) were prepared in a sterile hood using the NOUVAG chuck driller, with the NOUVAG motor set at 7000 rpm chuck driller and double hump Dermavac 3 mm equipment with 14G needles (2.05 mm deep from skin surface) and back vacuum containing 2 ml of saline. The MO's were flushed out from the needles with saline. Needles were replaced every 4-5 harvest. The MO's were incubated for one minute in saline. Then all the MO's were washed 3 times with DMEMO F-12 media without serum in a Petri dish (all the MO's were cleaned). Every wash was performed in a new Petri dish.

All the MOs were incubated with 1 ml growth media in 24 well/plate (SARSTEDT for Suspension Cells) at 5% CO₂ incubator 32° C. for 24 hrs. Certain MO's were transduced with HDdelta28E4-MAR-EF1α-Approaches 1, 2, 3, or 6 at 1.5×10¹⁰ vp/ml to produce GMMO's and other MO's were not transduced to serve as a negative control. The vector was diluted in Growth media containing 10% DCS serum to final concentration of 1.5×10¹⁰ vp/GMMO (approach 1-2.53 μl/GMMO; approach 2-2.01 μl/GMMO; approach 3-2.16 μl/GMMO; approach 6-2.27 μl/GMMO). Other MO's were not transduced as a negative control. In an open system (24 well/plate), 250 μl of transduction medium was added to each well using 1 ml pipettor. The plate was placed on a designated tray and incubated at 32° C., 5% CO₂, for 24 hours, with 300 rpm shaking for the first 4 hours. The viral wash was conducted in an open system (24 well/plate). GMMOs/MOs were washed from the transduction medium, and growth medium was added. The 250 μl of transduction medium was removed from the plate with a pipettor, and 2 ml of fresh growth medium was added (first wash). 3 ml of growth medium was added to wells of a new 6 well plate (“maintenance plate”) and the MO's were transferred into the wells from the plate in which the transduction was done (second wash). The 3 ml of media was removed from each well and fresh 3 ml media was added (third wash). Then another 3 washes were conducted.

The GMMOs/MOs were transferred to a new 24 well plate with fresh 1 ml growth media in each well. The plate was incubated at 32° C., 5% CO2 for 3 days. During a maintenance phase, the growth media were changed and collected for analyses by ELISA every Monday and Thursday.

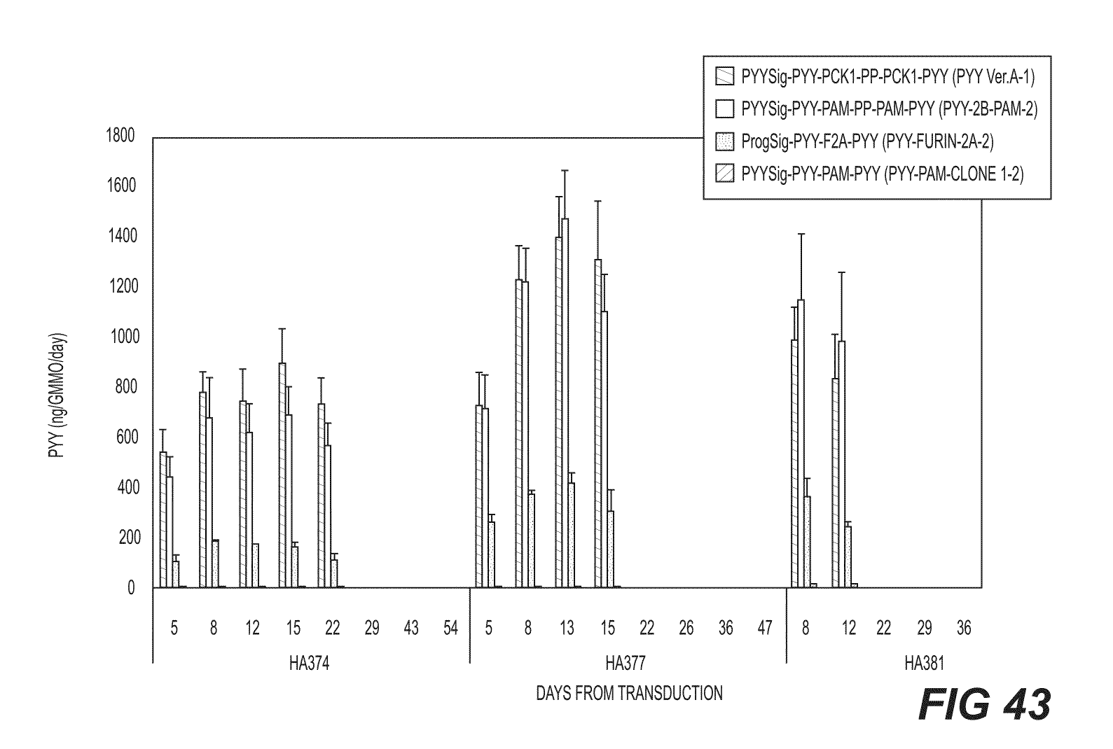

Data on secretion of PYY by three separate GMMOs (HA374, HA377, and HA381) transduced with vectors comprising PYY cassettes are shown in FIG. 43. Conditioned media was collected over a period of at least 36 days, and levels of PYY were assessed. Vectors comprising cassettes according to Approach 1 and Approach 3 produced substantially greater secretion of PYY when used to transduce GMMOs than other constructs, although each construct was successful. GMMOs transduced with these vectors were able to secrete from 400 to over 1200 ng/GMMO/day each. Peak level of expression for different GMMO transduced with vectors for PYY occurred between 8-15 days after transduction.

The effect of different media on secretion of PYY by GMMOs was also assessed. These media were collection media, collection media with 0.5% serum, collection media with 2% serum, FGM-2 medium, and Hyclone 10% RBS medium.

Sample of conditioned media from GMMOs transduced with vectors comprising the cassette PYY signal peptide-PYY-PCK1-propeptide-PCK1-PYY (i.e., Approach A) were analyzed by SDS PAGE (16% precast Tricine gel Product # EC66952BOX, Life Technologies). GMMO collection media samples were lyophilized followed by reconstitution with Urea 8M followed by 3 fold dilution to final urea concentration of 2.66M. Prior to loading on the gel, standard sample, as well as reconstituted samples of conditioned media from GMMOs were diluted with reducing sample buffer and incubated for 5 minutes at 95° C. The running conditions were 100V, 200 minutes in cooled Tricine buffer (Product# LC1675, Life Technologies). Molecular weight size marker was 7 μl of pre-stained Ultra-low Range Molecular Weight Marker (Product#1610377, Bio-Rad). Following electrophoresis, the proteins separated in the gel were electroblotted to a 0.1 um nitrocellulose membrane (Product #10600000, GE) using wet transfer system (Product # EI9051 Life Technologies). Blotting conditions of 100 mA for 35 minutes were used for small proteins. Next, the blotted membrane was blocked with PBS 0.2% Tween (PBST)/10% skim milk solution for overnight at 4° C., followed by incubation with Chicken anti-PYY antibody (Product#AB15666, millipore) diluted 200-fold in PBST/1% milk for 4 hours or overnight at room temperature. After three 5-minute washes with PBST, HRP conugated Donkey anti-Chicken antibody (Product #703-035-155, Jackson Immuno Research) diluted 1000-fold in PBST/1% milk was applied for 1 hour at room temperature. Detection was carried out using chemiluminescent substrate (Product # PIR-34095, Thermo Scientific). Imaging was conducted by Amersham Imager 600 chemiluminescent imager (Product#29-0834-61General Electric).

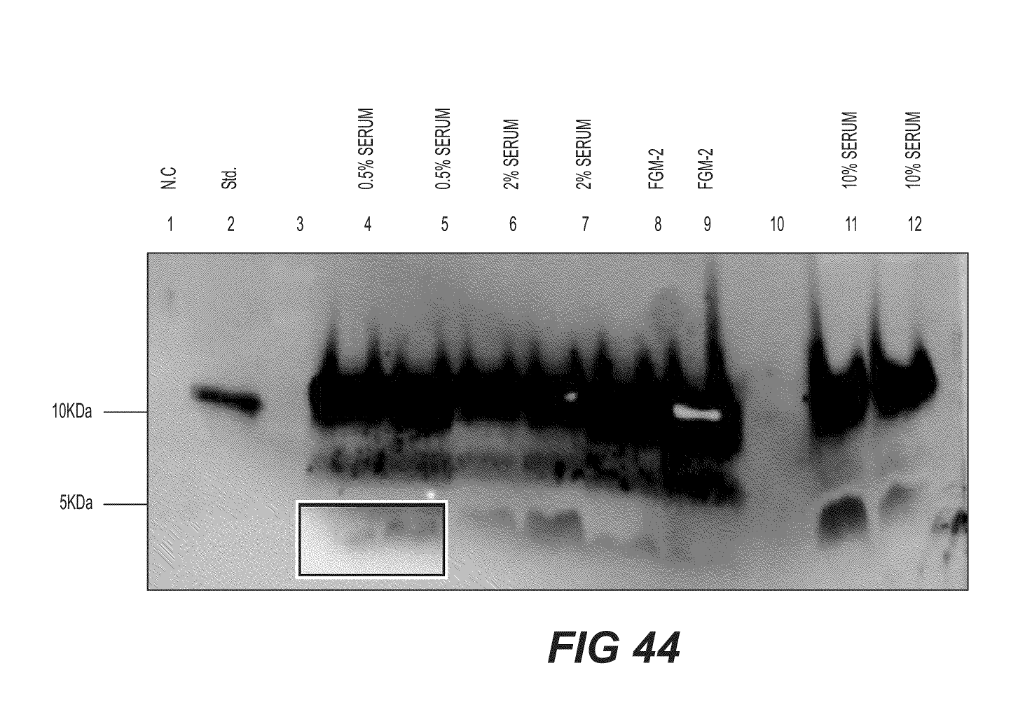

Western blot results with different media are shown in FIG. 44. “N.C” indicates a negative control sample of conditioned media from a GMMO transduced with a virus comprising an EPO-expressing construct. Lanes 3 and 10 are empty lanes. All experimental samples are from the same GMMO (HA369) maintained in different media. The predicted molecular weight of the PYY monomer is 4050.50 Da, and the predicted molecular weight of the PYY dimer is 10117.25 Da. Therefore, the lowest molecular weight bands (below 5 kDa) in all samples represent the monomer. The boxed area shows a section of blot with longer-exposure imaging, which may be due to lower protein concentration. The results presented in FIG. 44 indicate that PYY monomer and dimer forms are secreted under a variety of media conditions, with 0.5% serum-containing media generally exhibiting lower secretion levels of PYY. These in vitro data indicate that vectors comprising cassettes with signaling peptides, PYY, and cleavable linkers are appropriate for generation of GMMOs that can secrete PYY. Therefore, GMMOs are a system appropriate for therapies mediated by increased secretion of PYY.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). In some instances, the term about may include numerical values that are rounded to the nearest significant figure.

### DESCRIPTION OF THE SEQUENCES

Table 9 provides a listing of certain sequences referenced herein.

TABLE 9
Description of the Sequences

| | | SEQ |
|---|---|---|
| | | ID |
| Description | Sequences | NO |
| Human GLP-2 | HADGSFSDEMNTILDNLAARDFINWLIQTKITD | 1 |
| Wild Type Amino | | |
| Acid Sequence | | |
| Human GLP-2 | CATGCTGATG GTTCTTTCTC TGATGAGATG AACACCATTC TTGATAATCT TGCCGCCAGG | 2 |
| Wild Type Nucleic | GACTTTATAA ACTGGTTGAT TCAGACCAAA ATCACTGAC | |
| Acid Sequence | | |
| GLP-2 Variant | HGDGSFSDEMNTILDNLAARDFINWLIQTKITD | 3 |
| Amino Acid | | |
| Sequence (point | | |
| mutation in second | | |
| amino acid) | | |
| GLP-2 Wild Type | MKSIYFVAGLFVMLVQGSWQHADGSFSDEMNTILDNLAARDFINWLIQTKITDRHDFPEEVAIVEELGRR | 4 |
| Amino Acid | HADGSFSDEMNTILDNLAARDFINWLIQTKITD | |
| Sequence in | | |
| Approach B | | |
| Construct | | |
| (signal peptide- | | |
| GLP-2 wt-linker- | | |
| GLP-2 wt) | | |
| Codon Optimized | ATGAAGTCCA TTTACTTTGT GGCTGGACTG TTTGTCATGC TCGTGCAGGG AAGCTGGCAG | 5 |
| Nucleic Acid | CATGCTGACG GGAGTTTCTC CGACGAGATG AACACCATCC TGGATAATCT GGCAGCCCGG | |
| Sequence | GATTTCATCA ATTGGCTCAT ACAAACAAAG ATTACAGATC GGCACGACTT CCCAGAAGAG | |
| Encoding Wild | GTTGCCATAG TTGAAGAGCT GGGACGGCGG CATGCAGACG GCTCCTTCTC CGATGAGATG | |
| Type GLP-2 in | AATACCATTC TGGATAACCT TGCCGCAAGA GACTTCATCA ACTGGCTGAT TCAAACCAAG | |
| Approach B | ATCACCGAC | |
| Construct | | |
| Encoding SEQ ID | | |
| NO: 4 | | |
| GLP-2 Variant | MKSIYFVAGLFVMLVQGSWQHGDGSFSDEMNTILDNLAARDFINWLIQTKITDDFPEEVAIVEELGRRHG | 6 |
| Amino Acid | DGSFSDEMNTILDNLAARDFINWLIQTKITD | |
| Sequence in | | |
| Approach B | | |
| Construct | | |
| (signal peptide- | | |
| GLP-2 variant- | | |
| linker-GLP-2 | | |
| variant) | | |
| Codon Optimized | ATGAAATCTATATACTTCGTGGCCGGCCTCTTTGTGATGCTGGTGCAAGGGTCTTGGCAGCACGGGGATG | 7 |
| GLP-2 Variant | GCTCCTTCAGTGACGAGATGAACACTATACTGGATAACCTGGCGGCACGGGATTTCATCAATTGGCTCAT | |
| Nucleic Acid | CCAGACTAAGATTACTGATGACTTTCCTGAGGAGGTGGCGATTGTGGAGGAACTCGGGAGGCGCCATGGC | |
| Sequence in | GACGGCTCATTTTCCGACGAGATGAACACGATACTGGACAATCTGGCCGCCCGGGACTTTATAAATTGGC | |
| Approach B | TCATCCAGACAAAGATTACTGACTAA | |
| Construct | | |
| Encoding SEQ ID | | |
| NO: 6 | | |
| Signaling Peptide | ATGAAATCTATATACTTCGTGGCCGGCCTCTTTGTGATGCTGGTGCAAGGGTCTTGGCAG | 8 |
| Nucleic Acid | | |
| Encoding SEQ ID | | |
| NO: 9 | | |
| Human | MKSIYFVAGLFVMLVQGSWQ | 9 |
| Proglucagon | | |
| Signaling Peptide | | |
| Amino Acid | | |
| A First Codon | CACGGGGATGGCTCCTTCAGTGACGAGATGAACACTATACTGGATAACCTGGCGGCACGGGATTTCATCA | 10 |
| Optimized Nucleic | ATTGGCTCATCCAGACTAAGATTACTGAT | |
| Acid Sequence | | |
| Encoding GLP-2 | | |
| Variant SEQ ID | | |
| NO: 3 | | |
| A Second Codon | CATGGCGACGGCTCATTTTCCGACGAGATGAACACGATACTGGACAATCTGGCCGCCCGGGACTTTATAA | 11 |
| Optimized Nucleic | ATTGGCTCATCCAGACAAAGATTACTGAC | |
| Acid Sequence | | |
| Encoding GLP-2 | | |
| Variant SEQ ID | | |
| NO: 3 | | |
| IP-2 Linker | GACTTTCCTGAGGAGGTGGCGATTGTGGAGGAACTCGGGAGGCGC | 12 |
| Nucleic Acid | | |
| Sequence | | |
| Encoding SEQ ID | | |
| NO: 13 | | |
| IP-2 Linker Amino | DFPEEVAIVEELGRR | 13 |
| Acid Sequence | | |
| PCK1/2 linker, IP- | RHDFPEEVAIVEELGRR | 14 |
| 2 Linker Amino | | |
| Acid Sequence | | |
| with RH cleavage | | |
| site added | | |
| IP-1 Linker | NRNNIA | 15 |
| Sequence | | |
| GS Linker | GGGS | 16 |
| Sequence 1 | | |
| GS Linker | GSGGGS | 17 |
| Sequence 2 | | |
| EPO Signal | MGVHECPAWLWLLLSLLSLPLGLPVLG | 18 |
| Peptide Sequence | | |
| Trypsin 1 Signal | MNPLLILTFVAAALA | 19 |
| Peptide Sequence | | |
| Trypsin 2 Signal | MNLLLILTFVAAAVA | 20 |
| Peptide Sequence | | |
| HDAd-EF1a- | GGCCGATTCATTAATGCAGGGGCCGCTGCGGCCATCATCAATAATATACCTTATTTTGGATTGAAGCCAA | 21 |
| GLP-2 variant ver | TATGATAATGAGGGGGTGGAGTTTGTGACGTGGCGCGGGGCGTGGGAACGGGGCGGGTGACGTAGTAGTG | |
| B | TGGCGGAAGTGTGATGTTGCAAGTGTGGCGGAACACATGTAAGCGACGGATGTGGCAAAAGTGACGTTTT | |
| | TGGTGTGCGCCGGTGTACACAGGAAGTGACAATTTTCGCGCGGTTTTAGGCGGATGTTGTAGTAAATTTG | |
| | GGCGTAACCGAGTAAGATTTGGCCATTTTCGCGGGAAAACTGAATAAGAGGAAGTGAAATCTGAATAATT | |
| | TTGTGTTACTCATAGCGCGTAATATTTGTCTAGGGCCGCGGGGACTTTGACCGTTTACGTGGAGACTCGC | |
| | CCAGGTGTTTTTCTCAGGTGTTTTCCGCGTTCCGGGTCAAAGTTGGCGTTTTATTATTATAGTCAGCTGA | |
| | CGTGTAGTGTATTTATACCCGGTGAGTTCCTCAAGAGGCCACTCTTGAGTGCCAGCGAGTAGAGTTTTCT | |
| | CCTCCGAGCCGCTCCGACACCGGGAGGCGCGCCTTAATTAAAATTATCTCTAAGGCATGTGAACTGGCTG | |
| | TCTTGGTTTTCATCTGTACTTCATCTGCTACCTCTGTGACCTGAAACATATTTATAATTCCATTAAGCTG | |
| | TGCATATGATAGATTTATCATATGTATTTTCCTTAAAGGATTTTTGTAAGAACTAATTGAATTGATACCT | |
| | GTAAAGTCTTTATCACACTACCCAATAAATAATAAATCTCTTTGTTCAGCTCTCTGTTTCTATAAATATG | |
| | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGCATGCTGATGGTTCTTTCTCTGATGAGATGAACACCATTCTTGATAATCTTG | |
| | CCGCCAGGGACTTTATAAACTGGTTGATTCAGACCAAAATCACTGACGTTGGTGTACAGTAGTAGCAAGC | |
| | TTGCATGCCTGCAGGTCGACTCTAGACTGCCATGAAATCTATATACTTCGTGGCCGGCCTCTTTGTGATG | |
| | CTGGTGCAAGGGTCTTGGCAGCACGGGGATGGCTCCTTCAGTGACGAGATGAACACTATACTGGATAACC | |
| | TGGCGGCACGGGATTTCATCAATTGGCTCATCCAGACTAAGATTACTGATGACTTTCCTGAGGAGGTGGC | |
| | GATTGTGGAGGAACTCGGGAGGCGCCATGGCGACGGCTCATTTTCCGACGAGATGAACACGATACTGGAC | |
| | AATCTGGCCGCCCGGGACTTTATAAATTGGCTCATCCAGACAAAGATTACTGACTAAGGATCCCCGGGAG | |
| | ATATCCTAGGCTTGGCCAGACATGATAAGATACATTGATGAGTTTGGACAAACCACAACTAGAATGCAGT | |
| | GAAAAAAATGCTTTATTTGTGAAATTTGTGATGCTATTGCTTTATTTGTAACCATTATAAGCTGCAATAA | |
| | ACAAGTTAACAACAACAATTGCATTCATTTTATGTTTCAGGTTCAGGGGGAGGTGTGGGAGGTTTTTTAA | |
| | AGCAAGTAAAACCTCTACAAATGTGGTATGGAATTCAGTCAATATGTTCACCCCAAAAAAGCTGTTTGTT | |
| | AACTTGCCAACCTCATTCTAAAATGTATATAGAAGCCCAAAAGACAATAACAAAAATATTCTTGTAGAAC | |
| | AAAATGGGAAAGAATGTTCCACTAAATATCAAGATTTAGAGCAAAGCATGAGATGTGTGGGGATAGACAG | |
| | TGAGGCTGATAAAATAGAGTAGAGCTCAGAAACAGACCCATTGATATATGTAAGTGACCTATGAAAAAAA | |
| | TATGGCATTTTACAATGGGAAAATGATGGTCTTTTTCTTTTTTAGAAAAACAGGGAAATATATTTATATG | |
| | TAAAAAATAAAAGGGAACCCATATGTCATACCATACACACAAAAAAATTCCAGTGAATTATAAGTCTAAA | |
| | TGGAGAAGGCAAAACTTTAAATCTTTTAGAAAATAATATAGAAGCATGCCATCAAGACTTCAGTGTAGAG | |
| | AAAAATTTCTTATGACTCAAAGTCCTAACCACAAAGAAAAGATTGTTAATTAGATTGCATGAATATTAAG | |
| | ACTTATTTTTAAAATTAAAAAACCATTAAGAAAAGTCAGGCCATAGAATGACAGAAAATATTTGCAACAC | |
| | CCCAGTAAAGAGAATTGTAATATGCAGATTATAAAAAGAAGTCTTACAAATCAGTAAAAAATAAAACTAG | |
| | ACAAAAATTTGAACAGATGAAAGAGAAACTCTAAATAATCATTACACATGAGAAACTCAATCTCAGAAAT | |
| | CAGAGAACTATCATTGCATATACACTAAATTAGAGAAATATTAAAAGGCTAAGTAACATCTGTGGCTTAA | |
| | TTAAGGCGCGCCGGGCCCCTACGTCACCCGCCCCGTTCCCACGCCCCGCGCCACGTCACAAACTCCACCC | |
| | CCTCATTATCATATTGGCTTCAATCCAAAATAAGGTATATTATTGATGATGGCCGCAGCGGCCCTGGCGT | |
| | AATAGCGAAGAGGCCCGCACCGATCGCCCTTCCCAACAGTTGCGCAGCCTGAATGGCGAATGGGACGCGC | |
| | CCTGTAGCGGCGCATTAAGCGCGGCGGGTGTGGTGGTTACGCGCAGCGTGACCGCTACACTTGCCAGCGC | |
| | CCTAGCGCCCGCTCCTTTCGCTTTCTTCCCTTCCTTTCTCGCCACGTTCGCCGGCTTTCCCCGTCAAGCT | |
| | CTAAATCGGGGGCTCCCTTTAGGGTTCCGATTTAGTGCTTTACGGCACCTCGACCCCAAAAAACTTGATT | |
| | AGGGTGATGGTTCACGTAGTGGGCCATCGCCCTGATAGACGGTTTTTCGCCCTTTGACGTTGGAGTCCAC | |
| | GTTCTTTAATAGTGGACTCTTGTTCCAAACTGGAACAACACTCAACCCTATCTCGGTCTATTCTTTTGAT | |
| | TTATAAGGGATTTTGCCGATTTCGGCCTATTGGTTAAAAAATGAGCTGATTTAACAAAAATTTAACGCGA | |
| | ATTTTAACAAAATATTAACGCTTACAATTTAGGTGGCACTTTTCGGGGAAATGTGCGCGGAACCCCTATT | |
| | TGTTTATTTTTCTAAATACATTCAAATATGTATCCGCTCATGAGACAATAACCCTGATAAATGCTTCAAT | |
| | AATATTGAAAAAGGAAGAGTATGAGTATTCAACATTTCCGTGTCGCCCTTATTCCCTTTTTTGCGGCATT | |
| | TTGCCTTCCTGTTTTTGCTCACCCAGAAACGCTGGTGAAAGTAAAAGATGCTGAAGATCAGTTGGGTGCA | |
| | CGAGTGGGTTACATCGAACTGGATCTCAACAGCGGTAAGATCCTTGAGAGTTTTCGCCCCGAAGAACGTT | |
| | TTCCAATGATGAGCACTTTTAAAGTTCTGCTATGTGGCGCGGTATTATCCCGTATTGACGCCGGGCAAGA | |
| | GCAACTCGGTCGCCGCATACACTATTCTCAGAATGACTTGGTTGAGTACTCACCAGTCACAGAAAAGCAT | |
| | CTTACGGATGGCATGACAGTAAGAGAATTATGCAGTGCTGCCATAACCATGAGTGATAACACTGCGGCCA | |
| | ACTTACTTCTGACAACGATCGGAGGACCGAAGGAGCTAACCGCTTTTTTGCACAACATGGGGGATCATGT | |
| | AACTCGCCTTGATCGTTGGGAACCGGAGCTGAATGAAGCCATACCAAACGACGAGCGTGACACCACGATG | |
| | CCTGTAGCAATGGCAACAACGTTGCGCAAACTATTAACTGGCGAACTACTTACTCTAGCTTCCCGGCAAC | |
| | AATTAATAGACTGGATGGAGGCGGATAAAGTTGCAGGACCACTTCTGCGCTCGGCCCTTCCGGCTGGCTG | |
| | GTTTATTGCTGATAAATCTGGAGCCGGTGAGCGTGGGTCTCGCGGTATCATTGCAGCACTGGGGCCAGAT | |
| | GGTAAGCCCTCCCGTATCGTAGTTATCTACACGACGGGGAGTCAGGCAACTATGGATGAACGAAATAGAC | |
| | AGATCGCTGAGATAGGTGCCTCACTGATTAAGCATTGGTAACTGTCAGACCAAGTTTACTCATATATACT | |
| | TTAGATTGATTTAAAACTTCATTTTTAATTTAAAAGGATCTAGGTGAAGATCCTTTTTGATAATCTCATG | |
| | ACCAAAATCCCTTAACGTGAGTTTTCGTTCCACTGAGCGTCAGACCCCGTAGAAAAGATCAAAGGATCTT | |
| | CTTGAGATCCTTTTTTTCTGCGCGTAATCTGCTGCTTGCAAACAAAAAAACCACCGCTACCAGCGGTGGT | |
| | TTGTTTGCCGGATCAAGAGCTACCAACTCTTTTTCCGAAGGTAACTGGCTTCAGCAGAGCGCAGATACCA | |
| | AATACTGTTCTTCTAGTGTAGCCGTAGTTAGGCCACCACTTCAAGAACTCTGTAGCACCGCCTACATACC | |
| | TCGCTCTGCTAATCCTGTTACCAGTGGCTGCTGCCAGTGGCGATAAGTCGTGTCTTACCGGGTTGGACTC | |
| | AAGACGATAGTTACCGGATAAGGCGCAGCGGTCGGGCTGAACGGGGGGTTCGTGCACACAGCCCAGCTTG | |
| | GAGCGAACGACCTACACCGAACTGAGATACCTACAGCGTGAGCTATGAGAAAGCGCCACGCTTCCCGAAG | |
| | GGAGAAAGGCGGACAGGTATCCGGTAAGCGGCAGGGTCGGAACAGGAGAGCGCACGAGGGAGCTTCCAGG | |
| | GGGAAACGCCTGGTATCTTTATAGTCCTGTCGGGTTTCGCCACCTCTGACTTGAGCGTCGATTTTTGTGA | |
| | TGCTCGTCAGGGGGGCGGAGCCTATGGAAAAACGCCAGCAACGCGGCCTTTTTACGGTTCCTGGCCTTTT | |
| | GCTGGCCTTTTGCTCACATGTTCTTTCCTGCGTTATCCCCTGATTCTGTGGATAACCGTATTACCGCCTT | |
| | TGAGTGAGCTGATACCGCTCGCCGCAGCCGAACGACCGAGCGCAGCGAGTCAGTGAGCGAGGAAGCGGAA | |
| | GAGCGCCCAATACGCAAACCGCCTCTCCCCGCGCGTTGGCCGATTCATTAATGCAGGGGCCGCTGCGGCC | |
| | ATCATCAATAATATACCTTATTTTGGATTGAAGCCAATA | |
| Amino acid | HSQGTFTSDYSKYLDSRRAQDFVQWLMNTKRNRNNIA | 22 |
| sequence of | | |
| oxyntomodulin | | |
| Nucleic acid | CACTCACAGGGCACTTTCACTTCCGATTACTCTAAGTACCTGGACAGCAGGCGGGCCCAGGATTTCGTGC | 23 |
| sequence of | AGTGGCTTATGAACACAAAGAGAAACAGAAATAATATTGCC | |
| oxyntomodulin | | |
| Amino acid | RAKR | 24 |
| sequence of furin | | |
| linker | | |
| Amino acid | YPIKPEAPGEDASPEELNRYYASLRHYLNLVTRQRY | 25 |
| sequence of | | |
| protein YY (PYY) | | |
| Nucleic acid | TATCCTATTAAACCTGAGGCGCCAGGGGAGGATGCAAGCCCCGAGGAACTGAACCGCTATTATGCCAGTC | 26 |
| sequence of PYY | TGCGGCATTATTTGAACCTGGTGACCAGACAGCGCTAC | |
| Amino acid | MKSIYFVAGLFVMLVQGSWQ | 27 |
| sequence of | | |
| proglucagon | | |
| signaling peptide | | |
| Nucleic acid | ATGAAGTCTATCTACTTCGTGGCCGGATTGTTTGTTATGCTGGTGCAGGGTTCCTGGCAG | 28 |
| sequence of | | |
| proglucagon | | |
| signaling peptide | | |
| Amino acid | MVFVRRPWPALTTVLLALLVCLGALVDA | 29 |
| sequence of PYY | | |
| signaling peptide | | |
| WT Nucleic acid | atggtgttcgtgcgcaggccgtggcccgccttgaccacagtgcttctggccctgctcgtctgcctagggg | 30 |
| sequence of PYY | cgctggtcgacgcc | |
| signaling peptide | | |
| Amino acid | IKPEAPGEDASPEELNRYYASLRHYLNLVTRQRY | 31 |
| sequence of PYY | | |
| (3-36) | | |
| Peptide sequence | DFPEEVAIVEELGRR | 32 |
| of propeptide (PP) | | |
| linker | | |
| Amino acid | APVKQTLNFDLLKLAGDVESNPGP | 33 |
| sequence of 2A | | |
| linker | | |
| PYY (3-36) Amino | MVFVRRPWPALTTVLLALLVCLGALVDAIKPEAPGEDASPEELNRYYASLRHYLNLVTRQRYRHDFPEEV | 34 |
| Acid Sequence in | AIVEELGRRIKPEAPGEDASPEELNRYYASLRHYLNLVTRQRY | |
| Approach 1 | | |
| Construct | | |
| (PYY signal | | |
| peptide-PYY- | | |
| PCK1-Propeptide- | | |
| PCK1-PYY) | | |
| Codon Optimized | ATGGTATTTGTCAGACGACCTTGGCCTGCCCTCACCACGGTGTTGCTCGCCTTGCTCGTTTGCTTGGGGG | 35 |
| Nucleic Acid | CCTTGGTGGACGCTATCAAGCCTGAGGCACCCGGAGAAGACGCATCCCCCGAGGAATTGAACCGCTATTA | |
| Sequence | CGCATCCTTGCGGCACTACCTGAACCTCGTGACCAGGCAACGCTATCGGCACGACTTCCCTGAAGAAGTC | |
| Encoding PYY (3- | GCAATCGTCGAAGAGCTCGGCAGGAGAATCAAACCTGAGGCTCCTGGTGAGGACGCCAGTCCGGAGGAAC | |
| 36) Approach 1 | TGAATCGCTACTACGCGTCACTTAGGCACTACCTGAACCTGGTCACTCGCCAGAGATAC | |
| Construct | | |
| PYY (3-36) Amino | MVFVRRPWPALTTVLLALLVCLGALVDAIKPEAPGEDASPEELNRYYASLRHYLNLVTRQRYGKRIKPEA | 36 |
| Acid Sequence in | PGEDASPEELNRYYASLRHYLNLVTRQRY | |
| Approach 2 | | |
| Construct | | |
| (PYY signal | | |
| peptide-PYY- | | |
| PAM-PCK1-PYY) | | |
| Codon Optimized | ATGGTCTTTGTTAGACGACCGTGGCCGGCTCTGACAACTGTGCTGCTCGCCCTTCTCGTGTGCCTGGGAG | 37 |
| Nucleic Acid | CTCTTGTTGATGCCATCAAGCCTGAGGCACCTGGAGAAGACGCATCCCCTGAGGAACTGAACCGCTATTA | |
| Sequence | CGCATCATTGAGACACTACCTTAACCTCGTGACAAGACAGCGGTACGGTAAGAGAATCAAGCCAGAGGCC | |
| Encoding PYY (3- | CCCGGGGAGGATGCTTCCCCAGAGGAGTTGAATAGGTATTACGCTAGCCTGAGACATTACTTGAACCTTG | |
| 36) Approach 2 | TGACACGACAGCGATAT | |
| Construct | | |
| PYY (3-36) Amino | MVFVRRPWPALTTVLLALLVCLGALVDAIKPEAPGEDASPEELNRYYASLRHYLNLVTRQRYGKRDFPEE | 38 |
| Acid Sequence in | VAIVEELGRRGKRIKPEAPGEDASPEELNRYYASLRHYLNLVTRQRY | |
| Approach 3 | | |
| Construct | | |
| (PYY signal | | |
| peptide-PYY- | | |
| PAM-Propeptide- | | |
| PAM-PYY) | | |
| Codon Optimized | ATGGTATTTGTGAGACGCCCCTGGCCCGCGCTGACAACCGTGTTGCTGGCACTTCTTGTGTGCCTTGGAG | 39 |
| Nucleic Acid | CTCTCGTTGATGCCATCAAGCCTGAAGCACCGGGGGAAGACGCTTCCCCCGAGGAGCTCAATCGATACTA | |
| Sequence | TGCTAGTCTGCGACATTATCTGAACCTTGTGACCAGGCAGCGGTACGGCAAACGGGATTTTCCAGAGGAA | |
| Encoding PYY (3- | GTGGCGATCGTCGAGGAGCTCGGACGGCGGGGAAAACGCATCAAACCCGAGGCCCCAGGCGAGGATGCTT | |
| 36) Approach 3 | CACCTGAGGAACTTAACCGCTACTACGCAAGCCTGCGGCATTACCTTAACCTCGTGACCAGACAGCGCTA | |
| Construct | C | |
| PYY (3-36) Amino | MKSIYFVAGLFVMLVQGSWQIKPEAPGEDASPEELNRYYASLRHYLNLVTRQRYRAKRAPVKQTLNFDLL | 40 |
| Acid Sequence in | KLAGDVESNPGPIKPEAPGEDASPEELNRYYASLRHYLNLVTRQRY | |
| Approach 6 | | |
| Construct | | |
| (proGlucagon | | |
| signal peptide- | | |
| PYY-Furin-2A- | | |
| PYY) | | |
| Codon Optimized | ATGAAGTCTATCTACTTCGTGGCCGGATTGTTTGTTATGCTGGTGCAGGGTTCCTGGCAGATTAAGCCTG | 41 |
| Nucleic Acid | AGGCTCCCGGAGAAGACGCCAGCCCCGAAGAGCTCAACCGCTACTACGCTAGTCTGCGGCACTACCTTAA | |
| Sequence | CCTCGTAACAAGACAGCGGTATCGGGCCAAGAGGGCACCCGTGAAACAGACATTGAATTTCGACCTGCTG | |
| Encoding PYY (3- | AAACTCGCAGGAGACGTCGAGTCTAATCCTGGACCGATCAAACCTGAAGCCCCCGGTGAAGACGCATCAC | |
| 36) Approach 6 | CAGAAGAGTTGAACCGCTATTACGCTTCTCTTAGACACTACTTGAACTTGGTGACACGGCAACGGTAC | |
| Construct | | |
| Amino acid | DGPDRLLSKTFFPDGEDRPVRSRSEGPDLW | 42 |
| sequence of | | |
| peptide C | | |
| HDAd-EF1a-PYY | CATCATCAATAATATACCTTATTTTGGATTGAAGCCAATATGATAATGAGGGGGTGGAGTTTGTGACGTG | 43 |
| Approach 1 | GCGCGGGGCGTGGGAACGGGGCGGGTGACGTAGTAGTGTGGCGGAAGTGTGATGTTGCAAGTGTGGCGGA | |
| Codon | ACACATGTAAGCGACGGATGTGGCAAAAGTGACGTTTTTGGTGTGCGCCGGTGTACACAGGAAGTGACAA | |
| Optimized | TTTTCGCGCGGTTTTAGGCGGATGTTGTAGTAAATTTGGGCGTAACCGAGTAAGATTTGGCCATTTTCGC | |
| Nucleic Acid | GGGAAAACTGAATAAGAGGAAGTGAAATCTGAATAATTTTGTGTTACTCATAGCGCGTAATATTTGTCTA | |
| Sequence | GGGCCGCGGGGACTTTGACCGTTTACGTGGAGACTCGCCCAGGTGTTTTTCTCAGGTGTTTTCCGCGTTC | |
| Encoding PYY | CGGGTCAAAGTTGGCGTTTTGATATCAAGCTTATCGATACCGTAAACAAGTCTTTAATTCAAGCAAGACT | |
| (3-36) | TTAACAAGTTAAAAGGAGCTTATGGGTAGGAAGTAGTGTTATGATGTATGGGCATAAAGGGTTTTAATGG | |
| Approach 1 | GATAGTGAAAATGTCTATAATAATACTTAAATGGCTGCCCAATCACCTACAGGATTGATGTAAACATGGA | |
| Construct in | AAAGGTCAAAAACTTGGGTCACTAAAATAGATGATTAATGGAGAGGATGAGGTTGATAGTTAAATGTAGA | |
| HDdelta28E4- | TAAGTGGTCTTATTCTCAATAAAAATGTGAACATAAGGCGAGTTTCTACAAAGATGGACAGGACTCATTC | |
| MAR-EF1a | ATGAAACAGCAAAAACTGGACATTTGTTCTAATCTTTGAAGAGTATGAAAAATTCCTATTTTAAAGGAAA | |
| shuttle | ACAGTAACTCACAGGAAATACCAACCCAACATAAAATCAGAAACAATAGTCTAAAGTAATAAAAATCAAA | |
| vector | CGTTTGCACGATCAAATTATGAATGAAATTCACTACTAAAATTCACACTGATTTTGTTTCATCCACAGTG | |
| | TCAATGTTGTGATGCATTTCAATTGTGTGACACAGGCAGACTGTGGATCAAAAGTGGTTTCTGGTGCGAC | |
| | TTACTCTCTTGAGTATACCTGCAGTCCCCTTTCTTAAGTGTGTTAAAAAAAAAGGGGGATTTCTTCAATT | |
| | CGCCAATACTCTAGCTCTCCATGTGCTTTCTAGGAAACAAGTGTTAACCCACCTTATTTGTCAAACCTAG | |
| | CTCCAAAGGACTTTTGACTCCCCACAAACCGATGTAGCTCAAGAGAGGGTATCTGTCACCAGTATGTATA | |
| | GTGAAAAAAGTATCCCAAGTCCCAACAGCAATTCCTAAAAGGAGTTTATTTAAAAAACCACACACACCTG | |
| | TAAAATAAGTATATATCCTCCAAGGTGACTAGTITTAAAAAAACAGTATTGGCTTTGATGTAAAGTACTA | |
| | GTGAATATGTTAGAAAAATCTCACTGTAACCAAGTGAAATGAAAGCAAGTATGGTTTGCAGAGATTCAAA | |
| | GAAAATATAAGAAAACCTACTGTTGCCACTAAAAAGAATCATATATTAAATATACTCACACAATAGCTCT | |
| | TCAGTCTGATAAAATCTACAGTCATAGGAATGGATCTATCACTATTTCTATTCAGTGCTTTGATGTAATC | |
| | CAGCAGGTCAGCAAAGAATTTATAGCCCCCCTTGAGCACACAGAGGGCTACAATGTGATGGCCTCCCATC | |
| | TCCTTCATCACATCTCGAGCAAGACGTTCAGTCCTACAGAAATAAAATCAGGAATTTAATAGAAAGTTTC | |
| | ATACATTAAACTTTATAACAAACACCTCTTAGTCATTAAACTTCCACACCAACCTGGGCAATATAGTGAG | |
| | ACCCCATGCCTGCAAAAAAAAAAAAATTAGCCAGGCATGGTAGCATGTACCTGTAGTCCCAGCTACTTGA | |
| | GAGGTGAGGTGGGAAAATCACTTTAGTGCAGGATGTTGAGGCTGGAGTGAACTGTGATTGTGCCACTGCA | |
| | CTCCAGCCTGGACAATAGAGCAAGACCTTGTCTCAAAAAAATGCATTAAAAATTTTTTTTAAATCTTCCA | |
| | CGTAACACATCCTTTGCCCTCATGTTTCATAAGGTAAAAAATTTGATACCTTCAAAAAAACCAAGCATAC | |
| | CACTATCATAATTTTTTTTAAATGCAAATAAAAACAAGATACCATTTTCACCTATCAGACTGGCAGGTTC | |
| | TGATTAAATGAAATTTCTTGGATAATATACAATATTAAGAGAGACTGTAGAAACTGGGCCAGTGGCTCAT | |
| | GCCTGTAATCCCAGCACTTTGGGAGGCTGGGTAACATGGCGAACCCTGTTTCTACAAAATAAAAATATTA | |
| | GCTGGGAGTGGTGGCGCACACCTATAGTCCCAGCTACTCAGGAGGCTGAGGTGGAAGGATCGCTTGAACC | |
| | CAGGAGGTTGAGACTGCAGTGAACTGTGATCATTCTGCTGCACTGCACCCCAGCCTGGGCAACAGAGACC | |
| | TTGTCTCaAAAAAAAAAAAAAAAGAGACAAATTGTGAAGAGAAAGGTACTCTCATATAACATCAGGAGTA | |
| | TAAAATGATTCAACTTCTTAGAGGAAAATTTGGCAATACCAAAATATTCAATAAACTCTTTCCCCTTGAC | |
| | CCAGAAATTCCACTTGAATAAAGCTGAACAAGTACCAAACATGTAAAAGAATGTTTCTTCTAGTACAGTC | |
| | GGTAAGAACAAAATAGTGTCTATCAATAGTGGACTGGTTAAATCAGTTATGGTATCTCCATAAGACAGAA | |
| | TGCTATGCAACCTTTAAAATATATTAGATAGCTCTAGACACACTAATATTAAAAGTGTCCAATAACATTT | |
| | AAAACTATACTCATACGTTAAAATATAAATGTATATATGTACTTTTGCATATAGTATACATGCATAGCCA | |
| | GTGCTTGAGAAGAAATGTGTACAGAAGGCTGAAAGGAGAGAACTTTAGTCTTCTTGTTTATGGCCTCCAT | |
| | AGTTAGAATATTTTATAACACAAATATTTTGATATTATAATTTTAAAATAAAAACACAGAATAGCCAGAC | |
| | ATACAATGCAAGCATTCAATACCAGGTAAGGTTTTTCACTGTAATTGACTTAACAGAAAATTTTCAAGCT | |
| | AGATGTGCATAATAATAAAAATCTGACCTTGCCTTCATGTGATTCAGCCCCAGTCCATTACCCTGTTTAG | |
| | GACTGAGAAATGCAAGACTCTGGCTAGAGTTCCTTCTTCCATCTCCCTTCAATGTTTACTTTGTTCTGGT | |
| | CCCTACAGAGTCCCACTATACCACAACTGATACTAAGTAATTAGTAAGGCCCTCCTCTTTTATTTTTAAT | |
| | AAAGAAGATTTTAGAAAGCATCAGTTATTTAATAAGTTGGCCTAGTTTATGTTCAAATAGCAAGTACTCA | |
| | GAACAGCTGCTGATGTTTGAAATTAACACAAGAAAAAGTAAAAAACCTCATTTTAAGATCTTACTTACCT | |
| | GTCCATAATTAGTCCATGAGGAATAAACACCCTTTCCAAATCCTCAGCATAATGATTAGGTATGCAAAAT | |
| | AAATCAAGGTCATAACCTGGTTCATCATCACTAATCTGAAAAAGAAATATAGCTGTTTCAATGAGAGCAT | |
| | TACAGGATACAAACATTTGATTGGATTAAGATGTTAAAAAATAACCTTAGTCTATCAGAGAAATTTAGGT | |
| | GTAAGATGATATTAGTAACTGTTAACTTTGTAGGTATGATAATGAATTATGTAAGAAAACAACAGGCCGG | |
| | GCGGGTTGGTTCACACGTGTAATCCCAGCACTTTGGGAGGCTGAGGCAGGCAGACTGCCTGAGCTCAGGA | |
| | GTTCGAGACCAGCCTGGGCAACACGGTGAAATCCCGTCTCTACTAAAAATACAAAAAAATTAGCCGGGTG | |
| | TGGTGACACATGCCTGTAGTCCCAGCTACTTGGGAGGCTGAGGCAGGAGAATCACTTGAACCTGGGAGGT | |
| | GAAGGTTGCAGTGAGCCAAGAATGCGCCACTTCACTCCAGCCTGGGAAACAGAGCAAGACTCTGTCTCAA | |
| | AAAAAACAAAACAAACAAACAAAAAAACAGGCTGGGCGCGGTGGCTCACGCCTGTAATCCCAGCACTTTG | |
| | GGAGGCCGAGGCGGGTGGATCACCTGAGGTCAGGAGTTCCAGACCAGCCTTGTCAACATGGTGAAACCTC | |
| | CCCCCGCCGTCTCTACTAAAAATACAAAAATTAGCCAGGCGTGGTGGCAGGAGCCTGTAATCCCAGCTAC | |
| | TTGGGAGGCTGAGGCAGGAGAATCGCTTGTACCCAGAAGGCAGAGGTTGCACTGAGCTGAGATGGCACCA | |
| | TTGCACTCCAGCCTGGGGGACAAGAGCGAGATTTCGTCTTTAAAAAACAAAAAACAAAACAAAAAACCAT | |
| | GTAACTATATGTCTTAGTCATCTTAGTCAAGAATGTAGAAGTAAAGTGATAAGATATGGAATTTCCTTTA | |
| | GGTCACAAAGAGAAAAAGAAAAATTTTAAAGAGCTAAGACAAACGCAGCAAAATCTTTATATTTAATAAT | |
| | ATTCTAAACATGGGTGATGAACATACGGGTATTCATTATACTATTCTCTCCACTTTTGAGTATGTTTGAA | |
| | AATTTAGTAAAACAAGTTTTAACACACTGTAGTCTAACAAGATAAAATATCACACTGAACAGGAAAAACT | |
| | GGCATGGTGTGGTGGCTCACACTTGTAATCCCAGTGCTTTGGGAGGCTGAGACAGGAGAGTTGCTTGAGG | |
| | CCAGGAGTTCAAGACCGACATGGGGAATGTAGCAAGACCCCGTCCCTACAAAAAACTTTGTAAAAATTTG | |
| | CCAGGTATGGTGGTGCATACCTGTAGTCCCAGCTACTCGGGAGGCGGAGGCAGAAGGAATCACTTGAGCC | |
| | CAGGAGTTTGAGGCTGCAGTGAGCTACGATCATACCACAGCACTCCAGCGTGGACAACAGAGTAAGACCC | |
| | TATCTCAAAAACAAAACAAAACAAAACAAACAAAAAAAACCACAAGAAAAACTGCTGGCTGATGCAGCGG | |
| | CTCATGCCTGTAATCCCAGTATTTTGGGAGGCCCAGGTGGGCGTATCACCTGAGGTCAGGAGTTAGAGAC | |
| | CAGCCTGGCCAACATGGTGAAACCCCATCTCTACTAAAAATACAAAATTAGCCAGGCATGTGGCACGCGC | |
| | CTGTAGTCCCAGTTACTGGGGAGGCTGAAGCAGGAGGATCACCTGAGCCCGGGAGGTGGAGGTTGCAGTG | |
| | AGCCGAGATCACACCACTGCACTCCAGCCTGGGTGACACAGCAATACCCTACCTCAAAATAAAAAAGAAA | |
| | AAGAAAAGAAAAGTTGCTGTCCCCGCTACCCCAATCCCAAATCCAAACAGCCTCTCTCATCTCACAGTAA | |
| | GGGGGAAAAATCACCCAAAAAAGCTAAGTGATCTTTTGAAAACCCAAACTCTTAGAAGTCTAAGATTATT | |
| | ATAGTCAACTCATGAAGTGTCATCATAAAAGATACTCTAATATTATTTAAGTAGAACCACATATTGGTTG | |
| | TCTTGGTATGTCTAGCCCCTGGCATACAAAATATTTAATAACACTGATATGGTACCTGTGATGTGAAAAT | |
| | GTACTATGAGTACAGCTTTATAAATACTATATATGTACCTATATACAGAAAAAAATACAACAAAATCATA | |
| | AAAGCACTTATCTTTGAAAGAGGAGTTACAGCAATTTTATTTAGTTCTTTATTGCTTTGCTATATATTCT | |
| | AAATTTTTTTCAATGAATATATATCACTTTTAAAAAAATTCAATGGTCTTTCTTATAAATTATCTTTGGC | |
| | AGCATGCGTTTTTATATATACATATAAAATGTATGGGAAATTTTTAAAGGATACATTAAATTAAAGCAAA | |
| | ATATACAAACAAAAAATCAGAATACAAAAAGATAAAAAGATTGGGAAGGGAGGGAGGGAGTAAGGAGGAA | |
| | GGGTGGGTGGGTATAGAGAAATATACCAAATAATGGTAAGAAGTGGGGTCTTGACACTTTCTACACTTTT | |
| | TTTAAATAAAAAAAATTTTTTTCTCTCTCTTTTTTTTTTTTAGAGACGAAGTCTCGCTATGTTGCCCAGG | |
| | CTGGTCTTGAACTCCTGGGATCAAGAGATCCTCCTGCCTCAGCCTCCCAAGGTGCTTGGATTACAGGTGT | |
| | GAGCCACCACGCCTGGTCACTTTCTACACTTTAATATATATATTTTTTCATTTTCAATGTCATTTTTATT | |
| | AGTTAATTTATAATACCCATTCACCATTATATTCAAAGTCTATTTGAAGAAATAAACCAGAAAGAATGAA | |
| | ATACTCTAGCTCACATGCTATTCAATACTAAATTACCTTTCAAATCACATTCAAGAAGCTGATGATTTAA | |
| | GCTTTGGCGGTTTCCAATAAATATTGGTCAAACCATAATTAAATCTCAATATATCAGTTAGTACCTATTG | |
| | AGCATCTCCTTTTACAACCTAAGCATTGTATTAGGTGCTTAAATACAAGCAGCTTGACTTTTAATACATT | |
| | TAAAAATACATATTTAAGACTTAAAATCTTATTTATGGAATTCAGTTATATTTTGAGGTTTCCAGTGCTG | |
| | AGAAATTTGAGGTTTGTGCTGTCTTTCAGTCCCCAAAGCTCAGTTCTGAGTTCTCAGACTTTGGTGGAAC | |
| | TTCATGTATTGTCAGGTTGGCCCGTAATACCTGTGGGACAACTTCAGCCCCTGTGCACATGGCCAGGAGG | |
| | CTGGTTGCAAACATTTTCAGGTAGGTGGACCAGGACATGCCCCTGGTCATGGCCAGGTGGAGGCATAGTG | |
| | CTATACAGCAGGCAGAAGTCAATATTGATTTGTTTTTAAAGAAACATGTACTACTTTCATAAGCAGAAAA | |
| | AATTTCTATTCTTGGGGGAAAAGATTATGCCAGATCCTCTAGGATTAAATGCTGATGCATCTGCTAAACC | |
| | TTCACATATCAGAACATATTTACTATAGAAAGAATGAAAATGGGACATTTGTGTGTCACCTATGTGAACA | |
| | TTCCAAAAATATTTTACAACAACTAAGTATTTTATAAATTTTATGAACTGAAATTTAGTTCAAGTTCTAG | |
| | GAAAATACAAACCTTGCTAGATATTATAAAAATGATACAATATATATTCATTTCAGGCTCATCAGAATAT | |
| | ATCTGTTATCACTTGACAAGAATGAAAATGCACCATTTTGTAGTGCTTTAAAATCAGGAAGATCCAGAGT | |
| | ACTAAAAATGACTTCTTCCTTGAAGCTTACTCACCAACTTCCTCCCAGTTACTCACTGCTTCTGCCACAA | |
| | GCATAAACTAGGACCCAGCCAGAACTCCTTGAAATATACACTTGCAACGATTACTGCATCTATCAAAATG | |
| | GTTCAGTGCCTGGCTACAGGTTCTGCAGATCGACTAAGAATTTGAAAAGTCTTGTTTATTTCAAAGGAAG | |
| | CCCATGTGAATTCTGCCCAGAGTTCATCCCAGATATGCAGTCTAAGAATACAGACAGATCAGCAGAGATG | |
| | TATTCTAAAACAGGAATTCTGGCAATATAACAAATTGATTTCCAATCAAAACAGATTTACATACCATACT | |
| | TATGTCAAGAAGTTGTTTTGTTTTATTGCATCCTAGATTTTATTTTTTTGATTTATGGTTTACTTTAAGC | |
| | ATAAAAAATTTGTCAATACAACTCTTCCCAAAAGGCATAAACAAAAATTCATAAAACTTGCATCACTTGA | |
| | GATACTTCAGGTATGAATTCACAACTTTGTTACAACTTACTATATATATGCACACATATATATATATTTG | |
| | GGTATATTGGGGGGGTTCTAATTTAAGAAATGCATAATTGGCTATAGACAGACAGTTGTCAGAACTTGGC | |
| | AATGGGTACGTGCAGGTTCATTATACCAAGTCTACTTGTAGTTGTTCAAAATGTATCATAATACAAGGCC | |
| | GGGCGAGGTGGCTCACGCCTGTAATCCCAGCATTTTGGGAGGCTAAGGCAGGAGGATTGCTTGAGGTCAG | |
| | GAGTTTGTGACCAGCCTGGGCAACAGAGCAAGACCCTGTCTCCAAAAAGAAAAAAAATAATTTTTTACAA | |
| | AATAAAAACAAAATGTATCATCAGACGAAATTAAATAAGAGGCAATTCATTTAAATGACAACTTTTCCCA | |
| | GCTTGACATTTAACAAAAAGTCTAAGTCCTCTTAATTCATATTTAATGATCAAATATCAAATACTAATTT | |
| | TTTTTTTTTTTTTTTTTTTGAGACGGAGTCTCGCTCTGTCGCCCAGGCTGGAGTGCAGTGGCGCGATCCT | |
| | GGCTCACTGCAAGCTCCGCCTCCCGGGTTCACGCCATTCTCCTGCCTCAGCCTCCCGAGTAGCTGGGATT | |
| | ACAGACATGCGCCACCACGCCCGGCTAATTTTGTATTTTTAGTAGAGATGGGGTTTCTCCATGTTGGTCA | |
| | GGCTGGTCTTGAATTTCCCACCTCAGGTGATCTGCCTGCCTCAGCCTCACAAAGCAGTAGCTGGGACTAC | |
| | AGGCACCCACCACCACACTTGGTTAATTCTTTTGTATTTTTTTTGTAAAGACGGGATTTCACCATGTTAG | |
| | CCAGGATGGTCTCGATCTCCTGATCTCATGATCCGCCCGCCTCAGCCTCCCAAAGTGCTGGGATTACAGG | |
| | CGTGAGCCACCCCGCCCGGCCATCAAATACTAATTCTTAAATGGTAAGGACCCACTATTCAGAACCTGTA | |
| | TCCTTATCACTAATATGCAAATATTTATTGAATACTTACTATGTCATGCATACTAGAGAGAGTTAGATAA | |
| | ATTTGATACAGCTACCCTCACAGAACTTACAGTGTAATAGATGGCATGACATGTACATGAGTAACTGTGA | |
| | ACAGTGTTAAATTGCTATTTAAAAAAAAAGACGGCTGGGCGCTGTGGCTCATGCCTGTAATCCCAGCACT | |
| | TTGGGAGGCCAAGGCAAGTTGATCGCTCGAGGTCAAGAGTTCGAGACCAGCCTGGCCAACGTGGTAAAAC | |
| | CCCGTCTCTACTAAAAATACAAAAAAAAAATTAGCCAGGCATGGTGGCACAGGCCTGTAATCCCAGCTAC | |
| | TAGGGAGGCTGAGACATGGAGAACTGCTTGAATCCAGGAGGCAGAGGTTACAGTGAGCCGAGATCATACC | |
| | ACTACACTCCAGCCTGAGTGACAGAGCGAGACTCCGTCTAAAAAAAAAAAAAAAAAAAAAGATACAGGTT | |
| | AAGTGTTATGGTAGTTGAAGAGAGAACTCAAACTCTGTCTCAGAAGCCTCACTTGCATGTGGACCACTGA | |
| | TATGAAATAATATAAATAGGTATAATTCAATAAATAGGAACTTCAGTTTTAATCATCCCAAACACCAAAA | |
| | CTTCCTATCAAACAGGTCCAATAAACTCAATCTCTATAAGAGCTAGACAGAAATCTACTTGGTGGCCTAT | |
| | AATCTTATTAGCCCTTACTTGTCCCATCTGATATTAATTAACCCCATCTAATATGGATTAGTTAACAATC | |
| | CAGTGGCTGCTTTGACAGGAACAGTTGGAGAGAGTTGGGGATTGCAACATATTCAATTATACAAAAATGC | |
| | ATTCAGCATCTACCTTGATTAAGGCAGTGTGCAACAGAATTTGCAGGAGAGTAAAAGAATGATTATAAAT | |
| | TTACAACCCTTAAAGAGCTTATAGCTGGGCGTGGTGGCTCATGCCTGTAAATCCCAGCACTTTGGGAGGC | |
| | TGAGGCGGGTGGATCACCTGAGGCCAGAAGTTCAAGACCAGCCTAGCCAACATGGCGAAACCCTGTCTCT | |
| | ACAAAAAATACAAAAATTAGCCGGGTGTGGTGGCACGTGCCTGTAGTCCCAGTTACTTGGGAGGCCGAGG | |
| | CAGGAGAATCGCTTGAACCTAGGAGGTGGAGGCTGCAGTGAGCCGAGATTGTGCCACTGCACTCCACTTC | |
| | AGCCTGGGCGACAAGAGCAAGACTCCGTCACAAAAAAAAAAAAAAAAAAAAAGCTTAAAATCTAGTGGGA | |
| | AAGGCATATATACATACAACTAACTGTATAGCATAATAAAGCTCATAATCTGTAACAAAATCTAATTCGA | |
| | CAAGCCCAGAAACTTGTGATTTACCAAAAACAGTTATATATACACAAAAAGTAAACCTAGAACCCAAAGT | |
| | TACCCAGCACCAATGATTCTCTCCCTAAGCAGTATCAAGTTTAAAGCAGTGATTACATTCTACTGCCTAG | |
| | ATTGTAAACTGAGTAAAGGAGACCAGCACCTTTCTGCTACTGAACTAGCACAGCCGTGTAAACCAACAAG | |
| | GCAATGGCAGTGCCCAACTTTCTGTATGAATATAAGTTACATCTGTTTTATTATTTGTGACTTGGTGTTG | |
| | CATGTGGTTATTATCAACACCTTCTGAAAGAACAACTACCTGCTCAGGCTGCCATAACAAAATACCACAG | |
| | ACTGAGTGACTTAACAGAAACTTATTTCTCACAGTTTTGGAGGCTGGGAAGTCCAAAATTAAGGTACCTG | |
| | CAAGGTAGGTTTCAATCTCAGGCCTCTTCTTTGGCTTGAAGGTCTTCTAACTGTGTGCTCACATGACCTC | |
| | TTCTAACAAGCTCTCTGGTGTCTCTTTTTTTTTTTTTTTCTTTTTTGAGACAGAGTCTCACTCTGTCACC | |
| | CAGGCTGGAGTACAGTGGCACAATCTGGGCTCACTGCAACCTCCAACTCCCGGGTTCAAGTGATTCTCAT | |
| | GCCTCACCCTCCCGAGTAGCTTGGATGACAGGAGCCCGCTACCACACCCAGCTAATTTTTGTATTTTTAG | |
| | TAGAGATGGTGTTTCACTACATTGGCCAGGCTGGTCTCAAACTCCTGACCTCGTGATCCACCCACCTTGG | |
| | CCTCCCAAAGTGCTGGGATTACAGGTGTGAGCCACTGCGCCCGTCCTGGTGTCTTTTCATATAAGGGCAC | |
| | TAATCCAATCAGACCTGGGCCCAACCCTCCCGACTTCTTCTAACTGTAATTACCTTCCAAAGGCCCTGTC | |
| | TCCAAATACCATCACACTGGGGGTTAGGACTTCAAAAAAGGTATGGGGGGGGTGTGGGAGGACATAAATG | |
| | CTCAGTCCATAACAAGCACCCAACATAAAAATGGCTAGAACAGATCACAAAAAAAAGGTCCTGTATGGCT | |
| | TTGGGGAAGGGCTCAACCCCAAAATATCTGAGAGCTCTGGAGGGGCCTAGAAGTGGTAAATGAATGAAAA | |
| | CGTGGTTACTCTCCAGATCTGCCTTTCCCAAATATGGCCATTCTTGGCTGAATCAGAAATCAAAGGACAG | |
| | GTTATTAATTACTAGCTCTAAGTTACTTACCATTTGCTGAGACAGTTCAGAAATCTGACTGCATCTCCTC | |
| | AGAGATCTAGAACACAGTTCTCAAATTCTAACTTACTTGTGATATACTTGTGAATGATAAAAATCGCTAC | |
| | AGGTACTTTTATTAATCTGAAAGAGTATTGAGAAATTACCTTTCATTCTGACTTTTGTCTGGAATGAAAA | |
| | TCAATACTTTTGCTATAATCGATTACTGAAATAATTTTACTTTCCAGTAAAACTGGCATTATAATTTTTT | |
| | TTAATTTTTAAAACTTCATAATTTTTTGCCAGACTGACCCATGTAAACATACAAATTACTAATAATTATG | |
| | CACGTCACATCTGTAATAATGGCCTTCATGTAAACATTTTTGTGGTTTACACATAAAATCTCTAATTACA | |
| | AAGCTATATTATCTAAAATTACAGTAAGCAAGAAAATTAATCCAAGCTAAGACAATACTTGCAACATCAA | |
| | TTCATCATCTGTGACAAGGACTGCTTAAGTCTCTTTGTGGTTAAAAAGGAAAAAAAAAAAAAAGACATGT | |
| | TGGCCAGATGCGGTGGCTCACACCTGTAATCCCAGCACTTTGGGAGGCTGAGGTGGGCGGATCACCCCTG | |
| | GCCTGCCCAACATGGTGAAACCCCGTCTCTACTAAAAACACAAAAATTAGCTGGGCGTGGTGGCGGGCGC | |
| | CTGTAATTCCAGCTACTCGGGAGGCTGAGGCAGGAGAATTGCTAGAACCCAGGAGGCAGAGATTGCAGTG | |
| | AGCTGAGATTGCACCATTGCACTACAGTCTGGGCAACAAAAGTGAAACTCCATCTTAAAAAAAAAAAGAC | |
| | AATGTTCGTGGGTCCAAACAAGACTTAATGGAAGTGAGTCTAAAAATGAGCTATGTGGGCCAGGCGTAGT | |
| | GGCTCCCACCTGTAATCCCAGCACTTTGGGAGGCCGAAGCAGGCAGATCATGAGGTCAGGAGATGGAGAC | |
| | CATCCTGGCCAACACGGTGAAATCCTGTCTCTACAAAAATTAGCTGGGCGTGGTGGTGCCTGCCTGTAAT | |
| | CCCAGCTACTCAGAAGGCTCAGGCAGGAGAATCGCTTGAACCAGGGAGTCGGTGGCTAGAGTGAGCCGAG | |
| | ATTGCATCACTGCACTCCTGCCTGGTGACAGAGCAAGACTCCATCTCAAAAAAAACAAACAAAAATAAAA | |
| | GATAAAAATGAGCTATGTGAATTAAAAGAGGTATAACAATAGATAAACCATATTTTATTTAATTCCTAGT | |
| | AATGAGTAATATTTCCAAACTTCTGGAATGGGCAGAAATTGCTAGTTGGCATATTTTTACCTTTTATATT | |
| | CAGATACATTAAAATTCTCAAAAAAAAACACCTCAAAGCAGATGATCCGCCATCTCCTTGGATAATTTGT | |
| | GTTAACTCAGGATAACAGAAAACCAAAATTATGAGTTACTGATGCAATATTCCTAAATGTAAAAATAATT | |
| | AAAGCTAATAGTAGATTCATCTTCCAATTTCATATCAGTCTTACAAATAAACTACATATATAACTTGCTT | |
| | GCCTTCCCTTCTGAGGGATAAAGCTGTTAGAAGAATTAAAATCAGCATTCTTGACTATTCAACCAAGGGA | |
| | GGGATAAATTATTACTCATTCTAGGGACATGGGCTCATAACTACTACATGTGTAAGGACATGAATTTACC | |
| | CAATATTACAATTTTTCCTTTTATTAGTGTGTACAGTGGAAGAATAGACATGTTCACTCTGGACAAAAAA | |
| | AAAATTATACTTATCAGTTATCAGAAGCACAATGCTGAAGACAGTAGTTCCATAACAATTTGAAGTATGT | |
| | GATCGAACTAGTAGATTATCTTAGTAGTAGTGAATTATTGTAAATGTTAGTAATTTGGCAGCCACTGGGC | |
| | AGAAAAATAAGAATTGAGGCTCAATATTGATATTAATGGTGGTGATTGACACATAAATTTTATCAAGTCT | |
| | ACACAATATAAAATTACAGAAAGGTAGAAGAGTATACCAGTACAACTTCAACATATCTTCACTACAAGGG | |
| | AGTAAAATGACATGGCCTAGTTACTATCTAATGAACTGCAGAAAACTAAAAGAAAACTCCAAGGCAACTC | |
| | TTCTCTGCTGATCTGGTTGGTCCTTTTCCTACCTTTTGCAATACCCAGATACAAACAATGGATAGAAAAC | |
| | AAAGTAGACTTGTAGTATGCAGGTCACAGTGCTAAATTCACAGAAAGAAACCCCTGAACTGAACTGCTCT | |
| | ATTTCCTGGTGGTCACAAAGAGTAATTCTGGTTTACACCTACAGATTGATGTCAATCTACACCCTGTTGA | |
| | TAACAGTGTGGCCAAGGACAAAAAAAAGGTGCTCCGTTTTACCAATTCTGTAAAAAATTATTGGCAGGGT | |
| | AAGCTCGGCTAGGGCAGGATTACATTTCTAGGACTACCATCCCCGAAATTTAGAAGATATTATATCCACA | |
| | TAAAGCATATCTTTCACATTAATTTGCAAAAATCTAAAAGCTTTTTCTTAGCTCAAGTGTGTCCAAGTTT | |
| | ACCCTGGCAGTTTAAAACGATAGTTACAAGCAGCATGGGTTGTATCAGACACATTTGAGGGCCAATTTCA | |
| | TGTAAGTGATATTGGGCAAGTTACTTCAACTATCTGTGCCTCCAAGGTCATACTAGTGTTTATTTACCTA | |
| | AAGGGTACCTGTTATGTAACTTTAGGGTGTTTACATTAGATAATGCCTGCAAAATATTTACTTCAACGCC | |
| | TAAAACATAGTTAAGTATTCAATAAATACCTACTATTGTCACTACTAACTTAAAAGTTTAGAGATTAAGA | |
| | GCAGAATCTGGGGTGAGACAAACTTAGGTTCAAATCCTAGTATTGTTGGGTAATCTTGGGCAAGTTACTT | |
| | AACCTCTCTGATTTGTGTAATTTAAAAAATTAGTTAATATACATAACAGGGCTTAGAAGAGTATCTAGCA | |
| | CATAGCACCATTTAAGCATTTGTTATTGCTAACATGCAAACAATTTAAGGGAAAGAAATTTTTTAAAAAG | |
| | GAAGAGGGATTTGCAAACTAAAAACAATGAGTATCTTATGTTCAAAGAAAACTAACAAACAGCCAGCTCT | |
| | AGCAATAATTAAATTCACTATATACTGGGGCAGGCATCACACCCCAAAGCTAAAAGCGTCTACCTAGGCC | |
| | AGGCACGGTGGCTCATGCCTGTAATCCCAGCACTTTGGGAAGCAGAGGCGGGCAGATCGCTTGAGCTCAG | |
| | GAGTTCAAGACCAGCCTGGACAACATGGCAAAACACCATCTCTACAAAAAATACAAATATTAGGCCGGGC | |
| | GCAGTGGCTCACGCCTGTAATCCCAGCACTTTGGGAGGCCAAGGCGGGTGGATCACCTGAGATCAGGAGT | |
| | TCGAGAGTAGCCTGGCCAACATGGTGAAACCTCGTCTCTATTAAAAATACAAAAAATTAGCCAGGCATGG | |
| | TGGCAGGCGCCTGTAATCCCAGCTACTCAGGGGGATGAGGTAGGAGAATCGCTTGAACCCGGGAGGCAGA | |
| | GGTTGCACTGAGCCGAGATCATGCCACTGTACTCCAGCCCGGGCAACAAGAGCGAAACTCCATCTCAAAA | |
| | AATAAATAAATAAATAAATAAAATAAAGTACAAATATTAGCCAGGGATGGTGGTGCGCACCTGTAGTCCC | |
| | AGCTACTTGGGAGGCTGAAGTGGGAGAATCCCCTGAGCCTGGGGAGAATCACCCGAGCCCGGGAAGTCGA | |
| | GGCTGCAGTGAGCAGTGATTGTGCCACTGCACTCCATCCTAGGTGACAGAGTGAGACCCTGTCTCAAAAA | |
| | AAAGAAATTGGCAGAATTAAGTAAGTTGATGTTTAGAGATGAAAAATCAACATTTTTTCCTCAGCAACTG | |
| | AATAAAAACAACAGCCACTACCATTTTTTTGAGTACCTATTTGTAGCCTATTTTTTAACTGGTATTACTC | |
| | GAGAGAGAGAGAGCTAGGTTCGAGACAGAGCTCCTTCTCTTAATAACTGTATGACCTAGGGTATGTCTGT | |
| | TAGCCTCTCTGAGGCTTCAAAGGTTCCTCATCTGTAAAATGGTAATAATCATACCATTGCTACAGGGCTG | |
| | TTTTGAAGACTAATTAGGACTATGTAAGTAAACATGATGATGGCTATTATTACTGTTCCCCGCCAGGGGC | |
| | CATGCAAGGGTTGCTGATTCACATAGACTGTCTTATAATCCTCTCAATAACTCCAAGAGGTAGCCAGCAC | |
| | CTCAGATATACATAAAATGACTTAAGCCCAGAGAGGTGAAGTAAGTTGCCCACAGCCACACAACTAGTAA | |
| | ATAGCCCAAACAAGCTGGATTCCCAGTTAGACTCCGTTAATAGCACTGCTCTTTACCTTAAGTCATTACA | |
| | ATGCCTAATATGAAATAGAATCGCTTCTTTCTTAGGGTTCAAGTGGTTAATTATTTAATGTATTCATTCA | |
| | ACAAACCATCATCGAGGACCTCTTACAAGCCAAGTACTGTGCTAAGTGCTAGAGTTACGGCGGTGATTCC | |
| | TGCCCTTAAAAAGTTTTAGTGGGAGAAACAACAGGTAACCAGGTCATTGCCAAAACAACAAAAATAATCA | |
| | TAATAAAGCAGGCTAAAGCATATTTAACTGGCCGGGGTTTTGACTATTTTAGCAAGCATGATCAGAACGG | |
| | TTGAGGAGGGAGGCCAGCAGCTTGGCCGGTTCAACAAACAAGAAAAAACCAGTGAGGGTGGAGCTAAGAT | |
| | ACCAGAGGCTGATTACGGTTAAGAATGTTCTTGAAGGTAAGGACCAGATTCTCATTTTCTATATCCTGGG | |
| | GCATCGGTCAGCATGGAATCTGGATTCTAGCACATGTGAATTTCGGCTTGAAATGACCTAATGCCTTTTC | |
| | CCTAGTTCCTTCGTGTGTCAAATACGCATGGTTACCGCTACCAGAGCTGTAGTGGGGCTTCAATGAGGCC | |
| | ATGAGCATCTCCATAAAGATGAACTACAGTGTGTGCAAAACTAAAGGCAAAACCTGGTCCCCACACGCCC | |
| | TCCCAGGTGGTCGCTTTCCGTGCCGAGGCCCCTCCAGAGGTGCCCCGAGAACCTCACCATCGCACCCCAA | |
| | ACTTCCAGGGAAGGGCCTCTCCCGAGAAAGCCCCCACGCCCCCACCCCGCGCCATCATTCCCGAATCTGC | |
| | CCTCGGCCCCTCCCCGCAGCACGCTCGCAGGCGGCACATGTCAACCAAAACGCCATTTCCACCTTCTCTT | |
| | CCCACACGCAGTCCTCTTTTCCCAGGGCTCCCCCGAGGAGGGACCCACCCCAAACCCCGCCATTCCGTCC | |
| | TCCCTGCCGCCCTCGCGTGACGTAAAGCCGAACCCGGGAAACTGGCCGCCCCCGCCTGCGGGGTTCCCTG | |
| | GGCCCGGCCGCTCTAGAACTAGTGGATCCCAATTGAAGGCCTGGTCTAAATGACTCCAAAATCACCACTT | |
| | AATTCAAGAGACTGATTTCCCTGAGTCAGGCCCCTTAAAGCAGCTATTTCAATGGGACAGGGAAACAACC | |
| | CTAGGATCTGGATTAGAATCACTTGGGGGCTGCCACACCCCCAGGGCTCTGATCCTGCCCTTCTCCCACA | |
| | CGCACATTCACATACTGCTGCAGTGACCTTCCATTTCTAATGGGTTCCTGGGCCATCTGTCAGGTATAGG | |
| | GAATGGAAAAGGGGTTGGGGAGGCTCTGCTTCAGAAAGTTTGTGTCAGGGGCTCCCAGAGCCTCCACAGA | |
| | TAGATAGCAGGGGTCCCCACCCTACCATGGCAGCTATAAATGTGATCAACATTTATTGGCCTAGGATACA | |
| | GCAGTTAGCAAAATGCCTGATGTAGTTCCCACTCCGTGGAGGTTGCAGGCTAGCCAAGAAGTCATGAGTT | |
| | CAGCAACCCTTACGCACCAGTGGGATGAGATTGGACCAGGCCGAGGGTAGTCTTGGGAACACTCAGCATT | |
| | TGTCTGAGGGCCAGAAGAGGCTGCTTGCCCTCAGACAGGAGGTCAGCATCTTTATTGTAGCCCATGACAC | |
| | CTCTACACCATTGCTCTTCTGGTCTTATGGAAGACATCTTTGGGCCTGATAACAGCGGAGTCTGTGTCCC | |
| | ACTTGTCCAGGCTGGAGTGCCACATCAGGCACACTCCAGTTGCAGGGACAGCACAGACAAGTTTCAGGAA | |
| | GGCTGGTGGCCTCCAGGAGGTTAACCTTATAAGGCCAGATTGTAACCTAGTTGAAAAACATACACATGCC | |
| | ATGATAATAAAAGAACCTAGGCACCATTACAAGAGAAAAAATCATTTTTGTAGATACGAGCATGGATTCT | |
| | TGGGTGGGTCAGACACACTGGGCTTGTGCTCTGACTGCACTGTCTCCCCTACCTGACCTTGGGTAAACCA | |
| | TAAGACTGCTGCATGACTCAGTGTCCACCCCAAAAAAGTACCGGTAGATATTGGCCACAGTAGATATCAG | |
| | CTAGAGTGGACTCTCATGACAATGAGGGGAGATGTATTCCCCATCTTAGGCACCTGGGACTCTACCTTCC | |
| | ATCTTCTGCTCCGTGTCTCTCCATCCCCAGGCTCTTCAGAACTCAGGGAGTCCAGAATGTCAGCTCCCAG | |
| | ATTTCAGCCTTCAGAAAGGAAACCCATTACCGTTCAGTTGAACAAATGTTGTCTGAGCCCCAGATCTGGG | |
| | CTCAGAGGCCATCTAGGCTATGAGACAAGAGGGGAACAAAGCACCGTCTGCACTCACTCACCACACTCAC | |
| | TTGCTGTCCCAGGTCACATCCATCGGGTAGAGAATCTAAGAGGCTGAGCTAGCTCCCGCCACCAGCCCAG | |
| | CCCACCCCACCTGGCCCCTTCCTTCCTTCTACAAAATATGCACCACCTGTCAAAGGGTGGGCAGTGCCAG | |
| | GCCTGCATACAGAGCACTGAGTGTAAAAGCAGACATGGACCCTGACCTCCAGGAGCTTCCAATTTTCTTG | |
| | AAGAGACAAATCAGCTGGCATTTCAGTCCAGTGTGATCTGCTCTTGGTGAGCACAGACCTAGGGAGTTGG | |
| | GGCAGCTTCCCAGAAGAACTGCAGTCCAGGCTGAGGGCAGAGAAATGAGGGGAATGGCGAGGAATTGGGG | |
| | AGCAGGGGGGAGCTCAGTAGAGAGCCAAGGGCGGGAGGTGAGAAGTCCGTGTTGGGCCAGGAGCTACCCT | |
| | CCGGTGGCCACAGCCGAAGTCGAGGATGCCTTTGGAACTCATCCCCACTTCTCTCTTTCTGTATGTAGCC | |
| | GTCCAAGAACAAGTCACCTCCAAGTGTAGCCGGATCAAGGCAAGCCCCCCATCTAGCAAGCACTTGATGC | |
| | CACCCAGAACTGGGCTTCTTCAGAACAATCTGAGTCCAGGAATGATCCCACTCACCAGGCACCAGAGCTG | |
| | CGAGGGCATGGGAGTGATCTCACCAACTCTGGGGAAGCGGCAAGGAATTTTCACCTCCAGCCCCCAGTGT | |
| | CCCATCCTCTCACACTCAGGCCAGACTCCCCTGGGCAGACTTGACTCTGTCTGCCAGCATATGCAGAGCC | |
| | CCAAGGCCACCCCACCAGAAGTGCCCCTGCCTGGGTTCTGTCCCAGCTCCCTGGGCACCCAGTCCTTGAG | |
| | TCCCCACCAGCTCAGACGGCCTAGTGTGCCAAGAATGCCCACTGCGTTCAACAATGCTGCATGGGTCACA | |
| | GCGGCAGCAGCTGTGACCACAGCAGTTTCGGGGAAAACACCCCTCAGCCAAGTGGATAATAGCGTTCAGC | |
| | AGCACTCACCTTCTGGCCAGGCCTGCCTTCAGAGGCCATCTGATTGGGAGGCACAAGTGCCCGCTGCGAT | |
| | GGGAACACAAGTGCCCCTGGCCAACAACCCCAGCTTCAGCCTGCTGGGCAGCCAGAGCCTCAGGCAGAGC | |
| | CCGGTACAGGGCCCGGTGCCTGTAGCAAACACCACCAAGTTCCTCCAGCAGGGTATGGCCAGCTTTAGTC | |
| | CCCTGAGCCCCATACAGGGCATCGAGCCACCAAGCTATGTGGCTGCTGCTGCCACCGCTGCTGCTGCTTC | |
| | TGCCGTTGCTGCCAGCCAGTTCCCAGGTCCGTTCGACAGAACGGATATTCCCCCTGAGCTGCCACCTGCC | |
| | GACTTTTTGCGCCAGCCCCAACCCCCACTAAATGATCTGATTTCGTCACCTGACTGCAATGAGGTAGATT | |
| | TCATTGAAGCTCTCTTGAAAGGCTCCTGTGTGAGCCCAGATGAAGACTGGGTGTGCAACTTGAGGCTGAT | |
| | CGACGACATTTTGGAACAGCATGCTGCTGCTCAAAATGCCACAGCCCAGAATTCTGGGCAAGTCACCCAG | |
| | GATGCTGGGGCACTTTAAATCTGAGCAGGATGCCCATAGAAACCCCCATGGTGACATCACTCTAGGAAGT | |
| | GGTGTCGATCCATACCCGCAGTTGTCTCCCGTTACAATTTGAGTGGTGTTGTCAGCCCATGCTTATCCCT | |
| | CTCTCTACCTGTGACAAAATGGAAAGCTGGTGATTTTTCAAGCTACGTGTACATATTTGAAAATTTTGTA | |
| | AATGGTTTTCCTAAACATTAATGACAGAAGTATTTATACTTCATTTTGTGACTTTGTAAATAAAGCGACG | |
| | GCTTTTGTTTCAGTAGAGTTGTGTTTACTATGCATTGTTTTGTGTTTATTATACAATGTTACAAATATGC | |
| | AGACCGTGTTGTTTGCTCCAGTGATACCTTGTTAAGCTAGGTGGCTGAGTCGCTTATGGTTTTAATGCAA | |
| | TGAGCAATGTGGATATGACCAAGAGTTGTTGTGCAAGTTGACAAATGCCAAATAGAAAACCACTTGGCCA | |
| | TTTATTTCTATGTTCACTAAAAATCCTATTGCCTTGTGTGATTCTTAATCTCTTTTGCGAACCTTTCAGT | |
| | CTCCGCTAGCTCTTTCCTAATGAGCTTTACAGCAGAAGCTGTTTTATCGTTAAGTGCCCCACAGAGACAC | |
| | TTTACCAGGAGGCTGGGAGAGTTCTCCAGATTTGGGAGAGGCGCAGAGACAGTGTGTGAGCCGAGCCCTG | |
| | TCTCAGCAATCCACCTGGAGGAGCTAGAGTATCCTCCTCCCTTTACCATTCAGACCGAGAGAAAAAGCCC | |
| | AGCTTGTGTGCACCCTCGTGGGGTTAAGGCGAGCTGTTCCTGGTTTAAAGCCTTTCAGTATTTGTTTTGA | |
| | TGTAAGGCTCTGTGGTTTGGGGGGGAACATCTGTAAACATTATTAGTTGATTTGGGGTTTGTCTTTGATG | |
| | GTTTCTATCTGCAATTATCGTCATGTATATTTAAGTGTCTGTTATAGAAAACCCACACCCACTGTCCTGT | |
| | AAACTTTTCTCAGTGTCCAGACTTTCTGTAATCACATTTTAATTGCCACCTCGTATTTCACCTCTACATT | |
| | TGAAATCTGGCGTCTGTTTCAAGCCAGTGTGTTTTTTCTTCGTTCTGTAATAAACAGCCAGGAGAAAAGT | |
| | GCCTCTATGTTTTTATTTTTCAAGGGAGTATTCAGTACCTACAAACCCAAGTCAGGAAGCCTGCTAGTGG | |
| | CTTTGGTTCTTTCAGAGGCTGCTCGATGCCTTGTGTGTCAGAAAGAAAGATTCAGCAGTTTTGCATCATG | |
| | GCAAAGAAGCCTGTTATTTTGGGGCTCAGCCCCTCATTTTATAGAGGATGAAACAGAGGGGGATGGGAGG | |
| | TCACAAAGACAACTGCCCCGGGAGCAGGTGTGGGGGAGACTTGCCCTGAGGGTCTAGACGCTCTGCACCA | |
| | CCGTCCTGTCTCCCTTGCTGAAGACCACACATGCCCTTCTTTGACCAGACCCTGCCACCTGATAGGCCAG | |
| | GACCTGGTAGGCGGGTACCCAGGTTTCATGGATGGAACCACATCTCCCCAAAAGTGGGGAGGTAGCTACT | |
| | GGGATGCACGCCTCCCGCCATGTGCTATAGGAGAGCAGCTGAAGCAACAGTTGGGATCAGATGTAGTCAC | |
| | AATTGAATGCATCATCACATTTATCCCTCTAAGTGGCTGGGAGAGTTGATATCCTCATCCCTAAGGTACA | |
| | AAATGTTCCAATTTGATCAGTGGCTTTCAGGAGCTGAGAAAGGCATGTGCTCTGAGGCAGAGCTGTTATG | |
| | TCCCGCAGAGCCTAAAAATGCTCTAAGAACATGCTCCCTGCCAAAATTCTCAATGGCTGTGACAAGGGAC | |
| | AACGATCGACCAATGGGGGTGGAAGCAGACCTCCGCAGTCCAGGGGCCAGAGCTAGGACAGAGGGGTCGG | |
| | AGAAAGAGTCATTTTCCCAACACTCCAGCTCTTGGCCAGTCCTCACACAGTCCCCTCCTGCTTCCTGCTG | |
| | AGAGAGATATCCTCATAGGTCTGGGTAAAGTCCTTCAGTCAGCTTTCATTCCCTGTCACCAACTTTGTCT | |
| | CTGTTCTCCCTGCCCGTCTCAGGCAGCACTCCTCAGGAAACCTCTCCAAGAGCCAGCCTCACTGCAGCGC | |
| | CCACTATTGTCCCTCTGCCTCAAGTGTCCCATCCATGCCAGGCCCCAGGCAGGCTGCAGCTTTCCCTCAG | |
| | GGCCACACCAAAGCACTTGGGCTCAGCTGTGCTGTCCCCCTCCATCACTGAGCTCAGGGGCAGCAGGGGT | |
| | GGGGTGCCAGGAGGCCCATTCACCCTTCTCTGGCTCTGTGTTGGACCCACCTGCCCAGCCACTGCTGCTT | |
| | AGAACCTACCCGCTGGGAAAATGAAGCCCTCCCGGAGGGGCCACCTCAACCTGAGAGCCTCACGGATCAC | |
| | AGTTGTCCCCACTCAGCTCTGCCAGCCCTCAGAGACCCATAGATAAAAGCTGAGCTTGGCTCGCAGAGCT | |
| | GGTTCCATCTTCCATTCCCAGAGGGTTCAACTTCCTACCCCAACCACACAGGGAACCTCAAGGCTGAGCC | |
| | AGTGTGGGCTGCAGTGCAGACCAGCTTCCTGGACACGTCCTGCCACCTGACCCCAGGCTGGCCTCACTGC | |
| | CCCTGGCACTCCTGACCCTATCCTCATTCCTCCTGGCAGTGCGTGTTCTGCCATTCCGCTTTCCCTTAGC | |
| | TGTCCTCTCACTGTACTGTCAGCTTCTCCTTTTCCAGGTGCCCCCCAGGGGCTTTCCACATGACCCTGTC | |
| | ACCCCACAGCCCATCCAGCACCAATTCCAGCTCTCTGCCACCCTTCAAAGGAGTGACAGTGCCCTGCTTC | |
| | ACCTCCCACTCACCCCTCAACCCAGAGCAATCTGGCTCCAGTCTTGCCTCCTTCCCCCTAAGTACTCTAG | |
| | TCACAGTTCCAAATTCCTCCTGGTCATAAAGCCAAATGAAGCTTCCTGGTCCTCAGCGGACTTGCCACTT | |
| | CAGCAGTACTGGACTCTCTCCTCCCAGAAACCTGTTTCCCCTTGGCTCCTGGAGCCCACACTCTGCTGGA | |
| | ATCCTTCTGCCTCTCTGGCCTGTAGCCTGGCCCTCTCTCCCAACCTGAGGTCCATTCTCTCCTGCTCCTC | |
| | CACAAGATGTTGCTCCTTCCATTACTTCCTCCCTCTCAACCAAAGCTCCTTCATTAGCTCTTTATCTTCT | |
| | GGTTTCTTCCCCTGGGCAGACGAATGGATTCAAGAGCCTGTGGCCCAGCAGCCCAGCACTCCAGGATCTC | |
| | AGCACTTCAGCATCCCAGTACCCTAGCATCTCAATACCCCAGCACCCCAGCACCATAGTATTCCAGCACC | |
| | CCATTGTCCAAGCATCTCAGCACTCCAGCATCCCAGCACCCCAACACTCCAGCAGCCCAGAATCTCAGCA | |
| | CCCTAGCACTGCAGCATCTCAGGACCCCAGCACTTCAGCATCCCAGCACACTAGTACTCCAGCATCTCGG | |
| | CACCCCAGCACCTAGGCATCCCAACACCCAGCACCCCAGCACTTAAGCATCCCACCACTACAGTATCTCA | |
| | ACACTCCAGCACCCCAGCACCATAGTGTTCCAGCACCCCAGCATCCCAACACCCCAGCACTTAAGCATCC | |
| | CAACACCTCGGCATCCCAACACCCCAGCACTGCAGCATCTCAGCACCTTAGCATCCCAGTGCCCTAGCAT | |
| | CTCAATGCTCCAGCACACCAGTACTACAGTATTCCAGCACCCCAGCACTCCAGCATCTCAGCACTGCAGC | |
| | ACTGCAGCACTCCAGCATCCCAAAATCCCAGCATCCCAACACCCCAGCAGACCAGCAGACCAGCATCTCA | |
| | GCACCGCAGCATCCAAGGACTATCCCAGCATCCCAGCAACCCAGCACCTCAGCATCCCAACACCCCAGCA | |
| | TTTCAGCATGGCAACACCCCAGTACCCCAGCACTTCAGCACCCCAGTATCCCAGCATCTCAGCGACCCAG | |
| | TATCACAAAACCTCAGCATCCTAGCACCCCAGCACCCCAGCACCTTAGCACCTTAGCATCCCAGCATCTC | |
| | AGCGCCTCAGCATCTTGATATTCTGGCTGAGGTCAGCGTGGTGTATCTAGTCAGGGTCCTAACTTTCACT | |
| | TCGCAGGGAAATGCTGCTGGACTGGGTCTCATGTTGGGCTGAAGCTCTCTAGACCCCTTGAAGACAGCAT | |
| | AAAAGAGCTTGGAGACGCTGGGTGTCCCCCATGGAAGAGTTCACTCTCATCCTGCTTTGACAACAGCCTT | |
| | CTCTGGGGTCCCTCACGGGCCCCTCTTTCTTACTGCAAGTTTGTCTCTGAGAAGACTGTGATGCAGAAGT | |
| | CACTCAGCTGCCTGTGGCTCCTGAAGAGCTGAAGGTGGAGGCCTGTAGGCCTCCCTATGAGAGGCGCAGA | |
| | AAAAACCATGATTGCTAGTGGGGAGGTGCTCCCTCTACAACCCACTCCATAATCTGCCCCCGCCCAGCTC | |
| | TGAGGCCAGCCCCAGGGGAAAATGCCAGATCCCCAGGGAGGTGTGTGAGACCTCAGGGGCTCCCTCCTCC | |
| | CTTACAGCAGGCTCAGGCCCCTGGGGGCCTCAGGGCCAAGGTCTGTGGGTAAGCTACTATCTCTCACTTG | |
| | TCCTCTAGCCACAAAAGCCAGGGAGATCTGGCAATGGACATGAGGTTCTGAAGAAGCACATATGACTGGC | |
| | TTCCTAATGCGTGGTTGTTCAGTGATTCAATAAACACGCATGGGCCAGGCATGGGGAAATAGACAAACAT | |
| | GATCCCCAACCTCTCCCAGAGTGAACTGGGAGGGAGGAGTGTTCATCCCTCAGGATTACACCAGAGAAAC | |
| | AAACCAGCAGGAGATATATATGGTTTTGGGGGGTCAAGAAAGAGGAAAAACCTGGCAAGGCAAGTCCAAA | |
| | ATCATAGGACAGGCTGTCAGGAAGGGCAGCCTGGAACCTCTCAAGCAGGAGCTGATGCTGCAGTCCACAG | |
| | GCAGAATTTCTTCTTCCTCGGGGAAATCTCAGCTTTGTTCTTAAGGCCTTTCAACTGATTGGCTGAGGTC | |
| | TGCCCCTTCCCCCACATTCTCCAGGATAATCTTCCTTACTTAAAGTCAACTATTAATCACAGCTACAAAA | |
| | TCCCTTCACAGCTACACATAGATCAGTGTTTGATTGACGAACAGCCCCTACAGCCTAGCCAAGTTGACAC | |
| | ATAAAACTAACCATCACAGGGGGACAAATGATGTAAACACATCAACAAATAAAACAGTAACAAGTTAAGG | |
| | TCTATGGAAAAAACACAGAAGGGGCAGAGAGAAAGAAAGCAAGAAGGAGAGTCCCAGTTTGCTAGGGCTT | |
| | GTGGGAAGTGGGGAGCAGTTCTCTTTAGCTAGGATATTTGGGAAAGGCATATCTGAAGGAGTGATATTTG | |
| | AGCTTAGATTAAAAGATGGGAAGGAGCAAGCCATGCAAAGAGCTAGGATGTTCCAAGCAGAGACGGAACA | |
| | GCAAGTGCAAATGTCAGGAGGAATAGAAGGAGGCTGGTGGGTGGGGTCCAGTGAGCAAGAGGAGGGCAGG | |
| | CAGGAGAGGGGATGGGGAGGTGGGCAGGCCCAGACCACCCAGGGCCCTGGAGACTATCCTGATCCAACAA | |
| | GGGAAGCCTTGAGTCACTTCAGTGTCCATGTGGAGAATGGACCTCAGACTGAATGAGGGAGGCAGTAAGG | |
| | AGGGCCTCTACCTCCAGGGCTTCGCCCTGTGGACTGCGCATAGACATCTCCAACTCAGAAAGTCTGAACC | |
| | AAACTTTCCATAGTTCCCCCAAGTCTGGGCATCCTCCTACTCAGTGAAAGGCAGCCATCACACCTCCCTG | |
| | CCCTGCTCCCGGATGCCCCAAATCCTCTTGGTCTCCAAGTCCAGAACCTGAGACTTGTCCTTGATGTTTG | |
| | TCTTTCCCTCACCCTTTCTGTATTCTGGGAAGATGGGTTTTTTTCCCCCAGATGAATCTGTAAAACTTCT | |
| | GTGATCACAATAAAAATTCTGGCAGTATTATTTTCTGGAACATGACAAAGTGATTCAAAATTATTTATCT | |
| | GGAAGACTACAAAACAAGAATAGCCAGGAAATTTCTAAAAAGAAAGAAGAAGGAGGAGGAGAAAGAAGGA | |
| | GGAGGAAAAGGAGGAGAAGAAGAAAAGAAAAAGAACCAAGAAAGGGTTCTAGCTCTACCAAATATTAAAA | |
| | CATATCATGAAGCTATTTAAAACAATATGGTTGTGGATACTGAAAAAGATGTGAATAAAGTGGAAGGAAA | |
| | ATAAATAGAAATGCACATGGGGATTGAGACTGTGAAAAAGGCAGCATCTCACATCAGTGAGGGATGTTCA | |
| | ACACCTGGTGTTGGGAAAACTGGCTAGTCATTTAAACCAAACAACTGGGTCCTCTACCTCACTCCTGACA | |
| | TTAAGATACATTTAGATGATTCAAAGAGTAAGACAGAAAAAATAACACGTGAAAACACTATCAGAAAACA | |
| | ACGTGGGCCAGGTGTGGTGGGTCACGCCTGTAATCCCAGCACTTTGGGAGGCCGAGGCAGACAGATCACC | |
| | TGAGGTGGGGAGTTCAAGACCAGCCTGACCAACATGGTGAAATCCTGTCTCTACTAAAAATACAAAATTA | |
| | TTGAACCTGGGAGGCAGAGGTTGTGGTGAGCCGAGATCACGCCATTGCACTCCAGCCTGGGCAACAAGAG | |
| | TGAAAATCCATCTAAAAAAAAAAAAAAAAGCCAAGGTGGATATTTTTATAGTATCAGGGTAGATCAAGCT | |
| | TCTCCAATCATGACATGAAACCCAGAAACCATAAAAGAAAAGAATGATAAAATTGCCCACGTAAAGTAAA | |
| | AAGCTTGCACACAGAAAAACACCATACAGGTTACAAGATGAGCAGCAAAATCAGAGAAAAAACATTGCAA | |
| | TTCAGGACACACAGAGGCTATTGTTCCTAATATTTAAAAATAAAAGTAGTGGATTGTCTACAAAAAGATG | |
| | AAGACAAGAATTTCAGAAAACCAAATACTGCATGTTTTCACTTACAAGTGGAAGCTAAACACTGAGTACA | |
| | CGTGTACACAAAGAATGGAACCATAGGCCAGGCACCGTGGCTCACGCCTGTAATCCCAGTACTTTGCGAG | |
| | GCCGAAGCGGGCGGATCACCTGAGGTGAGGAGTTCGAGACCATCCTGGCCAACATGGTGAAACCCAGTCT | |
| | CTACTAAAAATACAAAAATTAGCCGGGCGTGGTGGTGGGTGCCTGTAATCCCAGCTACTCGGGAGGCTGC | |
| | GGCAGTAGAATCGCTTGAACCCTGGAGGTGGACCTTGCAGTGAGCCGAGATCGCACCACTGCACTCCAGC | |
| | CTGGGCAACAGAGTGAGACTCCATCTCAAAAAAAAAAAAAAGGAATAGAACAATAGACACTGGGGCCTAC | |
| | TTGAGGGAGGAGGGTGAGGATCAAAAACCTGCCTATCAGGTACTATGCTTATTACCTGGGTGGTGAAATA | |
| | ATCTGTACACCAAACCCCAGTGACATGCAATTTACCGATGTAACAAACCTGCCCATGTACCCGCTGAACC | |
| | TAAAATAAAAGTTGGAAAAAAATATAGAAATTTTCTTTGTAATAGCCAAAAACTGCAAACAGCCCAGGTG | |
| | TCTATTAGTAGAATGCATAAACAAACTCGGGCATGTTCATACAATGTAAAACTACTCATCAATAAAAAGT | |
| | GATACTTCTCAGCAATGAAAAGAAACTAGCTACTGATACCAGCTACAACATGGATGGATTTCAAGTGCTT | |
| | TATGATGAGAGCAAGAAGCCAGACACAAAAGTGTCTATATATATATACAGTATATATACGTATATATACA | |
| | CATATATACAGTATATATATACATATACATGTATATATATACTGTATATATACTGTATATATATACACAG | |
| | TATATATATACATATATACAGTGTATATATACTGTGTATATATACATGTATATATACTGAGTATATATAC | |
| | ATGTATATATATGTATACTGTATATATACTGTATATATATATACACATATATACAGTATATATATACAGT | |
| | ATATACTGTATATATACAGTATATACGTGTATATATACATATATACAGTATATATGTAAATATACATATA | |
| | TACAGTATATATGTAAATATACATATATACATGTATATATATACACTATATATATACATATATAGTGTAT | |
| | ATATACATATATACATGTATATATTTACTATATGATTCCATTTATATAAAGTGCCAAAACAGTCAAAAAT | |
| | AATCTATGTGGAAAAAATCAACAAAGGGATCCCCCGGGCTGCAGGAATTCGATGGCGCGCCTTAATTAAA | |
| | ATTATCTCTAAGGCATGTGAACTGGCTGTCTTGGTTTTCATCTGTACTTCATCTGCTACCTCTGTGACCT | |
| | GAAACATATTTATAATTCCATTAAGCTGTGCATATGATAGATTTATCATATGTATTTTCCTTAAAGGATT | |
| | TTTGTAAGAACTAATTGAATTGATACCTGTAAAGTCTTTATCACACTACCCAATAAATAATAAATCTCTT | |
| | TGTTCAGCTCTCTGTTTCTATAAATATGTACCAGTTTTATTGTTTTTAGTGGTAGTGATTTTATTCTCTT | |
| | TCTATATATATACACACACATGTGTGCATTCATAAATATATACAATTTTTATGAATAAAAAATTATTAGC | |
| | AATCAATATTGAAAACCACTGATTTTTGTTTATGTGAGCAAACAGCAGATTAAAAGGCTAGCCTGCAGGA | |
| | GTCAATGGGAAAAACCCATTGGAGCCAAGTACACTGACTCAATAGGGACTTTCCATTGGGTTTTGCCCAG | |
| | TACATAAGGTCAATAGGGGGTGAGTCAACAGGAAAGTCCCATTGGAGCCAAGTACATTGAGTCAATAGGG | |
| | ACTTTCCAATGGGTTTTGCCCAGTACATAAGGTCAATGGGAGGTAAGCCAATGGGTTTTTCCCATTACTG | |
| | ACATGTATACTGAGTCATTAGGGACTTTCCAATGGGTTTTGCCCAGTACATAAGGTCAATAGGGGTGAAT | |
| | CAACAGGAAAGTCCCATTGGAGCCAAGTACACTGAGTCAATAGGGACTTTCCATTGGGTTTTGCCCAGTA | |
| | CAAAAGGTCAATAGGGGGTGAGTCAATGGGTTTTTCCCATTATTGGCACATACATAAGGTCAATAGGGGT | |
| | GACTAGTGGAGAAGAGCATGCTTGAGGGCTGAGTGCCCCTCAGTGGGCAGAGAGCACATGGCCCACAGTC | |
| | CCTGAGAAGTTGGGGGGAGGGGTGGGCAATTGAACTGGTGCCTAGAGAAGGTGGGGCTTGGGTAAACTGG | |
| | GAAAGTGATGTGGTGTACTGGCTCCACCTTTTTCCCCAGGGTGGGGGAGAACCATATATAAGTGCAGTAG | |
| | TCTCTGTGAACATTCAAGCATCTGCCTTCTCCCTCCTGTGAGTTTGgtaagtcactgactgtctatgcct | |
| | gggaaagggtgggcaggaggtggggcagtgcaggaaaagtggcactgtgaaccctgcagccctagacaat | |
| | tgtactaaccttcttctctttcctctcctgacagGTTGGTGTACAGTAGTAGCAAGCTTGCATGCCTGCA | |
| | GGTCGACTCTAGACTGCCATGGTATTTGTCAGACGACCTTGGCCTGCCCTCACCACGGTGTTGCTCGCCT | |
| | TGCTCGTTTGCTTGGGGGCCTTGGTGGACGCTATCAAGCCTGAGGCACCCGGAGAAGACGCATCCCCCGA | |
| | GGAATTGAACCGCTATTACGCATCCTTGCGGCACTACCTGAACCTCGTGACCAGGCAACGCTATCGGCAC | |
| | GACTTCCCTGAAGAAGTCGCAATCGTCGAAGAGCTCGGCAGGAGAATCAAACCTGAGGCTCCTGGTGAGG | |
| | ACGCCAGTCCGGAGGAACTGAATCGCTACTACGCGTCACTTAGGCACTACCTGAACCTGGTCACTCGCCA | |
| | GAGATACTAAGGATCCCCGGGAGATATCCTAGGCTTGGCCAGACATGATAAGATACATTGATGAGTTTGG | |
| | ACAAACCACAACTAGAATGCAGTGAAAAAAATGCTTTATTTGTGAAATTTGTGATGCTATTGCTTTATTT | |
| | GTAACCATTATAAGCTGCAATAAACAAGTTAACAACAACAATTGCATTCATTTTATGTTTCAGGTTCAGG | |
| | GGGAGGTGTGGGAGGTTTTTTAAAGCAAGTAAAACCTCTACAAATGTGGTATGGAATTCAGTCAATATGT | |
| | TCACCCCAAAAAAGCTGTTTGTTAACTTGCCAACCTCATTCTAAAATGTATATAGAAGCCCAAAAGACAA | |
| | TAACAAAAATATTCTTGTAGAACAAAATGGGAAAGAATGTTCCACTAAATATCAAGATTTAGAGCAAAGC | |
| | ATGAGATGTGTGGGGATAGACAGTGAGGCTGATAAAATAGAGTAGAGCTCAGAAACAGACCCATTGATAT | |
| | ATGTAAGTGACCTATGAAAAAAATATGGCATTTTACAATGGGAAAATGATGGTCTTTTTCTTTTTTAGAA | |
| | AAACAGGGAAATATATTTATATGTAAAAAATAAAAGGGAACCCATATGTCATACCATACACACAAAAAAA | |
| | TTCCAGTGAATTATAAGTCTAAATGGAGAAGGCAAAACTTTAAATCTTTTAGAAAATAATATAGAAGCAT | |
| | GCCATCAAGACTTCAGTGTAGAGAAAAATTTCTTATGACTCAAAGTCCTAACCACAAAGAAAAGATTGTT | |
| | AATTAGATTGCATGAATATTAAGACTTATTTTTAAAATTAAAAAACCATTAAGAAAAGTCAGGCCATAGA | |
| | ATGACAGAAAATATTTGCAACACCCCAGTAAAGAGAATTGTAATATGCAGATTATAAAAAGAAGTCTTAC | |
| | AAATCAGTAAAAAATAAAACTAGACAAAAATTTGAACAGATGAAAGAGAAACTCTAAATAATCATTACAC | |
| | ATGAGAAACTCAATCTCAGAAATCAGAGAACTATCATTGCATATACACTAAATTAGAGAAATATTAAAAG | |
| | GCTAAGTAACATCTGTGGCTTAATTAAGGCGCGCCTACCAGTAAAAAAGAAAACCTATTAAAAAAACACC | |
| | ACTCGACACGGCACCAGCTCAATCAGTCACAGTGTAAAAAAGGGCCAAGTGCAGAGCGAGTATATATAGG | |
| | ACTAAAAAATGACGTAACGGTTAAAGTCCACAAAAAACACCCAGAAAACCGCACGCGAACCTACGCCCAG | |
| | AAACGAAAGCCAAAAAACCCACAACTTCCTCAAATCGTCACTTCCGTTTTCCCACGTTACGTCACTTCCC | |
| | ATTTTAAGAAAACTACAATTCCCAACACATACAAGTTACTCCGCCCTAAAACCTACGTCACCCGCCCCGT | |
| | TCCCACGCCCCGCGCCACGTCACAAACTCCACCCCCTCATTATCATATTGGCTTCAATCCAAAATAAGGT | |
| | ATATTATTGATGATGA | |
| | Description of the elements: | |
| | (1) . . . (103): inverted terminal repeat (ITR) | |
| | (104) . . . (27556): encodes the shuttle vector (P-delta-28) | |
| | (27557) . . . (27564): AscI Restriction enzyme site | |
| | (27565) . . . (27991): encodes CpG free MAR from human beta- | |
| | globin gene | |
| | (27992) . . . (28414): encodes CMV Enhancer | |
| | (28415) . . . (28669): encodes human EF1-alpha promotor | |
| | (28670) . . . (28797): encodes synthetic intron | |
| | (28798) . . . (28851): encodes a multiple cloning site | |
| | (28852) . . . (29193): encodes transgene (PYY) from ATG to stop | |
| | codon | |
| | (29194) . . . (29221): encodes a multiple cloning site | |
| | (29222) . . . (29452): encodes SV40 poly Adenylation signal | |
| | (29453) . . . (30260): encodes MAR 5′ region from human IFN- | |
| | beta gene | |
| | (30261) . . . (30268): AscI Restriction enzyme site | |
| | (30269) . . . (30565): encodes the shuttle vector (P-delta-28) | |
| | (30566) . . . (30669): inverted terminal repeat (ITR) | |
| HDAd-EF1a-PYY | CATCATCAATAATATACCTTATTTTGGATTGAAGCCAATATGATAATGAGGGGGTGGAGTTTGTGACGTG | 44 |
| Approach 2 | GCGCGGGGCGTGGGAACGGGGCGGGTGACGTAGTAGTGTGGCGGAAGTGTGATGTTGCAAGTGTGGCGGA | |
| Codon | ACACATGTAAGCGACGGATGTGGCAAAAGTGACGTTTTTGGTGTGCGCCGGTGTACACAGGAAGTGACAA | |
| Optimized | TTTTCGCGCGGTTTTAGGCGGATGTTGTAGTAAATTTGGGCGTAACCGAGTAAGATTTGGCCATTTTCGC | |
| Nucleic Acid | GGGAAAACTGAATAAGAGGAAGTGAAATCTGAATAATTTTGTGTTACTCATAGCGCGTAATATTTGTCTA | |
| Sequence | GGGCCGCGGGGACTTTGACCGTTTACGTGGAGACTCGCCCAGGTGTTTTTCTCAGGTGTTTTCCGCGTTC | |
| Encoding PYY | CGGGTCAAAGTTGGCGTTTTGATATCAAGCTTATCGATACCGTAAACAAGTCTTTAATTCAAGCAAGACT | |
| (3-36) | TTAACAAGTTAAAAGGAGCTTATGGGTAGGAAGTAGTGTTATGATGTATGGGCATAAAGGGTTTTAATGG | |
| Approach 2 | GATAGTGAAAATGTCTATAATAATACTTAAATGGCTGCCCAATCACCTACAGGATTGATGTAAACATGGA | |
| Construct in | AAAGGTCAAAAACTTGGGTCACTAAAATAGATGATTAATGGAGAGGATGAGGTTGATAGTTAAATGTAGA | |
| HDdelta28E4- | TAAGTGGTCTTATTCTCAATAAAAATGTGAACATAAGGCGAGTTTCTACAAAGATGGACAGGACTCATTC | |
| MAR-EF1a | ATGAAACAGCAAAAACTGGACATTTGTTCTAATCTTTGAAGAGTATGAAAAATTCCTATTTTAAAGGAAA | |
| shuttle | ACAGTAACTCACAGGAAATACCAACCCAACATAAAATCAGAAACAATAGTCTAAAGTAATAAAAATCAAA | |
| vector | CGTTTGCACGATCAAATTATGAATGAAATTCACTACTAAAATTCACACTGATTTTGTTTCATCCACAGTG | |
| | TCAATGTTGTGATGCATTTCAATTGTGTGACACAGGCAGACTGTGGATCAAAAGTGGTTTCTGGTGCGAC | |
| | TTACTCTCTTGAGTATACCTGCAGTCCCCTTTCTTAAGTGTGTTAAAAAAAAAGGGGGATTTCTTCAATT | |
| | CGCCAATACTCTAGCTCTCCATGTGCTTTCTAGGAAACAAGTGTTAACCCACCTTATTTGTCAAACCTAG | |
| | CTCCAAAGGACTTTTGACTCCCCACAAACCGATGTAGCTCAAGAGAGGGTATCTGTCACCAGTATGTATA | |
| | GTGAAAAAAGTATCCCAAGTCCCAACAGCAATTCCTAAAAGGAGTTTATTTAAAAAACCACACACACCTG | |
| | TAAAATAAGTATATATCCTCCAAGGTGACTAGTTTTAAAAAAACAGTATTGGCTTTGATGTAAAGTACTA | |
| | GTGAATATGTTAGAAAAATCTCACTGTAACCAAGTGAAATGAAAGCAAGTATGGTTTGCAGAGATTCAAA | |
| | GAAAATATAAGAAAACCTACTGTTGCCACTAAAAAGAATCATATATTAAATATACTCACACAATAGCTCT | |
| | TCAGTCTGATAAAATCTACAGTCATAGGAATGGATCTATCACTATTTCTATTCAGTGCTTTGATGTAATC | |
| | CAGCAGGTCAGCAAAGAATTTATAGCCCCCCTTGAGCACACAGAGGGCTACAATGTGATGGCCTCCCATC | |
| | TCCTTCATCACATCTCGAGCAAGACGTTCAGTCCTACAGAAATAAAATCAGGAATTTAATAGAAAGTTTC | |
| | ATACATTAAACTTTATAACAAACACCTCTTAGTCATTAAACTTCCACACCAACCTGGGCAATATAGTGAG | |
| | ACCCCATGCCTGCAAAAAAAAAAAAATTAGCCAGGCATGGTAGCATGTACCTGTAGTCCCAGCTACTTGA | |
| | GAGGTGAGGTGGGAAAATCACTTTAGTGCAGGATGTTGAGGCTGGAGTGAACTGTGATTGTGCCACTGCA | |
| | CTCCAGCCTGGACAATAGAGCAAGACCTTGTCTCAAAAAAATGCATTAAAAATTTTTTTTAAATCTTCCA | |
| | CGTAACACATCCTTTGCCCTCATGTTTCATAAGGTAAAAAATTTGATACCTTCAAAAAAACCAAGCATAC | |
| | CACTATCATAATTTTTTTTAAATGCAAATAAAAACAAGATACCATTTTCACCTATCAGACTGGCAGGTTC | |
| | TGATTAAATGAAATTTCTTGGATAATATACAATATTAAGAGAGACTGTAGAAACTGGGCCAGTGGCTCAT | |
| | GCCTGTAATCCCAGCACTTTGGGAGGCTGGGTAACATGGCGAACCCTGTTTCTACAAAATAAAAATATTA | |
| | GCTGGGAGTGGTGGCGCACACCTATAGTCCCAGCTACTCAGGAGGCTGAGGTGGAAGGATCGCTTGAACC | |
| | CAGGAGGTTGAGACTGCAGTGAACTGTGATCATTCTGCTGCACTGCACCCCAGCCTGGGCAACAGAGACC | |
| | TTGTCTCaAAAAAAAAAAAAAAAGAGACAAATTGTGAAGAGAAAGGTACTCTCATATAACATCAGGAGTA | |
| | TAAAATGATTCAACTTCTTAGAGGAAAATTTGGCAATACCAAAATATTCAATAAACTCTTTCCCCTTGAC | |
| | CCAGAAATTCCACTTGAATAAAGCTGAACAAGTACCAAACATGTAAAAGAATGTTTCTTCTAGTACAGTC | |
| | GGTAAGAACAAAATAGTGTCTATCAATAGTGGACTGGTTAAATCAGTTATGGTATCTCCATAAGACAGAA | |
| | TGCTATGCAACCTTTAAAATATATTAGATAGCTCTAGACACACTAATATTAAAAGTGTCCAATAACATTT | |
| | AAAACTATACTCATACGTTAAAATATAAATGTATATATGTACTTTTGCATATAGTATACATGCATAGCCA | |
| | GTGCTTGAGAAGAAATGTGTACAGAAGGCTGAAAGGAGAGAACTTTAGTCTTCTTGTTTATGGCCTCCAT | |
| | AGTTAGAATATTTTATAACACAAATATTTTGATATTATAATTTTAAAATAAAAACACAGAATAGCCAGAC | |
| | ATACAATGCAAGCATTCAATACCAGGTAAGGTTTTTCACTGTAATTGACTTAACAGAAAATTTTCAAGCT | |
| | AGATGTGCATAATAATAAAAATCTGACCTTGCCTTCATGTGATTCAGCCCCAGTCCATTACCCTGTTTAG | |
| | GACTGAGAAATGCAAGACTCTGGCTAGAGTTCCTTCTTCCATCTCCCTTCAATGTTTACTTTGTTCTGGT | |
| | CCCTACAGAGTCCCACTATACCACAACTGATACTAAGTAATTAGTAAGGCCCTCCTCTTTTATTTTTAAT | |
| | AAAGAAGATTTTAGAAAGCATCAGTTATTTAATAAGTTGGCCTAGTTTATGTTCAAATAGCAAGTACTCA | |
| | GAACAGCTGCTGATGTTTGAAATTAACACAAGAAAAAGTAAAAAACCTCATTTTAAGATCTTACTTACCT | |
| | GTCCATAATTAGTCCATGAGGAATAAACACCCTTTCCAAATCCTCAGCATAATGATTAGGTATGCAAAAT | |
| | AAATCAAGGTCATAACCTGGTTCATCATCACTAATCTGAAAAAGAAATATAGCTGTTTCAATGAGAGCAT | |
| | TACAGGATACAAACATTTGATTGGATTAAGATGTTAAAAAATAACCTTAGTCTATCAGAGAAATTTAGGT | |
| | GTAAGATGATATTAGTAACTGTTAACTTTGTAGGTATGATAATGAATTATGTAAGAAAACAACAGGCCGG | |
| | GCGGGTTGGTTCACACGTGTAATCCCAGCACTTTGGGAGGCTGAGGCAGGCAGACTGCCTGAGCTCAGGA | |
| | GTTCGAGACCAGCCTGGGCAACACGGTGAAATCCCGTCTCTACTAAAAATACAAAAAAATTAGCCGGGTG | |
| | TGGTGACACATGCCTGTAGTCCCAGCTACTTGGGAGGCTGAGGCAGGAGAATCACTTGAACCTGGGAGGT | |
| | GAAGGTTGCAGTGAGCCAAGAATGCGCCACTTCACTCCAGCCTGGGAAACAGAGCAAGACTCTGTCTCAA | |
| | AAAAAACAAAACAAACAAACAAAAAAACAGGCTGGGCGCGGTGGCTCACGCCTGTAATCCCAGCACTTTG | |
| | GGAGGCCGAGGCGGGTGGATCACCTGAGGTCAGGAGTTCCAGACCAGCCTTGTCAACATGGTGAAACCTC | |
| | CCCCCGCCGTCTCTACTAAAAATACAAAAATTAGCCAGGCGTGGTGGCAGGAGCCTGTAATCCCAGCTAC | |
| | TTGGGAGGCTGAGGCAGGAGAATCGCTTGTACCCAGAAGGCAGAGGTTGCACTGAGCTGAGATGGCACCA | |
| | TTGCACTCCAGCCTGGGGGACAAGAGCGAGATTTCGTCTTTAAAAAACAAAAAACAAAACAAAAAACCAT | |
| | GTAACTATATGTCTTAGTCATCTTAGTCAAGAATGTAGAAGTAAAGTGATAAGATATGGAATTTCCTTTA | |
| | GGTCACAAAGAGAAAAAGAAAAATTTTAAAGAGCTAAGACAAACGCAGCAAAATCTTTATATTTAATAAT | |
| | ATTCTAAACATGGGTGATGAACATACGGGTATTCATTATACTATTCTCTCCACTTTTGAGTATGTTTGAA | |
| | AATTTAGTAAAACAAGTTTTAACACACTGTAGTCTAACAAGATAAAATATCACACTGAACAGGAAAAACT | |
| | GGCATGGTGTGGTGGCTCACACTTGTAATCCCAGTGCTTTGGGAGGCTGAGACAGGAGAGTTGCTTGAGG | |
| | CCAGGAGTTCAAGACCGACATGGGGAATGTAGCAAGACCCCGTCCCTACAAAAAACTTTGTAAAAATTTG | |
| | CCAGGTATGGTGGTGCATACCTGTAGTCCCAGCTACTCGGGAGGCGGAGGCAGAAGGAATCACTTGAGCC | |
| | CAGGAGTTTGAGGCTGCAGTGAGCTACGATCATACCACAGCACTCCAGCGTGGACAACAGAGTAAGACCC | |
| | TATCTCAAAAACAAAACAAAACAAAACAAACAAAAAAAACCACAAGAAAAACTGCTGGCTGATGCAGCGG | |
| | CTCATGCCTGTAATCCCAGTATTTTGGGAGGCCCAGGTGGGCGTATCACCTGAGGTCAGGAGTTAGAGAC | |
| | CAGCCTGGCCAACATGGTGAAACCCCATCTCTACTAAAAATACAAAATTAGCCAGGCATGTGGCACGCGC | |
| | CTGTAGTCCCAGTTACTGGGGAGGCTGAAGCAGGAGGATCACCTGAGCCCGGGAGGTGGAGGTTGCAGTG | |
| | AGCCGAGATCACACCACTGCACTCCAGCCTGGGTGACACAGCAATACCCTACCTCAAAATAAAAAAGAAA | |
| | AAGAAAAGAAAAGTTGCTGTCCCCGCTACCCCAATCCCAAATCCAAACAGCCTCTCTCATCTCACAGTAA | |
| | GGGGGAAAAATCACCCAAAAAAGCTAAGTGATCTTTTGAAAACCCAAACTCTTAGAAGTCTAAGATTATT | |
| | ATAGTCAACTCATGAAGTGTCATCATAAAAGATACTCTAATATTATTTAAGTAGAACCACATATTGGTTG | |
| | TCTTGGTATGTCTAGCCCCTGGCATACAAAATATTTAATAACACTGATATGGTACCTGTGATGTGAAAAT | |
| | GTACTATGAGTACAGCTTTATAAATACTATATATGTACCTATATACAGAAAAAAATACAACAAAATCATA | |
| | AAAGCACTTATCTTTGAAAGAGGAGTTACAGCAATTTTATTTAGTTCTTTATTGCTTTGCTATATATTCT | |
| | AAATTTTTTTCAATGAATATATATCACTTTTAAAAAAATTCAATGGTCTTTCTTATAAATTATCTTTGGC | |
| | AGCATGCGTTTTTATATATACATATAAAATGTATGGGAAATTTTTAAAGGATACATTAAATTAAAGCAAA | |
| | ATATACAAACAAAAAATCAGAATACAAAAAGATAAAAAGATTGGGAAGGGAGGGAGGGAGTAAGGAGGAA | |
| | GGGTGGGTGGGTATAGAGAAATATACCAAATAATGGTAAGAAGTGGGGTCTTGACACTTTCTACACTTTT | |
| | TTTAAATAAAAAAAATTTTTTTCTCTCTCTTTTTTTTTTTTAGAGACGAAGTCTCGCTATGTTGCCCAGG | |
| | CTGGTCTTGAACTCCTGGGATCAAGAGATCCTCCTGCCTCAGCCTCCCAAGGTGCTTGGATTACAGGTGT | |
| | GAGCCACCACGCCTGGTCACTTTCTACACTTTAATATATATATTTTTTCATTTTCAATGTCATTTTTATT | |
| | AGTTAATTTATAATACCCATTCACCATTATATTCAAAGTCTATTTGAAGAAATAAACCAGAAAGAATGAA | |
| | ATACTCTAGCTCACATGCTATTCAATACTAAATTACCTTTCAAATCACATTCAAGAAGCTGATGATTTAA | |
| | GCTTTGGCGGTTTCCAATAAATATTGGTCAAACCATAATTAAATCTCAATATATCAGTTAGTACCTATTG | |
| | AGCATCTCCTTTTACAACCTAAGCATTGTATTAGGTGCTTAAATACAAGCAGCTTGACTTTTAATACATT | |
| | TAAAAATACATATTTAAGACTTAAAATCTTATTTATGGAATTCAGTTATATTTTGAGGTTTCCAGTGCTG | |
| | AGAAATTTGAGGTTTGTGCTGTCTTTCAGTCCCCAAAGCTCAGTTCTGAGTTCTCAGACTTTGGTGGAAC | |
| | TTCATGTATTGTCAGGTTGGCCCGTAATACCTGTGGGACAACTTCAGCCCCTGTGCACATGGCCAGGAGG | |
| | CTGGTTGCAAACATTTTCAGGTAGGTGGACCAGGACATGCCCCTGGTCATGGCCAGGTGGAGGCATAGTG | |
| | CTATACAGCAGGCAGAAGTCAATATTGATTTGTTTTTAAAGAAACATGTACTACTTTCATAAGCAGAAAA | |
| | AATTTCTATTCTTGGGGGAAAAGATTATGCCAGATCCTCTAGGATTAAATGCTGATGCATCTGCTAAACC | |
| | TTCACATATCAGAACATATTTACTATAGAAAGAATGAAAATGGGACATTTGTGTGTCACCTATGTGAACA | |
| | TTCCAAAAATATTTTACAACAACTAAGTATTTTATAAATTTTATGAACTGAAATTTAGTTCAAGTTCTAG | |
| | GAAAATACAAACCTTGCTAGATATTATAAAAATGATACAATATATATTCATTTCAGGCTCATCAGAATAT | |
| | ATCTGTTATCACTTGACAAGAATGAAAATGCACCATTTTGTAGTGCTTTAAAATCAGGAAGATCCAGAGT | |
| | ACTAAAAATGACTTCTTCCTTGAAGCTTACTCACCAACTTCCTCCCAGTTACTCACTGCTTCTGCCACAA | |
| | GCATAAACTAGGACCCAGCCAGAACTCCTTGAAATATACACTTGCAACGATTACTGCATCTATCAAAATG | |
| | GTTCAGTGCCTGGCTACAGGTTCTGCAGATCGACTAAGAATTTGAAAAGTCTTGTTTATTTCAAAGGAAG | |
| | CCCATGTGAATTCTGCCCAGAGTTCATCCCAGATATGCAGTCTAAGAATACAGACAGATCAGCAGAGATG | |
| | TATTCTAAAACAGGAATTCTGGCAATATAACAAATTGATTTCCAATCAAAACAGATTTACATACCATACT | |
| | TATGTCAAGAAGTTGTTTTGTTTTATTGCATCCTAGATTTTATTTTTTTGATTTATGGTTTACTTTAAGC | |
| | ATAAAAAATTTGTCAATACAACTCTTCCCAAAAGGCATAAACAAAAATTCATAAAACTTGCATCACTTGA | |
| | GATACTTCAGGTATGAATTCACAACTTTGTTACAACTTACTATATATATGCACACATATATATATATTTG | |
| | GGTATATTGGGGGGGTTCTAATTTAAGAAATGCATAATTGGCTATAGACAGACAGTTGTCAGAACTTGGC | |
| | AATGGGTACGTGCAGGTTCATTATACCAAGTCTACTTGTAGTTGTTCAAAATGTATCATAATACAAGGCC | |
| | GGGCGAGGTGGCTCACGCCTGTAATCCCAGCATTTTGGGAGGCTAAGGCAGGAGGATTGCTTGAGGTCAG | |
| | GAGTTTGTGACCAGCCTGGGCAACAGAGCAAGACCCTGTCTCCAAAAAGAAAAAAAATAATTTTTTACAA | |
| | AATAAAAACAAAATGTATCATCAGACGAAATTAAATAAGAGGCAATTCATTTAAATGACAACTTTTCCCA | |
| | GCTTGACATTTAACAAAAAGTCTAAGTCCTCTTAATTCATATTTAATGATCAAATATCAAATACTAATTT | |
| | TTTTTTTTTTTTTTTTTTTGAGACGGAGTCTCGCTCTGTCGCCCAGGCTGGAGTGCAGTGGCGCGATCCT | |
| | GGCTCACTGCAAGCTCCGCCTCCCGGGTTCACGCCATTCTCCTGCCTCAGCCTCCCGAGTAGCTGGGATT | |
| | ACAGACATGCGCCACCACGCCCGGCTAATTTTGTATTTTTAGTAGAGATGGGGTTTCTCCATGTTGGTCA | |
| | GGCTGGTCTTGAATTTCCCACCTCAGGTGATCTGCCTGCCTCAGCCTCACAAAGCAGTAGCTGGGACTAC | |
| | AGGCACCCACCACCACACTTGGTTAATTCTTTTGTATTTTTTTTGTAAAGACGGGATTTCACCATGTTAG | |
| | CCAGGATGGTCTCGATCTCCTGATCTCATGATCCGCCCGCCTCAGCCTCCCAAAGTGCTGGGATTACAGG | |
| | CGTGAGCCACCCCGCCCGGCCATCAAATACTAATTCTTAAATGGTAAGGACCCACTATTCAGAACCTGTA | |
| | TCCTTATCACTAATATGCAAATATTTATTGAATACTTACTATGTCATGCATACTAGAGAGAGTTAGATAA | |
| | ATTTGATACAGCTACCCTCACAGAACTTACAGTGTAATAGATGGCATGACATGTACATGAGTAACTGTGA | |
| | ACAGTGTTAAATTGCTATTTAAAAAAAAAGACGGCTGGGCGCTGTGGCTCATGCCTGTAATCCCAGCACT | |
| | TTGGGAGGCCAAGGCAAGTTGATCGCTCGAGGTCAAGAGTTCGAGACCAGCCTGGCCAACGTGGTAAAAC | |
| | CCCGTCTCTACTAAAAATACAAAAAAAAAATTAGCCAGGCATGGTGGCACAGGCCTGTAATCCCAGCTAC | |
| | TAGGGAGGCTGAGACATGGAGAACTGCTTGAATCCAGGAGGCAGAGGTTACAGTGAGCCGAGATCATACC | |
| | ACTACACTCCAGCCTGAGTGACAGAGCGAGACTCCGTCTAAAAAAAAAAAAAAAAAAAAAGATACAGGTT | |
| | AAGTGTTATGGTAGTTGAAGAGAGAACTCAAACTCTGTCTCAGAAGCCTCACTTGCATGTGGACCACTGA | |
| | TATGAAATAATATAAATAGGTATAATTCAATAAATAGGAACTTCAGTTTTAATCATCCCAAACACCAAAA | |
| | CTTCCTATCAAACAGGTCCAATAAACTCAATCTCTATAAGAGCTAGACAGAAATCTACTTGGTGGCCTAT | |
| | AATCTTATTAGCCCTTACTTGTCCCATCTGATATTAATTAACCCCATCTAATATGGATTAGTTAACAATC | |
| | CAGTGGCTGCTTTGACAGGAACAGTTGGAGAGAGTTGGGGATTGCAACATATTCAATTATACAAAAATGC | |
| | ATTCAGCATCTACCTTGATTAAGGCAGTGTGCAACAGAATTTGCAGGAGAGTAAAAGAATGATTATAAAT | |
| | TTACAACCCTTAAAGAGCTTATAGCTGGGCGTGGTGGCTCATGCCTGTAAATCCCAGCACTTTGGGAGGC | |
| | TGAGGCGGGTGGATCACCTGAGGCCAGAAGTTCAAGACCAGCCTAGCCAACATGGCGAAACCCTGTCTCT | |
| | ACAAAAAATACAAAAATTAGCCGGGTGTGGTGGCACGTGCCTGTAGTCCCAGTTACTTGGGAGGCCGAGG | |
| | CAGGAGAATCGCTTGAACCTAGGAGGTGGAGGCTGCAGTGAGCCGAGATTGTGCCACTGCACTCCACTTC | |
| | AGCCTGGGCGACAAGAGCAAGACTCCGTCACAAAAAAAAAAAAAAAAAAAAAGCTTAAAATCTAGTGGGA | |
| | AAGGCATATATACATACAACTAACTGTATAGCATAATAAAGCTCATAATCTGTAACAAAATCTAATTCGA | |
| | CAAGCCCAGAAACTTGTGATTTACCAAAAACAGTTATATATACACAAAAAGTAAACCTAGAACCCAAAGT | |
| | TACCCAGCACCAATGATTCTCTCCCTAAGCAGTATCAAGTTTAAAGCAGTGATTACATTCTACTGCCTAG | |
| | ATTGTAAACTGAGTAAAGGAGACCAGCACCTTTCTGCTACTGAACTAGCACAGCCGTGTAAACCAACAAG | |
| | GCAATGGCAGTGCCCAACTTTCTGTATGAATATAAGTTACATCTGTTTTATTATTTGTGACTTGGTGTTG | |
| | CATGTGGTTATTATCAACACCTTCTGAAAGAACAACTACCTGCTCAGGCTGCCATAACAAAATACCACAG | |
| | ACTGAGTGACTTAACAGAAACTTATTTCTCACAGTTTTGGAGGCTGGGAAGTCCAAAATTAAGGTACCTG | |
| | CAAGGTAGGTTTCAATCTCAGGCCTCTTCTTTGGCTTGAAGGTCTTCTAACTGTGTGCTCACATGACCTC | |
| | TTCTAACAAGCTCTCTGGTGTCTCTTTTTTTTTTTTTTTCTTTTTTGAGACAGAGTCTCACTCTGTCACC | |
| | CAGGCTGGAGTACAGTGGCACAATCTGGGCTCACTGCAACCTCCAACTCCCGGGTTCAAGTGATTCTCAT | |
| | GCCTCACCCTCCCGAGTAGCTTGGATGACAGGAGCCCGCTACCACACCCAGCTAATTTTTGTATTTTTAG | |
| | TAGAGATGGTGTTTCACTACATTGGCCAGGCTGGTCTCAAACTCCTGACCTCGTGATCCACCCACCTTGG | |
| | CCTCCCAAAGTGCTGGGATTACAGGTGTGAGCCACTGCGCCCGTCCTGGTGTCTTTTCATATAAGGGCAC | |
| | TAATCCAATCAGACCTGGGCCCAACCCTCCCGACTTCTTCTAACTGTAATTACCTTCCAAAGGCCCTGTC | |
| | TCCAAATACCATCACACTGGGGGTTAGGACTTCAAAAAAGGTATGGGGGGGGTGTGGGAGGACATAAATG | |
| | CTCAGTCCATAACAAGCACCCAACATAAAAATGGCTAGAACAGATCACAAAAAAAAGGTCCTGTATGGCT | |
| | TTGGGGAAGGGCTCAACCCCAAAATATCTGAGAGCTCTGGAGGGGCCTAGAAGTGGTAAATGAATGAAAA | |
| | CGTGGTTACTCTCCAGATCTGCCTTTCCCAAATATGGCCATTCTTGGCTGAATCAGAAATCAAAGGACAG | |
| | GTTATTAATTACTAGCTCTAAGTTACTTACCATTTGCTGAGACAGTTCAGAAATCTGACTGCATCTCCTC | |
| | AGAGATCTAGAACACAGTTCTCAAATTCTAACTTACTTGTGATATACTTGTGAATGATAAAAATCGCTAC | |
| | AGGTACTTTTATTAATCTGAAAGAGTATTGAGAAATTACCTTTCATTCTGACTTTTGTCTGGAATGAAAA | |
| | TCAATACTTTTGCTATAATCGATTACTGAAATAATTTTACTTTCCAGTAAAACTGGCATTATAATTTTTT | |
| | TTAATTTTTAAAACTTCATAATTTTTTGCCAGACTGACCCATGTAAACATACAAATTACTAATAATTATG | |
| | CACGTCACATCTGTAATAATGGCCTTCATGTAAACATTTTTGTGGTTTACACATAAAATCTCTAATTACA | |
| | AAGCTATATTATCTAAAATTACAGTAAGCAAGAAAATTAATCCAAGCTAAGACAATACTTGCAACATCAA | |
| | TTCATCATCTGTGACAAGGACTGCTTAAGTCTCTTTGTGGTTAAAAAGGAAAAAAAAAAAAAAGACATGT | |
| | TGGCCAGATGCGGTGGCTCACACCTGTAATCCCAGCACTTTGGGAGGCTGAGGTGGGCGGATCACCCCTG | |
| | GCCTGCCCAACATGGTGAAACCCCGTCTCTACTAAAAACACAAAAATTAGCTGGGCGTGGTGGCGGGCGC | |
| | CTGTAATTCCAGCTACTCGGGAGGCTGAGGCAGGAGAATTGCTAGAACCCAGGAGGCAGAGATTGCAGTG | |
| | AGCTGAGATTGCACCATTGCACTACAGTCTGGGCAACAAAAGTGAAACTCCATCTTAAAAAAAAAAAGAC | |
| | AATGTTCGTGGGTCCAAACAAGACTTAATGGAAGTGAGTCTAAAAATGAGCTATGTGGGCCAGGCGTAGT | |
| | GGCTCCCACCTGTAATCCCAGCACTTTGGGAGGCCGAAGCAGGCAGATCATGAGGTCAGGAGATGGAGAC | |
| | CATCCTGGCCAACACGGTGAAATCCTGTCTCTACAAAAATTAGCTGGGCGTGGTGGTGCCTGCCTGTAAT | |
| | CCCAGCTACTCAGAAGGCTCAGGCAGGAGAATCGCTTGAACCAGGGAGTCGGTGGCTAGAGTGAGCCGAG | |
| | ATTGCATCACTGCACTCCTGCCTGGTGACAGAGCAAGACTCCATCTCAAAAAAAACAAACAAAAATAAAA | |
| | GATAAAAATGAGCTATGTGAATTAAAAGAGGTATAACAATAGATAAACCATATTTTATTTAATTCCTAGT | |
| | AATGAGTAATATTTCCAAACTTCTGGAATGGGCAGAAATTGCTAGTTGGCATATTTTTACCTTTTATATT | |
| | CAGATACATTAAAATTCTCAAAAAAAAACACCTCAAAGCAGATGATCCGCCATCTCCTTGGATAATTTGT | |
| | GTTAACTCAGGATAACAGAAAACCAAAATTATGAGTTACTGATGCAATATTCCTAAATGTAAAAATAATT | |
| | AAAGCTAATAGTAGATTCATCTTCCAATTTCATATCAGTCTTACAAATAAACTACATATATAACTTGCTT | |
| | GCCTTCCCTTCTGAGGGATAAAGCTGTTAGAAGAATTAAAATCAGCATTCTTGACTATTCAACCAAGGGA | |
| | GGGATAAATTATTACTCATTCTAGGGACATGGGCTCATAACTACTACATGTGTAAGGACATGAATTTACC | |
| | CAATATTACAATTTTTCCTTTTATTAGTGTGTACAGTGGAAGAATAGACATGTTCACTCTGGACAAAAAA | |
| | AAAATTATACTTATCAGTTATCAGAAGCACAATGCTGAAGACAGTAGTTCCATAACAATTTGAAGTATGT | |
| | GATCGAACTAGTAGATTATCTTAGTAGTAGTGAATTATTGTAAATGTTAGTAATTTGGCAGCCACTGGGC | |
| | AGAAAAATAAGAATTGAGGCTCAATATTGATATTAATGGTGGTGATTGACACATAAATTTTATCAAGTCT | |
| | ACACAATATAAAATTACAGAAAGGTAGAAGAGTATACCAGTACAACTTCAACATATCTTCACTACAAGGG | |
| | AGTAAAATGACATGGCCTAGTTACTATCTAATGAACTGCAGAAAACTAAAAGAAAACTCCAAGGCAACTC | |
| | TTCTCTGCTGATCTGGTTGGTCCTTTTCCTACCTTTTGCAATACCCAGATACAAACAATGGATAGAAAAC | |
| | AAAGTAGACTTGTAGTATGCAGGTCACAGTGCTAAATTCACAGAAAGAAACCCCTGAACTGAACTGCTCT | |
| | ATTTCCTGGTGGTCACAAAGAGTAATTCTGGTTTACACCTACAGATTGATGTCAATCTACACCCTGTTGA | |
| | TAACAGTGTGGCCAAGGACAAAAAAAAGGTGCTCCGTTTTACCAATTCTGTAAAAAATTATTGGCAGGGT | |
| | AAGCTCGGCTAGGGCAGGATTACATTTCTAGGACTACCATCCCCGAAATTTAGAAGATATTATATCCACA | |
| | TAAAGCATATCTTTCACATTAATTTGCAAAAATCTAAAAGCTTTTTCTTAGCTCAAGTGTGTCCAAGTTT | |
| | ACCCTGGCAGTTTAAAACGATAGTTACAAGCAGCATGGGTTGTATCAGACACATTTGAGGGCCAATTTCA | |
| | TGTAAGTGATATTGGGCAAGTTACTTCAACTATCTGTGCCTCCAAGGTCATACTAGTGTTTATTTACCTA | |
| | AAGGGTACCTGTTATGTAACTTTAGGGTGTTTACATTAGATAATGCCTGCAAAATATTTACTTCAACGCC | |
| | TAAAACATAGTTAAGTATTCAATAAATACCTACTATTGTCACTACTAACTTAAAAGTTTAGAGATTAAGA | |
| | GCAGAATCTGGGGTGAGACAAACTTAGGTTCAAATCCTAGTATTGTTGGGTAATCTTGGGCAAGTTACTT | |
| | AACCTCTCTGATTTGTGTAATTTAAAAAATTAGTTAATATACATAACAGGGCTTAGAAGAGTATCTAGCA | |
| | CATAGCACCATTTAAGCATTTGTTATTGCTAACATGCAAACAATTTAAGGGAAAGAAATTTTTTAAAAAG | |
| | GAAGAGGGATTTGCAAACTAAAAACAATGAGTATCTTATGTTCAAAGAAAACTAACAAACAGCCAGCTCT | |
| | AGCAATAATTAAATTCACTATATACTGGGGCAGGCATCACACCCCAAAGCTAAAAGCGTCTACCTAGGCC | |
| | AGGCACGGTGGCTCATGCCTGTAATCCCAGCACTTTGGGAAGCAGAGGCGGGCAGATCGCTTGAGCTCAG | |
| | GAGTTCAAGACCAGCCTGGACAACATGGCAAAACACCATCTCTACAAAAAATACAAATATTAGGCCGGGC | |
| | GCAGTGGCTCACGCCTGTAATCCCAGCACTTTGGGAGGCCAAGGCGGGTGGATCACCTGAGATCAGGAGT | |
| | TCGAGAGTAGCCTGGCCAACATGGTGAAACCTCGTCTCTATTAAAAATACAAAAAATTAGCCAGGCATGG | |
| | TGGCAGGCGCCTGTAATCCCAGCTACTCAGGGGGATGAGGTAGGAGAATCGCTTGAACCCGGGAGGCAGA | |
| | GGTTGCACTGAGCCGAGATCATGCCACTGTACTCCAGCCCGGGCAACAAGAGCGAAACTCCATCTCAAAA | |
| | AATAAATAAATAAATAAATAAAATAAAGTACAAATATTAGCCAGGGATGGTGGTGCGCACCTGTAGTCCC | |
| | AGCTACTTGGGAGGCTGAAGTGGGAGAATCCCCTGAGCCTGGGGAGAATCACCCGAGCCCGGGAAGTCGA | |
| | GGCTGCAGTGAGCAGTGATTGTGCCACTGCACTCCATCCTAGGTGACAGAGTGAGACCCTGTCTCAAAAA | |
| | AAAGAAATTGGCAGAATTAAGTAAGTTGATGTTTAGAGATGAAAAATCAACATTTTTTCCTCAGCAACTG | |
| | AATAAAAACAACAGCCACTACCATTTTTTTGAGTACCTATTTGTAGCCTATTTTTTAACTGGTATTACTC | |
| | GAGAGAGAGAGAGCTAGGTTCGAGACAGAGCTCCTTCTCTTAATAACTGTATGACCTAGGGTATGTCTGT | |
| | TAGCCTCTCTGAGGCTTCAAAGGTTCCTCATCTGTAAAATGGTAATAATCATACCATTGCTACAGGGCTG | |
| | TTTTGAAGACTAATTAGGACTATGTAAGTAAACATGATGATGGCTATTATTACTGTTCCCCGCCAGGGGC | |
| | CATGCAAGGGTTGCTGATTCACATAGACTGTCTTATAATCCTCTCAATAACTCCAAGAGGTAGCCAGCAC | |
| | CTCAGATATACATAAAATGACTTAAGCCCAGAGAGGTGAAGTAAGTTGCCCACAGCCACACAACTAGTAA | |
| | ATAGCCCAAACAAGCTGGATTCCCAGTTAGACTCCGTTAATAGCACTGCTCTTTACCTTAAGTCATTACA | |
| | ATGCCTAATATGAAATAGAATCGCTTCTTTCTTAGGGTTCAAGTGGTTAATTATTTAATGTATTCATTCA | |
| | ACAAACCATCATCGAGGACCTCTTACAAGCCAAGTACTGTGCTAAGTGCTAGAGTTACGGCGGTGATTCC | |
| | TGCCCTTAAAAAGTTTTAGTGGGAGAAACAACAGGTAACCAGGTCATTGCCAAAACAACAAAAATAATCA | |
| | TAATAAAGCAGGCTAAAGCATATTTAACTGGCCGGGGTTTTGACTATTTTAGCAAGCATGATCAGAACGG | |
| | TTGAGGAGGGAGGCCAGCAGCTTGGCCGGTTCAACAAACAAGAAAAAACCAGTGAGGGTGGAGCTAAGAT | |
| | ACCAGAGGCTGATTACGGTTAAGAATGTTCTTGAAGGTAAGGACCAGATTCTCATTTTCTATATCCTGGG | |
| | GCATCGGTCAGCATGGAATCTGGATTCTAGCACATGTGAATTTCGGCTTGAAATGACCTAATGCCTTTTC | |
| | CCTAGTTCCTTCGTGTGTCAAATACGCATGGTTACCGCTACCAGAGCTGTAGTGGGGCTTCAATGAGGCC | |
| | ATGAGCATCTCCATAAAGATGAACTACAGTGTGTGCAAAACTAAAGGCAAAACCTGGTCCCCACACGCCC | |
| | TCCCAGGTGGTCGCTTTCCGTGCCGAGGCCCCTCCAGAGGTGCCCCGAGAACCTCACCATCGCACCCCAA | |
| | ACTTCCAGGGAAGGGCCTCTCCCGAGAAAGCCCCCACGCCCCCACCCCGCGCCATCATTCCCGAATCTGC | |
| | CCTCGGCCCCTCCCCGCAGCACGCTCGCAGGCGGCACATGTCAACCAAAACGCCATTTCCACCTTCTCTT | |
| | CCCACACGCAGTCCTCTTTTCCCAGGGCTCCCCCGAGGAGGGACCCACCCCAAACCCCGCCATTCCGTCC | |
| | TCCCTGCCGCCCTCGCGTGACGTAAAGCCGAACCCGGGAAACTGGCCGCCCCCGCCTGCGGGGTTCCCTG | |
| | GGCCCGGCCGCTCTAGAACTAGTGGATCCCAATTGAAGGCCTGGTCTAAATGACTCCAAAATCACCACTT | |
| | AATTCAAGAGACTGATTTCCCTGAGTCAGGCCCCTTAAAGCAGCTATTTCAATGGGACAGGGAAACAACC | |
| | CTAGGATCTGGATTAGAATCACTTGGGGGCTGCCACACCCCCAGGGCTCTGATCCTGCCCTTCTCCCACA | |
| | CGCACATTCACATACTGCTGCAGTGACCTTCCATTTCTAATGGGTTCCTGGGCCATCTGTCAGGTATAGG | |
| | GAATGGAAAAGGGGTTGGGGAGGCTCTGCTTCAGAAAGTTTGTGTCAGGGGCTCCCAGAGCCTCCACAGA | |
| | TAGATAGCAGGGGTCCCCACCCTACCATGGCAGCTATAAATGTGATCAACATTTATTGGCCTAGGATACA | |
| | GCAGTTAGCAAAATGCCTGATGTAGTTCCCACTCCGTGGAGGTTGCAGGCTAGCCAAGAAGTCATGAGTT | |
| | CAGCAACCCTTACGCACCAGTGGGATGAGATTGGACCAGGCCGAGGGTAGTCTTGGGAACACTCAGCATT | |
| | TGTCTGAGGGCCAGAAGAGGCTGCTTGCCCTCAGACAGGAGGTCAGCATCTTTATTGTAGCCCATGACAC | |
| | CTCTACACCATTGCTCTTCTGGTCTTATGGAAGACATCTTTGGGCCTGATAACAGCGGAGTCTGTGTCCC | |
| | ACTTGTCCAGGCTGGAGTGCCACATCAGGCACACTCCAGTTGCAGGGACAGCACAGACAAGTTTCAGGAA | |
| | GGCTGGTGGCCTCCAGGAGGTTAACCTTATAAGGCCAGATTGTAACCTAGTTGAAAAACATACACATGCC | |
| | ATGATAATAAAAGAACCTAGGCACCATTACAAGAGAAAAAATCATTTTTGTAGATACGAGCATGGATTCT | |
| | TGGGTGGGTCAGACACACTGGGCTTGTGCTCTGACTGCACTGTCTCCCCTACCTGACCTTGGGTAAACCA | |
| | TAAGACTGCTGCATGACTCAGTGTCCACCCCAAAAAAGTACCGGTAGATATTGGCCACAGTAGATATCAG | |
| | CTAGAGTGGACTCTCATGACAATGAGGGGAGATGTATTCCCCATCTTAGGCACCTGGGACTCTACCTTCC | |
| | ATCTTCTGCTCCGTGTCTCTCCATCCCCAGGCTCTTCAGAACTCAGGGAGTCCAGAATGTCAGCTCCCAG | |
| | ATTTCAGCCTTCAGAAAGGAAACCCATTACCGTTCAGTTGAACAAATGTTGTCTGAGCCCCAGATCTGGG | |
| | CTCAGAGGCCATCTAGGCTATGAGACAAGAGGGGAACAAAGCACCGTCTGCACTCACTCACCACACTCAC | |
| | TTGCTGTCCCAGGTCACATCCATCGGGTAGAGAATCTAAGAGGCTGAGCTAGCTCCCGCCACCAGCCCAG | |
| | CCCACCCCACCTGGCCCCTTCCTTCCTTCTACAAAATATGCACCACCTGTCAAAGGGTGGGCAGTGCCAG | |
| | GCCTGCATACAGAGCACTGAGTGTAAAAGCAGACATGGACCCTGACCTCCAGGAGCTTCCAATTTTCTTG | |
| | AAGAGACAAATCAGCTGGCATTTCAGTCCAGTGTGATCTGCTCTTGGTGAGCACAGACCTAGGGAGTTGG | |
| | GGCAGCTTCCCAGAAGAACTGCAGTCCAGGCTGAGGGCAGAGAAATGAGGGGAATGGCGAGGAATTGGGG | |
| | AGCAGGGGGGAGCTCAGTAGAGAGCCAAGGGCGGGAGGTGAGAAGTCCGTGTTGGGCCAGGAGCTACCCT | |
| | CCGGTGGCCACAGCCGAAGTCGAGGATGCCTTTGGAACTCATCCCCACTTCTCTCTTTCTGTATGTAGCC | |
| | GTCCAAGAACAAGTCACCTCCAAGTGTAGCCGGATCAAGGCAAGCCCCCCATCTAGCAAGCACTTGATGC | |
| | CACCCAGAACTGGGCTTCTTCAGAACAATCTGAGTCCAGGAATGATCCCACTCACCAGGCACCAGAGCTG | |
| | CGAGGGCATGGGAGTGATCTCACCAACTCTGGGGAAGCGGCAAGGAATTTTCACCTCCAGCCCCCAGTGT | |
| | CCCATCCTCTCACACTCAGGCCAGACTCCCCTGGGCAGACTTGACTCTGTCTGCCAGCATATGCAGAGCC | |
| | CCAAGGCCACCCCACCAGAAGTGCCCCTGCCTGGGTTCTGTCCCAGCTCCCTGGGCACCCAGTCCTTGAG | |
| | TCCCCACCAGCTCAGACGGCCTAGTGTGCCAAGAATGCCCACTGCGTTCAACAATGCTGCATGGGTCACA | |
| | GCGGCAGCAGCTGTGACCACAGCAGTTTCGGGGAAAACACCCCTCAGCCAAGTGGATAATAGCGTTCAGC | |
| | AGCACTCACCTTCTGGCCAGGCCTGCCTTCAGAGGCCATCTGATTGGGAGGCACAAGTGCCCGCTGCGAT | |
| | GGGAACACAAGTGCCCCTGGCCAACAACCCCAGCTTCAGCCTGCTGGGCAGCCAGAGCCTCAGGCAGAGC | |
| | CCGGTACAGGGCCCGGTGCCTGTAGCAAACACCACCAAGTTCCTCCAGCAGGGTATGGCCAGCTTTAGTC | |
| | CCCTGAGCCCCATACAGGGCATCGAGCCACCAAGCTATGTGGCTGCTGCTGCCACCGCTGCTGCTGCTTC | |
| | TGCCGTTGCTGCCAGCCAGTTCCCAGGTCCGTTCGACAGAACGGATATTCCCCCTGAGCTGCCACCTGCC | |
| | GACTTTTTGCGCCAGCCCCAACCCCCACTAAATGATCTGATTTCGTCACCTGACTGCAATGAGGTAGATT | |
| | TCATTGAAGCTCTCTTGAAAGGCTCCTGTGTGAGCCCAGATGAAGACTGGGTGTGCAACTTGAGGCTGAT | |
| | CGACGACATTTTGGAACAGCATGCTGCTGCTCAAAATGCCACAGCCCAGAATTCTGGGCAAGTCACCCAG | |
| | GATGCTGGGGCACTTTAAATCTGAGCAGGATGCCCATAGAAACCCCCATGGTGACATCACTCTAGGAAGT | |
| | GGTGTCGATCCATACCCGCAGTTGTCTCCCGTTACAATTTGAGTGGTGTTGTCAGCCCATGCTTATCCCT | |
| | CTCTCTACCTGTGACAAAATGGAAAGCTGGTGATTTTTCAAGCTACGTGTACATATTTGAAAATTTTGTA | |
| | AATGGTTTTCCTAAACATTAATGACAGAAGTATTTATACTTCATTTTGTGACTTTGTAAATAAAGCGACG | |
| | GCTTTTGTTTCAGTAGAGTTGTGTTTACTATGCATTGTTTTGTGTTTATTATACAATGTTACAAATATGC | |
| | AGACCGTGTTGTTTGCTCCAGTGATACCTTGTTAAGCTAGGTGGCTGAGTCGCTTATGGTTTTAATGCAA | |
| | TGAGCAATGTGGATATGACCAAGAGTTGTTGTGCAAGTTGACAAATGCCAAATAGAAAACCACTTGGCCA | |
| | TTTATTTCTATGTTCACTAAAAATCCTATTGCCTTGTGTGATTCTTAATCTCTTTTGCGAACCTTTCAGT | |
| | CTCCGCTAGCTCTTTCCTAATGAGCTTTACAGCAGAAGCTGTTTTATCGTTAAGTGCCCCACAGAGACAC | |
| | TTTACCAGGAGGCTGGGAGAGTTCTCCAGATTTGGGAGAGGCGCAGAGACAGTGTGTGAGCCGAGCCCTG | |
| | TCTCAGCAATCCACCTGGAGGAGCTAGAGTATCCTCCTCCCTTTACCATTCAGACCGAGAGAAAAAGCCC | |
| | AGCTTGTGTGCACCCTCGTGGGGTTAAGGCGAGCTGTTCCTGGTTTAAAGCCTTTCAGTATTTGTTTTGA | |
| | TGTAAGGCTCTGTGGTTTGGGGGGGAACATCTGTAAACATTATTAGTTGATTTGGGGTTTGTCTTTGATG | |
| | GTTTCTATCTGCAATTATCGTCATGTATATTTAAGTGTCTGTTATAGAAAACCCACACCCACTGTCCTGT | |
| | AAACTTTTCTCAGTGTCCAGACTTTCTGTAATCACATTTTAATTGCCACCTCGTATTTCACCTCTACATT | |
| | TGAAATCTGGCGTCTGTTTCAAGCCAGTGTGTTTTTTCTTCGTTCTGTAATAAACAGCCAGGAGAAAAGT | |
| | GCCTCTATGTTTTTATTTTTCAAGGGAGTATTCAGTACCTACAAACCCAAGTCAGGAAGCCTGCTAGTGG | |
| | CTTTGGTTCTTTCAGAGGCTGCTCGATGCCTTGTGTGTCAGAAAGAAAGATTCAGCAGTTTTGCATCATG | |
| | GCAAAGAAGCCTGTTATTTTGGGGCTCAGCCCCTCATTTTATAGAGGATGAAACAGAGGGGGATGGGAGG | |
| | TCACAAAGACAACTGCCCCGGGAGCAGGTGTGGGGGAGACTTGCCCTGAGGGTCTAGACGCTCTGCACCA | |
| | CCGTCCTGTCTCCCTTGCTGAAGACCACACATGCCCTTCTTTGACCAGACCCTGCCACCTGATAGGCCAG | |
| | GACCTGGTAGGCGGGTACCCAGGTTTCATGGATGGAACCACATCTCCCCAAAAGTGGGGAGGTAGCTACT | |
| | GGGATGCACGCCTCCCGCCATGTGCTATAGGAGAGCAGCTGAAGCAACAGTTGGGATCAGATGTAGTCAC | |
| | AATTGAATGCATCATCACATTTATCCCTCTAAGTGGCTGGGAGAGTTGATATCCTCATCCCTAAGGTACA | |
| | AAATGTTCCAATTTGATCAGTGGCTTTCAGGAGCTGAGAAAGGCATGTGCTCTGAGGCAGAGCTGTTATG | |
| | TCCCGCAGAGCCTAAAAATGCTCTAAGAACATGCTCCCTGCCAAAATTCTCAATGGCTGTGACAAGGGAC | |
| | AACGATCGACCAATGGGGGTGGAAGCAGACCTCCGCAGTCCAGGGGCCAGAGCTAGGACAGAGGGGTCGG | |
| | AGAAAGAGTCATTTTCCCAACACTCCAGCTCTTGGCCAGTCCTCACACAGTCCCCTCCTGCTTCCTGCTG | |
| | AGAGAGATATCCTCATAGGTCTGGGTAAAGTCCTTCAGTCAGCTTTCATTCCCTGTCACCAACTTTGTCT | |
| | CTGTTCTCCCTGCCCGTCTCAGGCAGCACTCCTCAGGAAACCTCTCCAAGAGCCAGCCTCACTGCAGCGC | |
| | CCACTATTGTCCCTCTGCCTCAAGTGTCCCATCCATGCCAGGCCCCAGGCAGGCTGCAGCTTTCCCTCAG | |
| | GGCCACACCAAAGCACTTGGGCTCAGCTGTGCTGTCCCCCTCCATCACTGAGCTCAGGGGCAGCAGGGGT | |
| | GGGGTGCCAGGAGGCCCATTCACCCTTCTCTGGCTCTGTGTTGGACCCACCTGCCCAGCCACTGCTGCTT | |
| | AGAACCTACCCGCTGGGAAAATGAAGCCCTCCCGGAGGGGCCACCTCAACCTGAGAGCCTCACGGATCAC | |
| | AGTTGTCCCCACTCAGCTCTGCCAGCCCTCAGAGACCCATAGATAAAAGCTGAGCTTGGCTCGCAGAGCT | |
| | GGTTCCATCTTCCATTCCCAGAGGGTTCAACTTCCTACCCCAACCACACAGGGAACCTCAAGGCTGAGCC | |
| | AGTGTGGGCTGCAGTGCAGACCAGCTTCCTGGACACGTCCTGCCACCTGACCCCAGGCTGGCCTCACTGC | |
| | CCCTGGCACTCCTGACCCTATCCTCATTCCTCCTGGCAGTGCGTGTTCTGCCATTCCGCTTTCCCTTAGC | |
| | TGTCCTCTCACTGTACTGTCAGCTTCTCCTTTTCCAGGTGCCCCCCAGGGGCTTTCCACATGACCCTGTC | |
| | ACCCCACAGCCCATCCAGCACCAATTCCAGCTCTCTGCCACCCTTCAAAGGAGTGACAGTGCCCTGCTTC | |
| | ACCTCCCACTCACCCCTCAACCCAGAGCAATCTGGCTCCAGTCTTGCCTCCTTCCCCCTAAGTACTCTAG | |
| | TCACAGTTCCAAATTCCTCCTGGTCATAAAGCCAAATGAAGCTTCCTGGTCCTCAGCGGACTTGCCACTT | |
| | CAGCAGTACTGGACTCTCTCCTCCCAGAAACCTGTTTCCCCTTGGCTCCTGGAGCCCACACTCTGCTGGA | |
| | ATCCTTCTGCCTCTCTGGCCTGTAGCCTGGCCCTCTCTCCCAACCTGAGGTCCATTCTCTCCTGCTCCTC | |
| | CACAAGATGTTGCTCCTTCCATTACTTCCTCCCTCTCAACCAAAGCTCCTTCATTAGCTCTTTATCTTCT | |
| | GGTTTCTTCCCCTGGGCAGACGAATGGATTCAAGAGCCTGTGGCCCAGCAGCCCAGCACTCCAGGATCTC | |
| | AGCACTTCAGCATCCCAGTACCCTAGCATCTCAATACCCCAGCACCCCAGCACCATAGTATTCCAGCACC | |
| | CCATTGTCCAAGCATCTCAGCACTCCAGCATCCCAGCACCCCAACACTCCAGCAGCCCAGAATCTCAGCA | |
| | CCCTAGCACTGCAGCATCTCAGGACCCCAGCACTTCAGCATCCCAGCACACTAGTACTCCAGCATCTCGG | |
| | CACCCCAGCACCTAGGCATCCCAACACCCAGCACCCCAGCACTTAAGCATCCCACCACTACAGTATCTCA | |
| | ACACTCCAGCACCCCAGCACCATAGTGTTCCAGCACCCCAGCATCCCAACACCCCAGCACTTAAGCATCC | |
| | CAACACCTCGGCATCCCAACACCCCAGCACTGCAGCATCTCAGCACCTTAGCATCCCAGTGCCCTAGCAT | |
| | CTCAATGCTCCAGCACACCAGTACTACAGTATTCCAGCACCCCAGCACTCCAGCATCTCAGCACTGCAGC | |
| | ACTGCAGCACTCCAGCATCCCAAAATCCCAGCATCCCAACACCCCAGCAGACCAGCAGACCAGCATCTCA | |
| | GCACCGCAGCATCCAAGGACTATCCCAGCATCCCAGCAACCCAGCACCTCAGCATCCCAACACCCCAGCA | |
| | TTTCAGCATGGCAACACCCCAGTACCCCAGCACTTCAGCACCCCAGTATCCCAGCATCTCAGCGACCCAG | |
| | TATCACAAAACCTCAGCATCCTAGCACCCCAGCACCCCAGCACCTTAGCACCTTAGCATCCCAGCATCTC | |
| | AGCGCCTCAGCATCTTGATATTCTGGCTGAGGTCAGCGTGGTGTATCTAGTCAGGGTCCTAACTTTCACT | |
| | TCGCAGGGAAATGCTGCTGGACTGGGTCTCATGTTGGGCTGAAGCTCTCTAGACCCCTTGAAGACAGCAT | |
| | AAAAGAGCTTGGAGACGCTGGGTGTCCCCCATGGAAGAGTTCACTCTCATCCTGCTTTGACAACAGCCTT | |
| | CTCTGGGGTCCCTCACGGGCCCCTCTTTCTTACTGCAAGTTTGTCTCTGAGAAGACTGTGATGCAGAAGT | |
| | CACTCAGCTGCCTGTGGCTCCTGAAGAGCTGAAGGTGGAGGCCTGTAGGCCTCCCTATGAGAGGCGCAGA | |
| | AAAAACCATGATTGCTAGTGGGGAGGTGCTCCCTCTACAACCCACTCCATAATCTGCCCCCGCCCAGCTC | |
| | TGAGGCCAGCCCCAGGGGAAAATGCCAGATCCCCAGGGAGGTGTGTGAGACCTCAGGGGCTCCCTCCTCC | |
| | CTTACAGCAGGCTCAGGCCCCTGGGGGCCTCAGGGCCAAGGTCTGTGGGTAAGCTACTATCTCTCACTTG | |
| | TCCTCTAGCCACAAAAGCCAGGGAGATCTGGCAATGGACATGAGGTTCTGAAGAAGCACATATGACTGGC | |
| | TTCCTAATGCGTGGTTGTTCAGTGATTCAATAAACACGCATGGGCCAGGCATGGGGAAATAGACAAACAT | |
| | GATCCCCAACCTCTCCCAGAGTGAACTGGGAGGGAGGAGTGTTCATCCCTCAGGATTACACCAGAGAAAC | |
| | AAACCAGCAGGAGATATATATGGTTTTGGGGGGTCAAGAAAGAGGAAAAACCTGGCAAGGCAAGTCCAAA | |
| | ATCATAGGACAGGCTGTCAGGAAGGGCAGCCTGGAACCTCTCAAGCAGGAGCTGATGCTGCAGTCCACAG | |
| | GCAGAATTTCTTCTTCCTCGGGGAAATCTCAGCTTTGTTCTTAAGGCCTTTCAACTGATTGGCTGAGGTC | |
| | TGCCCCTTCCCCCACATTCTCCAGGATAATCTTCCTTACTTAAAGTCAACTATTAATCACAGCTACAAAA | |
| | TCCCTTCACAGCTACACATAGATCAGTGTTTGATTGACGAACAGCCCCTACAGCCTAGCCAAGTTGACAC | |
| | ATAAAACTAACCATCACAGGGGGACAAATGATGTAAACACATCAACAAATAAAACAGTAACAAGTTAAGG | |
| | TCTATGGAAAAAACACAGAAGGGGCAGAGAGAAAGAAAGCAAGAAGGAGAGTCCCAGTTTGCTAGGGCTT | |
| | GTGGGAAGTGGGGAGCAGTTCTCTTTAGCTAGGATATTTGGGAAAGGCATATCTGAAGGAGTGATATTTG | |
| | AGCTTAGATTAAAAGATGGGAAGGAGCAAGCCATGCAAAGAGCTAGGATGTTCCAAGCAGAGACGGAACA | |
| | GCAAGTGCAAATGTCAGGAGGAATAGAAGGAGGCTGGTGGGTGGGGTCCAGTGAGCAAGAGGAGGGCAGG | |
| | CAGGAGAGGGGATGGGGAGGTGGGCAGGCCCAGACCACCCAGGGCCCTGGAGACTATCCTGATCCAACAA | |
| | GGGAAGCCTTGAGTCACTTCAGTGTCCATGTGGAGAATGGACCTCAGACTGAATGAGGGAGGCAGTAAGG | |
| | AGGGCCTCTACCTCCAGGGCTTCGCCCTGTGGACTGCGCATAGACATCTCCAACTCAGAAAGTCTGAACC | |
| | AAACTTTCCATAGTTCCCCCAAGTCTGGGCATCCTCCTACTCAGTGAAAGGCAGCCATCACACCTCCCTG | |
| | CCCTGCTCCCGGATGCCCCAAATCCTCTTGGTCTCCAAGTCCAGAACCTGAGACTTGTCCTTGATGTTTG | |
| | TCTTTCCCTCACCCTTTCTGTATTCTGGGAAGATGGGTTTTTTTCCCCCAGATGAATCTGTAAAACTTCT | |
| | GTGATCACAATAAAAATTCTGGCAGTATTATTTTCTGGAACATGACAAAGTGATTCAAAATTATTTATCT | |
| | GGAAGACTACAAAACAAGAATAGCCAGGAAATTTCTAAAAAGAAAGAAGAAGGAGGAGGAGAAAGAAGGA | |
| | GGAGGAAAAGGAGGAGAAGAAGAAAAGAAAAAGAACCAAGAAAGGGTTCTAGCTCTACCAAATATTAAAA | |
| | CATATCATGAAGCTATTTAAAACAATATGGTTGTGGATACTGAAAAAGATGTGAATAAAGTGGAAGGAAA | |
| | ATAAATAGAAATGCACATGGGGATTGAGACTGTGAAAAAGGCAGCATCTCACATCAGTGAGGGATGTTCA | |
| | ACACCTGGTGTTGGGAAAACTGGCTAGTCATTTAAACCAAACAACTGGGTCCTCTACCTCACTCCTGACA | |
| | TTAAGATACATTTAGATGATTCAAAGAGTAAGACAGAAAAAATAACACGTGAAAACACTATCAGAAAACA | |
| | ACGTGGGCCAGGTGTGGTGGGTCACGCCTGTAATCCCAGCACTTTGGGAGGCCGAGGCAGACAGATCACC | |
| | TGAGGTGGGGAGTTCAAGACCAGCCTGACCAACATGGTGAAATCCTGTCTCTACTAAAAATACAAAATTA | |
| | GCTGAGCGTGGTGGCGCATGCCTGTAATCCCAGCTACTCAGGAGGCCGAGGCAGGAGAATCACTTGAACC | |
| | TGGGAGGCAGAGGTTGTGGTGAGCCGAGATCACGCCATTGCACTCCAGCCTGGGCAACAAGAGTGAAAAT | |
| | CCATCTAAAAAAAAAAAAAAAAGCCAAGGTGGATATTTTTATAGTATCAGGGTAGATCAAGCTTCTCCAA | |
| | TCATGACATGAAACCCAGAAACCATAAAAGAAAAGAATGATAAAATTGCCCACGTAAAGTAAAAAGCTTG | |
| | CACACAGAAAAACACCATACAGGTTACAAGATGAGCAGCAAAATCAGAGAAAAAACATTGCAATTCAGGA | |
| | CACACAGAGGCTATTGTTCCTAATATTTAAAAATAAAAGTAGTGGATTGTCTACAAAAAGATGAAGACAA | |
| | GAATTTCAGAAAACCAAATACTGCATGTTTTCACTTACAAGTGGAAGCTAAACACTGAGTACACGTGTAC | |
| | ACAAAGAATGGAACCATAGGCCAGGCACCGTGGCTCACGCCTGTAATCCCAGTACTTTGCGAGGCCGAAG | |
| | CGGGCGGATCACCTGAGGTGAGGAGTTCGAGACCATCCTGGCCAACATGGTGAAACCCAGTCTCTACTAA | |
| | AAATACAAAAATTAGCCGGGCGTGGTGGTGGGTGCCTGTAATCCCAGCTACTCGGGAGGCTGCGGCAGTA | |
| | GAATCGCTTGAACCCTGGAGGTGGACCTTGCAGTGAGCCGAGATCGCACCACTGCACTCCAGCCTGGGCA | |
| | ACAGAGTGAGACTCCATCTCAAAAAAAAAAAAAAGGAATAGAACAATAGACACTGGGGCCTACTTGAGGG | |
| | AGGAGGGTGAGGATCAAAAACCTGCCTATCAGGTACTATGCTTATTACCTGGGTGGTGAAATAATCTGTA | |
| | CACCAAACCCCAGTGACATGCAATTTACCGATGTAACAAACCTGCCCATGTACCCGCTGAACCTAAAATA | |
| | AAAGTTGGAAAAAAATATAGAAATTTTCTTTGTAATAGCCAAAAACTGCAAACAGCCCAGGTGTCTATTA | |
| | GTAGAATGCATAAACAAACTCGGGCATGTTCATACAATGTAAAACTACTCATCAATAAAAAGTGATACTT | |
| | CTCAGCAATGAAAAGAAACTAGCTACTGATACCAGCTACAACATGGATGGATTTCAAGTGCTTTATGATG | |
| | AGAGCAAGAAGCCAGACACAAAAGTGTCTATATATATATACAGTATATATACGTATATATACACATATAT | |
| | ACAGTATATATATACATATACATGTATATATATACTGTATATATACTGTATATATATACACAGTATATAT | |
| | ATACATATATACAGTGTATATATACTGTGTATATATACATGTATATATACTGAGTATATATACATGTATA | |
| | TATATGTATACTGTATATATACTGTATATATATATACACATATATACAGTATATATATACAGTATATACT | |
| | GTATATATACAGTATATACGTGTATATATACATATATACAGTATATATGTAAATATACATATATACAGTA | |
| | TATATGTAAATATACATATATACATGTATATATATACACTATATATATACATATATAGTGTATATATACA | |
| | TATATACATGTATATATTTACTATATGATTCCATTTATATAAAGTGCCAAAACAGTCAAAAATAATCTAT | |
| | GTGGAAAAAATCAACAAAGGGATCCCCCGGGCTGCAGGAATTCGATGGCGCGCCTTAATTAAAATTATCT | |
| | CTAAGGCATGTGAACTGGCTGTCTTGGTTTTCATCTGTACTTCATCTGCTACCTCTGTGACCTGAAACAT | |
| | ATTTATAATTCCATTAAGCTGTGCATATGATAGATTTATCATATGTATTTTCCTTAAAGGATTTTTGTAA | |
| | GAACTAATTGAATTGATACCTGTAAAGTCTTTATCACACTACCCAATAAATAATAAATCTCTTTGTTCAG | |
| | CTCTCTGTTTCTATAAATATGTACCAGTTTTATTGTTTTTAGTGGTAGTGATTTTATTCTCTTTCTATAT | |
| | ATATACACACACATGTGTGCATTCATAAATATATACAATTTTTATGAATAAAAAATTATTAGCAATCAAT | |
| | ATTGAAAACCACTGATTTTTGTTTATGTGAGCAAACAGCAGATTAAAAGGCTAGCCTGCAGGAGTCAATG | |
| | GGAAAAACCCATTGGAGCCAAGTACACTGACTCAATAGGGACTTTCCATTGGGTTTTGCCCAGTACATAA | |
| | GGTCAATAGGGGGTGAGTCAACAGGAAAGTCCCATTGGAGCCAAGTACATTGAGTCAATAGGGACTTTCC | |
| | AATGGGTTTTGCCCAGTACATAAGGTCAATGGGAGGTAAGCCAATGGGTTTTTCCCATTACTGACATGTA | |
| | TACTGAGTCATTAGGGACTTTCCAATGGGTTTTGCCCAGTACATAAGGTCAATAGGGGTGAATCAACAGG | |
| | AAAGTCCCATTGGAGCCAAGTACACTGAGTCAATAGGGACTTTCCATTGGGTTTTGCCCAGTACAAAAGG | |
| | TCAATAGGGGGTGAGTCAATGGGTTTTTCCCATTATTGGCACATACATAAGGTCAATAGGGGTGACTAGT | |
| | GGAGAAGAGCATGCTTGAGGGCTGAGTGCCCCTCAGTGGGCAGAGAGCACATGGCCCACAGTCCCTGAGA | |
| | AGTTGGGGGGAGGGGTGGGCAATTGAACTGGTGCCTAGAGAAGGTGGGGCTTGGGTAAACTGGGAAAGTG | |
| | ATGTGGTGTACTGGCTCCACCTTTTTCCCCAGGGTGGGGGAGAACCATATATAAGTGCAGTAGTCTCTGT | |
| | GAACATTCAAGCATCTGCCTTCTCCCTCCTGTGAGTTTGgtaagtcactgactgtctatgcctgggaaag | |
| | ggtgggcaggaggtggggcagtgcaggaaaagtggcactgtgaaccctgcagccctagacaattgtacta | |
| | accttcttctctttcctctcctgacagGTTGGTGTACAGTAGTAGCAAGCTTGCATGCCTGCAGGTCGAC | |
| | TCTAGACTGCCATGGTCTTTGTTAGACGACCGTGGCCGGCTCTGACAACTGTGCTGCTCGCCCTTCTCGT | |
| | GTGCCTGGGAGCTCTTGTTGATGCCATCAAGCCTGAGGCACCTGGAGAAGACGCATCCCCTGAGGAACTG | |
| | AACCGCTATTACGCATCATTGAGACACTACCTTAACCTCGTGACAAGACAGCGGTACGGTAAGAGAATCA | |
| | AGCCAGAGGCCCCCGGGGAGGATGCTTCCCCAGAGGAGTTGAATAGGTATTACGCTAGCCTGAGACATTA | |
| | CTTGAACCTTGTGACACGACAGCGATATTGAGGATCCCCGGGAGATATCCTAGGCTTGGCCAGACATGAT | |
| | AAGATACATTGATGAGTTTGGACAAACCACAACTAGAATGCAGTGAAAAAAATGCTTTATTTGTGAAATT | |
| | TGTGATGCTATTGCTTTATTTGTAACCATTATAAGCTGCAATAAACAAGTTAACAACAACAATTGCATTC | |
| | ATTTTATGTTTCAGGTTCAGGGGGAGGTGTGGGAGGTTTTTTAAAGCAAGTAAAACCTCTACAAATGTGG | |
| | TATGGAATTCAGTCAATATGTTCACCCCAAAAAAGCTGTTTGTTAACTTGCCAACCTCATTCTAAAATGT | |
| | ATATAGAAGCCCAAAAGACAATAACAAAAATATTCTTGTAGAACAAAATGGGAAAGAATGTTCCACTAAA | |
| | TATCAAGATTTAGAGCAAAGCATGAGATGTGTGGGGATAGACAGTGAGGCTGATAAAATAGAGTAGAGCT | |
| | CAGAAACAGACCCATTGATATATGTAAGTGACCTATGAAAAAAATATGGCATTTTACAATGGGAAAATGA | |
| | TGGTCTTTTTCTTTTTTAGAAAAACAGGGAAATATATTTATATGTAAAAAATAAAAGGGAACCCATATGT | |
| | CATACCATACACACAAAAAAATTCCAGTGAATTATAAGTCTAAATGGAGAAGGCAAAACTTTAAATCTTT | |
| | TAGAAAATAATATAGAAGCATGCCATCAAGACTTCAGTGTAGAGAAAAATTTCTTATGACTCAAAGTCCT | |
| | AACCACAAAGAAAAGATTGTTAATTAGATTGCATGAATATTAAGACTTATTTTTAAAATTAAAAAACCAT | |
| | TAAGAAAAGTCAGGCCATAGAATGACAGAAAATATTTGCAACACCCCAGTAAAGAGAATTGTAATATGCA | |
| | GATTATAAAAAGAAGTCTTACAAATCAGTAAAAAATAAAACTAGACAAAAATTTGAACAGATGAAAGAGA | |
| | AACTCTAAATAATCATTACACATGAGAAACTCAATCTCAGAAATCAGAGAACTATCATTGCATATACACT | |
| | AAATTAGAGAAATATTAAAAGGCTAAGTAACATCTGTGGCTTAATTAAGGCGCGCCTACCAGTAAAAAAG | |
| | AAAACCTATTAAAAAAACACCACTCGACACGGCACCAGCTCAATCAGTCACAGTGTAAAAAAGGGCCAAG | |
| | TGCAGAGCGAGTATATATAGGACTAAAAAATGACGTAACGGTTAAAGTCCACAAAAAACACCCAGAAAAC | |
| | CGCACGCGAACCTACGCCCAGAAACGAAAGCCAAAAAACCCACAACTTCCTCAAATCGTCACTTCCGTTT | |
| | TCCCACGTTACGTCACTTCCCATTTTAAGAAAACTACAATTCCCAACACATACAAGTTACTCCGCCCTAA | |
| | AACCTACGTCACCCGCCCCGTTCCCACGCCCCGCGCCACGTCACAAACTCCACCCCCTCATTATCATATT | |
| | GGCTTCAATCCAAAATAAGGTATATTATTGATGATGA | |
| | Description of the elements: | |
| | (1) . . . (103): inverted terminal repeat (ITR) | |
| | (104) . . . (27556): encodes the shuttle vector (P-delta-28) | |
| | (27557) . . . (27564): AscI Restriction enzyme site | |
| | (27565) . . . (27991): encodes CpG free MAR from human beta-globin | |
| | gene | |
| | (27992) . . . (28414): encodes CMV Enhancer | |
| | (28415) . . . (28669): encodes human EF1-alpha promotor | |
| | (28670) . . . (28797): encodes synthetic intron | |
| | (28798) . . . (28851): encodes a multiple cloning site | |
| | (28852) . . . (29151): encodes transgene (PYY) from ATG to stop | |
| | codon | |
| | (29152) . . . (29179): encodes a multiple cloning site | |
| | (29180) . . . (29410): encodes SV40 poly Adenylation signal | |
| | (29411) . . . (30218): encodes MAR 5′ region from human IFN-beta | |
| | gene | |
| | (30219) . . . (30226): AscI Restriction enzyme site | |
| | (30227) . . . (30523): encodes the shuttle vector (P-delta-28) | |
| | (30524) . . . (30627): inverted terminal repeat (ITR) | |
| HDAd-EF1a-PYY | CATCATCAATAATATACCTTATTTTGGATTGAAGCCAATATGATAATGAGGGGGTGGAGTTTGTGACGTG | 45 |
| Approach 3 | GCGCGGGGCGTGGGAACGGGGCGGGTGACGTAGTAGTGTGGCGGAAGTGTGATGTTGCAAGTGTGGCGGA | |
| Codon | ACACATGTAAGCGACGGATGTGGCAAAAGTGACGTTTTTGGTGTGCGCCGGTGTACACAGGAAGTGACAA | |
| Optimized | TTTTCGCGCGGTTTTAGGCGGATGTTGTAGTAAATTTGGGCGTAACCGAGTAAGATTTGGCCATTTTCGC | |
| Nucleic Acid | GGGAAAACTGAATAAGAGGAAGTGAAATCTGAATAATTTTGTGTTACTCATAGCGCGTAATATTTGTCTA | |
| Sequence | GGGCCGCGGGGACTTTGACCGTTTACGTGGAGACTCGCCCAGGTGTTTTTCTCAGGTGTTTTCCGCGTTC | |
| Encoding PYY | CGGGTCAAAGTTGGCGTTTTGATATCAAGCTTATCGATACCGTAAACAAGTCTTTAATTCAAGCAAGACT | |
| (3-36) | TTAACAAGTTAAAAGGAGCTTATGGGTAGGAAGTAGTGTTATGATGTATGGGCATAAAGGGTTTTAATGG | |
| Approach 3 | GATAGTGAAAATGTCTATAATAATACTTAAATGGCTGCCCAATCACCTACAGGATTGATGTAAACATGGA | |
| Construct in | AAAGGTCAAAAACTTGGGTCACTAAAATAGATGATTAATGGAGAGGATGAGGTTGATAGTTAAATGTAGA | |
| HDdelta28E4- | TAAGTGGTCTTATTCTCAATAAAAATGTGAACATAAGGCGAGTTTCTACAAAGATGGACAGGACTCATTC | |
| MAR-EF1a | ATGAAACAGCAAAAACTGGACATTTGTTCTAATCTTTGAAGAGTATGAAAAATTCCTATTTTAAAGGAAA | |
| shuttle | ACAGTAACTCACAGGAAATACCAACCCAACATAAAATCAGAAACAATAGTCTAAAGTAATAAAAATCAAA | |
| vector | CGTTTGCACGATCAAATTATGAATGAAATTCACTACTAAAATTCACACTGATTTTGTTTCATCCACAGTG | |
| | TCAATGTTGTGATGCATTTCAATTGTGTGACACAGGCAGACTGTGGATCAAAAGTGGTTTCTGGTGCGAC | |
| | TTACTCTCTTGAGTATACCTGCAGTCCCCTTTCTTAAGTGTGTTAAAAAAAAAGGGGGATTTCTTCAATT | |
| | CGCCAATACTCTAGCTCTCCATGTGCTTTCTAGGAAACAAGTGTTAACCCACCTTATTTGTCAAACCTAG | |
| | CTCCAAAGGACTTTTGACTCCCCACAAACCGATGTAGCTCAAGAGAGGGTATCTGTCACCAGTATGTATA | |
| | GTGAAAAAAGTATCCCAAGTCCCAACAGCAATTCCTAAAAGGAGTTTATTTAAAAAACCACACACACCTG | |
| | TAAAATAAGTATATATCCTCCAAGGTGACTAGTTTTAAAAAAACAGTATTGGCTTTGATGTAAAGTACTA | |
| | GTGAATATGTTAGAAAAATCTCACTGTAACCAAGTGAAATGAAAGCAAGTATGGTTTGCAGAGATTCAAA | |
| | GAAAATATAAGAAAACCTACTGTTGCCACTAAAAAGAATCATATATTAAATATACTCACACAATAGCTCT | |
| | TCAGTCTGATAAAATCTACAGTCATAGGAATGGATCTATCACTATTTCTATTCAGTGCTTTGATGTAATC | |
| | CAGCAGGTCAGCAAAGAATTTATAGCCCCCCTTGAGCACACAGAGGGCTACAATGTGATGGCCTCCCATC | |
| | TCCTTCATCACATCTCGAGCAAGACGTTCAGTCCTACAGAAATAAAATCAGGAATTTAATAGAAAGTTTC | |
| | ATACATTAAACTTTATAACAAACACCTCTTAGTCATTAAACTTCCACACCAACCTGGGCAATATAGTGAG | |
| | ACCCCATGCCTGCAAAAAAAAAAAAATTAGCCAGGCATGGTAGCATGTACCTGTAGTCCCAGCTACTTGA | |
| | GAGGTGAGGTGGGAAAATCACTTTAGTGCAGGATGTTGAGGCTGGAGTGAACTGTGATTGTGCCACTGCA | |
| | CTCCAGCCTGGACAATAGAGCAAGACCTTGTCTCAAAAAAATGCATTAAAAATTTTTTTTAAATCTTCCA | |
| | CGTAACACATCCTTTGCCCTCATGTTTCATAAGGTAAAAAATTTGATACCTTCAAAAAAACCAAGCATAC | |
| | CACTATCATAATTTTTTTTAAATGCAAATAAAAACAAGATACCATTTTCACCTATCAGACTGGCAGGTTC | |
| | TGATTAAATGAAATTTCTTGGATAATATACAATATTAAGAGAGACTGTAGAAACTGGGCCAGTGGCTCAT | |
| | GCCTGTAATCCCAGCACTTTGGGAGGCTGGGTAACATGGCGAACCCTGTTTCTACAAAATAAAAATATTA | |
| | GCTGGGAGTGGTGGCGCACACCTATAGTCCCAGCTACTCAGGAGGCTGAGGTGGAAGGATCGCTTGAACC | |
| | CAGGAGGTTGAGACTGCAGTGAACTGTGATCATTCTGCTGCACTGCACCCCAGCCTGGGCAACAGAGACC | |
| | TTGTCTCaAAAAAAAAAAAAAAAGAGACAAATTGTGAAGAGAAAGGTACTCTCATATAACATCAGGAGTA | |
| | TAAAATGATTCAACTTCTTAGAGGAAAATTTGGCAATACCAAAATATTCAATAAACTCTTTCCCCTTGAC | |
| | CCAGAAATTCCACTTGAATAAAGCTGAACAAGTACCAAACATGTAAAAGAATGTTTCTTCTAGTACAGTC | |
| | GGTAAGAACAAAATAGTGTCTATCAATAGTGGACTGGTTAAATCAGTTATGGTATCTCCATAAGACAGAA | |
| | TGCTATGCAACCTTTAAAATATATTAGATAGCTCTAGACACACTAATATTAAAAGTGTCCAATAACATTT | |
| | AAAACTATACTCATACGTTAAAATATAAATGTATATATGTACTTTTGCATATAGTATACATGCATAGCCA | |
| | GTGCTTGAGAAGAAATGTGTACAGAAGGCTGAAAGGAGAGAACTTTAGTCTTCTTGTTTATGGCCTCCAT | |
| | AGTTAGAATATTTTATAACACAAATATTTTGATATTATAATTTTAAAATAAAAACACAGAATAGCCAGAC | |
| | ATACAATGCAAGCATTCAATACCAGGTAAGGTTTTTCACTGTAATTGACTTAACAGAAAATTTTCAAGCT | |
| | AGATGTGCATAATAATAAAAATCTGACCTTGCCTTCATGTGATTCAGCCCCAGTCCATTACCCTGTTTAG | |
| | GACTGAGAAATGCAAGACTCTGGCTAGAGTTCCTTCTTCCATCTCCCTTCAATGTTTACTTTGTTCTGGT | |
| | CCCTACAGAGTCCCACTATACCACAACTGATACTAAGTAATTAGTAAGGCCCTCCTCTTTTATTTTTAAT | |
| | AAAGAAGATTTTAGAAAGCATCAGTTATTTAATAAGTTGGCCTAGTTTATGTTCAAATAGCAAGTACTCA | |
| | GAACAGCTGCTGATGTTTGAAATTAACACAAGAAAAAGTAAAAAACCTCATTTTAAGATCTTACTTACCT | |
| | GTCCATAATTAGTCCATGAGGAATAAACACCCTTTCCAAATCCTCAGCATAATGATTAGGTATGCAAAAT | |
| | AAATCAAGGTCATAACCTGGTTCATCATCACTAATCTGAAAAAGAAATATAGCTGTTTCAATGAGAGCAT | |
| | TACAGGATACAAACATTTGATTGGATTAAGATGTTAAAAAATAACCTTAGTCTATCAGAGAAATTTAGGT | |
| | GTAAGATGATATTAGTAACTGTTAACTTTGTAGGTATGATAATGAATTATGTAAGAAAACAACAGGCCGG | |
| | GCGGGTTGGTTCACACGTGTAATCCCAGCACTTTGGGAGGCTGAGGCAGGCAGACTGCCTGAGCTCAGGA | |
| | GTTCGAGACCAGCCTGGGCAACACGGTGAAATCCCGTCTCTACTAAAAATACAAAAAAATTAGCCGGGTG | |
| | TGGTGACACATGCCTGTAGTCCCAGCTACTTGGGAGGCTGAGGCAGGAGAATCACTTGAACCTGGGAGGT | |
| | GAAGGTTGCAGTGAGCCAAGAATGCGCCACTTCACTCCAGCCTGGGAAACAGAGCAAGACTCTGTCTCAA | |
| | AAAAAACAAAACAAACAAACAAAAAAACAGGCTGGGCGCGGTGGCTCACGCCTGTAATCCCAGCACTTTG | |
| | GGAGGCCGAGGCGGGTGGATCACCTGAGGTCAGGAGTTCCAGACCAGCCTTGTCAACATGGTGAAACCTC | |
| | CCCCCGCCGTCTCTACTAAAAATACAAAAATTAGCCAGGCGTGGTGGCAGGAGCCTGTAATCCCAGCTAC | |
| | TTGGGAGGCTGAGGCAGGAGAATCGCTTGTACCCAGAAGGCAGAGGTTGCACTGAGCTGAGATGGCACCA | |
| | TTGCACTCCAGCCTGGGGGACAAGAGCGAGATTTCGTCTTTAAAAAACAAAAAACAAAACAAAAAACCAT | |
| | GTAACTATATGTCTTAGTCATCTTAGTCAAGAATGTAGAAGTAAAGTGATAAGATATGGAATTTCCTTTA | |
| | GGTCACAAAGAGAAAAAGAAAAATTTTAAAGAGCTAAGACAAACGCAGCAAAATCTTTATATTTAATAAT | |
| | ATTCTAAACATGGGTGATGAACATACGGGTATTCATTATACTATTCTCTCCACTTTTGAGTATGTTTGAA | |
| | AATTTAGTAAAACAAGTTTTAACACACTGTAGTCTAACAAGATAAAATATCACACTGAACAGGAAAAACT | |
| | GGCATGGTGTGGTGGCTCACACTTGTAATCCCAGTGCTTTGGGAGGCTGAGACAGGAGAGTTGCTTGAGG | |
| | CCAGGAGTTCAAGACCGACATGGGGAATGTAGCAAGACCCCGTCCCTACAAAAAACTTTGTAAAAATTTG | |
| | CCAGGTATGGTGGTGCATACCTGTAGTCCCAGCTACTCGGGAGGCGGAGGCAGAAGGAATCACTTGAGCC | |
| | CAGGAGTTTGAGGCTGCAGTGAGCTACGATCATACCACAGCACTCCAGCGTGGACAACAGAGTAAGACCC | |
| | TATCTCAAAAACAAAACAAAACAAAACAAACAAAAAAAACCACAAGAAAAACTGCTGGCTGATGCAGCGG | |
| | CTCATGCCTGTAATCCCAGTATTTTGGGAGGCCCAGGTGGGCGTATCACCTGAGGTCAGGAGTTAGAGAC | |
| | CAGCCTGGCCAACATGGTGAAACCCCATCTCTACTAAAAATACAAAATTAGCCAGGCATGTGGCACGCGC | |
| | CTGTAGTCCCAGTTACTGGGGAGGCTGAAGCAGGAGGATCACCTGAGCCCGGGAGGTGGAGGTTGCAGTG | |
| | AGCCGAGATCACACCACTGCACTCCAGCCTGGGTGACACAGCAATACCCTACCTCAAAATAAAAAAGAAA | |
| | AAGAAAAGAAAAGTTGCTGTCCCCGCTACCCCAATCCCAAATCCAAACAGCCTCTCTCATCTCACAGTAA | |
| | GGGGGAAAAATCACCCAAAAAAGCTAAGTGATCTTTTGAAAACCCAAACTCTTAGAAGTCTAAGATTATT | |
| | ATAGTCAACTCATGAAGTGTCATCATAAAAGATACTCTAATATTATTTAAGTAGAACCACATATTGGTTG | |
| | TCTTGGTATGTCTAGCCCCTGGCATACAAAATATTTAATAACACTGATATGGTACCTGTGATGTGAAAAT | |
| | GTACTATGAGTACAGCTTTATAAATACTATATATGTACCTATATACAGAAAAAAATACAACAAAATCATA | |
| | AAAGCACTTATCTTTGAAAGAGGAGTTACAGCAATTTTATTTAGTTCTTTATTGCTTTGCTATATATTCT | |
| | AAATTTTTTTCAATGAATATATATCACTTTTAAAAAAATTCAATGGTCTTTCTTATAAATTATCTTTGGC | |
| | AGCATGCGTTTTTATATATACATATAAAATGTATGGGAAATTTTTAAAGGATACATTAAATTAAAGCAAA | |
| | ATATACAAACAAAAAATCAGAATACAAAAAGATAAAAAGATTGGGAAGGGAGGGAGGGAGTAAGGAGGAA | |
| | GGGTGGGTGGGTATAGAGAAATATACCAAATAATGGTAAGAAGTGGGGTCTTGACACTTTCTACACTTTT | |
| | TTTAAATAAAAAAAATTTTTTTCTCTCTCTTTTTTTTTTTTAGAGACGAAGTCTCGCTATGTTGCCCAGG | |
| | CTGGTCTTGAACTCCTGGGATCAAGAGATCCTCCTGCCTCAGCCTCCCAAGGTGCTTGGATTACAGGTGT | |
| | GAGCCACCACGCCTGGTCACTTTCTACACTTTAATATATATATTTTTTCATTTTCAATGTCATTTTTATT | |
| | AGTTAATTTATAATACCCATTCACCATTATATTCAAAGTCTATTTGAAGAAATAAACCAGAAAGAATGAA | |
| | ATACTCTAGCTCACATGCTATTCAATACTAAATTACCTTTCAAATCACATTCAAGAAGCTGATGATTTAA | |
| | GCTTTGGCGGTTTCCAATAAATATTGGTCAAACCATAATTAAATCTCAATATATCAGTTAGTACCTATTG | |
| | AGCATCTCCTTTTACAACCTAAGCATTGTATTAGGTGCTTAAATACAAGCAGCTTGACTTTTAATACATT | |
| | TAAAAATACATATTTAAGACTTAAAATCTTATTTATGGAATTCAGTTATATTTTGAGGTTTCCAGTGCTG | |
| | AGAAATTTGAGGTTTGTGCTGTCTTTCAGTCCCCAAAGCTCAGTTCTGAGTTCTCAGACTTTGGTGGAAC | |
| | TTCATGTATTGTCAGGTTGGCCCGTAATACCTGTGGGACAACTTCAGCCCCTGTGCACATGGCCAGGAGG | |
| | CTGGTTGCAAACATTTTCAGGTAGGTGGACCAGGACATGCCCCTGGTCATGGCCAGGTGGAGGCATAGTG | |
| | CTATACAGCAGGCAGAAGTCAATATTGATTTGTTTTTAAAGAAACATGTACTACTTTCATAAGCAGAAAA | |
| | AATTTCTATTCTTGGGGGAAAAGATTATGCCAGATCCTCTAGGATTAAATGCTGATGCATCTGCTAAACC | |
| | TTCACATATCAGAACATATTTACTATAGAAAGAATGAAAATGGGACATTTGTGTGTCACCTATGTGAACA | |
| | TTCCAAAAATATTTTACAACAACTAAGTATTTTATAAATTTTATGAACTGAAATTTAGTTCAAGTTCTAG | |
| | GAAAATACAAACCTTGCTAGATATTATAAAAATGATACAATATATATTCATTTCAGGCTCATCAGAATAT | |
| | ATCTGTTATCACTTGACAAGAATGAAAATGCACCATTTTGTAGTGCTTTAAAATCAGGAAGATCCAGAGT | |
| | ACTAAAAATGACTTCTTCCTTGAAGCTTACTCACCAACTTCCTCCCAGTTACTCACTGCTTCTGCCACAA | |
| | GCATAAACTAGGACCCAGCCAGAACTCCTTGAAATATACACTTGCAACGATTACTGCATCTATCAAAATG | |
| | GTTCAGTGCCTGGCTACAGGTTCTGCAGATCGACTAAGAATTTGAAAAGTCTTGTTTATTTCAAAGGAAG | |
| | CCCATGTGAATTCTGCCCAGAGTTCATCCCAGATATGCAGTCTAAGAATACAGACAGATCAGCAGAGATG | |
| | TATTCTAAAACAGGAATTCTGGCAATATAACAAATTGATTTCCAATCAAAACAGATTTACATACCATACT | |
| | TATGTCAAGAAGTTGTTTTGTTTTATTGCATCCTAGATTTTATTTTTTTGATTTATGGTTTACTTTAAGC | |
| | ATAAAAAATTTGTCAATACAACTCTTCCCAAAAGGCATAAACAAAAATTCATAAAACTTGCATCACTTGA | |
| | GATACTTCAGGTATGAATTCACAACTTTGTTACAACTTACTATATATATGCACACATATATATATATTTG | |
| | GGTATATTGGGGGGGTTCTAATTTAAGAAATGCATAATTGGCTATAGACAGACAGTTGTCAGAACTTGGC | |
| | AATGGGTACGTGCAGGTTCATTATACCAAGTCTACTTGTAGTTGTTCAAAATGTATCATAATACAAGGCC | |
| | GGGCGAGGTGGCTCACGCCTGTAATCCCAGCATTTTGGGAGGCTAAGGCAGGAGGATTGCTTGAGGTCAG | |
| | GAGTTTGTGACCAGCCTGGGCAACAGAGCAAGACCCTGTCTCCAAAAAGAAAAAAAATAATTTTTTACAA | |
| | AATAAAAACAAAATGTATCATCAGACGAAATTAAATAAGAGGCAATTCATTTAAATGACAACTTTTCCCA | |
| | GCTTGACATTTAACAAAAAGTCTAAGTCCTCTTAATTCATATTTAATGATCAAATATCAAATACTAATTT | |
| | TTTTTTTTTTTTTTTTTTTGAGACGGAGTCTCGCTCTGTCGCCCAGGCTGGAGTGCAGTGGCGCGATCCT | |
| | GGCTCACTGCAAGCTCCGCCTCCCGGGTTCACGCCATTCTCCTGCCTCAGCCTCCCGAGTAGCTGGGATT | |
| | ACAGACATGCGCCACCACGCCCGGCTAATTTTGTATTTTTAGTAGAGATGGGGTTTCTCCATGTTGGTCA | |
| | GGCTGGTCTTGAATTTCCCACCTCAGGTGATCTGCCTGCCTCAGCCTCACAAAGCAGTAGCTGGGACTAC | |
| | AGGCACCCACCACCACACTTGGTTAATTCTTTTGTATTTTTTTTGTAAAGACGGGATTTCACCATGTTAG | |
| | CCAGGATGGTCTCGATCTCCTGATCTCATGATCCGCCCGCCTCAGCCTCCCAAAGTGCTGGGATTACAGG | |
| | CGTGAGCCACCCCGCCCGGCCATCAAATACTAATTCTTAAATGGTAAGGACCCACTATTCAGAACCTGTA | |
| | TCCTTATCACTAATATGCAAATATTTATTGAATACTTACTATGTCATGCATACTAGAGAGAGTTAGATAA | |
| | ATTTGATACAGCTACCCTCACAGAACTTACAGTGTAATAGATGGCATGACATGTACATGAGTAACTGTGA | |
| | ACAGTGTTAAATTGCTATTTAAAAAAAAAGACGGCTGGGCGCTGTGGCTCATGCCTGTAATCCCAGCACT | |
| | TTGGGAGGCCAAGGCAAGTTGATCGCTCGAGGTCAAGAGTTCGAGACCAGCCTGGCCAACGTGGTAAAAC | |
| | CCCGTCTCTACTAAAAATACAAAAAAAAAATTAGCCAGGCATGGTGGCACAGGCCTGTAATCCCAGCTAC | |
| | TAGGGAGGCTGAGACATGGAGAACTGCTTGAATCCAGGAGGCAGAGGTTACAGTGAGCCGAGATCATACC | |
| | ACTACACTCCAGCCTGAGTGACAGAGCGAGACTCCGTCTAAAAAAAAAAAAAAAAAAAAAGATACAGGTT | |
| | AAGTGTTATGGTAGTTGAAGAGAGAACTCAAACTCTGTCTCAGAAGCCTCACTTGCATGTGGACCACTGA | |
| | TATGAAATAATATAAATAGGTATAATTCAATAAATAGGAACTTCAGTTTTAATCATCCCAAACACCAAAA | |
| | CTTCCTATCAAACAGGTCCAATAAACTCAATCTCTATAAGAGCTAGACAGAAATCTACTTGGTGGCCTAT | |
| | AATCTTATTAGCCCTTACTTGTCCCATCTGATATTAATTAACCCCATCTAATATGGATTAGTTAACAATC | |
| | CAGTGGCTGCTTTGACAGGAACAGTTGGAGAGAGTTGGGGATTGCAACATATTCAATTATACAAAAATGC | |
| | ATTCAGCATCTACCTTGATTAAGGCAGTGTGCAACAGAATTTGCAGGAGAGTAAAAGAATGATTATAAAT | |
| | TTACAACCCTTAAAGAGCTTATAGCTGGGCGTGGTGGCTCATGCCTGTAAATCCCAGCACTTTGGGAGGC | |
| | TGAGGCGGGTGGATCACCTGAGGCCAGAAGTTCAAGACCAGCCTAGCCAACATGGCGAAACCCTGTCTCT | |
| | ACAAAAAATACAAAAATTAGCCGGGTGTGGTGGCACGTGCCTGTAGTCCCAGTTACTTGGGAGGCCGAGG | |
| | CAGGAGAATCGCTTGAACCTAGGAGGTGGAGGCTGCAGTGAGCCGAGATTGTGCCACTGCACTCCACTTC | |
| | AGCCTGGGCGACAAGAGCAAGACTCCGTCACAAAAAAAAAAAAAAAAAAAAAGCTTAAAATCTAGTGGGA | |
| | AAGGCATATATACATACAACTAACTGTATAGCATAATAAAGCTCATAATCTGTAACAAAATCTAATTCGA | |
| | CAAGCCCAGAAACTTGTGATTTACCAAAAACAGTTATATATACACAAAAAGTAAACCTAGAACCCAAAGT | |
| | TACCCAGCACCAATGATTCTCTCCCTAAGCAGTATCAAGTTTAAAGCAGTGATTACATTCTACTGCCTAG | |
| | ATTGTAAACTGAGTAAAGGAGACCAGCACCTTTCTGCTACTGAACTAGCACAGCCGTGTAAACCAACAAG | |
| | GCAATGGCAGTGCCCAACTTTCTGTATGAATATAAGTTACATCTGTTTTATTATTTGTGACTTGGTGTTG | |
| | CATGTGGTTATTATCAACACCTTCTGAAAGAACAACTACCTGCTCAGGCTGCCATAACAAAATACCACAG | |
| | ACTGAGTGACTTAACAGAAACTTATTTCTCACAGTTTTGGAGGCTGGGAAGTCCAAAATTAAGGTACCTG | |
| | CAAGGTAGGTTTCAATCTCAGGCCTCTTCTTTGGCTTGAAGGTCTTCTAACTGTGTGCTCACATGACCTC | |
| | TTCTAACAAGCTCTCTGGTGTCTCTTTTTTTTTTTTTTTCTTTTTTGAGACAGAGTCTCACTCTGTCACC | |
| | CAGGCTGGAGTACAGTGGCACAATCTGGGCTCACTGCAACCTCCAACTCCCGGGTTCAAGTGATTCTCAT | |
| | GCCTCACCCTCCCGAGTAGCTTGGATGACAGGAGCCCGCTACCACACCCAGCTAATTTTTGTATTTTTAG | |
| | TAGAGATGGTGTTTCACTACATTGGCCAGGCTGGTCTCAAACTCCTGACCTCGTGATCCACCCACCTTGG | |
| | CCTCCCAAAGTGCTGGGATTACAGGTGTGAGCCACTGCGCCCGTCCTGGTGTCTTTTCATATAAGGGCAC | |
| | TAATCCAATCAGACCTGGGCCCAACCCTCCCGACTTCTTCTAACTGTAATTACCTTCCAAAGGCCCTGTC | |
| | TCCAAATACCATCACACTGGGGGTTAGGACTTCAAAAAAGGTATGGGGGGGGTGTGGGAGGACATAAATG | |
| | CTCAGTCCATAACAAGCACCCAACATAAAAATGGCTAGAACAGATCACAAAAAAAAGGTCCTGTATGGCT | |
| | TTGGGGAAGGGCTCAACCCCAAAATATCTGAGAGCTCTGGAGGGGCCTAGAAGTGGTAAATGAATGAAAA | |
| | CGTGGTTACTCTCCAGATCTGCCTTTCCCAAATATGGCCATTCTTGGCTGAATCAGAAATCAAAGGACAG | |
| | GTTATTAATTACTAGCTCTAAGTTACTTACCATTTGCTGAGACAGTTCAGAAATCTGACTGCATCTCCTC | |
| | AGAGATCTAGAACACAGTTCTCAAATTCTAACTTACTTGTGATATACTTGTGAATGATAAAAATCGCTAC | |
| | AGGTACTTTTATTAATCTGAAAGAGTATTGAGAAATTACCTTTCATTCTGACTTTTGTCTGGAATGAAAA | |
| | TCAATACTTTTGCTATAATCGATTACTGAAATAATTTTACTTTCCAGTAAAACTGGCATTATAATTTTTT | |
| | TTAATTTTTAAAACTTCATAATTTTTTGCCAGACTGACCCATGTAAACATACAAATTACTAATAATTATG | |
| | CACGTCACATCTGTAATAATGGCCTTCATGTAAACATTTTTGTGGTTTACACATAAAATCTCTAATTACA | |
| | AAGCTATATTATCTAAAATTACAGTAAGCAAGAAAATTAATCCAAGCTAAGACAATACTTGCAACATCAA | |
| | TTCATCATCTGTGACAAGGACTGCTTAAGTCTCTTTGTGGTTAAAAAGGAAAAAAAAAAAAAAGACATGT | |
| | TGGCCAGATGCGGTGGCTCACACCTGTAATCCCAGCACTTTGGGAGGCTGAGGTGGGCGGATCACCCCTG | |
| | GCCTGCCCAACATGGTGAAACCCCGTCTCTACTAAAAACACAAAAATTAGCTGGGCGTGGTGGCGGGCGC | |
| | CTGTAATTCCAGCTACTCGGGAGGCTGAGGCAGGAGAATTGCTAGAACCCAGGAGGCAGAGATTGCAGTG | |
| | AGCTGAGATTGCACCATTGCACTACAGTCTGGGCAACAAAAGTGAAACTCCATCTTAAAAAAAAAAAGAC | |
| | AATGTTCGTGGGTCCAAACAAGACTTAATGGAAGTGAGTCTAAAAATGAGCTATGTGGGCCAGGCGTAGT | |
| | GGCTCCCACCTGTAATCCCAGCACTTTGGGAGGCCGAAGCAGGCAGATCATGAGGTCAGGAGATGGAGAC | |
| | CATCCTGGCCAACACGGTGAAATCCTGTCTCTACAAAAATTAGCTGGGCGTGGTGGTGCCTGCCTGTAAT | |
| | CCCAGCTACTCAGAAGGCTCAGGCAGGAGAATCGCTTGAACCAGGGAGTCGGTGGCTAGAGTGAGCCGAG | |
| | ATTGCATCACTGCACTCCTGCCTGGTGACAGAGCAAGACTCCATCTCAAAAAAAACAAACAAAAATAAAA | |
| | GATAAAAATGAGCTATGTGAATTAAAAGAGGTATAACAATAGATAAACCATATTTTATTTAATTCCTAGT | |
| | AATGAGTAATATTTCCAAACTTCTGGAATGGGCAGAAATTGCTAGTTGGCATATTTTTACCTTTTATATT | |
| | CAGATACATTAAAATTCTCAAAAAAAAACACCTCAAAGCAGATGATCCGCCATCTCCTTGGATAATTTGT | |
| | GTTAACTCAGGATAACAGAAAACCAAAATTATGAGTTACTGATGCAATATTCCTAAATGTAAAAATAATT | |
| | AAAGCTAATAGTAGATTCATCTTCCAATTTCATATCAGTCTTACAAATAAACTACATATATAACTTGCTT | |
| | GCCTTCCCTTCTGAGGGATAAAGCTGTTAGAAGAATTAAAATCAGCATTCTTGACTATTCAACCAAGGGA | |
| | GGGATAAATTATTACTCATTCTAGGGACATGGGCTCATAACTACTACATGTGTAAGGACATGAATTTACC | |
| | CAATATTACAATTTTTCCTTTTATTAGTGTGTACAGTGGAAGAATAGACATGTTCACTCTGGACAAAAAA | |
| | AAAATTATACTTATCAGTTATCAGAAGCACAATGCTGAAGACAGTAGTTCCATAACAATTTGAAGTATGT | |
| | GATCGAACTAGTAGATTATCTTAGTAGTAGTGAATTATTGTAAATGTTAGTAATTTGGCAGCCACTGGGC | |
| | AGAAAAATAAGAATTGAGGCTCAATATTGATATTAATGGTGGTGATTGACACATAAATTTTATCAAGTCT | |
| | ACACAATATAAAATTACAGAAAGGTAGAAGAGTATACCAGTACAACTTCAACATATCTTCACTACAAGGG | |
| | AGTAAAATGACATGGCCTAGTTACTATCTAATGAACTGCAGAAAACTAAAAGAAAACTCCAAGGCAACTC | |
| | TTCTCTGCTGATCTGGTTGGTCCTTTTCCTACCTTTTGCAATACCCAGATACAAACAATGGATAGAAAAC | |
| | AAAGTAGACTTGTAGTATGCAGGTCACAGTGCTAAATTCACAGAAAGAAACCCCTGAACTGAACTGCTCT | |
| | ATTTCCTGGTGGTCACAAAGAGTAATTCTGGTTTACACCTACAGATTGATGTCAATCTACACCCTGTTGA | |
| | TAACAGTGTGGCCAAGGACAAAAAAAAGGTGCTCCGTTTTACCAATTCTGTAAAAAATTATTGGCAGGGT | |
| | AAGCTCGGCTAGGGCAGGATTACATTTCTAGGACTACCATCCCCGAAATTTAGAAGATATTATATCCACA | |
| | TAAAGCATATCTTTCACATTAATTTGCAAAAATCTAAAAGCTTTTTCTTAGCTCAAGTGTGTCCAAGTTT | |
| | ACCCTGGCAGTTTAAAACGATAGTTACAAGCAGCATGGGTTGTATCAGACACATTTGAGGGCCAATTTCA | |
| | TGTAAGTGATATTGGGCAAGTTACTTCAACTATCTGTGCCTCCAAGGTCATACTAGTGTTTATTTACCTA | |
| | AAGGGTACCTGTTATGTAACTTTAGGGTGTTTACATTAGATAATGCCTGCAAAATATTTACTTCAACGCC | |
| | TAAAACATAGTTAAGTATTCAATAAATACCTACTATTGTCACTACTAACTTAAAAGTTTAGAGATTAAGA | |
| | GCAGAATCTGGGGTGAGACAAACTTAGGTTCAAATCCTAGTATTGTTGGGTAATCTTGGGCAAGTTACTT | |
| | AACCTCTCTGATTTGTGTAATTTAAAAAATTAGTTAATATACATAACAGGGCTTAGAAGAGTATCTAGCA | |
| | CATAGCACCATTTAAGCATTTGTTATTGCTAACATGCAAACAATTTAAGGGAAAGAAATTTTTTAAAAAG | |
| | GAAGAGGGATTTGCAAACTAAAAACAATGAGTATCTTATGTTCAAAGAAAACTAACAAACAGCCAGCTCT | |
| | AGCAATAATTAAATTCACTATATACTGGGGCAGGCATCACACCCCAAAGCTAAAAGCGTCTACCTAGGCC | |
| | AGGCACGGTGGCTCATGCCTGTAATCCCAGCACTTTGGGAAGCAGAGGCGGGCAGATCGCTTGAGCTCAG | |
| | GAGTTCAAGACCAGCCTGGACAACATGGCAAAACACCATCTCTACAAAAAATACAAATATTAGGCCGGGC | |
| | GCAGTGGCTCACGCCTGTAATCCCAGCACTTTGGGAGGCCAAGGCGGGTGGATCACCTGAGATCAGGAGT | |
| | TCGAGAGTAGCCTGGCCAACATGGTGAAACCTCGTCTCTATTAAAAATACAAAAAATTAGCCAGGCATGG | |
| | TGGCAGGCGCCTGTAATCCCAGCTACTCAGGGGGATGAGGTAGGAGAATCGCTTGAACCCGGGAGGCAGA | |
| | GGTTGCACTGAGCCGAGATCATGCCACTGTACTCCAGCCCGGGCAACAAGAGCGAAACTCCATCTCAAAA | |
| | AATAAATAAATAAATAAATAAAATAAAGTACAAATATTAGCCAGGGATGGTGGTGCGCACCTGTAGTCCC | |
| | AGCTACTTGGGAGGCTGAAGTGGGAGAATCCCCTGAGCCTGGGGAGAATCACCCGAGCCCGGGAAGTCGA | |
| | GGCTGCAGTGAGCAGTGATTGTGCCACTGCACTCCATCCTAGGTGACAGAGTGAGACCCTGTCTCAAAAA | |
| | AAAGAAATTGGCAGAATTAAGTAAGTTGATGTTTAGAGATGAAAAATCAACATTTTTTCCTCAGCAACTG | |
| | AATAAAAACAACAGCCACTACCATTTTTTTGAGTACCTATTTGTAGCCTATTTTTTAACTGGTATTACTC | |
| | GAGAGAGAGAGAGCTAGGTTCGAGACAGAGCTCCTTCTCTTAATAACTGTATGACCTAGGGTATGTCTGT | |
| | TAGCCTCTCTGAGGCTTCAAAGGTTCCTCATCTGTAAAATGGTAATAATCATACCATTGCTACAGGGCTG | |
| | TTTTGAAGACTAATTAGGACTATGTAAGTAAACATGATGATGGCTATTATTACTGTTCCCCGCCAGGGGC | |
| | CATGCAAGGGTTGCTGATTCACATAGACTGTCTTATAATCCTCTCAATAACTCCAAGAGGTAGCCAGCAC | |
| | CTCAGATATACATAAAATGACTTAAGCCCAGAGAGGTGAAGTAAGTTGCCCACAGCCACACAACTAGTAA | |
| | ATAGCCCAAACAAGCTGGATTCCCAGTTAGACTCCGTTAATAGCACTGCTCTTTACCTTAAGTCATTACA | |
| | ATGCCTAATATGAAATAGAATCGCTTCTTTCTTAGGGTTCAAGTGGTTAATTATTTAATGTATTCATTCA | |
| | ACAAACCATCATCGAGGACCTCTTACAAGCCAAGTACTGTGCTAAGTGCTAGAGTTACGGCGGTGATTCC | |
| | TGCCCTTAAAAAGTTTTAGTGGGAGAAACAACAGGTAACCAGGTCATTGCCAAAACAACAAAAATAATCA | |
| | TAATAAAGCAGGCTAAAGCATATTTAACTGGCCGGGGTTTTGACTATTTTAGCAAGCATGATCAGAACGG | |
| | TTGAGGAGGGAGGCCAGCAGCTTGGCCGGTTCAACAAACAAGAAAAAACCAGTGAGGGTGGAGCTAAGAT | |
| | ACCAGAGGCTGATTACGGTTAAGAATGTTCTTGAAGGTAAGGACCAGATTCTCATTTTCTATATCCTGGG | |
| | GCATCGGTCAGCATGGAATCTGGATTCTAGCACATGTGAATTTCGGCTTGAAATGACCTAATGCCTTTTC | |
| | CCTAGTTCCTTCGTGTGTCAAATACGCATGGTTACCGCTACCAGAGCTGTAGTGGGGCTTCAATGAGGCC | |
| | ATGAGCATCTCCATAAAGATGAACTACAGTGTGTGCAAAACTAAAGGCAAAACCTGGTCCCCACACGCCC | |
| | TCCCAGGTGGTCGCTTTCCGTGCCGAGGCCCCTCCAGAGGTGCCCCGAGAACCTCACCATCGCACCCCAA | |
| | ACTTCCAGGGAAGGGCCTCTCCCGAGAAAGCCCCCACGCCCCCACCCCGCGCCATCATTCCCGAATCTGC | |
| | CCTCGGCCCCTCCCCGCAGCACGCTCGCAGGCGGCACATGTCAACCAAAACGCCATTTCCACCTTCTCTT | |
| | CCCACACGCAGTCCTCTTTTCCCAGGGCTCCCCCGAGGAGGGACCCACCCCAAACCCCGCCATTCCGTCC | |
| | TCCCTGCCGCCCTCGCGTGACGTAAAGCCGAACCCGGGAAACTGGCCGCCCCCGCCTGCGGGGTTCCCTG | |
| | GGCCCGGCCGCTCTAGAACTAGTGGATCCCAATTGAAGGCCTGGTCTAAATGACTCCAAAATCACCACTT | |
| | AATTCAAGAGACTGATTTCCCTGAGTCAGGCCCCTTAAAGCAGCTATTTCAATGGGACAGGGAAACAACC | |
| | CTAGGATCTGGATTAGAATCACTTGGGGGCTGCCACACCCCCAGGGCTCTGATCCTGCCCTTCTCCCACA | |
| | CGCACATTCACATACTGCTGCAGTGACCTTCCATTTCTAATGGGTTCCTGGGCCATCTGTCAGGTATAGG | |
| | GAATGGAAAAGGGGTTGGGGAGGCTCTGCTTCAGAAAGTTTGTGTCAGGGGCTCCCAGAGCCTCCACAGA | |
| | TAGATAGCAGGGGTCCCCACCCTACCATGGCAGCTATAAATGTGATCAACATTTATTGGCCTAGGATACA | |
| | GCAGTTAGCAAAATGCCTGATGTAGTTCCCACTCCGTGGAGGTTGCAGGCTAGCCAAGAAGTCATGAGTT | |
| | CAGCAACCCTTACGCACCAGTGGGATGAGATTGGACCAGGCCGAGGGTAGTCTTGGGAACACTCAGCATT | |
| | TGTCTGAGGGCCAGAAGAGGCTGCTTGCCCTCAGACAGGAGGTCAGCATCTTTATTGTAGCCCATGACAC | |
| | CTCTACACCATTGCTCTTCTGGTCTTATGGAAGACATCTTTGGGCCTGATAACAGCGGAGTCTGTGTCCC | |
| | ACTTGTCCAGGCTGGAGTGCCACATCAGGCACACTCCAGTTGCAGGGACAGCACAGACAAGTTTCAGGAA | |
| | GGCTGGTGGCCTCCAGGAGGTTAACCTTATAAGGCCAGATTGTAACCTAGTTGAAAAACATACACATGCC | |
| | ATGATAATAAAAGAACCTAGGCACCATTACAAGAGAAAAAATCATTTTTGTAGATACGAGCATGGATTCT | |
| | TGGGTGGGTCAGACACACTGGGCTTGTGCTCTGACTGCACTGTCTCCCCTACCTGACCTTGGGTAAACCA | |
| | TAAGACTGCTGCATGACTCAGTGTCCACCCCAAAAAAGTACCGGTAGATATTGGCCACAGTAGATATCAG | |
| | CTAGAGTGGACTCTCATGACAATGAGGGGAGATGTATTCCCCATCTTAGGCACCTGGGACTCTACCTTCC | |
| | ATCTTCTGCTCCGTGTCTCTCATCCCCAGGCTCTTCAGAACTCAGGGAGTCCAGAATGTCAGCTCCCAGC | |
| | ATTTCAGCCTTCAGAAAGGAAACCCATTACCGTTCAGTTGAACAAATGTTGTCTGAGCCCCAGATCTGGG | |
| | CTCAGAGGCCATCTAGGCTATGAGACAAGAGGGGAACAAAGCACCGTCTGCACTCACTCACCACACTCAC | |
| | TTGCTGTCCCAGGTCACATCCATCGGGTAGAGAATCTAAGAGGCTGAGCTAGCTCCCGCCACCAGCCCAG | |
| | CCCACCCCACCTGGCCCCTTCCTTCCTTCTACAAAATATGCACCACCTGTCAAAGGGTGGGCAGTGCCAG | |
| | GCCTGCATACAGAGCACTGAGTGTAAAAGCAGACATGGACCCTGACCTCCAGGAGCTTCCAATTTTCTTG | |
| | AAGAGACAAATCAGCTGGCATTTCAGTCCAGTGTGATCTGCTCTTGGTGAGCACAGACCTAGGGAGTTGG | |
| | GGCAGCTTCCCAGAAGAACTGCAGTCCAGGCTGAGGGCAGAGAAATGAGGGGAATGGCGAGGAATTGGGG | |
| | AGCAGGGGGGAGCTCAGTAGAGAGCCAAGGGCGGGAGGTGAGAAGTCCGTGTTGGGCCAGGAGCTACCCT | |
| | CCGGTGGCCACAGCCGAAGTCGAGGATGCCTTTGGAACTCATCCCCACTTCTCTCTTTCTGTATGTAGCC | |
| | GTCCAAGAACAAGTCACCTCCAAGTGTAGCCGGATCAAGGCAAGCCCCCCATCTAGCAAGCACTTGATGC | |
| | CACCCAGAACTGGGCTTCTTCAGAACAATCTGAGTCCAGGAATGATCCCACTCACCAGGCACCAGAGCTG | |
| | CGAGGGCATGGGAGTGATCTCACCAACTCTGGGGAAGCGGCAAGGAATTTTCACCTCCAGCCCCCAGTGT | |
| | CCCATCCTCTCACACTCAGGCCAGACTCCCCTGGGCAGACTTGACTCTGTCTGCCAGCATATGCAGAGCC | |
| | CCAAGGCCACCCCACCAGAAGTGCCCCTGCCTGGGTTCTGTCCCAGCTCCCTGGGCACCCAGTCCTTGAG | |
| | TCCCCACCAGCTCAGACGGCCTAGTGTGCCAAGAATGCCCACTGCGTTCAACAATGCTGCATGGGTCACA | |
| | GCGGCAGCAGCTGTGACCACAGCAGTTTCGGGGAAAACACCCCTCAGCCAAGTGGATAATAGCGTTCAGC | |
| | AGCACTCACCTTCTGGCCAGGCCTGCCTTCAGAGGCCATCTGATTGGGAGGCACAAGTGCCCGCTGCGAT | |
| | GGGAACACAAGTGCCCCTGGCCAACAACCCCAGCTTCAGCCTGCTGGGCAGCCAGAGCCTCAGGCAGAGC | |
| | CCGGTACAGGGCCCGGTGCCTGTAGCAAACACCACCAAGTTCCTCCAGCAGGGTATGGCCAGCTTTAGTC | |
| | CCCTGAGCCCCATACAGGGCATCGAGCCACCAAGCTATGTGGCTGCTGCTGCCACCGCTGCTGCTGCTTC | |
| | TGCCGTTGCTGCCAGCCAGTTCCCAGGTCCGTTCGACAGAACGGATATTCCCCCTGAGCTGCCACCTGCC | |
| | GACTTTTTGCGCCAGCCCCAACCCCCACTAAATGATCTGATTTCGTCACCTGACTGCAATGAGGTAGATT | |
| | TCATTGAAGCTCTCTTGAAAGGCTCCTGTGTGAGCCCAGATGAAGACTGGGTGTGCAACTTGAGGCTGAT | |
| | CGACGACATTTTGGAACAGCATGCTGCTGCTCAAAATGCCACAGCCCAGAATTCTGGGCAAGTCACCCAG | |
| | GATGCTGGGGCACTTTAAATCTGAGCAGGATGCCCATAGAAACCCCCATGGTGACATCACTCTAGGAAGT | |
| | GGTGTCGATCCATACCCGCAGTTGTCTCCCGTTACAATTTGAGTGGTGTTGTCAGCCCATGCTTATCCCT | |
| | CTCTCTACCTGTGACAAAATGGAAAGCTGGTGATTTTTCAAGCTACGTGTACATATTTGAAAATTTTGTA | |
| | AATGGTTTTCCTAAACATTAATGACAGAAGTATTTATACTTCATTTTGTGACTTTGTAAATAAAGCGACG | |
| | GCTTTTGTTTCAGTAGAGTTGTGTTTACTATGCATTGTTTTGTGTTTATTATACAATGTTACAAATATGC | |
| | AGACCGTGTTGTTTGCTCCAGTGATACCTTGTTAAGCTAGGTGGCTGAGTCGCTTATGGTTTTAATGCAA | |
| | TGAGCAATGTGGATATGACCAAGAGTTGTTGTGCAAGTTGACAAATGCCAAATAGAAAACCACTTGGCCA | |
| | TTTATTTCTATGTTCACTAAAAATCCTATTGCCTTGTGTGATTCTTAATCTCTTTTGCGAACCTTTCAGT | |
| | CTCCGCTAGCTCTTTCCTAATGAGCTTTACAGCAGAAGCTGTTTTATCGTTAAGTGCCCCACAGAGACAC | |
| | TTTACCAGGAGGCTGGGAGAGTTCTCCAGATTTGGGAGAGGCGCAGAGACAGTGTGTGAGCCGAGCCCTG | |
| | TCTCAGCAATCCACCTGGAGGAGCTAGAGTATCCTCCTCCCTTTACCATTCAGACCGAGAGAAAAAGCCC | |
| | AGCTTGTGTGCACCCTCGTGGGGTTAAGGCGAGCTGTTCCTGGTTTAAAGCCTTTCAGTATTTGTTTTGA | |
| | TGTAAGGCTCTGTGGTTTGGGGGGGAACATCTGTAAACATTATTAGTTGATTTGGGGTTTGTCTTTGATG | |
| | GTTTCTATCTGCAATTATCGTCATGTATATTTAAGTGTCTGTTATAGAAAACCCACACCCACTGTCCTGT | |
| | AAACTTTTCTCAGTGTCCAGACTTTCTGTAATCACATTTTAATTGCCACCTCGTATTTCACCTCTACATT | |
| | TGAAATCTGGCGTCTGTTTCAAGCCAGTGTGTTTTTTCTTCGTTCTGTAATAAACAGCCAGGAGAAAAGT | |
| | GCCTCTATGTTTTTATTTTTCAAGGGAGTATTCAGTACCTACAAACCCAAGTCAGGAAGCCTGCTAGTGG | |
| | CTTTGGTTCTTTCAGAGGCTGCTCGATGCCTTGTGTGTCAGAAAGAAAGATTCAGCAGTTTTGCATCATG | |
| | GCAAAGAAGCCTGTTATTTTGGGGCTCAGCCCCTCATTTTATAGAGGATGAAACAGAGGGGGATGGGAGG | |
| | TCACAAAGACAACTGCCCCGGGAGCAGGTGTGGGGGAGACTTGCCCTGAGGGTCTAGACGCTCTGCACCA | |
| | CCGTCCTGTCTCCCTTGCTGAAGACCACACATGCCCTTCTTTGACCAGACCCTGCCACCTGATAGGCCAG | |
| | GACCTGGTAGGCGGGTACCCAGGTTTCATGGATGGAACCACATCTCCCCAAAAGTGGGGAGGTAGCTACT | |
| | GGGATGCACGCCTCCCGCCATGTGCTATAGGAGAGCAGCTGAAGCAACAGTTGGGATCAGATGTAGTCAC | |
| | AATTGAATGCATCATCACATTTATCCCTCTAAGTGGCTGGGAGAGTTGATATCCTCATCCCTAAGGTACA | |
| | AAATGTTCCAATTTGATCAGTGGCTTTCAGGAGCTGAGAAAGGCATGTGCTCTGAGGCAGAGCTGTTATG | |
| | TCCCGCAGAGCCTAAAAATGCTCTAAGAACATGCTCCCTGCCAAAATTCTCAATGGCTGTGACAAGGGAC | |
| | AACGATCGACCAATGGGGGTGGAAGCAGACCTCCGCAGTCCAGGGGCCAGAGCTAGGACAGAGGGGTCGG | |
| | AGAAAGAGTCATTTTCCCAACACTCCAGCTCTTGGCCAGTCCTCACACAGTCCCCTCCTGCTTCCTGCTG | |
| | AGAGAGATATCCTCATAGGTCTGGGTAAAGTCCTTCAGTCAGCTTTCATTCCCTGTCACCAACTTTGTCT | |
| | CTGTTCTCCCTGCCCGTCTCAGGCAGCACTCCTCAGGAAACCTCTCCAAGAGCCAGCCTCACTGCAGCGC | |
| | CCACTATTGTCCCTCTGCCTCAAGTGTCCCATCCATGCCAGGCCCCAGGCAGGCTGCAGCTTTCCCTCAG | |
| | GGCCACACCAAAGCACTTGGGCTCAGCTGTGCTGTCCCCCTCCATCACTGAGCTCAGGGGCAGCAGGGGT | |
| | GGGGTGCCAGGAGGCCCATTCACCCTTCTCTGGCTCTGTGTTGGACCCACCTGCCCAGCCACTGCTGCTT | |
| | AGAACCTACCCGCTGGGAAAATGAAGCCCTCCCGGAGGGGCCACCTCAACCTGAGAGCCTCACGGATCAC | |
| | AGTTGTCCCCACTCAGCTCTGCCAGCCCTCAGAGACCCATAGATAAAAGCTGAGCTTGGCTCGCAGAGCT | |
| | GGTTCCATCTTCCATTCCCAGAGGGTTCAACTTCCTACCCCAACCACACAGGGAACCTCAAGGCTGAGCC | |
| | AGTGTGGGCTGCAGTGCAGACCAGCTTCCTGGACACGTCCTGCCACCTGACCCCAGGCTGGCCTCACTGC | |
| | CCCTGGCACTCCTGACCCTATCCTCATTCCTCCTGGCAGTGCGTGTTCTGCCATTCCGCTTTCCCTTAGC | |
| | TGTCCTCTCACTGTACTGTCAGCTTCTCCTTTTCCAGGTGCCCCCCAGGGGCTTTCCACATGACCCTGTC | |
| | ACCCCACAGCCCATCCAGCACCAATTCCAGCTCTCTGCCACCCTTCAAAGGAGTGACAGTGCCCTGCTTC | |
| | ACCTCCCACTCACCCCTCAACCCAGAGCAATCTGGCTCCAGTCTTGCCTCCTTCCCCCTAAGTACTCTAG | |
| | TCACAGTTCCAAATTCCTCCTGGTCATAAAGCCAAATGAAGCTTCCTGGTCCTCAGCGGACTTGCCACTT | |
| | CAGCAGTACTGGACTCTCTCCTCCCAGAAACCTGTTTCCCCTTGGCTCCTGGAGCCCACACTCTGCTGGA | |
| | ATCCTTCTGCCTCTCTGGCCTGTAGCCTGGCCCTCTCTCCCAACCTGAGGTCCATTCTCTCCTGCTCCTC | |
| | CACAAGATGTTGCTCCTTCCATTACTTCCTCCCTCTCAACCAAAGCTCCTTCATTAGCTCTTTATCTTCT | |
| | GGTTTCTTCCCCTGGGCAGACGAATGGATTCAAGAGCCTGTGGCCCAGCAGCCCAGCACTCCAGGATCTC | |
| | AGCACTTCAGCATCCCAGTACCCTAGCATCTCAATACCCCAGCACCCCAGCACCATAGTATTCCAGCACC | |
| | CCATTGTCCAAGCATCTCAGCACTCCAGCATCCCAGCACCCCAACACTCCAGCAGCCCAGAATCTCAGCA | |
| | CCCTAGCACTGCAGCATCTCAGGACCCCAGCACTTCAGCATCCCAGCACACTAGTACTCCAGCATCTCGG | |
| | CACCCCAGCACCTAGGCATCCCAACACCCAGCACCCCAGCACTTAAGCATCCCACCACTACAGTATCTCA | |
| | ACACTCCAGCACCCCAGCACCATAGTGTTCCAGCACCCCAGCATCCCAACACCCCAGCACTTAAGCATCC | |
| | CAACACCTCGGCATCCCAACACCCCAGCACTGCAGCATCTCAGCACCTTAGCATCCCAGTGCCCTAGCAT | |
| | CTCAATGCTCCAGCACACCAGTACTACAGTATTCCAGCACCCCAGCACTCCAGCATCTCAGCACTGCAGC | |
| | ACTGCAGCACTCCAGCATCCCAAAATCCCAGCATCCCAACACCCCAGCAGACCAGCAGACCAGCATCTCA | |
| | GCACCGCAGCATCCAAGGACTATCCCAGCATCCCAGCAACCCAGCACCTCAGCATCCCAACACCCCAGCA | |
| | TTTCAGCATGGCAACACCCCAGTACCCCAGCACTTCAGCACCCCAGTATCCCAGCATCTCAGCGACCCAG | |
| | TATCACAAAACCTCAGCATCCTAGCACCCCAGCACCCCAGCACCTTAGCACCTTAGCATCCCAGCATCTC | |
| | AGCGCCTCAGCATCTTGATATTCTGGCTGAGGTCAGCGTGGTGTATCTAGTCAGGGTCCTAACTTTCACT | |
| | TCGCAGGGAAATGCTGCTGGACTGGGTCTCATGTTGGGCTGAAGCTCTCTAGACCCCTTGAAGACAGCAT | |
| | AAAAGAGCTTGGAGACGCTGGGTGTCCCCCATGGAAGAGTTCACTCTCATCCTGCTTTGACAACAGCCTT | |
| | CTCTGGGGTCCCTCACGGGCCCCTCTTTCTTACTGCAAGTTTGTCTCTGAGAAGACTGTGATGCAGAAGT | |
| | CACTCAGCTGCCTGTGGCTCCTGAAGAGCTGAAGGTGGAGGCCTGTAGGCCTCCCTATGAGAGGCGCAGA | |
| | AAAAACCATGATTGCTAGTGGGGAGGTGCTCCCTCTACAACCCACTCCATAATCTGCCCCCGCCCAGCTC | |
| | TGAGGCCAGCCCCAGGGGAAAATGCCAGATCCCCAGGGAGGTGTGTGAGACCTCAGGGGCTCCCTCCTCC | |
| | CTTACAGCAGGCTCAGGCCCCTGGGGGCCTCAGGGCCAAGGTCTGTGGGTAAGCTACTATCTCTCACTTG | |
| | TCCTCTAGCCACAAAAGCCAGGGAGATCTGGCAATGGACATGAGGTTCTGAAGAAGCACATATGACTGGC | |
| | TTCCTAATGCGTGGTTGTTCAGTGATTCAATAAACACGCATGGGCCAGGCATGGGGAAATAGACAAACAT | |
| | GATCCCCAACCTCTCCCAGAGTGAACTGGGAGGGAGGAGTGTTCATCCCTCAGGATTACACCAGAGAAAC | |
| | AAACCAGCAGGAGATATATATGGTTTTGGGGGGTCAAGAAAGAGGAAAAACCTGGCAAGGCAAGTCCAAA | |
| | ATCATAGGACAGGCTGTCAGGAAGGGCAGCCTGGAACCTCTCAAGCAGGAGCTGATGCTGCAGTCCACAG | |
| | GCAGAATTTCTTCTTCCTCGGGGAAATCTCAGCTTTGTTCTTAAGGCCTTTCAACTGATTGGCTGAGGTC | |
| | TGCCCCTTCCCCCACATTCTCCAGGATAATCTTCCTTACTTAAAGTCAACTATTAATCACAGCTACAAAA | |
| | TCCCTTCACAGCTACACATAGATCAGTGTTTGATTGACGAACAGCCCCTACAGCCTAGCCAAGTTGACAC | |
| | ATAAAACTAACCATCACAGGGGGACAAATGATGTAAACACATCAACAAATAAAACAGTAACAAGTTAAGG | |
| | TCTATGGAAAAAACACAGAAGGGGCAGAGAGAAAGAAAGCAAGAAGGAGAGTCCCAGTTTGCTAGGGCTT | |
| | GTGGGAAGTGGGGAGCAGTTCTCTTTAGCTAGGATATTTGGGAAAGGCATATCTGAAGGAGTGATATTTG | |
| | AGCTTAGATTAAAAGATGGGAAGGAGCAAGCCATGCAAAGAGCTAGGATGTTCCAAGCAGAGACGGAACA | |
| | GCAAGTGCAAATGTCAGGAGGAATAGAAGGAGGCTGGTGGGTGGGGTCCAGTGAGCAAGAGGAGGGCAGG | |
| | CAGGAGAGGGGATGGGGAGGTGGGCAGGCCCAGACCACCCAGGGCCCTGGAGACTATCCTGATCCAACAA | |
| | GGGAAGCCTTGAGTCACTTCAGTGTCCATGTGGAGAATGGACCTCAGACTGAATGAGGGAGGCAGTAAGG | |
| | AGGGCCTCTACCTCCAGGGCTTCGCCCTGTGGACTGCGCATAGACATCTCCAACTCAGAAAGTCTGAACC | |
| | AAACTTTCCATAGTTCCCCCAAGTCTGGGCATCCTCCTACTCAGTGAAAGGCAGCCATCACACCTCCCTG | |
| | CCCTGCTCCCGGATGCCCCAAATCCTCTTGGTCTCCAAGTCCAGAACCTGAGACTTGTCCTTGATGTTTG | |
| | TCTTTCCCTCACCCTTTCTGTATTCTGGGAAGATGGGTTTTTTTCCCCCAGATGAATCTGTAAAACTTCT | |
| | GTGATCACAATAAAAATTCTGGCAGTATTATTTTCTGGAACATGACAAAGTGATTCAAAATTATTTATCT | |
| | GGAAGACTACAAAACAAGAATAGCCAGGAAATTTCTAAAAAGAAAGAAGAAGGAGGAGGAGAAAGAAGGA | |
| | GGAGGAAAAGGAGGAGAAGAAGAAAAGAAAAAGAACCAAGAAAGGGTTCTAGCTCTACCAAATATTAAAA | |
| | CATATCATGAAGCTATTTAAAACAATATGGTTGTGGATACTGAAAAAGATGTGAATAAAGTGGAAGGAAA | |
| | ATAAATAGAAATGCACATGGGGATTGAGACTGTGAAAAAGGCAGCATCTCACATCAGTGAGGGATGTTCA | |
| | ACACCTGGTGTTGGGAAAACTGGCTAGTCATTTAAACCAAACAACTGGGTCCTCTACCTCACTCCTGACA | |
| | TTAAGATACATTTAGATGATTCAAAGAGTAAGACAGAAAAAATAACACGTGAAAACACTATCAGAAAACA | |
| | ACGTGGGCCAGGTGTGGTGGGTCACGCCTGTAATCCCAGCACTTTGGGAGGCCGAGGCAGACAGATCACC | |
| | TGAGGTGGGGAGTTCAAGACCAGCCTGACCAACATGGTGAAATCCTGTCTCTACTAAAAATACAAAATTA | |
| | GCTGAGCGTGGTGGCGCATGCCTGTAATCCCAGCTACTCAGGAGGCCGAGGCAGGAGAATCACTTGAACC | |
| | TGGGAGGCAGAGGTTGTGGTGAGCCGAGATCACGCCATTGCACTCCAGCCTGGGCAACAAGAGTGAAAAT | |
| | CCATCTAAAAAAAAAAAAAAAAGCCAAGGTGGATATTTTTATAGTATCAGGGTAGATCAAGCTTCTCCAA | |
| | TCATGACATGAAACCCAGAAACCATAAAAGAAAAGAATGATAAAATTGCCCACGTAAAGTAAAAAGCTTG | |
| | CACACAGAAAAACACCATACAGGTTACAAGATGAGCAGCAAAATCAGAGAAAAAACATTGCAATTCAGGA | |
| | CACACAGAGGCTATTGTTCCTAATATTTAAAAATAAAAGTAGTGGATTGTCTACAAAAAGATGAAGACAA | |
| | GAATTTCAGAAAACCAAATACTGCATGTTTTCACTTACAAGTGGAAGCTAAACACTGAGTACACGTGTAC | |
| | ACAAAGAATGGAACCATAGGCCAGGCACCGTGGCTCACGCCTGTAATCCCAGTACTTTGCGAGGCCGAAG | |
| | CGGGCGGATCACCTGAGGTGAGGAGTTCGAGACCATCCTGGCCAACATGGTGAAACCCAGTCTCTACTAA | |
| | AAATACAAAAATTAGCCGGGCGTGGTGGTGGGTGCCTGTAATCCCAGCTACTCGGGAGGCTGCGGCAGTA | |
| | GAATCGCTTGAACCCTGGAGGTGGACCTTGCAGTGAGCCGAGATCGCACCACTGCACTCCAGCCTGGGCA | |
| | ACAGAGTGAGACTCCATCTCAAAAAAAAAAAAAAGGAATAGAACAATAGACACTGGGGCCTACTTGAGGG | |
| | AGGAGGGTGAGGATCAAAAACCTGCCTATCAGGTACTATGCTTATTACCTGGGTGGTGAAATAATCTGTA | |
| | CACCAAACCCCAGTGACATGCAATTTACCGATGTAACAAACCTGCCCATGTACCCGCTGAACCTAAAATA | |
| | AAAGTTGGAAAAAAATATAGAAATTTTCTTTGTAATAGCCAAAAACTGCAAACAGCCCAGGTGTCTATTA | |
| | GTAGAATGCATAAACAAACTCGGGCATGTTCATACAATGTAAAACTACTCATCAATAAAAAGTGATACTT | |
| | CTCAGCAATGAAAAGAAACTAGCTACTGATACCAGCTACAACATGGATGGATTTCAAGTGCTTTATGATG | |
| | AGAGCAAGAAGCCAGACACAAAAGTGTCTATATATATATACAGTATATATACGTATATATACACATATAT | |
| | ACAGTATATATATACATATACATGTATATATATACTGTATATATACTGTATATATATACACAGTATATAT | |
| | ATACATATATACAGTGTATATATACTGTGTATATATACATGTATATATACTGAGTATATATACATGTATA | |
| | TATATGTATACTGTATATATACTGTATATATATATACACATATATACAGTATATATATACAGTATATACT | |
| | GTATATATACAGTATATACGTGTATATATACATATATACAGTATATATGTAAATATACATATATACAGTA | |
| | TATATGTAAATATACATATATACATGTATATATATACACTATATATATACATATATAGTGTATATATACA | |
| | TATATACATGTATATATTTACTATATGATTCCATTTATATAAAGTGCCAAAACAGTCAAAAATAATCTAT | |
| | GTGGAAAAAATCAACAAAGGGATCCCCCGGGCTGCAGGAATTCGATGGCGCGCCTTAATTAAAATTATCT | |
| | CTAAGGCATGTGAACTGGCTGTCTTGGTTTTCATCTGTACTTCATCTGCTACCTCTGTGACCTGAAACAT | |
| | ATTTATAATTCCATTAAGCTGTGCATATGATAGATTTATCATATGTATTTTCCTTAAAGGATTTTTGTAA | |
| | GAACTAATTGAATTGATACCTGTAAAGTCTTTATCACACTACCCAATAAATAATAAATCTCTTTGTTCAG | |
| | CTCTCTGTTTCTATAAATATGTACCAGTTTTATTGTTTTTAGTGGTAGTGATTTTATTCTCTTTCTATAT | |
| | ATATACACACACATGTGTGCATTCATAAATATATACAATTTTTATGAATAAAAAATTATTAGCAATCAAT | |
| | ATTGAAAACCACTGATTTTTGTTTATGTGAGCAAACAGCAGATTAAAAGGCTAGCCTGCAGGAGTCAATG | |
| | GGAAAAACCCATTGGAGCCAAGTACACTGACTCAATAGGGACTTTCCATTGGGTTTTGCCCAGTACATAA | |
| | GGTCAATAGGGGGTGAGTCAACAGGAAAGTCCCATTGGAGCCAAGTACATTGAGTCAATAGGGACTTTCC | |
| | AATGGGTTTTGCCCAGTACATAAGGTCAATGGGAGGTAAGCCAATGGGTTTTTCCCATTACTGACATGTA | |
| | TACTGAGTCATTAGGGACTTTCCAATGGGTTTTGCCCAGTACATAAGGTCAATAGGGGTGAATCAACAGG | |
| | AAAGTCCCATTGGAGCCAAGTACACTGAGTCAATAGGGACTTTCCATTGGGTTTTGCCCAGTACAAAAGG | |
| | TCAATAGGGGGTGAGTCAATGGGTTTTTCCCATTATTGGCACATACATAAGGTCAATAGGGGTGACTAGT | |
| | GGAGAAGAGCATGCTTGAGGGCTGAGTGCCCCTCAGTGGGCAGAGAGCACATGGCCCACAGTCCCTGAGA | |
| | AGTTGGGGGGAGGGGTGGGCAATTGAACTGGTGCCTAGAGAAGGTGGGGCTTGGGTAAACTGGGAAAGTG | |
| | ATGTGGTGTACTGGCTCCACCTTTTTCCCCAGGGTGGGGGAGAACCATATATAAGTGCAGTAGTCTCTGT | |
| | GAACATTCAAGCATCTGCCTTCTCCCTCCTGTGAGTTTGgtaagtcactgactgtctatgcctgggaaag | |
| | ggtgggcaggaggtggggcagtgcaggaaaagtggcactgtgaaccctgcagccctagacaattgtacta | |
| | accttcttctctttcctctcctgacagGTTGGTGTACAGTAGTAGCAAGCTTGCATGCCTGCAGGTCGAC | |
| | TCTAGACTGCCATGGTATTTGTGAGACGCCCCTGGCCCGCGCTGACAACCGTGTTGCTGGCACTTCTTGT | |
| | GTGCCTTGGAGCTCTCGTTGATGCCATCAAGCCTGAAGCACCGGGGGAAGACGCTTCCCCCGAGGAGCTC | |
| | AATCGATACTATGCTAGTCTGCGACATTATCTGAACCTTGTGACCAGGCAGCGGTACGGCAAACGGGATT | |
| | TTCCAGAGGAAGTGGCGATCGTCGAGGAGCTCGGACGGCGGGGAAAACGCATCAAACCCGAGGCCCCAGG | |
| | CGAGGATGCTTCACCTGAGGAACTTAACCGCTACTACGCAAGCCTGCGGCATTACCTTAACCTCGTGACC | |
| | AGACAGCGCTACTAAGGATCCCCGGGAGATATCCTAGGCTTGGCCAGACATGATAAGATACATTGATGAG | |
| | TTTGGACAAACCACAACTAGAATGCAGTGAAAAAAATGCTTTATTTGTGAAATTTGTGATGCTATTGCTT | |
| | TATTTGTAACCATTATAAGCTGCAATAAACAAGTTAACAACAACAATTGCATTCATTTTATGTTTCAGGT | |
| | TCAGGGGGAGGTGTGGGAGGTTTTTTAAAGCAAGTAAAACCTCTACAAATGTGGTATGGAATTCAGTCAA | |
| | TATGTTCACCCCAAAAAAGCTGTTTGTTAACTTGCCAACCTCATTCTAAAATGTATATAGAAGCCCAAAA | |
| | GACAATAACAAAAATATTCTTGTAGAACAAAATGGGAAAGAATGTTCCACTAAATATCAAGATTTAGAGC | |
| | AAAGCATGAGATGTGTGGGGATAGACAGTGAGGCTGATAAAATAGAGTAGAGCTCAGAAACAGACCCATT | |
| | GATATATGTAAGTGACCTATGAAAAAAATATGGCATTTTACAATGGGAAAATGATGGTCTTTTTCTTTTT | |
| | TAGAAAAACAGGGAAATATATTTATATGTAAAAAATAAAAGGGAACCCATATGTCATACCATACACACAA | |
| | AAAAATTCCAGTGAATTATAAGTCTAAATGGAGAAGGCAAAACTTTAAATCTTTTAGAAAATAATATAGA | |
| | AGCATGCCATCAAGACTTCAGTGTAGAGAAAAATTTCTTATGACTCAAAGTCCTAACCACAAAGAAAAGA | |
| | TTGTTAATTAGATTGCATGAATATTAAGACTTATTTTTAAAATTAAAAAACCATTAAGAAAAGTCAGGCC | |
| | ATAGAATGACAGAAAATATTTGCAACACCCCAGTAAAGAGAATTGTAATATGCAGATTATAAAAAGAAGT | |
| | CTTACAAATCAGTAAAAAATAAAACTAGACAAAAATTTGAACAGATGAAAGAGAAACTCTAAATAATCAT | |
| | TACACATGAGAAACTCAATCTCAGAAATCAGAGAACTATCATTGCATATACACTAAATTAGAGAAATATT | |
| | AAAAGGCTAAGTAACATCTGTGGCTTAATTAAGGCGCGCCTACCAGTAAAAAAGAAAACCTATTAAAAAA | |
| | ACACCACTCGACACGGCACCAGCTCAATCAGTCACAGTGTAAAAAAGGGCCAAGTGCAGAGCGAGTATAT | |
| | ATAGGACTAAAAAATGACGTAACGGTTAAAGTCCACAAAAAACACCCAGAAAACCGCACGCGAACCTACG | |
| | CCCAGAAACGAAAGCCAAAAAACCCACAACTTCCTCAAATCGTCACTTCCGTTTTCCCACGTTACGTCAC | |
| | TTCCCATTTTAAGAAAACTACAATTCCCAACACATACAAGTTACTCCGCCCTAAAACCTACGTCACCCGC | |
| | CCCGTTCCCACGCCCCGCGCCACGTCACAAACTCCACCCCCTCATTATCATATTGGCTTCAATCCAAAAT | |
| | AAGGTATATTATTGATGATGA | |
| | Description of the elements: | |
| | (1) . . . (103): inverted terminal repeat (ITR) | |
| | (104) . . . (27556): encodes the shuttle vector (P-delta-28) | |
| | (27557) . . . (27564): AscI Restriction enzyme site | |
| | (27565) . . . (27991): encodes CpG free MAR from human beta-globin | |
| | gene | |
| | (27992) . . . (28414): encodes CMV Enhancer | |
| | (28415) . . . (28669): encodes human EF1-alpha promotor | |
| | (28670) . . . (28797): encodes synthetic intron | |
| | (28798) . . . (28851): encodes a multiple cloning site | |
| | (28852) . . . (29205): encodes transgene (PYY) from ATG to stop | |
| | codon | |
| | (29206) . . . (29233): encodes a multiple cloning site | |
| | (29234) . . . (29464): encodes SV40 poly Adenylation signal | |
| | (29465) . . . (30272): encodes MAR 5′ region from human IFN-beta | |
| | gene | |
| | (30273) . . . (30280): AscI Restriction enzyme site | |
| | (30281) . . . (30577): encodes the shuttle vector (P-delta-28) | |
| | (30578) . . . (30681): inverted terminal repeat (ITR) | |
| HDAd-EF1a-PYY | CATCATCAATAATATACCTTATTTTGGATTGAAGCCAATATGATAATGAGGGGGTGGAGTTTGTGACGTG | 46 |
| Approach 6 | GCGCGGGGCGTGGGAACGGGGCGGGTGACGTAGTAGTGTGGCGGAAGTGTGATGTTGCAAGTGTGGCGGA | |
| Codon | ACACATGTAAGCGACGGATGTGGCAAAAGTGACGTTTTTGGTGTGCGCCGGTGTACACAGGAAGTGACAA | |
| Optimized | TTTTCGCGCGGTTTTAGGCGGATGTTGTAGTAAATTTGGGCGTAACCGAGTAAGATTTGGCCATTTTCGC | |
| Nucleic Acid | GGGAAAACTGAATAAGAGGAAGTGAAATCTGAATAATTTTGTGTTACTCATAGCGCGTAATATTTGTCTA | |
| Sequence | GGGCCGCGGGGACTTTGACCGTTTACGTGGAGACTCGCCCAGGTGTTTTTCTCAGGTGTTTTCCGCGTTC | |
| Encoding PYY | CGGGTCAAAGTTGGCGTTTTGATATCAAGCTTATCGATACCGTAAACAAGTCTTTAATTCAAGCAAGACT | |
| (3- | TTAACAAGTTAAAAGGAGCTTATGGGTAGGAAGTAGTGTTATGATGTATGGGCATAAAGGGTTTTAATGG | |
| 36) Approach 6 | GATAGTGAAAATGTCTATAATAATACTTAAATGGCTGCCCAATCACCTACAGGATTGATGTAAACATGGA | |
| in | AAAGGTCAAAAACTTGGGTCACTAAAATAGATGATTAATGGAGAGGATGAGGTTGATAGTTAAATGTAGA | |
| HDdelta28E4- | TAAGTGGTCTTATTCTCAATAAAAATGTGAACATAAGGCGAGTTTCTACAAAGATGGACAGGACTCATTC | |
| MAR-EF1a | ATGAAACAGCAAAAACTGGACATTTGTTCTAATCTTTGAAGAGTATGAAAAATTCCTATTTTAAAGGAAA | |
| shuttle | ACAGTAACTCACAGGAAATACCAACCCAACATAAAATCAGAAACAATAGTCTAAAGTAATAAAAATCAAA | |
| vector | CGTTTGCACGATCAAATTATGAATGAAATTCACTACTAAAATTCACACTGATTTTGTTTCATCCACAGTG | |
| | TCAATGTTGTGATGCATTTCAATTGTGTGACACAGGCAGACTGTGGATCAAAAGTGGTTTCTGGTGCGAC | |
| | TTACTCTCTTGAGTATACCTGCAGTCCCCTTTCTTAAGTGTGTTAAAAAAAAAGGGGGATTTCTTCAATT | |
| | CGCCAATACTCTAGCTCTCCATGTGCTTTCTAGGAAACAAGTGTTAACCCACCTTATTTGTCAAACCTAG | |
| | CTCCAAAGGACTTTTGACTCCCCACAAACCGATGTAGCTCAAGAGAGGGTATCTGTCACCAGTATGTATA | |
| | GTGAAAAAAGTATCCCAAGTCCCAACAGCAATTCCTAAAAGGAGTTTATTTAAAAAACCACACACACCTG | |
| | TAAAATAAGTATATATCCTCCAAGGTGACTAGTTTTAAAAAAACAGTATTGGCTTTGATGTAAAGTACTA | |
| | GTGAATATGTTAGAAAAATCTCACTGTAACCAAGTGAAATGAAAGCAAGTATGGTTTGCAGAGATTCAAA | |
| | GAAAATATAAGAAAACCTACTGTTGCCACTAAAAAGAATCATATATTAAATATACTCACACAATAGCTCT | |
| | TCAGTCTGATAAAATCTACAGTCATAGGAATGGATCTATCACTATTTCTATTCAGTGCTTTGATGTAATC | |
| | CAGCAGGTCAGCAAAGAATTTATAGCCCCCCTTGAGCACACAGAGGGCTACAATGTGATGGCCTCCCATC | |
| | TCCTTCATCACATCTCGAGCAAGACGTTCAGTCCTACAGAAATAAAATCAGGAATTTAATAGAAAGTTTC | |
| | ATACATTAAACTTTATAACAAACACCTCTTAGTCATTAAACTTCCACACCAACCTGGGCAATATAGTGAG | |
| | ACCCCATGCCTGCAAAAAAAAAAAAATTAGCCAGGCATGGTAGCATGTACCTGTAGTCCCAGCTACTTGA | |
| | GAGGTGAGGTGGGAAAATCACTTTAGTGCAGGATGTTGAGGCTGGAGTGAACTGTGATTGTGCCACTGCA | |
| | CTCCAGCCTGGACAATAGAGCAAGACCTTGTCTCAAAAAAATGCATTAAAAATTTTTTTTAAATCTTCCA | |
| | CGTAACACATCCTTTGCCCTCATGTTTCATAAGGTAAAAAATTTGATACCTTCAAAAAAACCAAGCATAC | |
| | CACTATCATAATTTTTTTTAAATGCAAATAAAAACAAGATACCATTTTCACCTATCAGACTGGCAGGTTC | |
| | TGATTAAATGAAATTTCTTGGATAATATACAATATTAAGAGAGACTGTAGAAACTGGGCCAGTGGCTCAT | |
| | GCCTGTAATCCCAGCACTTTGGGAGGCTGGGTAACATGGCGAACCCTGTTTCTACAAAATAAAAATATTA | |
| | GCTGGGAGTGGTGGCGCACACCTATAGTCCCAGCTACTCAGGAGGCTGAGGTGGAAGGATCGCTTGAACC | |
| | CAGGAGGTTGAGACTGCAGTGAACTGTGATCATTCTGCTGCACTGCACCCCAGCCTGGGCAACAGAGACC | |
| | TTGTCTCaAAAAAAAAAAAAAAAGAGACAAATTGTGAAGAGAAAGGTACTCTCATATAACATCAGGAGTA | |
| | TAAAATGATTCAACTTCTTAGAGGAAAATTTGGCAATACCAAAATATTCAATAAACTCTTTCCCCTTGAC | |
| | CCAGAAATTCCACTTGAATAAAGCTGAACAAGTACCAAACATGTAAAAGAATGTTTCTTCTAGTACAGTC | |
| | GGTAAGAACAAAATAGTGTCTATCAATAGTGGACTGGTTAAATCAGTTATGGTATCTCCATAAGACAGAA | |
| | TGCTATGCAACCTTTAAAATATATTAGATAGCTCTAGACACACTAATATTAAAAGTGTCCAATAACATTT | |
| | AAAACTATACTCATACGTTAAAATATAAATGTATATATGTACTTTTGCATATAGTATACATGCATAGCCA | |
| | GTGCTTGAGAAGAAATGTGTACAGAAGGCTGAAAGGAGAGAACTTTAGTCTTCTTGTTTATGGCCTCCAT | |
| | AGTTAGAATATTTTATAACACAAATATTTTGATATTATAATTTTAAAATAAAAACACAGAATAGCCAGAC | |
| | ATACAATGCAAGCATTCAATACCAGGTAAGGTTTTTCACTGTAATTGACTTAACAGAAAATTTTCAAGCT | |
| | AGATGTGCATAATAATAAAAATCTGACCTTGCCTTCATGTGATTCAGCCCCAGTCCATTACCCTGTTTAG | |
| | GACTGAGAAATGCAAGACTCTGGCTAGAGTTCCTTCTTCCATCTCCCTTCAATGTTTACTTTGTTCTGGT | |
| | CCCTACAGAGTCCCACTATACCACAACTGATACTAAGTAATTAGTAAGGCCCTCCTCTTTTATTTTTAAT | |
| | AAAGAAGATTTTAGAAAGCATCAGTTATTTAATAAGTTGGCCTAGTTTATGTTCAAATAGCAAGTACTCA | |
| | GAACAGCTGCTGATGTTTGAAATTAACACAAGAAAAAGTAAAAAACCTCATTTTAAGATCTTACTTACCT | |
| | GTCCATAATTAGTCCATGAGGAATAAACACCCTTTCCAAATCCTCAGCATAATGATTAGGTATGCAAAAT | |
| | AAATCAAGGTCATAACCTGGTTCATCATCACTAATCTGAAAAAGAAATATAGCTGTTTCAATGAGAGCAT | |
| | TACAGGATACAAACATTTGATTGGATTAAGATGTTAAAAAATAACCTTAGTCTATCAGAGAAATTTAGGT | |
| | GTAAGATGATATTAGTAACTGTTAACTTTGTAGGTATGATAATGAATTATGTAAGAAAACAACAGGCCGG | |
| | GCGGGTTGGTTCACACGTGTAATCCCAGCACTTTGGGAGGCTGAGGCAGGCAGACTGCCTGAGCTCAGGA | |
| | GTTCGAGACCAGCCTGGGCAACACGGTGAAATCCCGTCTCTACTAAAAATACAAAAAAATTAGCCGGGTG | |
| | TGGTGACACATGCCTGTAGTCCCAGCTACTTGGGAGGCTGAGGCAGGAGAATCACTTGAACCTGGGAGGT | |
| | GAAGGTTGCAGTGAGCCAAGAATGCGCCACTTCACTCCAGCCTGGGAAACAGAGCAAGACTCTGTCTCAA | |
| | AAAAAACAAAACAAACAAACAAAAAAACAGGCTGGGCGCGGTGGCTCACGCCTGTAATCCCAGCACTTTG | |
| | GGAGGCCGAGGCGGGTGGATCACCTGAGGTCAGGAGTTCCAGACCAGCCTTGTCAACATGGTGAAACCTC | |
| | CCCCCGCCGTCTCTACTAAAAATACAAAAATTAGCCAGGCGTGGTGGCAGGAGCCTGTAATCCCAGCTAC | |
| | TTGGGAGGCTGAGGCAGGAGAATCGCTTGTACCCAGAAGGCAGAGGTTGCACTGAGCTGAGATGGCACCA | |
| | TTGCACTCCAGCCTGGGGGACAAGAGCGAGATTTCGTCTTTAAAAAACAAAAAACAAAACAAAAAACCAT | |
| | GTAACTATATGTCTTAGTCATCTTAGTCAAGAATGTAGAAGTAAAGTGATAAGATATGGAATTTCCTTTA | |
| | GGTCACAAAGAGAAAAAGAAAAATTTTAAAGAGCTAAGACAAACGCAGCAAAATCTTTATATTTAATAAT | |
| | ATTCTAAACATGGGTGATGAACATACGGGTATTCATTATACTATTCTCTCCACTTTTGAGTATGTTTGAA | |
| | AATTTAGTAAAACAAGTTTTAACACACTGTAGTCTAACAAGATAAAATATCACACTGAACAGGAAAAACT | |
| | GGCATGGTGTGGTGGCTCACACTTGTAATCCCAGTGCTTTGGGAGGCTGAGACAGGAGAGTTGCTTGAGG | |
| | CCAGGAGTTCAAGACCGACATGGGGAATGTAGCAAGACCCCGTCCCTACAAAAAACTTTGTAAAAATTTG | |
| | CCAGGTATGGTGGTGCATACCTGTAGTCCCAGCTACTCGGGAGGCGGAGGCAGAAGGAATCACTTGAGCC | |
| | CAGGAGTTTGAGGCTGCAGTGAGCTACGATCATACCACAGCACTCCAGCGTGGACAACAGAGTAAGACCC | |
| | TATCTCAAAAACAAAACAAAACAAAACAAACAAAAAAAACCACAAGAAAAACTGCTGGCTGATGCAGCGG | |
| | CTCATGCCTGTAATCCCAGTATTTTGGGAGGCCCAGGTGGGCGTATCACCTGAGGTCAGGAGTTAGAGAC | |
| | CAGCCTGGCCAACATGGTGAAACCCCATCTCTACTAAAAATACAAAATTAGCCAGGCATGTGGCACGCGC | |
| | CTGTAGTCCCAGTTACTGGGGAGGCTGAAGCAGGAGGATCACCTGAGCCCGGGAGGTGGAGGTTGCAGTG | |
| | AGCCGAGATCACACCACTGCACTCCAGCCTGGGTGACACAGCAATACCCTACCTCAAAATAAAAAAGAAA | |
| | AAGAAAAGAAAAGTTGCTGTCCCCGCTACCCCAATCCCAAATCCAAACAGCCTCTCTCATCTCACAGTAA | |
| | GGGGGAAAAATCACCCAAAAAAGCTAAGTGATCTTTTGAAAACCCAAACTCTTAGAAGTCTAAGATTATT | |
| | ATAGTCAACTCATGAAGTGTCATCATAAAAGATACTCTAATATTATTTAAGTAGAACCACATATTGGTTG | |
| | TCTTGGTATGTCTAGCCCCTGGCATACAAAATATTTAATAACACTGATATGGTACCTGTGATGTGAAAAT | |
| | GTACTATGAGTACAGCTTTATAAATACTATATATGTACCTATATACAGAAAAAAATACAACAAAATCATA | |
| | AAAGCACTTATCTTTGAAAGAGGAGTTACAGCAATTTTATTTAGTTCTTTATTGCTTTGCTATATATTCT | |
| | AAATTTTTTTCAATGAATATATATCACTTTTAAAAAAATTCAATGGTCTTTCTTATAAATTATCTTTGGC | |
| | AGCATGCGTTTTTATATATACATATAAAATGTATGGGAAATTTTTAAAGGATACATTAAATTAAAGCAAA | |
| | ATATACAAACAAAAAATCAGAATACAAAAAGATAAAAAGATTGGGAAGGGAGGGAGGGAGTAAGGAGGAA | |
| | GGGTGGGTGGGTATAGAGAAATATACCAAATAATGGTAAGAAGTGGGGTCTTGACACTTTCTACACTTTT | |
| | TTTAAATAAAAAAAATTTTTTTCTCTCTCTTTTTTTTTTTTAGAGACGAAGTCTCGCTATGTTGCCCAGG | |
| | CTGGTCTTGAACTCCTGGGATCAAGAGATCCTCCTGCCTCAGCCTCCCAAGGTGCTTGGATTACAGGTGT | |
| | GAGCCACCACGCCTGGTCACTTTCTACACTTTAATATATATATTTTTTCATTTTCAATGTCATTTTTATT | |
| | AGTTAATTTATAATACCCATTCACCATTATATTCAAAGTCTATTTGAAGAAATAAACCAGAAAGAATGAA | |
| | ATACTCTAGCTCACATGCTATTCAATACTAAATTACCTTTCAAATCACATTCAAGAAGCTGATGATTTAA | |
| | GCTTTGGCGGTTTCCAATAAATATTGGTCAAACCATAATTAAATCTCAATATATCAGTTAGTACCTATTG | |
| | AGCATCTCCTTTTACAACCTAAGCATTGTATTAGGTGCTTAAATACAAGCAGCTTGACTTTTAATACATT | |
| | TAAAAATACATATTTAAGACTTAAAATCTTATTTATGGAATTCAGTTATATTTTGAGGTTTCCAGTGCTG | |
| | AGAAATTTGAGGTTTGTGCTGTCTTTCAGTCCCCAAAGCTCAGTTCTGAGTTCTCAGACTTTGGTGGAAC | |
| | TTCATGTATTGTCAGGTTGGCCCGTAATACCTGTGGGACAACTTCAGCCCCTGTGCACATGGCCAGGAGG | |
| | CTGGTTGCAAACATTTTCAGGTAGGTGGACCAGGACATGCCCCTGGTCATGGCCAGGTGGAGGCATAGTG | |
| | CTATACAGCAGGCAGAAGTCAATATTGATTTGTTTTTAAAGAAACATGTACTACTTTCATAAGCAGAAAA | |
| | AATTTCTATTCTTGGGGGAAAAGATTATGCCAGATCCTCTAGGATTAAATGCTGATGCATCTGCTAAACC | |
| | TTCACATATCAGAACATATTTACTATAGAAAGAATGAAAATGGGACATTTGTGTGTCACCTATGTGAACA | |
| | TTCCAAAAATATTTTACAACAACTAAGTATTTTATAAATTTTATGAACTGAAATTTAGTTCAAGTTCTAG | |
| | GAAAATACAAACCTTGCTAGATATTATAAAAATGATACAATATATATTCATTTCAGGCTCATCAGAATAT | |
| | ATCTGTTATCACTTGACAAGAATGAAAATGCACCATTTTGTAGTGCTTTAAAATCAGGAAGATCCAGAGT | |
| | ACTAAAAATGACTTCTTCCTTGAAGCTTACTCACCAACTTCCTCCCAGTTACTCACTGCTTCTGCCACAA | |
| | GCATAAACTAGGACCCAGCCAGAACTCCTTGAAATATACACTTGCAACGATTACTGCATCTATCAAAATG | |
| | GTTCAGTGCCTGGCTACAGGTTCTGCAGATCGACTAAGAATTTGAAAAGTCTTGTTTATTTCAAAGGAAG | |
| | CCCATGTGAATTCTGCCCAGAGTTCATCCCAGATATGCAGTCTAAGAATACAGACAGATCAGCAGAGATG | |
| | TATTCTAAAACAGGAATTCTGGCAATATAACAAATTGATTTCCAATCAAAACAGATTTACATACCATACT | |
| | TATGTCAAGAAGTTGTTTTGTTTTATTGCATCCTAGATTTTATTTTTTTGATTTATGGTTTACTTTAAGC | |
| | ATAAAAAATTTGTCAATACAACTCTTCCCAAAAGGCATAAACAAAAATTCATAAAACTTGCATCACTTGA | |
| | GATACTTCAGGTATGAATTCACAACTTTGTTACAACTTACTATATATATGCACACATATATATATATTTG | |
| | GGTATATTGGGGGGGTTCTAATTTAAGAAATGCATAATTGGCTATAGACAGACAGTTGTCAGAACTTGGC | |
| | AATGGGTACGTGCAGGTTCATTATACCAAGTCTACTTGTAGTTGTTCAAAATGTATCATAATACAAGGCC | |
| | GGGCGAGGTGGCTCACGCCTGTAATCCCAGCATTTTGGGAGGCTAAGGCAGGAGGATTGCTTGAGGTCAG | |
| | GAGTTTGTGACCAGCCTGGGCAACAGAGCAAGACCCTGTCTCCAAAAAGAAAAAAAATAATTTTTTACAA | |
| | AATAAAAACAAAATGTATCATCAGACGAAATTAAATAAGAGGCAATTCATTTAAATGACAACTTTTCCCA | |
| | GCTTGACATTTAACAAAAAGTCTAAGTCCTCTTAATTCATATTTAATGATCAAATATCAAATACTAATTT | |
| | TTTTTTTTTTTTTTTTTTTGAGACGGAGTCTCGCTCTGTCGCCCAGGCTGGAGTGCAGTGGCGCGATCCT | |
| | GGCTCACTGCAAGCTCCGCCTCCCGGGTTCACGCCATTCTCCTGCCTCAGCCTCCCGAGTAGCTGGGATT | |
| | ACAGACATGCGCCACCACGCCCGGCTAATTTTGTATTTTTAGTAGAGATGGGGTTTCTCCATGTTGGTCA | |
| | GGCTGGTCTTGAATTTCCCACCTCAGGTGATCTGCCTGCCTCAGCCTCACAAAGCAGTAGCTGGGACTAC | |
| | AGGCACCCACCACCACACTTGGTTAATTCTTTTGTATTTTTTTTGTAAAGACGGGATTTCACCATGTTAG | |
| | CCAGGATGGTCTCGATCTCCTGATCTCATGATCCGCCCGCCTCAGCCTCCCAAAGTGCTGGGATTACAGG | |
| | CGTGAGCCACCCCGCCCGGCCATCAAATACTAATTCTTAAATGGTAAGGACCCACTATTCAGAACCTGTA | |
| | TCCTTATCACTAATATGCAAATATTTATTGAATACTTACTATGTCATGCATACTAGAGAGAGTTAGATAA | |
| | ATTTGATACAGCTACCCTCACAGAACTTACAGTGTAATAGATGGCATGACATGTACATGAGTAACTGTGA | |
| | ACAGTGTTAAATTGCTATTTAAAAAAAAAGACGGCTGGGCGCTGTGGCTCATGCCTGTAATCCCAGCACT | |
| | TTGGGAGGCCAAGGCAAGTTGATCGCTCGAGGTCAAGAGTTCGAGACCAGCCTGGCCAACGTGGTAAAAC | |
| | CCCGTCTCTACTAAAAATACAAAAAAAAAATTAGCCAGGCATGGTGGCACAGGCCTGTAATCCCAGCTAC | |
| | TAGGGAGGCTGAGACATGGAGAACTGCTTGAATCCAGGAGGCAGAGGTTACAGTGAGCCGAGATCATACC | |
| | ACTACACTCCAGCCTGAGTGACAGAGCGAGACTCCGTCTAAAAAAAAAAAAAAAAAAAAAGATACAGGTT | |
| | AAGTGTTATGGTAGTTGAAGAGAGAACTCAAACTCTGTCTCAGAAGCCTCACTTGCATGTGGACCACTGA | |
| | TATGAAATAATATAAATAGGTATAATTCAATAAATAGGAACTTCAGTTTTAATCATCCCAAACACCAAAA | |
| | CTTCCTATCAAACAGGTCCAATAAACTCAATCTCTATAAGAGCTAGACAGAAATCTACTTGGTGGCCTAT | |
| | AATCTTATTAGCCCTTACTTGTCCCATCTGATATTAATTAACCCCATCTAATATGGATTAGTTAACAATC | |
| | CAGTGGCTGCTTTGACAGGAACAGTTGGAGAGAGTTGGGGATTGCAACATATTCAATTATACAAAAATGC | |
| | ATTCAGCATCTACCTTGATTAAGGCAGTGTGCAACAGAATTTGCAGGAGAGTAAAAGAATGATTATAAAT | |
| | TTACAACCCTTAAAGAGCTTATAGCTGGGCGTGGTGGCTCATGCCTGTAAATCCCAGCACTTTGGGAGGC | |
| | TGAGGCGGGTGGATCACCTGAGGCCAGAAGTTCAAGACCAGCCTAGCCAACATGGCGAAACCCTGTCTCT | |
| | ACAAAAAATACAAAAATTAGCCGGGTGTGGTGGCACGTGCCTGTAGTCCCAGTTACTTGGGAGGCCGAGG | |
| | CAGGAGAATCGCTTGAACCTAGGAGGTGGAGGCTGCAGTGAGCCGAGATTGTGCCACTGCACTCCACTTC | |
| | AGCCTGGGCGACAAGAGCAAGACTCCGTCACAAAAAAAAAAAAAAAAAAAAAGCTTAAAATCTAGTGGGA | |
| | AAGGCATATATACATACAACTAACTGTATAGCATAATAAAGCTCATAATCTGTAACAAAATCTAATTCGA | |
| | CAAGCCCAGAAACTTGTGATTTACCAAAAACAGTTATATATACACAAAAAGTAAACCTAGAACCCAAAGT | |
| | TACCCAGCACCAATGATTCTCTCCCTAAGCAGTATCAAGTTTAAAGCAGTGATTACATTCTACTGCCTAG | |
| | ATTGTAAACTGAGTAAAGGAGACCAGCACCTTTCTGCTACTGAACTAGCACAGCCGTGTAAACCAACAAG | |
| | GCAATGGCAGTGCCCAACTTTCTGTATGAATATAAGTTACATCTGTTTTATTATTTGTGACTTGGTGTTG | |
| | CATGTGGTTATTATCAACACCTTCTGAAAGAACAACTACCTGCTCAGGCTGCCATAACAAAATACCACAG | |
| | ACTGAGTGACTTAACAGAAACTTATTTCTCACAGTTTTGGAGGCTGGGAAGTCCAAAATTAAGGTACCTG | |
| | CAAGGTAGGTTTCAATCTCAGGCCTCTTCTTTGGCTTGAAGGTCTTCTAACTGTGTGCTCACATGACCTC | |
| | TTCTAACAAGCTCTCTGGTGTCTCTTTTTTTTTTTTTTTCTTTTTTGAGACAGAGTCTCACTCTGTCACC | |
| | CAGGCTGGAGTACAGTGGCACAATCTGGGCTCACTGCAACCTCCAACTCCCGGGTTCAAGTGATTCTCAT | |
| | GCCTCACCCTCCCGAGTAGCTTGGATGACAGGAGCCCGCTACCACACCCAGCTAATTTTTGTATTTTTAG | |
| | TAGAGATGGTGTTTCACTACATTGGCCAGGCTGGTCTCAAACTCCTGACCTCGTGATCCACCCACCTTGG | |
| | CCTCCCAAAGTGCTGGGATTACAGGTGTGAGCCACTGCGCCCGTCCTGGTGTCTTTTCATATAAGGGCAC | |
| | TAATCCAATCAGACCTGGGCCCAACCCTCCCGACTTCTTCTAACTGTAATTACCTTCCAAAGGCCCTGTC | |
| | TCCAAATACCATCACACTGGGGGTTAGGACTTCAAAAAAGGTATGGGGGGGGTGTGGGAGGACATAAATG | |
| | CTCAGTCCATAACAAGCACCCAACATAAAAATGGCTAGAACAGATCACAAAAAAAAGGTCCTGTATGGCT | |
| | TTGGGGAAGGGCTCAACCCCAAAATATCTGAGAGCTCTGGAGGGGCCTAGAAGTGGTAAATGAATGAAAA | |
| | CGTGGTTACTCTCCAGATCTGCCTTTCCCAAATATGGCCATTCTTGGCTGAATCAGAAATCAAAGGACAG | |
| | GTTATTAATTACTAGCTCTAAGTTACTTACCATTTGCTGAGACAGTTCAGAAATCTGACTGCATCTCCTC | |
| | AGAGATCTAGAACACAGTTCTCAAATTCTAACTTACTTGTGATATACTTGTGAATGATAAAAATCGCTAC | |
| | AGGTACTTTTATTAATCTGAAAGAGTATTGAGAAATTACCTTTCATTCTGACTTTTGTCTGGAATGAAAA | |
| | TCAATACTTTTGCTATAATCGATTACTGAAATAATTTTACTTTCCAGTAAAACTGGCATTATAATTTTTT | |
| | TTAATTTTTAAAACTTCATAATTTTTTGCCAGACTGACCCATGTAAACATACAAATTACTAATAATTATG | |
| | CACGTCACATCTGTAATAATGGCCTTCATGTAAACATTTTTGTGGTTTACACATAAAATCTCTAATTACA | |
| | AAGCTATATTATCTAAAATTACAGTAAGCAAGAAAATTAATCCAAGCTAAGACAATACTTGCAACATCAA | |
| | TTCATCATCTGTGACAAGGACTGCTTAAGTCTCTTTGTGGTTAAAAAGGAAAAAAAAAAAAAAGACATGT | |
| | TGGCCAGATGCGGTGGCTCACACCTGTAATCCCAGCACTTTGGGAGGCTGAGGTGGGCGGATCACCCCTG | |
| | GCCTGCCCAACATGGTGAAACCCCGTCTCTACTAAAAACACAAAAATTAGCTGGGCGTGGTGGCGGGCGC | |
| | CTGTAATTCCAGCTACTCGGGAGGCTGAGGCAGGAGAATTGCTAGAACCCAGGAGGCAGAGATTGCAGTG | |
| | AGCTGAGATTGCACCATTGCACTACAGTCTGGGCAACAAAAGTGAAACTCCATCTTAAAAAAAAAAAGAC | |
| | AATGTTCGTGGGTCCAAACAAGACTTAATGGAAGTGAGTCTAAAAATGAGCTATGTGGGCCAGGCGTAGT | |
| | GGCTCCCACCTGTAATCCCAGCACTTTGGGAGGCCGAAGCAGGCAGATCATGAGGTCAGGAGATGGAGAC | |
| | CATCCTGGCCAACACGGTGAAATCCTGTCTCTACAAAAATTAGCTGGGCGTGGTGGTGCCTGCCTGTAAT | |
| | CCCAGCTACTCAGAAGGCTCAGGCAGGAGAATCGCTTGAACCAGGGAGTCGGTGGCTAGAGTGAGCCGAG | |
| | ATTGCATCACTGCACTCCTGCCTGGTGACAGAGCAAGACTCCATCTCAAAAAAAACAAACAAAAATAAAA | |
| | GATAAAAATGAGCTATGTGAATTAAAAGAGGTATAACAATAGATAAACCATATTTTATTTAATTCCTAGT | |
| | AATGAGTAATATTTCCAAACTTCTGGAATGGGCAGAAATTGCTAGTTGGCATATTTTTACCTTTTATATT | |
| | CAGATACATTAAAATTCTCAAAAAAAAACACCTCAAAGCAGATGATCCGCCATCTCCTTGGATAATTTGT | |
| | GTTAACTCAGGATAACAGAAAACCAAAATTATGAGTTACTGATGCAATATTCCTAAATGTAAAAATAATT | |
| | AAAGCTAATAGTAGATTCATCTTCCAATTTCATATCAGTCTTACAAATAAACTACATATATAACTTGCTT | |
| | GCCTTCCCTTCTGAGGGATAAAGCTGTTAGAAGAATTAAAATCAGCATTCTTGACTATTCAACCAAGGGA | |
| | GGGATAAATTATTACTCATTCTAGGGACATGGGCTCATAACTACTACATGTGTAAGGACATGAATTTACC | |
| | CAATATTACAATTTTTCCTTTTATTAGTGTGTACAGTGGAAGAATAGACATGTTCACTCTGGACAAAAAA | |
| | AAAATTATACTTATCAGTTATCAGAAGCACAATGCTGAAGACAGTAGTTCCATAACAATTTGAAGTATGT | |
| | GATCGAACTAGTAGATTATCTTAGTAGTAGTGAATTATTGTAAATGTTAGTAATTTGGCAGCCACTGGGC | |
| | AGAAAAATAAGAATTGAGGCTCAATATTGATATTAATGGTGGTGATTGACACATAAATTTTATCAAGTCT | |
| | ACACAATATAAAATTACAGAAAGGTAGAAGAGTATACCAGTACAACTTCAACATATCTTCACTACAAGGG | |
| | AGTAAAATGACATGGCCTAGTTACTATCTAATGAACTGCAGAAAACTAAAAGAAAACTCCAAGGCAACTC | |
| | TTCTCTGCTGATCTGGTTGGTCCTTTTCCTACCTTTTGCAATACCCAGATACAAACAATGGATAGAAAAC | |
| | AAAGTAGACTTGTAGTATGCAGGTCACAGTGCTAAATTCACAGAAAGAAACCCCTGAACTGAACTGCTCT | |
| | ATTTCCTGGTGGTCACAAAGAGTAATTCTGGTTTACACCTACAGATTGATGTCAATCTACACCCTGTTGA | |
| | TAACAGTGTGGCCAAGGACAAAAAAAAGGTGCTCCGTTTTACCAATTCTGTAAAAAATTATTGGCAGGGT | |
| | AAGCTCGGCTAGGGCAGGATTACATTTCTAGGACTACCATCCCCGAAATTTAGAAGATATTATATCCACA | |
| | TAAAGCATATCTTTCACATTAATTTGCAAAAATCTAAAAGCTTTTTCTTAGCTCAAGTGTGTCCAAGTTT | |
| | ACCCTGGCAGTTTAAAACGATAGTTACAAGCAGCATGGGTTGTATCAGACACATTTGAGGGCCAATTTCA | |
| | TGTAAGTGATATTGGGCAAGTTACTTCAACTATCTGTGCCTCCAAGGTCATACTAGTGTTTATTTACCTA | |
| | AAGGGTACCTGTTATGTAACTTTAGGGTGTTTACATTAGATAATGCCTGCAAAATATTTACTTCAACGCC | |
| | TAAAACATAGTTAAGTATTCAATAAATACCTACTATTGTCACTACTAACTTAAAAGTTTAGAGATTAAGA | |
| | GCAGAATCTGGGGTGAGACAAACTTAGGTTCAAATCCTAGTATTGTTGGGTAATCTTGGGCAAGTTACTT | |
| | AACCTCTCTGATTTGTGTAATTTAAAAAATTAGTTAATATACATAACAGGGCTTAGAAGAGTATCTAGCA | |
| | CATAGCACCATTTAAGCATTTGTTATTGCTAACATGCAAACAATTTAAGGGAAAGAAATTTTTTAAAAAG | |
| | GAAGAGGGATTTGCAAACTAAAAACAATGAGTATCTTATGTTCAAAGAAAACTAACAAACAGCCAGCTCT | |
| | AGCAATAATTAAATTCACTATATACTGGGGCAGGCATCACACCCCAAAGCTAAAAGCGTCTACCTAGGCC | |
| | AGGCACGGTGGCTCATGCCTGTAATCCCAGCACTTTGGGAAGCAGAGGCGGGCAGATCGCTTGAGCTCAG | |
| | GAGTTCAAGACCAGCCTGGACAACATGGCAAAACACCATCTCTACAAAAAATACAAATATTAGGCCGGGC | |
| | GCAGTGGCTCACGCCTGTAATCCCAGCACTTTGGGAGGCCAAGGCGGGTGGATCACCTGAGATCAGGAGT | |
| | TCGAGAGTAGCCTGGCCAACATGGTGAAACCTCGTCTCTATTAAAAATACAAAAAATTAGCCAGGCATGG | |
| | TGGCAGGCGCCTGTAATCCCAGCTACTCAGGGGGATGAGGTAGGAGAATCGCTTGAACCCGGGAGGCAGA | |
| | GGTTGCACTGAGCCGAGATCATGCCACTGTACTCCAGCCCGGGCAACAAGAGCGAAACTCCATCTCAAAA | |
| | AATAAATAAATAAATAAATAAAATAAAGTACAAATATTAGCCAGGGATGGTGGTGCGCACCTGTAGTCCC | |
| | AGCTACTTGGGAGGCTGAAGTGGGAGAATCCCCTGAGCCTGGGGAGAATCACCCGAGCCCGGGAAGTCGA | |
| | GGCTGCAGTGAGCAGTGATTGTGCCACTGCACTCCATCCTAGGTGACAGAGTGAGACCCTGTCTCAAAAA | |
| | AAAGAAATTGGCAGAATTAAGTAAGTTGATGTTTAGAGATGAAAAATCAACATTTTTTCCTCAGCAACTG | |
| | AATAAAAACAACAGCCACTACCATTTTTTTGAGTACCTATTTGTAGCCTATTTTTTAACTGGTATTACTC | |
| | GAGAGAGAGAGAGCTAGGTTCGAGACAGAGCTCCTTCTCTTAATAACTGTATGACCTAGGGTATGTCTGT | |
| | TAGCCTCTCTGAGGCTTCAAAGGTTCCTCATCTGTAAAATGGTAATAATCATACCATTGCTACAGGGCTG | |
| | TTTTGAAGACTAATTAGGACTATGTAAGTAAACATGATGATGGCTATTATTACTGTTCCCCGCCAGGGGC | |
| | CATGCAAGGGTTGCTGATTCACATAGACTGTCTTATAATCCTCTCAATAACTCCAAGAGGTAGCCAGCAC | |
| | CTCAGATATACATAAAATGACTTAAGCCCAGAGAGGTGAAGTAAGTTGCCCACAGCCACACAACTAGTAA | |
| | ATAGCCCAAACAAGCTGGATTCCCAGTTAGACTCCGTTAATAGCACTGCTCTTTACCTTAAGTCATTACA | |
| | ATGCCTAATATGAAATAGAATCGCTTCTTTCTTAGGGTTCAAGTGGTTAATTATTTAATGTATTCATTCA | |
| | ACAAACCATCATCGAGGACCTCTTACAAGCCAAGTACTGTGCTAAGTGCTAGAGTTACGGCGGTGATTCC | |
| | TGCCCTTAAAAAGTTTTAGTGGGAGAAACAACAGGTAACCAGGTCATTGCCAAAACAACAAAAATAATCA | |
| | TAATAAAGCAGGCTAAAGCATATTTAACTGGCCGGGGTTTTGACTATTTTAGCAAGCATGATCAGAACGG | |
| | TTGAGGAGGGAGGCCAGCAGCTTGGCCGGTTCAACAAACAAGAAAAAACCAGTGAGGGTGGAGCTAAGAT | |
| | ACCAGAGGCTGATTACGGTTAAGAATGTTCTTGAAGGTAAGGACCAGATTCTCATTTTCTATATCCTGGG | |
| | GCATCGGTCAGCATGGAATCTGGATTCTAGCACATGTGAATTTCGGCTTGAAATGACCTAATGCCTTTTC | |
| | CCTAGTTCCTTCGTGTGTCAAATACGCATGGTTACCGCTACCAGAGCTGTAGTGGGGCTTCAATGAGGCC | |
| | ATGAGCATCTCCATAAAGATGAACTACAGTGTGTGCAAAACTAAAGGCAAAACCTGGTCCCCACACGCCC | |
| | TCCCAGGTGGTCGCTTTCCGTGCCGAGGCCCCTCCAGAGGTGCCCCGAGAACCTCACCATCGCACCCCAA | |
| | ACTTCCAGGGAAGGGCCTCTCCCGAGAAAGCCCCCACGCCCCCACCCCGCGCCATCATTCCCGAATCTGC | |
| | CCTCGGCCCCTCCCCGCAGCACGCTCGCAGGCGGCACATGTCAACCAAAACGCCATTTCCACCTTCTCTT | |
| | CCCACACGCAGTCCTCTTTTCCCAGGGCTCCCCCGAGGAGGGACCCACCCCAAACCCCGCCATTCCGTCC | |
| | TCCCTGCCGCCCTCGCGTGACGTAAAGCCGAACCCGGGAAACTGGCCGCCCCCGCCTGCGGGGTTCCCTG | |
| | GGCCCGGCCGCTCTAGAACTAGTGGATCCCAATTGAAGGCCTGGTCTAAATGACTCCAAAATCACCACTT | |
| | AATTCAAGAGACTGATTTCCCTGAGTCAGGCCCCTTAAAGCAGCTATTTCAATGGGACAGGGAAACAACC | |
| | CTAGGATCTGGATTAGAATCACTTGGGGGCTGCCACACCCCCAGGGCTCTGATCCTGCCCTTCTCCCACA | |
| | CGCACATTCACATACTGCTGCAGTGACCTTCCATTTCTAATGGGTTCCTGGGCCATCTGTCAGGTATAGG | |
| | GAATGGAAAAGGGGTTGGGGAGGCTCTGCTTCAGAAAGTTTGTGTCAGGGGCTCCCAGAGCCTCCACAGA | |
| | TAGATAGCAGGGGTCCCCACCCTACCATGGCAGCTATAAATGTGATCAACATTTATTGGCCTAGGATACA | |
| | GCAGTTAGCAAAATGCCTGATGTAGTTCCCACTCCGTGGAGGTTGCAGGCTAGCCAAGAAGTCATGAGTT | |
| | CAGCAACCCTTACGCACCAGTGGGATGAGATTGGACCAGGCCGAGGGTAGTCTTGGGAACACTCAGCATT | |
| | TGTCTGAGGGCCAGAAGAGGCTGCTTGCCCTCAGACAGGAGGTCAGCATCTTTATTGTAGCCCATGACAC | |
| | CTCTACACCATTGCTCTTCTGGTCTTATGGAAGACATCTTTGGGCCTGATAACAGCGGAGTCTGTGTCCC | |
| | ACTTGTCCAGGCTGGAGTGCCACATCAGGCACACTCCAGTTGCAGGGACAGCACAGACAAGTTTCAGGAA | |
| | GGCTGGTGGCCTCCAGGAGGTTAACCTTATAAGGCCAGATTGTAACCTAGTTGAAAAACATACACATGCC | |
| | ATGATAATAAAAGAACCTAGGCACCATTACAAGAGAAAAAATCATTTTTGTAGATACGAGCATGGATTCT | |
| | TGGGTGGGTCAGACACACTGGGCTTGTGCTCTGACTGCACTGTCTCCCCTACCTGACCTTGGGTAAACCA | |
| | TAAGACTGCTGCATGACTCAGTGTCCACCCCAAAAAAGTACCGGTAGATATTGGCCACAGTAGATATCAG | |
| | CTAGAGTGGACTCTCATGACAATGAGGGGAGATGTATTCCCCATCTTAGGCACCTGGGACTCTACCTTCC | |
| | ATCTTCTGCTCCGTGTCTCTCCATCCCCAGGCTCTTCAGAACTCAGGGAGTCCAGAATGTCAGCTCCCAG | |
| | ATTTCAGCCTTCAGAAAGGAAACCCATTACCGTTCAGTTGAACAAATGTTGTCTGAGCCCCAGATCTGGG | |
| | CTCAGAGGCCATCTAGGCTATGAGACAAGAGGGGAACAAAGCACCGTCTGCACTCACTCACCACACTCAC | |
| | TTGCTGTCCCAGGTCACATCCATCGGGTAGAGAATCTAAGAGGCTGAGCTAGCTCCCGCCACCAGCCCAG | |
| | CCCACCCCACCTGGCCCCTTCCTTCCTTCTACAAAATATGCACCACCTGTCAAAGGGTGGGCAGTGCCAG | |
| | GCCTGCATACAGAGCACTGAGTGTAAAAGCAGACATGGACCCTGACCTCCAGGAGCTTCCAATTTTCTTG | |
| | AAGAGACAAATCAGCTGGCATTTCAGTCCAGTGTGATCTGCTCTTGGTGAGCACAGACCTAGGGAGTTGG | |
| | GGCAGCTTCCCAGAAGAACTGCAGTCCAGGCTGAGGGCAGAGAAATGAGGGGAATGGCGAGGAATTGGGG | |
| | AGCAGGGGGGAGCTCAGTAGAGAGCCAAGGGCGGGAGGTGAGAAGTCCGTGTTGGGCCAGGAGCTACCCT | |
| | CCGGTGGCCACAGCCGAAGTCGAGGATGCCTTTGGAACTCATCCCCACTTCTCTCTTTCTGTATGTAGCC | |
| | GTCCAAGAACAAGTCACCTCCAAGTGTAGCCGGATCAAGGCAAGCCCCCCATCTAGCAAGCACTTGATGC | |
| | CACCCAGAACTGGGCTTCTTCAGAACAATCTGAGTCCAGGAATGATCCCACTCACCAGGCACCAGAGCTG | |
| | CGAGGGCATGGGAGTGATCTCACCAACTCTGGGGAAGCGGCAAGGAATTTTCACCTCCAGCCCCCAGTGT | |
| | CCCATCCTCTCACACTCAGGCCAGACTCCCCTGGGCAGACTTGACTCTGTCTGCCAGCATATGCAGAGCC | |
| | CCAAGGCCACCCCACCAGAAGTGCCCCTGCCTGGGTTCTGTCCCAGCTCCCTGGGCACCCAGTCCTTGAG | |
| | TCCCCACCAGCTCAGACGGCCTAGTGTGCCAAGAATGCCCACTGCGTTCAACAATGCTGCATGGGTCACA | |
| | GCGGCAGCAGCTGTGACCACAGCAGTTTCGGGGAAAACACCCCTCAGCCAAGTGGATAATAGCGTTCAGC | |
| | AGCACTCACCTTCTGGCCAGGCCTGCCTTCAGAGGCCATCTGATTGGGAGGCACAAGTGCCCGCTGCGAT | |
| | GGGAACACAAGTGCCCCTGGCCAACAACCCCAGCTTCAGCCTGCTGGGCAGCCAGAGCCTCAGGCAGAGC | |
| | CCGGTACAGGGCCCGGTGCCTGTAGCAAACACCACCAAGTTCCTCCAGCAGGGTATGGCCAGCTTTAGTC | |
| | CCCTGAGCCCCATACAGGGCATCGAGCCACCAAGCTATGTGGCTGCTGCTGCCACCGCTGCTGCTGCTTC | |
| | TGCCGTTGCTGCCAGCCAGTTCCCAGGTCCGTTCGACAGAACGGATATTCCCCCTGAGCTGCCACCTGCC | |
| | GACTTTTTGCGCCAGCCCCAACCCCCACTAAATGATCTGATTTCGTCACCTGACTGCAATGAGGTAGATT | |
| | TCATTGAAGCTCTCTTGAAAGGCTCCTGTGTGAGCCCAGATGAAGACTGGGTGTGCAACTTGAGGCTGAT | |
| | CGACGACATTTTGGAACAGCATGCTGCTGCTCAAAATGCCACAGCCCAGAATTCTGGGCAAGTCACCCAG | |
| | GATGCTGGGGCACTTTAAATCTGAGCAGGATGCCCATAGAAACCCCCATGGTGACATCACTCTAGGAAGT | |
| | GGTGTCGATCCATACCCGCAGTTGTCTCCCGTTACAATTTGAGTGGTGTTGTCAGCCCATGCTTATCCCT | |
| | CTCTCTACCTGTGACAAAATGGAAAGCTGGTGATTTTTCAAGCTACGTGTACATATTTGAAAATTTTGTA | |
| | AATGGTTTTCCTAAACATTAATGACAGAAGTATTTATACTTCATTTTGTGACTTTGTAAATAAAGCGACG | |
| | GCTTTTGTTTCAGTAGAGTTGTGTTTACTATGCATTGTTTTGTGTTTATTATACAATGTTACAAATATGC | |
| | AGACCGTGTTGTTTGCTCCAGTGATACCTTGTTAAGCTAGGTGGCTGAGTCGCTTATGGTTTTAATGCAA | |
| | TGAGCAATGTGGATATGACCAAGAGTTGTTGTGCAAGTTGACAAATGCCAAATAGAAAACCACTTGGCCA | |
| | TTTATTTCTATGTTCACTAAAAATCCTATTGCCTTGTGTGATTCTTAATCTCTTTTGCGAACCTTTCAGT | |
| | CTCCGCTAGCTCTTTCCTAATGAGCTTTACAGCAGAAGCTGTTTTATCGTTAAGTGCCCCACAGAGACAC | |
| | TTTACCAGGAGGCTGGGAGAGTTCTCCAGATTTGGGAGAGGCGCAGAGACAGTGTGTGAGCCGAGCCCTG | |
| | TCTCAGCAATCCACCTGGAGGAGCTAGAGTATCCTCCTCCCTTTACCATTCAGACCGAGAGAAAAAGCCC | |
| | AGCTTGTGTGCACCCTCGTGGGGTTAAGGCGAGCTGTTCCTGGTTTAAAGCCTTTCAGTATTTGTTTTGA | |
| | TGTAAGGCTCTGTGGTTTGGGGGGGAACATCTGTAAACATTATTAGTTGATTTGGGGTTTGTCTTTGATG | |
| | GTTTCTATCTGCAATTATCGTCATGTATATTTAAGTGTCTGTTATAGAAAACCCACACCCACTGTCCTGT | |
| | AAACTTTTCTCAGTGTCCAGACTTTCTGTAATCACATTTTAATTGCCACCTCGTATTTCACCTCTACATT | |
| | TGAAATCTGGCGTCTGTTTCAAGCCAGTGTGTTTTTTCTTCGTTCTGTAATAAACAGCCAGGAGAAAAGT | |
| | GCCTCTATGTTTTTATTTTTCAAGGGAGTATTCAGTACCTACAAACCCAAGTCAGGAAGCCTGCTAGTGG | |
| | CTTTGGTTCTTTCAGAGGCTGCTCGATGCCTTGTGTGTCAGAAAGAAAGATTCAGCAGTTTTGCATCATG | |
| | GCAAAGAAGCCTGTTATTTTGGGGCTCAGCCCCTCATTTTATAGAGGATGAAACAGAGGGGGATGGGAGG | |
| | TCACAAAGACAACTGCCCCGGGAGCAGGTGTGGGGGAGACTTGCCCTGAGGGTCTAGACGCTCTGCACCA | |
| | CCGTCCTGTCTCCCTTGCTGAAGACCACACATGCCCTTCTTTGACCAGACCCTGCCACCTGATAGGCCAG | |
| | GACCTGGTAGGCGGGTACCCAGGTTTCATGGATGGAACCACATCTCCCCAAAAGTGGGGAGGTAGCTACT | |
| | GGGATGCACGCCTCCCGCCATGTGCTATAGGAGAGCAGCTGAAGCAACAGTTGGGATCAGATGTAGTCAC | |
| | AATTGAATGCATCATCACATTTATCCCTCTAAGTGGCTGGGAGAGTTGATATCCTCATCCCTAAGGTACA | |
| | AAATGTTCCAATTTGATCAGTGGCTTTCAGGAGCTGAGAAAGGCATGTGCTCTGAGGCAGAGCTGTTATG | |
| | TCCCGCAGAGCCTAAAAATGCTCTAAGAACATGCTCCCTGCCAAAATTCTCAATGGCTGTGACAAGGGAC | |
| | AACGATCGACCAATGGGGGTGGAAGCAGACCTCCGCAGTCCAGGGGCCAGAGCTAGGACAGAGGGGTCGG | |
| | AGAAAGAGTCATTTTCCCAACACTCCAGCTCTTGGCCAGTCCTCACACAGTCCCCTCCTGCTTCCTGCTG | |
| | AGAGAGATATCCTCATAGGTCTGGGTAAAGTCCTTCAGTCAGCTTTCATTCCCTGTCACCAACTTTGTCT | |
| | CTGTTCTCCCTGCCCGTCTCAGGCAGCACTCCTCAGGAAACCTCTCCAAGAGCCAGCCTCACTGCAGCGC | |
| | CCACTATTGTCCCTCTGCCTCAAGTGTCCCATCCATGCCAGGCCCCAGGCAGGCTGCAGCTTTCCCTCAG | |
| | GGCCACACCAAAGCACTTGGGCTCAGCTGTGCTGTCCCCCTCCATCACTGAGCTCAGGGGCAGCAGGGGT | |
| | GGGGTGCCAGGAGGCCCATTCACCCTTCTCTGGCTCTGTGTTGGACCCACCTGCCCAGCCACTGCTGCTT | |
| | AGAACCTACCCGCTGGGAAAATGAAGCCCTCCCGGAGGGGCCACCTCAACCTGAGAGCCTCACGGATCAC | |
| | AGTTGTCCCCACTCAGCTCTGCCAGCCCTCAGAGACCCATAGATAAAAGCTGAGCTTGGCTCGCAGAGCT | |
| | GGTTCCATCTTCCATTCCCAGAGGGTTCAACTTCCTACCCCAACCACACAGGGAACCTCAAGGCTGAGCC | |
| | AGTGTGGGCTGCAGTGCAGACCAGCTTCCTGGACACGTCCTGCCACCTGACCCCAGGCTGGCCTCACTGC | |
| | CCCTGGCACTCCTGACCCTATCCTCATTCCTCCTGGCAGTGCGTGTTCTGCCATTCCGCTTTCCCTTAGC | |
| | TGTCCTCTCACTGTACTGTCAGCTTCTCCTTTTCCAGGTGCCCCCCAGGGGCTTTCCACATGACCCTGTC | |
| | ACCCCACAGCCCATCCAGCACCAATTCCAGCTCTCTGCCACCCTTCAAAGGAGTGACAGTGCCCTGCTTC | |
| | ACCTCCCACTCACCCCTCAACCCAGAGCAATCTGGCTCCAGTCTTGCCTCCTTCCCCCTAAGTACTCTAG | |
| | TCACAGTTCCAAATTCCTCCTGGTCATAAAGCCAAATGAAGCTTCCTGGTCCTCAGCGGACTTGCCACTT | |
| | CAGCAGTACTGGACTCTCTCCTCCCAGAAACCTGTTTCCCCTTGGCTCCTGGAGCCCACACTCTGCTGGA | |
| | ATCCTTCTGCCTCTCTGGCCTGTAGCCTGGCCCTCTCTCCCAACCTGAGGTCCATTCTCTCCTGCTCCTC | |
| | CACAAGATGTTGCTCCTTCCATTACTTCCTCCCTCTCAACCAAAGCTCCTTCATTAGCTCTTTATCTTCT | |
| | GGTTTCTTCCCCTGGGCAGACGAATGGATTCAAGAGCCTGTGGCCCAGCAGCCCAGCACTCCAGGATCTC | |
| | AGCACTTCAGCATCCCAGTACCCTAGCATCTCAATACCCCAGCACCCCAGCACCATAGTATTCCAGCACC | |
| | CCATTGTCCAAGCATCTCAGCACTCCAGCATCCCAGCACCCCAACACTCCAGCAGCCCAGAATCTCAGCA | |
| | CCCTAGCACTGCAGCATCTCAGGACCCCAGCACTTCAGCATCCCAGCACACTAGTACTCCAGCATCTCGG | |
| | CACCCCAGCACCTAGGCATCCCAACACCCAGCACCCCAGCACTTAAGCATCCCACCACTACAGTATCTCA | |
| | ACACTCCAGCACCCCAGCACCATAGTGTTCCAGCACCCCAGCATCCCAACACCCCAGCACTTAAGCATCC | |
| | CAACACCTCGGCATCCCAACACCCCAGCACTGCAGCATCTCAGCACCTTAGCATCCCAGTGCCCTAGCAT | |
| | CTCAATGCTCCAGCACACCAGTACTACAGTATTCCAGCACCCCAGCACTCCAGCATCTCAGCACTGCAGC | |
| | ACTGCAGCACTCCAGCATCCCAAAATCCCAGCATCCCAACACCCCAGCAGACCAGCAGACCAGCATCTCA | |
| | GCACCGCAGCATCCAAGGACTATCCCAGCATCCCAGCAACCCAGCACCTCAGCATCCCAACACCCCAGCA | |
| | TTTCAGCATGGCAACACCCCAGTACCCCAGCACTTCAGCACCCCAGTATCCCAGCATCTCAGCGACCCAG | |
| | TATCACAAAACCTCAGCATCCTAGCACCCCAGCACCCCAGCACCTTAGCACCTTAGCATCCCAGCATCTC | |
| | AGCGCCTCAGCATCTTGATATTCTGGCTGAGGTCAGCGTGGTGTATCTAGTCAGGGTCCTAACTTTCACT | |
| | TCGCAGGGAAATGCTGCTGGACTGGGTCTCATGTTGGGCTGAAGCTCTCTAGACCCCTTGAAGACAGCAT | |
| | AAAAGAGCTTGGAGACGCTGGGTGTCCCCCATGGAAGAGTTCACTCTCATCCTGCTTTGACAACAGCCTT | |
| | CTCTGGGGTCCCTCACGGGCCCCTCTTTCTTACTGCAAGTTTGTCTCTGAGAAGACTGTGATGCAGAAGT | |
| | CACTCAGCTGCCTGTGGCTCCTGAAGAGCTGAAGGTGGAGGCCTGTAGGCCTCCCTATGAGAGGCGCAGA | |
| | AAAAACCATGATTGCTAGTGGGGAGGTGCTCCCTCTACAACCCACTCCATAATCTGCCCCCGCCCAGCTC | |
| | TGAGGCCAGCCCCAGGGGAAAATGCCAGATCCCCAGGGAGGTGTGTGAGACCTCAGGGGCTCCCTCCTCC | |
| | CTTACAGCAGGCTCAGGCCCCTGGGGGCCTCAGGGCCAAGGTCTGTGGGTAAGCTACTATCTCTCACTTG | |
| | TCCTCTAGCCACAAAAGCCAGGGAGATCTGGCAATGGACATGAGGTTCTGAAGAAGCACATATGACTGGC | |
| | TTCCTAATGCGTGGTTGTTCAGTGATTCAATAAACACGCATGGGCCAGGCATGGGGAAATAGACAAACAT | |
| | GATCCCCAACCTCTCCCAGAGTGAACTGGGAGGGAGGAGTGTTCATCCCTCAGGATTACACCAGAGAAAC | |
| | AAACCAGCAGGAGATATATATGGTTTTGGGGGGTCAAGAAAGAGGAAAAACCTGGCAAGGCAAGTCCAAA | |
| | ATCATAGGACAGGCTGTCAGGAAGGGCAGCCTGGAACCTCTCAAGCAGGAGCTGATGCTGCAGTCCACAG | |
| | GCAGAATTTCTTCTTCCTCGGGGAAATCTCAGCTTTGTTCTTAAGGCCTTTCAACTGATTGGCTGAGGTC | |
| | TGCCCCTTCCCCCACATTCTCCAGGATAATCTTCCTTACTTAAAGTCAACTATTAATCACAGCTACAAAA | |
| | TCCCTTCACAGCTACACATAGATCAGTGTTTGATTGACGAACAGCCCCTACAGCCTAGCCAAGTTGACAC | |
| | ATAAAACTAACCATCACAGGGGGACAAATGATGTAAACACATCAACAAATAAAACAGTAACAAGTTAAGG | |
| | TCTATGGAAAAAACACAGAAGGGGCAGAGAGAAAGAAAGCAAGAAGGAGAGTCCCAGTTTGCTAGGGCTT | |
| | GTGGGAAGTGGGGAGCAGTTCTCTTTAGCTAGGATATTTGGGAAAGGCATATCTGAAGGAGTGATATTTG | |
| | AGCTTAGATTAAAAGATGGGAAGGAGCAAGCCATGCAAAGAGCTAGGATGTTCCAAGCAGAGACGGAACA | |
| | GCAAGTGCAAATGTCAGGAGGAATAGAAGGAGGCTGGTGGGTGGGGTCCAGTGAGCAAGAGGAGGGCAGG | |
| | CAGGAGAGGGGATGGGGAGGTGGGCAGGCCCAGACCACCCAGGGCCCTGGAGACTATCCTGATCCAACAA | |
| | GGGAAGCCTTGAGTCACTTCAGTGTCCATGTGGAGAATGGACCTCAGACTGAATGAGGGAGGCAGTAAGG | |
| | AGGGCCTCTACCTCCAGGGCTTCGCCCTGTGGACTGCGCATAGACATCTCCAACTCAGAAAGTCTGAACC | |
| | AAACTTTCCATAGTTCCCCCAAGTCTGGGCATCCTCCTACTCAGTGAAAGGCAGCCATCACACCTCCCTG | |
| | CCCTGCTCCCGGATGCCCCAAATCCTCTTGGTCTCCAAGTCCAGAACCTGAGACTTGTCCTTGATGTTTG | |
| | TCTTTCCCTCACCCTTTCTGTATTCTGGGAAGATGGGTTTTTTTCCCCCAGATGAATCTGTAAAACTTCT | |
| | GTGATCACAATAAAAATTCTGGCAGTATTATTTTCTGGAACATGACAAAGTGATTCAAAATTATTTATCT | |
| | GGAAGACTACAAAACAAGAATAGCCAGGAAATTTCTAAAAAGAAAGAAGAAGGAGGAGGAGAAAGAAGGA | |
| | GGAGGAAAAGGAGGAGAAGAAGAAAAGAAAAAGAACCAAGAAAGGGTTCTAGCTCTACCAAATATTAAAA | |
| | CATATCATGAAGCTATTTAAAACAATATGGTTGTGGATACTGAAAAAGATGTGAATAAAGTGGAAGGAAA | |
| | ATAAATAGAAATGCACATGGGGATTGAGACTGTGAAAAAGGCAGCATCTCACATCAGTGAGGGATGTTCA | |
| | ACACCTGGTGTTGGGAAAACTGGCTAGTCATTTAAACCAAACAACTGGGTCCTCTACCTCACTCCTGACA | |
| | TTAAGATACATTTAGATGATTCAAAGAGTAAGACAGAAAAAATAACACGTGAAAACACTATCAGAAAACA | |
| | ACGTGGGCCAGGTGTGGTGGGTCACGCCTGTAATCCCAGCACTTTGGGAGGCCGAGGCAGACAGATCACC | |
| | TGAGGTGGGGAGTTCAAGACCAGCCTGACCAACATGGTGAAATCCTGTCTCTACTAAAAATACAAAATTA | |
| | GCTGAGCGTGGTGGCGCATGCCTGTAATCCCAGCTACTCAGGAGGCCGAGGCAGGAGAATCACTTGAACC | |
| | TGGGAGGCAGAGGTTGTGGTGAGCCGAGATCACGCCATTGCACTCCAGCCTGGGCAACAAGAGTGAAAAT | |
| | CCATCTAAAAAAAAAAAAAAAAGCCAAGGTGGATATTTTTATAGTATCAGGGTAGATCAAGCTTCTCCAA | |
| | TCATGACATGAAACCCAGAAACCATAAAAGAAAAGAATGATAAAATTGCCCACGTAAAGTAAAAAGCTTG | |
| | CACACAGAAAAACACCATACAGGTTACAAGATGAGCAGCAAAATCAGAGAAAAAACATTGCAATTCAGGA | |
| | CACACAGAGGCTATTGTTCCTAATATTTAAAAATAAAAGTAGTGGATTGTCTACAAAAAGATGAAGACAA | |
| | GAATTTCAGAAAACCAAATACTGCATGTTTTCACTTACAAGTGGAAGCTAAACACTGAGTACACGTGTAC | |
| | ACAAAGAATGGAACCATAGGCCAGGCACCGTGGCTCACGCCTGTAATCCCAGTACTTTGCGAGGCCGAAG | |
| | CGGGCGGATCACCTGAGGTGAGGAGTTCGAGACCATCCTGGCCAACATGGTGAAACCCAGTCTCTACTAA | |
| | AAATACAAAAATTAGCCGGGCGTGGTGGTGGGTGCCTGTAATCCCAGCTACTCGGGAGGCTGCGGCAGTA | |
| | GAATCGCTTGAACCCTGGAGGTGGACCTTGCAGTGAGCCGAGATCGCACCACTGCACTCCAGCCTGGGCA | |
| | ACAGAGTGAGACTCCATCTCAAAAAAAAAAAAAAGGAATAGAACAATAGACACTGGGGCCTACTTGAGGG | |
| | AGGAGGGTGAGGATCAAAAACCTGCCTATCAGGTACTATGCTTATTACCTGGGTGGTGAAATAATCTGTA | |
| | CACCAAACCCCAGTGACATGCAATTTACCGATGTAACAAACCTGCCCATGTACCCGCTGAACCTAAAATA | |
| | AAAGTTGGAAAAAAATATAGAAATTTTCTTTGTAATAGCCAAAAACTGCAAACAGCCCAGGTGTCTATTA | |
| | GTAGAATGCATAAACAAACTCGGGCATGTTCATACAATGTAAAACTACTCATCAATAAAAAGTGATACTT | |
| | CTCAGCAATGAAAAGAAACTAGCTACTGATACCAGCTACAACATGGATGGATTTCAAGTGCTTTATGATG | |
| | AGAGCAAGAAGCCAGACACAAAAGTGTCTATATATATATACAGTATATATACGTATATATACACATATAT | |
| | ACAGTATATATATACATATACATGTATATATATACTGTATATATACTGTATATATATACACAGTATATAT | |
| | ATACATATATACAGTGTATATATACTGTGTATATATACATGTATATATACTGAGTATATATACATGTATA | |
| | TATATGTATACTGTATATATACTGTATATATATATACACATATATACAGTATATATATACAGTATATACT | |
| | GTATATATACAGTATATACGTGTATATATACATATATACAGTATATATGTAAATATACATATATACAGTA | |
| | TATATGTAAATATACATATATACATGTATATATATACACTATATATATACATATATAGTGTATATATACA | |
| | TATATACATGTATATATTTACTATATGATTCCATTTATATAAAGTGCCAAAACAGTCAAAAATAATCTAT | |
| | GTGGAAAAAATCAACAAAGGGATCCCCCGGGCTGCAGGAATTCGATGGCGCGCCTTAATTAAAATTATCT | |
| | CTAAGGCATGTGAACTGGCTGTCTTGGTTTTCATCTGTACTTCATCTGCTACCTCTGTGACCTGAAACAT | |
| | ATTTATAATTCCATTAAGCTGTGCATATGATAGATTTATCATATGTATTTTCCTTAAAGGATTTTTGTAA | |
| | GAACTAATTGAATTGATACCTGTAAAGTCTTTATCACACTACCCAATAAATAATAAATCTCTTTGTTCAG | |
| | CTCTCTGTTTCTATAAATATGTACCAGTTTTATTGTTTTTAGTGGTAGTGATTTTATTCTCTTTCTATAT | |
| | ATATACACACACATGTGTGCATTCATAAATATATACAATTTTTATGAATAAAAAATTATTAGCAATCAAT | |
| | ATTGAAAACCACTGATTTTTGTTTATGTGAGCAAACAGCAGATTAAAAGGCTAGCCTGCAGGAGTCAATG | |
| | GGAAAAACCCATTGGAGCCAAGTACACTGACTCAATAGGGACTTTCCATTGGGTTTTGCCCAGTACATAA | |
| | GGTCAATAGGGGGTGAGTCAACAGGAAAGTCCCATTGGAGCCAAGTACATTGAGTCAATAGGGACTTTCC | |
| | AATGGGTTTTGCCCAGTACATAAGGTCAATGGGAGGTAAGCCAATGGGTTTTTCCCATTACTGACATGTA | |
| | TACTGAGTCATTAGGGACTTTCCAATGGGTTTTGCCCAGTACATAAGGTCAATAGGGGTGAATCAACAGG | |
| | AAAGTCCCATTGGAGCCAAGTACACTGAGTCAATAGGGACTTTCCATTGGGTTTTGCCCAGTACAAAAGG | |
| | TCAATAGGGGGTGAGTCAATGGGTTTTTCCCATTATTGGCACATACATAAGGTCAATAGGGGTGACTAGT | |
| | GGAGAAGAGCATGCTTGAGGGCTGAGTGCCCCTCAGTGGGCAGAGAGCACATGGCCCACAGTCCCTGAGA | |
| | AGTTGGGGGGAGGGGTGGGCAATTGAACTGGTGCCTAGAGAAGGTGGGGCTTGGGTAAACTGGGAAAGTG | |
| | ATGTGGTGTACTGGCTCCACCTTTTTCCCCAGGGTGGGGGAGAACCATATATAAGTGCAGTAGTCTCTGT | |
| | GAACATTCAAGCATCTGCCTTCTCCCTCCTGTGAGTTTGgtaagtcactgactgtctatgcctgggaaag | |
| | ggtgggcaggaggtggggcagtgcaggaaaagtggcactgtgaaccctgcagccctagacaattgtacta | |
| | accttcttctctttcctctcctgacagGTTGGTGTACAGTAGTAGCAAGCTTGCATGCCTGCAGGTCGAC | |
| | TCTAGACTGCCATGAAGTCTATCTACTTCGTGGCCGGATTGTTTGTTATGCTGGTGCAGGGTTCCTGGCA | |
| | GATTAAGCCTGAGGCTCCCGGAGAAGACGCCAGCCCCGAAGAGCTCAACCGCTACTACGCTAGTCTGCGG | |
| | CACTACCTTAACCTCGTAACAAGACAGCGGTATCGGGCCAAGAGGGCACCCGTGAAACAGACATTGAATT | |
| | TCGACCTGCTGAAACTCGCAGGAGACGTCGAGTCTAATCCTGGACCGATCAAACCTGAAGCCCCCGGTGA | |
| | AGACGCATCACCAGAAGAGTTGAACCGCTATTACGCTTCTCTTAGACACTACTTGAACTTGGTGACACGG | |
| | CAACGGTACTAAGGATCCCCGGGAGATATCCTAGGCTTGGCCAGACATGATAAGATACATTGATGAGTTT | |
| | GGACAAACCACAACTAGAATGCAGTGAAAAAAATGCTTTATTTGTGAAATTTGTGATGCTATTGCTTTAT | |
| | TTGTAACCATTATAAGCTGCAATAAACAAGTTAACAACAACAATTGCATTCATTTTATGTTTCAGGTTCA | |
| | GGGGGAGGTGTGGGAGGTTTTTTAAAGCAAGTAAAACCTCTACAAATGTGGTATGGAATTCAGTCAATAT | |
| | GTTCACCCCAAAAAAGCTGTTTGTTAACTTGCCAACCTCATTCTAAAATGTATATAGAAGCCCAAAAGAC | |
| | AATAACAAAAATATTCTTGTAGAACAAAATGGGAAAGAATGTTCCACTAAATATCAAGATTTAGAGCAAA | |
| | GCATGAGATGTGTGGGGATAGACAGTGAGGCTGATAAAATAGAGTAGAGCTCAGAAACAGACCCATTGAT | |
| | ATATGTAAGTGACCTATGAAAAAAATATGGCATTTTACAATGGGAAAATGATGGTCTTTTTCTTTTTTAG | |
| | AAAAACAGGGAAATATATTTATATGTAAAAAATAAAAGGGAACCCATATGTCATACCATACACACAAAAA | |
| | AATTCCAGTGAATTATAAGTCTAAATGGAGAAGGCAAAACTTTAAATCTTTTAGAAAATAATATAGAAGC | |
| | ATGCCATCAAGACTTCAGTGTAGAGAAAAATTTCTTATGACTCAAAGTCCTAACCACAAAGAAAAGATTG | |
| | TTAATTAGATTGCATGAATATTAAGACTTATTTTTAAAATTAAAAAACCATTAAGAAAAGTCAGGCCATA | |
| | GAATGACAGAAAATATTTGCAACACCCCAGTAAAGAGAATTGTAATATGCAGATTATAAAAAGAAGTCTT | |
| | ACAAATCAGTAAAAAATAAAACTAGACAAAAATTTGAACAGATGAAAGAGAAACTCTAAATAATCATTAC | |
| | ACATGAGAAACTCAATCTCAGAAATCAGAGAACTATCATTGCATATACACTAAATTAGAGAAATATTAAA | |
| | AGGCTAAGTAACATCTGTGGCTTAATTAAGGCGCGCCTACCAGTAAAAAAGAAAACCTATTAAAAAAACA | |
| | CCACTCGACACGGCACCAGCTCAATCAGTCACAGTGTAAAAAAGGGCCAAGTGCAGAGCGAGTATATATA | |
| | GGACTAAAAAATGACGTAACGGTTAAAGTCCACAAAAAACACCCAGAAAACCGCACGCGAACCTACGCCC | |
| | AGAAACGAAAGCCAAAAAACCCACAACTTCCTCAAATCGTCACTTCCGTTTTCCCACGTTACGTCACTTC | |
| | CCATTTTAAGAAAACTACAATTCCCAACACATACAAGTTACTCCGCCCTAAAACCTACGTCACCCGCCCC | |
| | GTTCCCACGCCCCGCGCCACGTCACAAACTCCACCCCCTCATTATCATATTGGCTTCAATCCAAAATAAG | |
| | GTATATTATTGATGATGA | |
| | Description of the elements: | |
| | (1) . . . (103): inverted terminal repeat (ITR) | |
| | (104) . . . (27556): encodes the shuttle vector (P-delta-28) | |
| | (27557) . . . (27564): AscI Restriction enzyme site | |
| | (27565) . . . (27991): encodes CpG free MAR from human beta-globin | |
| | gene | |
| | (27992) . . . (28414): encodes CMV Enhancer | |
| | (28415) . . . (28669): encodes human EF1-alpha promotor | |
| | (28670) . . . (28797): encodes synthetic intron | |
| | (28798) . . . (28851): encodes a multiple cloning site | |
| | (28852) . . . (29202): encodes transgene (PYY) from ATG to stop | |
| | codon | |
| | (29203) . . . (29230): encodes a multiple cloning site | |
| | (29231) . . . (29461): encodes SV40 poly Adenylation signal | |
| | (29462) . . . (30269): encodes MAR 5′ region from human IFN-beta | |
| | gene | |
| | (30270) . . . (30277): AscI Restriction enzyme site | |
| | (30278) . . . (30574): encodes the shuttle vector (P-delta-28) | |
| | (30575) . . . (30678): inverted terminal repeat (ITR) | |
| HDAd-EF1a-PYY | GGCCGATTCATTAATGCAGGGGCCGCTGCGGCCATCATCAATAATATACCTTATTTTGGATTGAAGCCAA | 47 |
| Approach 1 | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding PYY | CCAGGTGTTTTTCTCAGGTGTTTTCCGCGTTCCGGGTCAAAGTTGGCGTTTTATTATTATAGTCAGCTGA | |
| (3-36) | CGTGTAGTGTATTTATACCCGGTGAGTTCCTCAAGAGGCCACTCTTGAGTGCCAGCGAGTAGAGTTTTCT | |
| Approach 1 | CCTCCGAGCCGCTCCGACACCGGGAGGCGCGCCTTAATTAAAATTATCTCTAAGGCATGTGAACTGGCTG | |
| Construct in | TCTTGGTTTTCATCTGTACTTCATCTGCTACCTCTGTGACCTGAAACATATTTATAATTCCATTAAGCTG | |
| HD-MAR-EF1a | TGCATATGATAGATTTATCATATGTATTTTCCTTAAAGGATTTTTGTAAGAACTAATTGAATTGATACCT | |
| shuttle | GTAAAGTCTTTATCACACTACCCAATAAATAATAAATCTCTTTGTTCAGCTCTCTGTTTCTATAAATATG | |
| vector | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGGTATTTG | |
| | TCAGACGACCTTGGCCTGCCCTCACCACGGTGTTGCTCGCCTTGCTCGTTTGCTTGGGGGCCTTGGTGGA | |
| | CGCTATCAAGCCTGAGGCACCCGGAGAAGACGCATCCCCCGAGGAATTGAACCGCTATTACGCATCCTTG | |
| | CGGCACTACCTGAACCTCGTGACCAGGCAACGCTATCGGCACGACTTCCCTGAAGAAGTCGCAATCGTCG | |
| | AAGAGCTCGGCAGGAGAATCAAACCTGAGGCTCCTGGTGAGGACGCCAGTCCGGAGGAACTGAATCGCTA | |
| | CTACGCGTCACTTAGGCACTACCTGAACCTGGTCACTCGCCAGAGATACTAAGGATCCCCGGGAGATATC | |
| | CTAGGCTTGGCCAGACATGATAAGATACATTGATGAGTTTGGACAAACCACAACTAGAATGCAGTGAAAA | |
| | AAATGCTTTATTTGTGAAATTTGTGATGCTATTGCTTTATTTGTAACCATTATAAGCTGCAATAAACAAG | |
| | TTAACAACAACAATTGCATTCATTTTATGTTTCAGGTTCAGGGGGAGGTGTGGGAGGTTTTTTAAAGCAA | |
| | GTAAAACCTCTACAAATGTGGTATGGAATTCAGTCAATATGTTCACCCCAAAAAAGCTGTTTGTTAACTT | |
| | GCCAACCTCATTCTAAAATGTATATAGAAGCCCAAAAGACAATAACAAAAATATTCTTGTAGAACAAAAT | |
| | GGGAAAGAATGTTCCACTAAATATCAAGATTTAGAGCAAAGCATGAGATGTGTGGGGATAGACAGTGAGG | |
| | CTGATAAAATAGAGTAGAGCTCAGAAACAGACCCATTGATATATGTAAGTGACCTATGAAAAAAATATGG | |
| | CATTTTACAATGGGAAAATGATGGTCTTTTTCTTTTTTAGAAAAACAGGGAAATATATTTATATGTAAAA | |
| | AATAAAAGGGAACCCATATGTCATACCATACACACAAAAAAATTCCAGTGAATTATAAGTCTAAATGGAG | |
| | AAGGCAAAACTTTAAATCTTTTAGAAAATAATATAGAAGCATGCCATCAAGACTTCAGTGTAGAGAAAAA | |
| | TTTCTTATGACTCAAAGTCCTAACCACAAAGAAAAGATTGTTAATTAGATTGCATGAATATTAAGACTTA | |
| | TTTTTAAAATTAAAAAACCATTAAGAAAAGTCAGGCCATAGAATGACAGAAAATATTTGCAACACCCCAG | |
| | TAAAGAGAATTGTAATATGCAGATTATAAAAAGAAGTCTTACAAATCAGTAAAAAATAAAACTAGACAAA | |
| | AATTTGAACAGATGAAAGAGAAACTCTAAATAATCATTACACATGAGAAACTCAATCTCAGAAATCAGAG | |
| | AACTATCATTGCATATACACTAAATTAGAGAAATATTAAAAGGCTAAGTAACATCTGTGGCTTAATTAAG | |
| | GCGCGCCGGGCCCCTACGTCACCCGCCCCGTTCCCACGCCCCGCGCCACGTCACAAACTCCACCCCCTCA | |
| | TTATCATATTGGCTTCAATCCAAAATAAGGTATATTATTGATGATGGCCGCAGCGGCCCTGGCGTAATAG | |
| | CGAAGAGGCCCGCACCGATCGCCCTTCCCAACAGTTGCGCAGCCTGAATGGCGAATGGGACGCGCCCTGT | |
| | AGCGGCGCATTAAGCGCGGCGGGTGTGGTGGTTACGCGCAGCGTGACCGCTACACTTGCCAGCGCCCTAG | |
| | CGCCCGCTCCTTTCGCTTTCTTCCCTTCCTTTCTCGCCACGTTCGCCGGCTTTCCCCGTCAAGCTCTAAA | |
| | TCGGGGGCTCCCTTTAGGGTTCCGATTTAGTGCTTTACGGCACCTCGACCCCAAAAAACTTGATTAGGGT | |
| | GATGGTTCACGTAGTGGGCCATCGCCCTGATAGACGGTTTTTCGCCCTTTGACGTTGGAGTCCACGTTCT | |
| | TTAATAGTGGACTCTTGTTCCAAACTGGAACAACACTCAACCCTATCTCGGTCTATTCTTTTGATTTATA | |
| | AGGGATTTTGCCGATTTCGGCCTATTGGTTAAAAAATGAGCTGATTTAACAAAAATTTAACGCGAATTTT | |
| | AACAAAATATTAACGCTTACAATTTAGGTGGCACTTTTCGGGGAAATGTGCGCGGAACCCCTATTTGTTT | |
| | ATTTTTCTAAATACATTCAAATATGTATCCGCTCATGAGACAATAACCCTGATAAATGCTTCAATAATAT | |
| | TGAAAAAGGAAGAGTATGAGTATTCAACATTTCCGTGTCGCCCTTATTCCCTTTTTTGCGGCATTTTGCC | |
| | TTCCTGTTTTTGCTCACCCAGAAACGCTGGTGAAAGTAAAAGATGCTGAAGATCAGTTGGGTGCACGAGT | |
| | GGGTTACATCGAACTGGATCTCAACAGCGGTAAGATCCTTGAGAGTTTTCGCCCCGAAGAACGTTTTCCA | |
| | ATGATGAGCACTTTTAAAGTTCTGCTATGTGGCGCGGTATTATCCCGTATTGACGCCGGGCAAGAGCAAC | |
| | TCGGTCGCCGCATACACTATTCTCAGAATGACTTGGTTGAGTACTCACCAGTCACAGAAAAGCATCTTAC | |
| | GGATGGCATGACAGTAAGAGAATTATGCAGTGCTGCCATAACCATGAGTGATAACACTGCGGCCAACTTA | |
| | CTTCTGACAACGATCGGAGGACCGAAGGAGCTAACCGCTTTTTTGCACAACATGGGGGATCATGTAACTC | |
| | GCCTTGATCGTTGGGAACCGGAGCTGAATGAAGCCATACCAAACGACGAGCGTGACACCACGATGCCTGT | |
| | AGCAATGGCAACAACGTTGCGCAAACTATTAACTGGCGAACTACTTACTCTAGCTTCCCGGCAACAATTA | |
| | ATAGACTGGATGGAGGCGGATAAAGTTGCAGGACCACTTCTGCGCTCGGCCCTTCCGGCTGGCTGGTTTA | |
| | TTGCTGATAAATCTGGAGCCGGTGAGCGTGGGTCTCGCGGTATCATTGCAGCACTGGGGCCAGATGGTAA | |
| | GCCCTCCCGTATCGTAGTTATCTACACGACGGGGAGTCAGGCAACTATGGATGAACGAAATAGACAGATC | |
| | GCTGAGATAGGTGCCTCACTGATTAAGCATTGGTAACTGTCAGACCAAGTTTACTCATATATACTTTAGA | |
| | TTGATTTAAAACTTCATTTTTAATTTAAAAGGATCTAGGTGAAGATCCTTTTTGATAATCTCATGACCAA | |
| | AATCCCTTAACGTGAGTTTTCGTTCCACTGAGCGTCAGACCCCGTAGAAAAGATCAAAGGATCTTCTTGA | |
| | GATCCTTTTTTTCTGCGCGTAATCTGCTGCTTGCAAACAAAAAAACCACCGCTACCAGCGGTGGTTTGTT | |
| | TGCCGGATCAAGAGCTACCAACTCTTTTTCCGAAGGTAACTGGCTTCAGCAGAGCGCAGATACCAAATAC | |
| | TGTTCTTCTAGTGTAGCCGTAGTTAGGCCACCACTTCAAGAACTCTGTAGCACCGCCTACATACCTCGCT | |
| | CTGCTAATCCTGTTACCAGTGGCTGCTGCCAGTGGCGATAAGTCGTGTCTTACCGGGTTGGACTCAAGAC | |
| | GATAGTTACCGGATAAGGCGCAGCGGTCGGGCTGAACGGGGGGTTCGTGCACACAGCCCAGCTTGGAGCG | |
| | AACGACCTACACCGAACTGAGATACCTACAGCGTGAGCTATGAGAAAGCGCCACGCTTCCCGAAGGGAGA | |
| | AAGGCGGACAGGTATCCGGTAAGCGGCAGGGTCGGAACAGGAGAGCGCACGAGGGAGCTTCCAGGGGGAA | |
| | ACGCCTGGTATCTTTATAGTCCTGTCGGGTTTCGCCACCTCTGACTTGAGCGTCGATTTTTGTGATGCTC | |
| | GTCAGGGGGGCGGAGCCTATGGAAAAACGCCAGCAACGCGGCCTTTTTACGGTTCCTGGCCTTTTGCTGG | |
| | CCTTTTGCTCACATGTTCTTTCCTGCGTTATCCCCTGATTCTGTGGATAACCGTATTACCGCCTTTGAGT | |
| | GAGCTGATACCGCTCGCCGCAGCCGAACGACCGAGCGCAGCGAGTCAGTGAGCGAGGAAGCGGAAGAGCG | |
| | CCCAATACGCAAACCGCCTCTCCCCGCGCGTTGGCCGATTCATTAATGCAGGGGCCGCTGCGGCCATCAT | |
| | CAATAATATACCTTATTTTGGATTGAAGCCAATA | |
| | Description of the elements: | |
| | (1) . . . (585): encodes the shuttle vector (pAd) | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2222): encodes transgene (PYY) from ATG to stop codon | |
| | (2223) . . . (2250): encodes a multiple cloning site | |
| | (2251) . . . (2481): encodes SV40 poly Adenylation signal | |
| | (2482) . . . (3289): encodes MAR 5′ region from human IFN-beta gene | |
| | (3290) . . . (3297): AscI Restriction enzyme site | |
| | (3298) . . . (5984): encodes the shuttle vector (pAd) | |
| HDAd-EF1a-PYY | GGCCGATTCATTAATGCAGGGGCCGCTGCGGCCATCATCAATAATATACCTTATTTTGGATTGAAGCCAA | 48 |
| Approach 2 | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding PYY | CCAGGTGTTTTTCTCAGGTGTTTTCCGCGTTCCGGGTCAAAGTTGGCGTTTTATTATTATAGTCAGCTGA | |
| (3-36) | CGTGTAGTGTATTTATACCCGGTGAGTTCCTCAAGAGGCCACTCTTGAGTGCCAGCGAGTAGAGTTTTCT | |
| Approach 2 | CCTCCGAGCCGCTCCGACACCGGGAGGCGCGCCTTAATTAAAATTATCTCTAAGGCATGTGAACTGGCTG | |
| Construct in | TCTTGGTTTTCATCTGTACTTCATCTGCTACCTCTGTGACCTGAAACATATTTATAATTCCATTAAGCTG | |
| HD-MAR-EF1a | TGCATATGATAGATTTATCATATGTATTTTCCTTAAAGGATTTTTGTAAGAACTAATTGAATTGATACCT | |
| shuttle | GTAAAGTCTTTATCACACTACCCAATAAATAATAAATCTCTTTGTTCAGCTCTCTGTTTCTATAAATATG | |
| vector | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGGTCTTTG | |
| | TTAGACGACCGTGGCCGGCTCTGACAACTGTGCTGCTCGCCCTTCTCGTGTGCCTGGGAGCTCTTGTTGA | |
| | TGCCATCAAGCCTGAGGCACCTGGAGAAGACGCATCCCCTGAGGAACTGAACCGCTATTACGCATCATTG | |
| | AGACACTACCTTAACCTCGTGACAAGACAGCGGTACGGTAAGAGAATCAAGCCAGAGGCCCCCGGGGAGG | |
| | ATGCTTCCCCAGAGGAGTTGAATAGGTATTACGCTAGCCTGAGACATTACTTGAACCTTGTGACACGACA | |
| | GCGATATTGAGGATCCCCGGGAGATATCCTAGGCTTGGCCAGACATGATAAGATACATTGATGAGTTTGG | |
| | ACAAACCACAACTAGAATGCAGTGAAAAAAATGCTTTATTTGTGAAATTTGTGATGCTATTGCTTTATTT | |
| | GTAACCATTATAAGCTGCAATAAACAAGTTAACAACAACAATTGCATTCATTTTATGTTTCAGGTTCAGG | |
| | GGGAGGTGTGGGAGGTTTTTTAAAGCAAGTAAAACCTCTACAAATGTGGTATGGAATTCAGTCAATATGT | |
| | TCACCCCAAAAAAGCTGTTTGTTAACTTGCCAACCTCATTCTAAAATGTATATAGAAGCCCAAAAGACAA | |
| | TAACAAAAATATTCTTGTAGAACAAAATGGGAAAGAATGTTCCACTAAATATCAAGATTTAGAGCAAAGC | |
| | ATGAGATGTGTGGGGATAGACAGTGAGGCTGATAAAATAGAGTAGAGCTCAGAAACAGACCCATTGATAT | |
| | ATGTAAGTGACCTATGAAAAAAATATGGCATTTTACAATGGGAAAATGATGGTCTTTTTCTTTTTTAGAA | |
| | AAACAGGGAAATATATTTATATGTAAAAAATAAAAGGGAACCCATATGTCATACCATACACACAAAAAAA | |
| | TTCCAGTGAATTATAAGTCTAAATGGAGAAGGCAAAACTTTAAATCTTTTAGAAAATAATATAGAAGCAT | |
| | GCCATCAAGACTTCAGTGTAGAGAAAAATTTCTTATGACTCAAAGTCCTAACCACAAAGAAAAGATTGTT | |
| | AATTAGATTGCATGAATATTAAGACTTATTTTTAAAATTAAAAAACCATTAAGAAAAGTCAGGCCATAGA | |
| | ATGACAGAAAATATTTGCAACACCCCAGTAAAGAGAATTGTAATATGCAGATTATAAAAAGAAGTCTTAC | |
| | AAATCAGTAAAAAATAAAACTAGACAAAAATTTGAACAGATGAAAGAGAAACTCTAAATAATCATTACAC | |
| | ATGAGAAACTCAATCTCAGAAATCAGAGAACTATCATTGCATATACACTAAATTAGAGAAATATTAAAAG | |
| | GCTAAGTAACATCTGTGGCTTAATTAAGGCGCGCCGGGCCCCTACGTCACCCGCCCCGTTCCCACGCCCC | |
| | GCGCCACGTCACAAACTCCACCCCCTCATTATCATATTGGCTTCAATCCAAAATAAGGTATATTATTGAT | |
| | GATGGCCGCAGCGGCCCTGGCGTAATAGCGAAGAGGCCCGCACCGATCGCCCTTCCCAACAGTTGCGCAG | |
| | CCTGAATGGCGAATGGGACGCGCCCTGTAGCGGCGCATTAAGCGCGGCGGGTGTGGTGGTTACGCGCAGC | |
| | GTGACCGCTACACTTGCCAGCGCCCTAGCGCCCGCTCCTTTCGCTTTCTTCCCTTCCTTTCTCGCCACGT | |
| | TCGCCGGCTTTCCCCGTCAAGCTCTAAATCGGGGGCTCCCTTTAGGGTTCCGATTTAGTGCTTTACGGCA | |
| | CCTCGACCCCAAAAAACTTGATTAGGGTGATGGTTCACGTAGTGGGCCATCGCCCTGATAGACGGTTTTT | |
| | CGCCCTTTGACGTTGGAGTCCACGTTCTTTAATAGTGGACTCTTGTTCCAAACTGGAACAACACTCAACC | |
| | CTATCTCGGTCTATTCTTTTGATTTATAAGGGATTTTGCCGATTTCGGCCTATTGGTTAAAAAATGAGCT | |
| | GATTTAACAAAAATTTAACGCGAATTTTAACAAAATATTAACGCTTACAATTTAGGTGGCACTTTTCGGG | |
| | GAAATGTGCGCGGAACCCCTATTTGTTTATTTTTCTAAATACATTCAAATATGTATCCGCTCATGAGACA | |
| | ATAACCCTGATAAATGCTTCAATAATATTGAAAAAGGAAGAGTATGAGTATTCAACATTTCCGTGTCGCC | |
| | CTTATTCCCTTTTTTGCGGCATTTTGCCTTCCTGTTTTTGCTCACCCAGAAACGCTGGTGAAAGTAAAAG | |
| | ATGCTGAAGATCAGTTGGGTGCACGAGTGGGTTACATCGAACTGGATCTCAACAGCGGTAAGATCCTTGA | |
| | GAGTTTTCGCCCCGAAGAACGTTTTCCAATGATGAGCACTTTTAAAGTTCTGCTATGTGGCGCGGTATTA | |
| | TCCCGTATTGACGCCGGGCAAGAGCAACTCGGTCGCCGCATACACTATTCTCAGAATGACTTGGTTGAGT | |
| | ACTCACCAGTCACAGAAAAGCATCTTACGGATGGCATGACAGTAAGAGAATTATGCAGTGCTGCCATAAC | |
| | CATGAGTGATAACACTGCGGCCAACTTACTTCTGACAACGATCGGAGGACCGAAGGAGCTAACCGCTTTT | |
| | TTGCACAACATGGGGGATCATGTAACTCGCCTTGATCGTTGGGAACCGGAGCTGAATGAAGCCATACCAA | |
| | ACGACGAGCGTGACACCACGATGCCTGTAGCAATGGCAACAACGTTGCGCAAACTATTAACTGGCGAACT | |
| | ACTTACTCTAGCTTCCCGGCAACAATTAATAGACTGGATGGAGGCGGATAAAGTTGCAGGACCACTTCTG | |
| | CGCTCGGCCCTTCCGGCTGGCTGGTTTATTGCTGATAAATCTGGAGCCGGTGAGCGTGGGTCTCGCGGTA | |
| | TCATTGCAGCACTGGGGCCAGATGGTAAGCCCTCCCGTATCGTAGTTATCTACACGACGGGGAGTCAGGC | |
| | AACTATGGATGAACGAAATAGACAGATCGCTGAGATAGGTGCCTCACTGATTAAGCATTGGTAACTGTCA | |
| | GACCAAGTTTACTCATATATACTTTAGATTGATTTAAAACTTCATTTTTAATTTAAAAGGATCTAGGTGA | |
| | AGATCCTTTTTGATAATCTCATGACCAAAATCCCTTAACGTGAGTTTTCGTTCCACTGAGCGTCAGACCC | |
| | CGTAGAAAAGATCAAAGGATCTTCTTGAGATCCTTTTTTTCTGCGCGTAATCTGCTGCTTGCAAACAAAA | |
| | AAACCACCGCTACCAGCGGTGGTTTGTTTGCCGGATCAAGAGCTACCAACTCTTTTTCCGAAGGTAACTG | |
| | GCTTCAGCAGAGCGCAGATACCAAATACTGTTCTTCTAGTGTAGCCGTAGTTAGGCCACCACTTCAAGAA | |
| | CTCTGTAGCACCGCCTACATACCTCGCTCTGCTAATCCTGTTACCAGTGGCTGCTGCCAGTGGCGATAAG | |
| | TCGTGTCTTACCGGGTTGGACTCAAGACGATAGTTACCGGATAAGGCGCAGCGGTCGGGCTGAACGGGGG | |
| | GTTCGTGCACACAGCCCAGCTTGGAGCGAACGACCTACACCGAACTGAGATACCTACAGCGTGAGCTATG | |
| | AGAAAGCGCCACGCTTCCCGAAGGGAGAAAGGCGGACAGGTATCCGGTAAGCGGCAGGGTCGGAACAGGA | |
| | GAGCGCACGAGGGAGCTTCCAGGGGGAAACGCCTGGTATCTTTATAGTCCTGTCGGGTTTCGCCACCTCT | |
| | GACTTGAGCGTCGATTTTTGTGATGCTCGTCAGGGGGGCGGAGCCTATGGAAAAACGCCAGCAACGCGGC | |
| | CTTTTTACGGTTCCTGGCCTTTTGCTGGCCTTTTGCTCACATGTTCTTTCCTGCGTTATCCCCTGATTCT | |
| | GTGGATAACCGTATTACCGCCTTTGAGTGAGCTGATACCGCTCGCCGCAGCCGAACGACCGAGCGCAGCG | |
| | AGTCAGTGAGCGAGGAAGCGGAAGAGCGCCCAATACGCAAACCGCCTCTCCCCGCGCGTTGGCCGATTCA | |
| | TTAATGCAGGGGCCGCTGCGGCCATCATCAATAATATACCTTATTTTGGATTGAAGCCAATA | |
| | Description of the elements: | |
| | (1) . . . (585): encodes the shuttle vector (pAd) | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2180): encodes transgene (PYY) from ATG to stop codon | |
| | (2182) . . . (2208): encodes a multiple cloning site | |
| | (2209) . . . (2439): encodes SV40 poly Adenylation signal | |
| | (2440) . . . (3247): encodes MAR 5′ region from human IFN-beta gene | |
| | (3248) . . . (3255): AscI Restriction enzyme site | |
| | (3256) . . . (5942): encodes the shuttle vector (pAd) | |
| HDAd-EF1a-PYY | GGCCGATTCATTAATGCAGGGGCCGCTGCGGCCATCATCAATAATATACCTTATTTTGGATTGAAGCCAA | 49 |
| Approach 3 | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding PYY | CCAGGTGTTTTTCTCAGGTGTTTTCCGCGTTCCGGGTCAAAGTTGGCGTTTTATTATTATAGTCAGCTGA | |
| (3-36) | CGTGTAGTGTATTTATACCCGGTGAGTTCCTCAAGAGGCCACTCTTGAGTGCCAGCGAGTAGAGTTTTCT | |
| Approach 3 | CCTCCGAGCCGCTCCGACACCGGGAGGCGCGCCTTAATTAAAATTATCTCTAAGGCATGTGAACTGGCTG | |
| Construct in | TCTTGGTTTTCATCTGTACTTCATCTGCTACCTCTGTGACCTGAAACATATTTATAATTCCATTAAGCTG | |
| HD-MAR-EF1a | TGCATATGATAGATTTATCATATGTATTTTCCTTAAAGGATTTTTGTAAGAACTAATTGAATTGATACCT | |
| shuttle | GTAAAGTCTTTATCACACTACCCAATAAATAATAAATCTCTTTGTTCAGCTCTCTGTTTCTATAAATATG | |
| vector | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGGTATTTG | |
| | TGAGACGCCCCTGGCCCGCGCTGACAACCGTGTTGCTGGCACTTCTTGTGTGCCTTGGAGCTCTCGTTGA | |
| | TGCCATCAAGCCTGAAGCACCGGGGGAAGACGCTTCCCCCGAGGAGCTCAATCGATACTATGCTAGTCTG | |
| | CGACATTATCTGAACCTTGTGACCAGGCAGCGGTACGGCAAACGGGATTTTCCAGAGGAAGTGGCGATCG | |
| | TCGAGGAGCTCGGACGGCGGGGAAAACGCATCAAACCCGAGGCCCCAGGCGAGGATGCTTCACCTGAGGA | |
| | ACTTAACCGCTACTACGCAAGCCTGCGGCATTACCTTAACCTCGTGACCAGACAGCGCTACTAAGGATCC | |
| | CCGGGAGATATCCTAGGCTTGGCCAGACATGATAAGATACATTGATGAGTTTGGACAAACCACAACTAGA | |
| | ATGCAGTGAAAAAAATGCTTTATTTGTGAAATTTGTGATGCTATTGCTTTATTTGTAACCATTATAAGCT | |
| | GCAATAAACAAGTTAACAACAACAATTGCATTCATTTTATGTTTCAGGTTCAGGGGGAGGTGTGGGAGGT | |
| | TTTTTAAAGCAAGTAAAACCTCTACAAATGTGGTATGGAATTCAGTCAATATGTTCACCCCAAAAAAGCT | |
| | GTTTGTTAACTTGCCAACCTCATTCTAAAATGTATATAGAAGCCCAAAAGACAATAACAAAAATATTCTT | |
| | GTAGAACAAAATGGGAAAGAATGTTCCACTAAATATCAAGATTTAGAGCAAAGCATGAGATGTGTGGGGA | |
| | TAGACAGTGAGGCTGATAAAATAGAGTAGAGCTCAGAAACAGACCCATTGATATATGTAAGTGACCTATG | |
| | AAAAAAATATGGCATTTTACAATGGGAAAATGATGGTCTTTTTCTTTTTTAGAAAAACAGGGAAATATAT | |
| | TTATATGTAAAAAATAAAAGGGAACCCATATGTCATACCATACACACAAAAAAATTCCAGTGAATTATAA | |
| | GTCTAAATGGAGAAGGCAAAACTTTAAATCTTTTAGAAAATAATATAGAAGCATGCCATCAAGACTTCAG | |
| | TGTAGAGAAAAATTTCTTATGACTCAAAGTCCTAACCACAAAGAAAAGATTGTTAATTAGATTGCATGAA | |
| | TATTAAGACTTATTTTTAAAATTAAAAAACCATTAAGAAAAGTCAGGCCATAGAATGACAGAAAATATTT | |
| | GCAACACCCCAGTAAAGAGAATTGTAATATGCAGATTATAAAAAGAAGTCTTACAAATCAGTAAAAAATA | |
| | AAACTAGACAAAAATTTGAACAGATGAAAGAGAAACTCTAAATAATCATTACACATGAGAAACTCAATCT | |
| | CAGAAATCAGAGAACTATCATTGCATATACACTAAATTAGAGAAATATTAAAAGGCTAAGTAACATCTGT | |
| | GGCTTAATTAAGGCGCGCCGGGCCCCTACGTCACCCGCCCCGTTCCCACGCCCCGCGCCACGTCACAAAC | |
| | TCCACCCCCTCATTATCATATTGGCTTCAATCCAAAATAAGGTATATTATTGATGATGGCCGCAGCGGCC | |
| | CTGGCGTAATAGCGAAGAGGCCCGCACCGATCGCCCTTCCCAACAGTTGCGCAGCCTGAATGGCGAATGG | |
| | GACGCGCCCTGTAGCGGCGCATTAAGCGCGGCGGGTGTGGTGGTTACGCGCAGCGTGACCGCTACACTTG | |
| | CCAGCGCCCTAGCGCCCGCTCCTTTCGCTTTCTTCCCTTCCTTTCTCGCCACGTTCGCCGGCTTTCCCCG | |
| | TCAAGCTCTAAATCGGGGGCTCCCTTTAGGGTTCCGATTTAGTGCTTTACGGCACCTCGACCCCAAAAAA | |
| | CTTGATTAGGGTGATGGTTCACGTAGTGGGCCATCGCCCTGATAGACGGTTTTTCGCCCTTTGACGTTGG | |
| | AGTCCACGTTCTTTAATAGTGGACTCTTGTTCCAAACTGGAACAACACTCAACCCTATCTCGGTCTATTC | |
| | TTTTGATTTATAAGGGATTTTGCCGATTTCGGCCTATTGGTTAAAAAATGAGCTGATTTAACAAAAATTT | |
| | AACGCGAATTTTAACAAAATATTAACGCTTACAATTTAGGTGGCACTTTTCGGGGAAATGTGCGCGGAAC | |
| | CCCTATTTGTTTATTTTTCTAAATACATTCAAATATGTATCCGCTCATGAGACAATAACCCTGATAAATG | |
| | CTTCAATAATATTGAAAAAGGAAGAGTATGAGTATTCAACATTTCCGTGTCGCCCTTATTCCCTTTTTTG | |
| | CGGCATTTTGCCTTCCTGTTTTTGCTCACCCAGAAACGCTGGTGAAAGTAAAAGATGCTGAAGATCAGTT | |
| | GGGTGCACGAGTGGGTTACATCGAACTGGATCTCAACAGCGGTAAGATCCTTGAGAGTTTTCGCCCCGAA | |
| | GAACGTTTTCCAATGATGAGCACTTTTAAAGTTCTGCTATGTGGCGCGGTATTATCCCGTATTGACGCCG | |
| | GGCAAGAGCAACTCGGTCGCCGCATACACTATTCTCAGAATGACTTGGTTGAGTACTCACCAGTCACAGA | |
| | AAAGCATCTTACGGATGGCATGACAGTAAGAGAATTATGCAGTGCTGCCATAACCATGAGTGATAACACT | |
| | GCGGCCAACTTACTTCTGACAACGATCGGAGGACCGAAGGAGCTAACCGCTTTTTTGCACAACATGGGGG | |
| | ATCATGTAACTCGCCTTGATCGTTGGGAACCGGAGCTGAATGAAGCCATACCAAACGACGAGCGTGACAC | |
| | CACGATGCCTGTAGCAATGGCAACAACGTTGCGCAAACTATTAACTGGCGAACTACTTACTCTAGCTTCC | |
| | CGGCAACAATTAATAGACTGGATGGAGGCGGATAAAGTTGCAGGACCACTTCTGCGCTCGGCCCTTCCGG | |
| | CTGGCTGGTTTATTGCTGATAAATCTGGAGCCGGTGAGCGTGGGTCTCGCGGTATCATTGCAGCACTGGG | |
| | GCCAGATGGTAAGCCCTCCCGTATCGTAGTTATCTACACGACGGGGAGTCAGGCAACTATGGATGAACGA | |
| | AATAGACAGATCGCTGAGATAGGTGCCTCACTGATTAAGCATTGGTAACTGTCAGACCAAGTTTACTCAT | |
| | ATATACTTTAGATTGATTTAAAACTTCATTTTTAATTTAAAAGGATCTAGGTGAAGATCCTTTTTGATAA | |
| | TCTCATGACCAAAATCCCTTAACGTGAGTTTTCGTTCCACTGAGCGTCAGACCCCGTAGAAAAGATCAAA | |
| | GGATCTTCTTGAGATCCTTTTTTTCTGCGCGTAATCTGCTGCTTGCAAACAAAAAAACCACCGCTACCAG | |
| | CGGTGGTTTGTTTGCCGGATCAAGAGCTACCAACTCTTTTTCCGAAGGTAACTGGCTTCAGCAGAGCGCA | |
| | GATACCAAATACTGTTCTTCTAGTGTAGCCGTAGTTAGGCCACCACTTCAAGAACTCTGTAGCACCGCCT | |
| | ACATACCTCGCTCTGCTAATCCTGTTACCAGTGGCTGCTGCCAGTGGCGATAAGTCGTGTCTTACCGGGT | |
| | TGGACTCAAGACGATAGTTACCGGATAAGGCGCAGCGGTCGGGCTGAACGGGGGGTTCGTGCACACAGCC | |
| | CAGCTTGGAGCGAACGACCTACACCGAACTGAGATACCTACAGCGTGAGCTATGAGAAAGCGCCACGCTT | |
| | CCCGAAGGGAGAAAGGCGGACAGGTATCCGGTAAGCGGCAGGGTCGGAACAGGAGAGCGCACGAGGGAGC | |
| | TTCCAGGGGGAAACGCCTGGTATCTTTATAGTCCTGTCGGGTTTCGCCACCTCTGACTTGAGCGTCGATT | |
| | TTTGTGATGCTCGTCAGGGGGGCGGAGCCTATGGAAAAACGCCAGCAACGCGGCCTTTTTACGGTTCCTG | |
| | GCCTTTTGCTGGCCTTTTGCTCACATGTTCTTTCCTGCGTTATCCCCTGATTCTGTGGATAACCGTATTA | |
| | CCGCCTTTGAGTGAGCTGATACCGCTCGCCGCAGCCGAACGACCGAGCGCAGCGAGTCAGTGAGCGAGGA | |
| | AGCGGAAGAGCGCCCAATACGCAAACCGCCTCTCCCCGCGCGTTGGCCGATTCATTAATGCAGGGGCCGC | |
| | TGCGGCCATCATCAATAATATACCTTATTTTGGATTGAAGCCAATA | |
| | Description of the elements: | |
| | (1) . . . (585): encodes the shuttle vector (pAd) | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2234): encodes transgene (PYY) from ATG to stop codon | |
| | (2182) . . . (2262): encodes a multiple cloning site | |
| | (2263) . . . (2493): encodes SV40 poly Adenylation signal | |
| | (2494) . . . (3301): encodes MAR 5′ region from human IFN-beta gene | |
| | (3302) . . . (3309): AscI Restriction enzyme site | |
| | (3310) . . . (5996): encodes the shuttle vector (pAd) | |
| HDAd-EF1a-PYY | GGCCGATTCATTAATGCAGGGGCCGCTGCGGCCATCATCAATAATATACCTTATTTTGGATTGAAGCCAA | 50 |
| Approach 4 | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding PYY | CCAGGTGTTTTTCTCAGGTGTTTTCCGCGTTCCGGGTCAAAGTTGGCGTTTTATTATTATAGTCAGCTGA | |
| (3-36) | CGTGTAGTGTATTTATACCCGGTGAGTTCCTCAAGAGGCCACTCTTGAGTGCCAGCGAGTAGAGTTTTCT | |
| Approach 4 | CCTCCGAGCCGCTCCGACACCGGGAGGCGCGCCTTAATTAAAATTATCTCTAAGGCATGTGAACTGGCTG | |
| Construct in | TCTTGGTTTTCATCTGTACTTCATCTGCTACCTCTGTGACCTGAAACATATTTATAATTCCATTAAGCTG | |
| HD-MAR-EF1a | TGCATATGATAGATTTATCATATGTATTTTCCTTAAAGGATTTTTGTAAGAACTAATTGAATTGATACCT | |
| shuttle | GTAAAGTCTTTATCACACTACCCAATAAATAATAAATCTCTTTGTTCAGCTCTCTGTTTCTATAAATATG | |
| vector | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGAAGTCCA | |
| | TCTACTTCGTGGCTGGCCTGTTTGTGATGCTGGTGCAGGGTAGCTGGCAGATCAAGCCAGAGGCACCTGG | |
| | GGAGGACGCATCACCAGAAGAATTGAACCGGTATTACGCGTCACTCCGCCACTATCTTAACCTGGTCACG | |
| | AGGCAGAGATACCGCGCAAAGAGAGCTCCTGTCAAGCAGACACTCAACTTTGACCTGCTGAAGCTGGCAG | |
| | GAGACGTAGAGTCCAATCCCGGTCCTATGAAGAGCATCTACTTTGTGGCTGGGTTGTTTGTGATGCTGGT | |
| | CCAAGGGTCCTGGCAGATCAAGCCCGAGGCCCCCGGGGAGGATGCCAGTCCAGAGGAGCTGAATCGATAT | |
| | TACGCCAGCCTGCGGCACTACCTCAACCTTGTAACACGACAGAGATACTAAGGATCCCCGGGAGATATCC | |
| | TAGGCTTGGCCAGACATGATAAGATACATTGATGAGTTTGGACAAACCACAACTAGAATGCAGTGAAAAA | |
| | AATGCTTTATTTGTGAAATTTGTGATGCTATTGCTTTATTTGTAACCATTATAAGCTGCAATAAACAAGT | |
| | TAACAACAACAATTGCATTCATTTTATGTTTCAGGTTCAGGGGGAGGTGTGGGAGGTTTTTTAAAGCAAG | |
| | TAAAACCTCTACAAATGTGGTATGGAATTCAGTCAATATGTTCACCCCAAAAAAGCTGTTTGTTAACTTG | |
| | CCAACCTCATTCTAAAATGTATATAGAAGCCCAAAAGACAATAACAAAAATATTCTTGTAGAACAAAATG | |
| | GGAAAGAATGTTCCACTAAATATCAAGATTTAGAGCAAAGCATGAGATGTGTGGGGATAGACAGTGAGGC | |
| | TGATAAAATAGAGTAGAGCTCAGAAACAGACCCATTGATATATGTAAGTGACCTATGAAAAAAATATGGC | |
| | ATTTTACAATGGGAAAATGATGGTCTTTTTCTTTTTTAGAAAAACAGGGAAATATATTTATATGTAAAAA | |
| | ATAAAAGGGAACCCATATGTCATACCATACACACAAAAAAATTCCAGTGAATTATAAGTCTAAATGGAGA | |
| | AGGCAAAACTTTAAATCTTTTAGAAAATAATATAGAAGCATGCCATCAAGACTTCAGTGTAGAGAAAAAT | |
| | TTCTTATGACTCAAAGTCCTAACCACAAAGAAAAGATTGTTAATTAGATTGCATGAATATTAAGACTTAT | |
| | TTTTAAAATTAAAAAACCATTAAGAAAAGTCAGGCCATAGAATGACAGAAAATATTTGCAACACCCCAGT | |
| | AAAGAGAATTGTAATATGCAGATTATAAAAAGAAGTCTTACAAATCAGTAAAAAATAAAACTAGACAAAA | |
| | ATTTGAACAGATGAAAGAGAAACTCTAAATAATCATTACACATGAGAAACTCAATCTCAGAAATCAGAGA | |
| | ACTATCATTGCATATACACTAAATTAGAGAAATATTAAAAGGCTAAGTAACATCTGTGGCTTAATTAAGG | |
| | CGCGCCGGGCCCCTACGTCACCCGCCCCGTTCCCACGCCCCGCGCCACGTCACAAACTCCACCCCCTCAT | |
| | TATCATATTGGCTTCAATCCAAAATAAGGTATATTATTGATGATGGCCGCAGCGGCCCTGGCGTAATAGC | |
| | GAAGAGGCCCGCACCGATCGCCCTTCCCAACAGTTGCGCAGCCTGAATGGCGAATGGGACGCGCCCTGTA | |
| | GCGGCGCATTAAGCGCGGCGGGTGTGGTGGTTACGCGCAGCGTGACCGCTACACTTGCCAGCGCCCTAGC | |
| | GCCCGCTCCTTTCGCTTTCTTCCCTTCCTTTCTCGCCACGTTCGCCGGCTTTCCCCGTCAAGCTCTAAAT | |
| | CGGGGGCTCCCTTTAGGGTTCCGATTTAGTGCTTTACGGCACCTCGACCCCAAAAAACTTGATTAGGGTG | |
| | ATGGTTCACGTAGTGGGCCATCGCCCTGATAGACGGTTTTTCGCCCTTTGACGTTGGAGTCCACGTTCTT | |
| | TAATAGTGGACTCTTGTTCCAAACTGGAACAACACTCAACCCTATCTCGGTCTATTCTTTTGATTTATAA | |
| | GGGATTTTGCCGATTTCGGCCTATTGGTTAAAAAATGAGCTGATTTAACAAAAATTTAACGCGAATTTTA | |
| | ACAAAATATTAACGCTTACAATTTAGGTGGCACTTTTCGGGGAAATGTGCGCGGAACCCCTATTTGTTTA | |
| | TTTTTCTAAATACATTCAAATATGTATCCGCTCATGAGACAATAACCCTGATAAATGCTTCAATAATATT | |
| | GAAAAAGGAAGAGTATGAGTATTCAACATTTCCGTGTCGCCCTTATTCCCTTTTTTGCGGCATTTTGCCT | |
| | TCCTGTTTTTGCTCACCCAGAAACGCTGGTGAAAGTAAAAGATGCTGAAGATCAGTTGGGTGCACGAGTG | |
| | GGTTACATCGAACTGGATCTCAACAGCGGTAAGATCCTTGAGAGTTTTCGCCCCGAAGAACGTTTTCCAA | |
| | TGATGAGCACTTTTAAAGTTCTGCTATGTGGCGCGGTATTATCCCGTATTGACGCCGGGCAAGAGCAACT | |
| | CGGTCGCCGCATACACTATTCTCAGAATGACTTGGTTGAGTACTCACCAGTCACAGAAAAGCATCTTACG | |
| | GATGGCATGACAGTAAGAGAATTATGCAGTGCTGCCATAACCATGAGTGATAACACTGCGGCCAACTTAC | |
| | TTCTGACAACGATCGGAGGACCGAAGGAGCTAACCGCTTTTTTGCACAACATGGGGGATCATGTAACTCG | |
| | CCTTGATCGTTGGGAACCGGAGCTGAATGAAGCCATACCAAACGACGAGCGTGACACCACGATGCCTGTA | |
| | GCAATGGCAACAACGTTGCGCAAACTATTAACTGGCGAACTACTTACTCTAGCTTCCCGGCAACAATTAA | |
| | TAGACTGGATGGAGGCGGATAAAGTTGCAGGACCACTTCTGCGCTCGGCCCTTCCGGCTGGCTGGTTTAT | |
| | TGCTGATAAATCTGGAGCCGGTGAGCGTGGGTCTCGCGGTATCATTGCAGCACTGGGGCCAGATGGTAAG | |
| | CCCTCCCGTATCGTAGTTATCTACACGACGGGGAGTCAGGCAACTATGGATGAACGAAATAGACAGATCG | |
| | CTGAGATAGGTGCCTCACTGATTAAGCATTGGTAACTGTCAGACCAAGTTTACTCATATATACTTTAGAT | |
| | TGATTTAAAACTTCATTTTTAATTTAAAAGGATCTAGGTGAAGATCCTTTTTGATAATCTCATGACCAAA | |
| | ATCCCTTAACGTGAGTTTTCGTTCCACTGAGCGTCAGACCCCGTAGAAAAGATCAAAGGATCTTCTTGAG | |
| | ATCCTTTTTTTCTGCGCGTAATCTGCTGCTTGCAAACAAAAAAACCACCGCTACCAGCGGTGGTTTGTTT | |
| | GCCGGATCAAGAGCTACCAACTCTTTTTCCGAAGGTAACTGGCTTCAGCAGAGCGCAGATACCAAATACT | |
| | GTTCTTCTAGTGTAGCCGTAGTTAGGCCACCACTTCAAGAACTCTGTAGCACCGCCTACATACCTCGCTC | |
| | TGCTAATCCTGTTACCAGTGGCTGCTGCCAGTGGCGATAAGTCGTGTCTTACCGGGTTGGACTCAAGACG | |
| | ATAGTTACCGGATAAGGCGCAGCGGTCGGGCTGAACGGGGGGTTCGTGCACACAGCCCAGCTTGGAGCGA | |
| | ACGACCTACACCGAACTGAGATACCTACAGCGTGAGCTATGAGAAAGCGCCACGCTTCCCGAAGGGAGAA | |
| | AGGCGGACAGGTATCCGGTAAGCGGCAGGGTCGGAACAGGAGAGCGCACGAGGGAGCTTCCAGGGGGAAA | |
| | CGCCTGGTATCTTTATAGTCCTGTCGGGTTTCGCCACCTCTGACTTGAGCGTCGATTTTTGTGATGCTCG | |
| | TCAGGGGGGCGGAGCCTATGGAAAAACGCCAGCAACGCGGCCTTTTTACGGTTCCTGGCCTTTTGCTGGC | |
| | CTTTTGCTCACATGTTCTTTCCTGCGTTATCCCCTGATTCTGTGGATAACCGTATTACCGCCTTTGAGTG | |
| | AGCTGATACCGCTCGCCGCAGCCGAACGACCGAGCGCAGCGAGTCAGTGAGCGAGGAAGCGGAAGAGCGC | |
| | CCAATACGCAAACCGCCTCTCCCCGCGCGTTGGCCGATTCATTAATGCAGGGGCCGCTGCGGCCATCATC | |
| | AATAATATACCTTATTTTGGATTGAAGCCAATA | |
| | Description of the elements: | |
| | (1) . . . (585): encodes the shuttle vector (pAd) | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2291): encodes transgene (PYY) from ATG to stop codon | |
| | (2292) . . . (2319): encodes a multiple cloning site | |
| | (2320) . . . (2550): encodes SV40 poly Adenylation signal | |
| | (2551) . . . (3358): encodes MAR 5′ region from human IFN-beta gene | |
| | (3359) . . . (3366): AscI Restriction enzyme site | |
| | (3367) . . . (6053): encodes the shuttle vector (pAd) | |
| HDAd-EF1a-PYY | GGCCGATTCATTAATGCAGGGGCCGCTGCGGCCATCATCAATAATATACCTTATTTTGGATTGAAGCCAA | 51 |
| Approach 5 | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding PYY | CCAGGTGTTTTTCTCAGGTGTTTTCCGCGTTCCGGGTCAAAGTTGGCGTTTTATTATTATAGTCAGCTGA | |
| (3-36) | CGTGTAGTGTATTTATACCCGGTGAGTTCCTCAAGAGGCCACTCTTGAGTGCCAGCGAGTAGAGTTTTCT | |
| Approach 5 | CCTCCGAGCCGCTCCGACACCGGGAGGCGCGCCTTAATTAAAATTATCTCTAAGGCATGTGAACTGGCTG | |
| Construct in | TCTTGGTTTTCATCTGTACTTCATCTGCTACCTCTGTGACCTGAAACATATTTATAATTCCATTAAGCTG | |
| HD-MAR-EF1a | TGCATATGATAGATTTATCATATGTATTTTCCTTAAAGGATTTTTGTAAGAACTAATTGAATTGATACCT | |
| shuttle | GTAAAGTCTTTATCACACTACCCAATAAATAATAAATCTCTTTGTTCAGCTCTCTGTTTCTATAAATATG | |
| vector | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGAAGTCAA | |
| | TCTATTTCGTGGCGGGCCTGTTCGTAATGCTGGTGCAGGGCTCTTGGCAGATCAAGCCTGAAGCGCCTGG | |
| | AGAGGACGCATCCCCTGAAGAACTGAATCGCTACTATGCCAGCTTGCGCCATTATCTGAACTTGGTGACT | |
| | AGACAGCGCTATAGAGCCAAGAGGGCTCCCGTGAAACAAACACTGAATTTTGATTTGCTTAAGCTCGCCG | |
| | GGGACGTGGAAAGCAATCCTGGCCCCGACTTTCCTGAGGAAGTAGCTATCGTGGAAGAGCTCGGAAGGCG | |
| | AATGAAATCCATTTATTTTGTCGCTGGCCTGTTTGTGATGCTGGTTCAGGGGAGTTGGCAAATCAAGCCC | |
| | GAGGCGCCCGGCGAAGACGCGTCCCCTGAAGAGTTGAACAGGTACTATGCCAGCCTGAGGCACTATCTTA | |
| | ACCTGGTGACGAGGCAAAGATACTAAGGATCCCCGGGAGATATCCTAGGCTTGGCCAGACATGATAAGAT | |
| | ACATTGATGAGTTTGGACAAACCACAACTAGAATGCAGTGAAAAAAATGCTTTATTTGTGAAATTTGTGA | |
| | TGCTATTGCTTTATTTGTAACCATTATAAGCTGCAATAAACAAGTTAACAACAACAATTGCATTCATTTT | |
| | ATGTTTCAGGTTCAGGGGGAGGTGTGGGAGGTTTTTTAAAGCAAGTAAAACCTCTACAAATGTGGTATGG | |
| | AATTCAGTCAATATGTTCACCCCAAAAAAGCTGTTTGTTAACTTGCCAACCTCATTCTAAAATGTATATA | |
| | GAAGCCCAAAAGACAATAACAAAAATATTCTTGTAGAACAAAATGGGAAAGAATGTTCCACTAAATATCA | |
| | AGATTTAGAGCAAAGCATGAGATGTGTGGGGATAGACAGTGAGGCTGATAAAATAGAGTAGAGCTCAGAA | |
| | ACAGACCCATTGATATATGTAAGTGACCTATGAAAAAAATATGGCATTTTACAATGGGAAAATGATGGTC | |
| | TTTTTCTTTTTTAGAAAAACAGGGAAATATATTTATATGTAAAAAATAAAAGGGAACCCATATGTCATAC | |
| | CATACACACAAAAAAATTCCAGTGAATTATAAGTCTAAATGGAGAAGGCAAAACTTTAAATCTTTTAGAA | |
| | AATAATATAGAAGCATGCCATCAAGACTTCAGTGTAGAGAAAAATTTCTTATGACTCAAAGTCCTAACCA | |
| | CAAAGAAAAGATTGTTAATTAGATTGCATGAATATTAAGACTTATTTTTAAAATTAAAAAACCATTAAGA | |
| | AAAGTCAGGCCATAGAATGACAGAAAATATTTGCAACACCCCAGTAAAGAGAATTGTAATATGCAGATTA | |
| | TAAAAAGAAGTCTTACAAATCAGTAAAAAATAAAACTAGACAAAAATTTGAACAGATGAAAGAGAAACTC | |
| | TAAATAATCATTACACATGAGAAACTCAATCTCAGAAATCAGAGAACTATCATTGCATATACACTAAATT | |
| | AGAGAAATATTAAAAGGCTAAGTAACATCTGTGGCTTAATTAAGGCGCGCCGGGCCCCTACGTCACCCGC | |
| | CCCGTTCCCACGCCCCGCGCCACGTCACAAACTCCACCCCCTCATTATCATATTGGCTTCAATCCAAAAT | |
| | AAGGTATATTATTGATGATGGCCGCAGCGGCCCTGGCGTAATAGCGAAGAGGCCCGCACCGATCGCCCTT | |
| | CCCAACAGTTGCGCAGCCTGAATGGCGAATGGGACGCGCCCTGTAGCGGCGCATTAAGCGCGGCGGGTGT | |
| | GGTGGTTACGCGCAGCGTGACCGCTACACTTGCCAGCGCCCTAGCGCCCGCTCCTTTCGCTTTCTTCCCT | |
| | TCCTTTCTCGCCACGTTCGCCGGCTTTCCCCGTCAAGCTCTAAATCGGGGGCTCCCTTTAGGGTTCCGAT | |
| | TTAGTGCTTTACGGCACCTCGACCCCAAAAAACTTGATTAGGGTGATGGTTCACGTAGTGGGCCATCGCC | |
| | CTGATAGACGGTTTTTCGCCCTTTGACGTTGGAGTCCACGTTCTTTAATAGTGGACTCTTGTTCCAAACT | |
| | GGAACAACACTCAACCCTATCTCGGTCTATTCTTTTGATTTATAAGGGATTTTGCCGATTTCGGCCTATT | |
| | GGTTAAAAAATGAGCTGATTTAACAAAAATTTAACGCGAATTTTAACAAAATATTAACGCTTACAATTTA | |
| | GGTGGCACTTTTCGGGGAAATGTGCGCGGAACCCCTATTTGTTTATTTTTCTAAATACATTCAAATATGT | |
| | ATCCGCTCATGAGACAATAACCCTGATAAATGCTTCAATAATATTGAAAAAGGAAGAGTATGAGTATTCA | |
| | ACATTTCCGTGTCGCCCTTATTCCCTTTTTTGCGGCATTTTGCCTTCCTGTTTTTGCTCACCCAGAAACG | |
| | CTGGTGAAAGTAAAAGATGCTGAAGATCAGTTGGGTGCACGAGTGGGTTACATCGAACTGGATCTCAACA | |
| | GCGGTAAGATCCTTGAGAGTTTTCGCCCCGAAGAACGTTTTCCAATGATGAGCACTTTTAAAGTTCTGCT | |
| | ATGTGGCGCGGTATTATCCCGTATTGACGCCGGGCAAGAGCAACTCGGTCGCCGCATACACTATTCTCAG | |
| | AATGACTTGGTTGAGTACTCACCAGTCACAGAAAAGCATCTTACGGATGGCATGACAGTAAGAGAATTAT | |
| | GCAGTGCTGCCATAACCATGAGTGATAACACTGCGGCCAACTTACTTCTGACAACGATCGGAGGACCGAA | |
| | GGAGCTAACCGCTTTTTTGCACAACATGGGGGATCATGTAACTCGCCTTGATCGTTGGGAACCGGAGCTG | |
| | AATGAAGCCATACCAAACGACGAGCGTGACACCACGATGCCTGTAGCAATGGCAACAACGTTGCGCAAAC | |
| | TATTAACTGGCGAACTACTTACTCTAGCTTCCCGGCAACAATTAATAGACTGGATGGAGGCGGATAAAGT | |
| | TGCAGGACCACTTCTGCGCTCGGCCCTTCCGGCTGGCTGGTTTATTGCTGATAAATCTGGAGCCGGTGAG | |
| | CGTGGGTCTCGCGGTATCATTGCAGCACTGGGGCCAGATGGTAAGCCCTCCCGTATCGTAGTTATCTACA | |
| | CGACGGGGAGTCAGGCAACTATGGATGAACGAAATAGACAGATCGCTGAGATAGGTGCCTCACTGATTAA | |
| | GCATTGGTAACTGTCAGACCAAGTTTACTCATATATACTTTAGATTGATTTAAAACTTCATTTTTAATTT | |
| | AAAAGGATCTAGGTGAAGATCCTTTTTGATAATCTCATGACCAAAATCCCTTAACGTGAGTTTTCGTTCC | |
| | ACTGAGCGTCAGACCCCGTAGAAAAGATCAAAGGATCTTCTTGAGATCCTTTTTTTCTGCGCGTAATCTG | |
| | CTGCTTGCAAACAAAAAAACCACCGCTACCAGCGGTGGTTTGTTTGCCGGATCAAGAGCTACCAACTCTT | |
| | TTTCCGAAGGTAACTGGCTTCAGCAGAGCGCAGATACCAAATACTGTTCTTCTAGTGTAGCCGTAGTTAG | |
| | GCCACCACTTCAAGAACTCTGTAGCACCGCCTACATACCTCGCTCTGCTAATCCTGTTACCAGTGGCTGC | |
| | TGCCAGTGGCGATAAGTCGTGTCTTACCGGGTTGGACTCAAGACGATAGTTACCGGATAAGGCGCAGCGG | |
| | TCGGGCTGAACGGGGGGTTCGTGCACACAGCCCAGCTTGGAGCGAACGACCTACACCGAACTGAGATACC | |
| | TACAGCGTGAGCTATGAGAAAGCGCCACGCTTCCCGAAGGGAGAAAGGCGGACAGGTATCCGGTAAGCGG | |
| | CAGGGTCGGAACAGGAGAGCGCACGAGGGAGCTTCCAGGGGGAAACGCCTGGTATCTTTATAGTCCTGTC | |
| | GGGTTTCGCCACCTCTGACTTGAGCGTCGATTTTTGTGATGCTCGTCAGGGGGGCGGAGCCTATGGAAAA | |
| | ACGCCAGCAACGCGGCCTTTTTACGGTTCCTGGCCTTTTGCTGGCCTTTTGCTCACATGTTCTTTCCTGC | |
| | GTTATCCCCTGATTCTGTGGATAACCGTATTACCGCCTTTGAGTGAGCTGATACCGCTCGCCGCAGCCGA | |
| | ACGACCGAGCGCAGCGAGTCAGTGAGCGAGGAAGCGGAAGAGCGCCCAATACGCAAACCGCCTCTCCCCG | |
| | CGCGTTGGCCGATTCATTAATGCAGGGGCCGCTGCGGCCATCATCAATAATATACCTTATTTTGGATTGA | |
| | AGCCAATA | |
| | Description of the elements: | |
| | (1) . . . (585): encodes the shuttle vector (pAd) | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2336): encodes transgene (PYY) from ATG to stop codon | |
| | (2337) . . . (2364): encodes a multiple cloning site | |
| | (2365) . . . (2595): encodes SV40 poly Adenylation signal | |
| | (2596) . . . (3403): encodes MAR 5′ region from human IFN-beta gene | |
| | (3404) . . . (3411): AscI Restriction enzyme site | |
| | (3412) . . . (6098): encodes the shuttle vector (pAd) | |
| HDAd-EF1a-PYY | GGCCGATTCATTAATGCAGGGGCCGCTGCGGCCATCATCAATAATATACCTTATTTTGGATTGAAGCCAA | 52 |
| Approach 6 | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding PYY | CCAGGTGTTTTTCTCAGGTGTTTTCCGCGTTCCGGGTCAAAGTTGGCGTTTTATTATTATAGTCAGCTGA | |
| (3-36) | CGTGTAGTGTATTTATACCCGGTGAGTTCCTCAAGAGGCCACTCTTGAGTGCCAGCGAGTAGAGTTTTCT | |
| Approach 6 | CCTCCGAGCCGCTCCGACACCGGGAGGCGCGCCTTAATTAAAATTATCTCTAAGGCATGTGAACTGGCTG | |
| Construct in | TCTTGGTTTTCATCTGTACTTCATCTGCTACCTCTGTGACCTGAAACATATTTATAATTCCATTAAGCTG | |
| HD-MAR-EF1a | TGCATATGATAGATTTATCATATGTATTTTCCTTAAAGGATTTTTGTAAGAACTAATTGAATTGATACCT | |
| shuttle | GTAAAGTCTTTATCACACTACCCAATAAATAATAAATCTCTTTGTTCAGCTCTCTGTTTCTATAAATATG | |
| vector | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGAAGTCTA | |
| | TCTACTTCGTGGCCGGATTGTTTGTTATGCTGGTGCAGGGTTCCTGGCAGATTAAGCCTGAGGCTCCCGG | |
| | AGAAGACGCCAGCCCCGAAGAGCTCAACCGCTACTACGCTAGTCTGCGGCACTACCTTAACCTCGTAACA | |
| | AGACAGCGGTATCGGGCCAAGAGGGCACCCGTGAAACAGACATTGAATTTCGACCTGCTGAAACTCGCAG | |
| | GAGACGTCGAGTCTAATCCTGGACCGATCAAACCTGAAGCCCCCGGTGAAGACGCATCACCAGAAGAGTT | |
| | GAACCGCTATTACGCTTCTCTTAGACACTACTTGAACTTGGTGACACGGCAACGGTACTAAGGATCCCCG | |
| | GGAGATATCCTAGGCTTGGCCAGACATGATAAGATACATTGATGAGTTTGGACAAACCACAACTAGAATG | |
| | CAGTGAAAAAAATGCTTTATTTGTGAAATTTGTGATGCTATTGCTTTATTTGTAACCATTATAAGCTGCA | |
| | ATAAACAAGTTAACAACAACAATTGCATTCATTTTATGTTTCAGGTTCAGGGGGAGGTGTGGGAGGTTTT | |
| | TTAAAGCAAGTAAAACCTCTACAAATGTGGTATGGAATTCAGTCAATATGTTCACCCCAAAAAAGCTGTT | |
| | TGTTAACTTGCCAACCTCATTCTAAAATGTATATAGAAGCCCAAAAGACAATAACAAAAATATTCTTGTA | |
| | GAACAAAATGGGAAAGAATGTTCCACTAAATATCAAGATTTAGAGCAAAGCATGAGATGTGTGGGGATAG | |
| | ACAGTGAGGCTGATAAAATAGAGTAGAGCTCAGAAACAGACCCATTGATATATGTAAGTGACCTATGAAA | |
| | AAAATATGGCATTTTACAATGGGAAAATGATGGTCTTTTTCTTTTTTAGAAAAACAGGGAAATATATTTA | |
| | TATGTAAAAAATAAAAGGGAACCCATATGTCATACCATACACACAAAAAAATTCCAGTGAATTATAAGTC | |
| | TAAATGGAGAAGGCAAAACTTTAAATCTTTTAGAAAATAATATAGAAGCATGCCATCAAGACTTCAGTGT | |
| | AGAGAAAAATTTCTTATGACTCAAAGTCCTAACCACAAAGAAAAGATTGTTAATTAGATTGCATGAATAT | |
| | TAAGACTTATTTTTAAAATTAAAAAACCATTAAGAAAAGTCAGGCCATAGAATGACAGAAAATATTTGCA | |
| | ACACCCCAGTAAAGAGAATTGTAATATGCAGATTATAAAAAGAAGTCTTACAAATCAGTAAAAAATAAAA | |
| | CTAGACAAAAATTTGAACAGATGAAAGAGAAACTCTAAATAATCATTACACATGAGAAACTCAATCTCAG | |
| | AAATCAGAGAACTATCATTGCATATACACTAAATTAGAGAAATATTAAAAGGCTAAGTAACATCTGTGGC | |
| | TTAATTAAGGCGCGCCGGGCCCCTACGTCACCCGCCCCGTTCCCACGCCCCGCGCCACGTCACAAACTCC | |
| | ACCCCCTCATTATCATATTGGCTTCAATCCAAAATAAGGTATATTATTGATGATGGCCGCAGCGGCCCTG | |
| | GCGTAATAGCGAAGAGGCCCGCACCGATCGCCCTTCCCAACAGTTGCGCAGCCTGAATGGCGAATGGGAC | |
| | GCGCCCTGTAGCGGCGCATTAAGCGCGGCGGGTGTGGTGGTTACGCGCAGCGTGACCGCTACACTTGCCA | |
| | GCGCCCTAGCGCCCGCTCCTTTCGCTTTCTTCCCTTCCTTTCTCGCCACGTTCGCCGGCTTTCCCCGTCA | |
| | AGCTCTAAATCGGGGGCTCCCTTTAGGGTTCCGATTTAGTGCTTTACGGCACCTCGACCCCAAAAAACTT | |
| | GATTAGGGTGATGGTTCACGTAGTGGGCCATCGCCCTGATAGACGGTTTTTCGCCCTTTGACGTTGGAGT | |
| | CCACGTTCTTTAATAGTGGACTCTTGTTCCAAACTGGAACAACACTCAACCCTATCTCGGTCTATTCTTT | |
| | TGATTTATAAGGGATTTTGCCGATTTCGGCCTATTGGTTAAAAAATGAGCTGATTTAACAAAAATTTAAC | |
| | GCGAATTTTAACAAAATATTAACGCTTACAATTTAGGTGGCACTTTTCGGGGAAATGTGCGCGGAACCCC | |
| | TATTTGTTTATTTTTCTAAATACATTCAAATATGTATCCGCTCATGAGACAATAACCCTGATAAATGCTT | |
| | CAATAATATTGAAAAAGGAAGAGTATGAGTATTCAACATTTCCGTGTCGCCCTTATTCCCTTTTTTGCGG | |
| | CATTTTGCCTTCCTGTTTTTGCTCACCCAGAAACGCTGGTGAAAGTAAAAGATGCTGAAGATCAGTTGGG | |
| | TGCACGAGTGGGTTACATCGAACTGGATCTCAACAGCGGTAAGATCCTTGAGAGTTTTCGCCCCGAAGAA | |
| | CGTTTTCCAATGATGAGCACTTTTAAAGTTCTGCTATGTGGCGCGGTATTATCCCGTATTGACGCCGGGC | |
| | AAGAGCAACTCGGTCGCCGCATACACTATTCTCAGAATGACTTGGTTGAGTACTCACCAGTCACAGAAAA | |
| | GCATCTTACGGATGGCATGACAGTAAGAGAATTATGCAGTGCTGCCATAACCATGAGTGATAACACTGCG | |
| | GCCAACTTACTTCTGACAACGATCGGAGGACCGAAGGAGCTAACCGCTTTTTTGCACAACATGGGGGATC | |
| | ATGTAACTCGCCTTGATCGTTGGGAACCGGAGCTGAATGAAGCCATACCAAACGACGAGCGTGACACCAC | |
| | GATGCCTGTAGCAATGGCAACAACGTTGCGCAAACTATTAACTGGCGAACTACTTACTCTAGCTTCCCGG | |
| | CAACAATTAATAGACTGGATGGAGGCGGATAAAGTTGCAGGACCACTTCTGCGCTCGGCCCTTCCGGCTG | |
| | GCTGGTTTATTGCTGATAAATCTGGAGCCGGTGAGCGTGGGTCTCGCGGTATCATTGCAGCACTGGGGCC | |
| | AGATGGTAAGCCCTCCCGTATCGTAGTTATCTACACGACGGGGAGTCAGGCAACTATGGATGAACGAAAT | |
| | AGACAGATCGCTGAGATAGGTGCCTCACTGATTAAGCATTGGTAACTGTCAGACCAAGTTTACTCATATA | |
| | TACTTTAGATTGATTTAAAACTTCATTTTTAATTTAAAAGGATCTAGGTGAAGATCCTTTTTGATAATCT | |
| | CATGACCAAAATCCCTTAACGTGAGTTTTCGTTCCACTGAGCGTCAGACCCCGTAGAAAAGATCAAAGGA | |
| | TCTTCTTGAGATCCTTTTTTTCTGCGCGTAATCTGCTGCTTGCAAACAAAAAAACCACCGCTACCAGCGG | |
| | TGGTTTGTTTGCCGGATCAAGAGCTACCAACTCTTTTTCCGAAGGTAACTGGCTTCAGCAGAGCGCAGAT | |
| | ACCAAATACTGTTCTTCTAGTGTAGCCGTAGTTAGGCCACCACTTCAAGAACTCTGTAGCACCGCCTACA | |
| | TACCTCGCTCTGCTAATCCTGTTACCAGTGGCTGCTGCCAGTGGCGATAAGTCGTGTCTTACCGGGTTGG | |
| | ACTCAAGACGATAGTTACCGGATAAGGCGCAGCGGTCGGGCTGAACGGGGGGTTCGTGCACACAGCCCAG | |
| | CTTGGAGCGAACGACCTACACCGAACTGAGATACCTACAGCGTGAGCTATGAGAAAGCGCCACGCTTCCC | |
| | GAAGGGAGAAAGGCGGACAGGTATCCGGTAAGCGGCAGGGTCGGAACAGGAGAGCGCACGAGGGAGCTTC | |
| | CAGGGGGAAACGCCTGGTATCTTTATAGTCCTGTCGGGTTTCGCCACCTCTGACTTGAGCGTCGATTTTT | |
| | GTGATGCTCGTCAGGGGGGCGGAGCCTATGGAAAAACGCCAGCAACGCGGCCTTTTTACGGTTCCTGGCC | |
| | TTTTGCTGGCCTTTTGCTCACATGTTCTTTCCTGCGTTATCCCCTGATTCTGTGGATAACCGTATTACCG | |
| | CCTTTGAGTGAGCTGATACCGCTCGCCGCAGCCGAACGACCGAGCGCAGCGAGTCAGTGAGCGAGGAAGC | |
| | GGAAGAGCGCCCAATACGCAAACCGCCTCTCCCCGCGCGTTGGCCGATTCATTAATGCAGGGGCCGCTGC | |
| | GGCCATCATCAATAATATACCTTATTTTGGATTGAAGCCAATA | |
| | Description of the elements: | |
| | (1) . . . (585): encodes the shuttle vector (pAd) | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2231): encodes transgene (PYY) from ATG to stop codon | |
| | (2232) . . . (2259): encodes a multiple cloning site | |
| | (2260) . . . (2490): encodes SV40 poly Adenylation signal | |
| | (2491) . . . (3298): encodes MAR 5′ region from human IFN-beta gene | |
| | (3299) . . . (3306): AscI Restriction enzyme site | |
| | (3307) . . . (5993): encodes the shuttle vector (pAd) | |
| HDAd-EF1a-PYY | GGCCGATTCATTAATGCAGGGGCCGCTGCGGCCATCATCAATAATATACCTTATTTTGGATTGAAGCCAA | 53 |
| Approach 7 | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding PYY | CCAGGTGTTTTTCTCAGGTGTTTTCCGCGTTCCGGGTCAAAGTTGGCGTTTTATTATTATAGTCAGCTGA | |
| (3-36) | CGTGTAGTGTATTTATACCCGGTGAGTTCCTCAAGAGGCCACTCTTGAGTGCCAGCGAGTAGAGTTTTCT | |
| Approach 7 | CCTCCGAGCCGCTCCGACACCGGGAGGCGCGCCTTAATTAAAATTATCTCTAAGGCATGTGAACTGGCTG | |
| Construct in | TCTTGGTTTTCATCTGTACTTCATCTGCTACCTCTGTGACCTGAAACATATTTATAATTCCATTAAGCTG | |
| HD-MAR-EF1a | TGCATATGATAGATTTATCATATGTATTTTCCTTAAAGGATTTTTGTAAGAACTAATTGAATTGATACCT | |
| shuttle | GTAAAGTCTTTATCACACTACCCAATAAATAATAAATCTCTTTGTTCAGCTCTCTGTTTCTATAAATATG | |
| vector | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGAAATCTA | |
| | TATACTTTGTCGCTGGCCTCTTTGTGATGCTCGTTCAGGGCTCATGGCAGATCAAGCCCGAGGCTCCTGG | |
| | AGAAGATGCATCTCCAGAGGAACTCAATAGATATTATGCCTCACTGCGCCATTACCTGAACCTGGTCACA | |
| | CGCCAGCGGTATCGGGCCAAGCGGGCTCCCGTAAAGCAAACCCTGAACTTCGATCTGCTGAAACTGGCCG | |
| | GAGACGTAGAGTCCAATCCCGGCCCCGATTTCCCTGAGGAGGTCGCTATCGTCGAAGAGTTGGGCAGGAG | |
| | GATCAAGCCCGAAGCACCGGGGGAGGACGCTAGCCCCGAGGAACTTAACCGATACTACGCTAGCCTGAGA | |
| | CATTATCTCAATCTGGTGACACGCCAACGGTATTAAGGATCCCCGGGAGATATCCTAGGCTTGGCCAGAC | |
| | ATGATAAGATACATTGATGAGTTTGGACAAACCACAACTAGAATGCAGTGAAAAAAATGCTTTATTTGTG | |
| | AAATTTGTGATGCTATTGCTTTATTTGTAACCATTATAAGCTGCAATAAACAAGTTAACAACAACAATTG | |
| | CATTCATTTTATGTTTCAGGTTCAGGGGGAGGTGTGGGAGGTTTTTTAAAGCAAGTAAAACCTCTACAAA | |
| | TGTGGTATGGAATTCAGTCAATATGTTCACCCCAAAAAAGCTGTTTGTTAACTTGCCAACCTCATTCTAA | |
| | AATGTATATAGAAGCCCAAAAGACAATAACAAAAATATTCTTGTAGAACAAAATGGGAAAGAATGTTCCA | |
| | CTAAATATCAAGATTTAGAGCAAAGCATGAGATGTGTGGGGATAGACAGTGAGGCTGATAAAATAGAGTA | |
| | GAGCTCAGAAACAGACCCATTGATATATGTAAGTGACCTATGAAAAAAATATGGCATTTTACAATGGGAA | |
| | AATGATGGTCTTTTTCTTTTTTAGAAAAACAGGGAAATATATTTATATGTAAAAAATAAAAGGGAACCCA | |
| | TATGTCATACCATACACACAAAAAAATTCCAGTGAATTATAAGTCTAAATGGAGAAGGCAAAACTTTAAA | |
| | TCTTTTAGAAAATAATATAGAAGCATGCCATCAAGACTTCAGTGTAGAGAAAAATTTCTTATGACTCAAA | |
| | GTCCTAACCACAAAGAAAAGATTGTTAATTAGATTGCATGAATATTAAGACTTATTTTTAAAATTAAAAA | |
| | ACCATTAAGAAAAGTCAGGCCATAGAATGACAGAAAATATTTGCAACACCCCAGTAAAGAGAATTGTAAT | |
| | ATGCAGATTATAAAAAGAAGTCTTACAAATCAGTAAAAAATAAAACTAGACAAAAATTTGAACAGATGAA | |
| | AGAGAAACTCTAAATAATCATTACACATGAGAAACTCAATCTCAGAAATCAGAGAACTATCATTGCATAT | |
| | ACACTAAATTAGAGAAATATTAAAAGGCTAAGTAACATCTGTGGCTTAATTAAGGCGCGCCGGGCCCCTA | |
| | CGTCACCCGCCCCGTTCCCACGCCCCGCGCCACGTCACAAACTCCACCCCCTCATTATCATATTGGCTTC | |
| | AATCCAAAATAAGGTATATTATTGATGATGGCCGCAGCGGCCCTGGCGTAATAGCGAAGAGGCCCGCACC | |
| | GATCGCCCTTCCCAACAGTTGCGCAGCCTGAATGGCGAATGGGACGCGCCCTGTAGCGGCGCATTAAGCG | |
| | CGGCGGGTGTGGTGGTTACGCGCAGCGTGACCGCTACACTTGCCAGCGCCCTAGCGCCCGCTCCTTTCGC | |
| | TTTCTTCCCTTCCTTTCTCGCCACGTTCGCCGGCTTTCCCCGTCAAGCTCTAAATCGGGGGCTCCCTTTA | |
| | GGGTTCCGATTTAGTGCTTTACGGCACCTCGACCCCAAAAAACTTGATTAGGGTGATGGTTCACGTAGTG | |
| | GGCCATCGCCCTGATAGACGGTTTTTCGCCCTTTGACGTTGGAGTCCACGTTCTTTAATAGTGGACTCTT | |
| | GTTCCAAACTGGAACAACACTCAACCCTATCTCGGTCTATTCTTTTGATTTATAAGGGATTTTGCCGATT | |
| | TCGGCCTATTGGTTAAAAAATGAGCTGATTTAACAAAAATTTAACGCGAATTTTAACAAAATATTAACGC | |
| | TTACAATTTAGGTGGCACTTTTCGGGGAAATGTGCGCGGAACCCCTATTTGTTTATTTTTCTAAATACAT | |
| | TCAAATATGTATCCGCTCATGAGACAATAACCCTGATAAATGCTTCAATAATATTGAAAAAGGAAGAGTA | |
| | TGAGTATTCAACATTTCCGTGTCGCCCTTATTCCCTTTTTTGCGGCATTTTGCCTTCCTGTTTTTGCTCA | |
| | CCCAGAAACGCTGGTGAAAGTAAAAGATGCTGAAGATCAGTTGGGTGCACGAGTGGGTTACATCGAACTG | |
| | GATCTCAACAGCGGTAAGATCCTTGAGAGTTTTCGCCCCGAAGAACGTTTTCCAATGATGAGCACTTTTA | |
| | AAGTTCTGCTATGTGGCGCGGTATTATCCCGTATTGACGCCGGGCAAGAGCAACTCGGTCGCCGCATACA | |
| | CTATTCTCAGAATGACTTGGTTGAGTACTCACCAGTCACAGAAAAGCATCTTACGGATGGCATGACAGTA | |
| | AGAGAATTATGCAGTGCTGCCATAACCATGAGTGATAACACTGCGGCCAACTTACTTCTGACAACGATCG | |
| | GAGGACCGAAGGAGCTAACCGCTTTTTTGCACAACATGGGGGATCATGTAACTCGCCTTGATCGTTGGGA | |
| | ACCGGAGCTGAATGAAGCCATACCAAACGACGAGCGTGACACCACGATGCCTGTAGCAATGGCAACAACG | |
| | TTGCGCAAACTATTAACTGGCGAACTACTTACTCTAGCTTCCCGGCAACAATTAATAGACTGGATGGAGG | |
| | CGGATAAAGTTGCAGGACCACTTCTGCGCTCGGCCCTTCCGGCTGGCTGGTTTATTGCTGATAAATCTGG | |
| | AGCCGGTGAGCGTGGGTCTCGCGGTATCATTGCAGCACTGGGGCCAGATGGTAAGCCCTCCCGTATCGTA | |
| | GTTATCTACACGACGGGGAGTCAGGCAACTATGGATGAACGAAATAGACAGATCGCTGAGATAGGTGCCT | |
| | CACTGATTAAGCATTGGTAACTGTCAGACCAAGTTTACTCATATATACTTTAGATTGATTTAAAACTTCA | |
| | TTTTTAATTTAAAAGGATCTAGGTGAAGATCCTTTTTGATAATCTCATGACCAAAATCCCTTAACGTGAG | |
| | TTTTCGTTCCACTGAGCGTCAGACCCCGTAGAAAAGATCAAAGGATCTTCTTGAGATCCTTTTTTTCTGC | |
| | GCGTAATCTGCTGCTTGCAAACAAAAAAACCACCGCTACCAGCGGTGGTTTGTTTGCCGGATCAAGAGCT | |
| | ACCAACTCTTTTTCCGAAGGTAACTGGCTTCAGCAGAGCGCAGATACCAAATACTGTTCTTCTAGTGTAG | |
| | CCGTAGTTAGGCCACCACTTCAAGAACTCTGTAGCACCGCCTACATACCTCGCTCTGCTAATCCTGTTAC | |
| | CAGTGGCTGCTGCCAGTGGCGATAAGTCGTGTCTTACCGGGTTGGACTCAAGACGATAGTTACCGGATAA | |
| | GGCGCAGCGGTCGGGCTGAACGGGGGGTTCGTGCACACAGCCCAGCTTGGAGCGAACGACCTACACCGAA | |
| | CTGAGATACCTACAGCGTGAGCTATGAGAAAGCGCCACGCTTCCCGAAGGGAGAAAGGCGGACAGGTATC | |
| | CGGTAAGCGGCAGGGTCGGAACAGGAGAGCGCACGAGGGAGCTTCCAGGGGGAAACGCCTGGTATCTTTA | |
| | TAGTCCTGTCGGGTTTCGCCACCTCTGACTTGAGCGTCGATTTTTGTGATGCTCGTCAGGGGGGCGGAGC | |
| | CTATGGAAAAACGCCAGCAACGCGGCCTTTTTACGGTTCCTGGCCTTTTGCTGGCCTTTTGCTCACATGT | |
| | TCTTTCCTGCGTTATCCCCTGATTCTGTGGATAACCGTATTACCGCCTTTGAGTGAGCTGATACCGCTCG | |
| | CCGCAGCCGAACGACCGAGCGCAGCGAGTCAGTGAGCGAGGAAGCGGAAGAGCGCCCAATACGCAAACCG | |
| | CCTCTCCCCGCGCGTTGGCCGATTCATTAATGCAGGGGCCGCTGCGGCCATCATCAATAATATACCTTAT | |
| | TTTGGATTGAAGCCAATA | |
| | Description of the elements: | |
| | (1) . . . (585): encodes the shuttle vector (pAd) | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2276): encodes transgene (PYY) from ATG to stop codon | |
| | (2277) . . . (2304): encodes a multiple cloning site | |
| | (2305) . . . (2535): encodes SV40 poly Adenylation signal | |
| | (2536) . . . (3343): encodes MAR 5′ region from human IFN-beta gene | |
| | (3344) . . . (3351): AscI Restriction enzyme site | |
| | (3352) . . . (4964): encodes the shuttle vector (pAd) | |
| Oxyntomudulin Amino Acid | 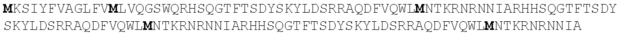 | 54 |
| Sequence in | (1) . . . (20): SP-Glucagon | |
| Approach A | (22) . . . (58): OXM | |
| Construct | (59) . . . (60): PCSK1/2 | |
| (Proglucagon | (61) . . . (97): OXM | |
| signal | (98) . . . (99): PCSK1/2 | |
| peptide-Oxy- | (100) . . . (136): OXM | |
| PCK1/2-Oxy- | | |
| PCK1/2-Oxy) | | |
| Codon | ATGAAATCTATCTACTTTGTCGCTGGTCTCTTTGTGATGCTCGTCCAAGGGTCCTGGCAGAGACACAGTC | 55 |
| Optomized | AGGGGACTTTTACATCAGATTATAGTAAGTATCTCGACAGCAGACGCGCGCAGGACTTTGTGCAGTGGCT | |
| Nucleic Acid | GATGAATACTAAACGAAACCGAAATAACATCGCCAGACATCACAGCCAGGGCACTTTTACAAGCGACTAT | |
| Sequence | AGCAAATATCTGGACAGTAGGCGAGCCCAAGATTTCGTGCAATGGCTGATGAATACAAAGAGAAACCGCA | |
| Encoding | ATAACATAGCGCGACATCACTCTCAGGGCACCTTCACCAGTGATTACTCCAAGTACCTGGATTCTCGGAG | |
| Oxyntomodulin | AGCGCAAGATTTCGTGCAGTGGCTGATGAATACTAAGAGAAATCGCAACAACATCGCATGA | |
| Approach A | | |
| Construct | | |
| Oxyntomodulin Amino Acid | 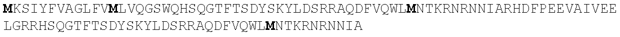 | 56 |
| Sequence in | (1) . . . (20): SP-Glucagon | |
| Approach B | (21) . . . (57): OXM | |
| Construct | (58) . . . (59): PCSK1/2 | |
| (Proglucagon | (60) . . . (74): Propeptide | |
| signal | (75) . . . (111): OXM | |
| peptide-Oxy- | | |
| IP-2-Oxy) | | |
| Codon | AAGCTTGCATGCCTGCAGGTCGACTCTAGACTGCCATGAAGTCCATTTACTTCGTGGCAGGTCTCTTTGT | 57 |
| Optimized | GATGTTGGTTCAGGGTAGCTGGCAGCACTCACAGGGCACTTTCACTTCCGATTACTCTAAGTACCTGGAC | |
| Nucleic Acid | AGCAGGCGGGCCCAGGATTTCGTGCAGTGGCTTATGAACACAAAGAGAAACAGAAATAATATTGCCCGGC | |
| Sequence | ATGACTTTCCTGAAGAGGTGGCTATCGTTGAGGAGCTGGGAAGACGGCACTCTCAGGGCACCTTTACATC | |
| Encoding | CGACTACTCAAAATACCTGGATTCAAGAAGGGCGCAGGATTTTGTGCAGTGGCTTATGAACACAAAACGC | |
| Oxyntomodulin | AACCGCAATAACATCGCCTAAGGATCCCCGGGAGATATCCTAGGCTTGG | |
| Approach B | (No key provided) | |
| Construct | | |
| HDAd-EF1a-Oxy | GGCCGATTCATTAATGCAGGGGCCGCTGCGGCCATCATCAATAATATACCTTATTTTGGATTGAAGCCAA | 58 |
| Approach A | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding Oxy | CCAGGTGTTTTTCTCAGGTGTTTTCCGCGTTCCGGGTCAAAGTTGGCGTTTTATTATTATAGTCAGCTGA | |
| Approach A | CGTGTAGTGTATTTATACCCGGTGAGTTCCTCAAGAGGCCACTCTTGAGTGCCAGCGAGTAGAGTTTTCT | |
| Construct in | CCTCCGAGCCGCTCCGACACCGGGAGGCGCGCCTTAATTAAAATTATCTCTAAGGCATGTGAACTGGCTG | |
| HD-MAR-EF1a | TCTTGGTTTTCATCTGTACTTCATCTGCTACCTCTGTGACCTGAAACATATTTATAATTCCATTAAGCTG | |
| shuttle | TGCATATGATAGATTTATCATATGTATTTTCCTTAAAGGATTTTTGTAAGAACTAATTGAATTGATACCT | |
| vector | GTAAAGTCTTTATCACACTACCCAATAAATAATAAATCTCTTTGTTCAGCTCTCTGTTTCTATAAATATG | |
| | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGAAATCTA | |
| | TCTACTTTGTCGCTGGTCTCTTTGTGATGCTCGTCCAAGGGTCCTGGCAGAGACACAGTCAGGGGACTTT | |
| | TACATCAGATTATAGTAAGTATCTCGACAGCAGACGCGCGCAGGACTTTGTGCAGTGGCTGATGAATACT | |
| | AAACGAAACCGAAATAACATCGCCAGACATCACAGCCAGGGCACTTTTACAAGCGACTATAGCAAATATC | |
| | TGGACAGTAGGCGAGCCCAAGATTTCGTGCAATGGCTGATGAATACAAAGAGAAACCGCAATAACATAGC | |
| | GCGACATCACTCTCAGGGCACCTTCACCAGTGATTACTCCAAGTACCTGGATTCTCGGAGAGCGCAAGAT | |
| | TTCGTGCAGTGGCTGATGAATACTAAGAGAAATCGCAACAACATCGCATGAGGATCCCCGGGAGATATCC | |
| | TAGGCTTGGCCAGACATGATAAGATACATTGATGAGTTTGGACAAACCACAACTAGAATGCAGTGAAAAA | |
| | AATGCTTTATTTGTGAAATTTGTGATGCTATTGCTTTATTTGTAACCATTATAAGCTGCAATAAACAAGT | |
| | TAACAACAACAATTGCATTCATTTTATGTTTCAGGTTCAGGGGGAGGTGTGGGAGGTTTTTTAAAGCAAG | |
| | TAAAACCTCTACAAATGTGGTATGGAATTCAGTCAATATGTTCACCCCAAAAAAGCTGTTTGTTAACTTG | |
| | CCAACCTCATTCTAAAATGTATATAGAAGCCCAAAAGACAATAACAAAAATATTCTTGTAGAACAAAATG | |
| | GGAAAGAATGTTCCACTAAATATCAAGATTTAGAGCAAAGCATGAGATGTGTGGGGATAGACAGTGAGGC | |
| | TGATAAAATAGAGTAGAGCTCAGAAACAGACCCATTGATATATGTAAGTGACCTATGAAAAAAATATGGC | |
| | ATTTTACAATGGGAAAATGATGGTCTTTTTCTTTTTTAGAAAAACAGGGAAATATATTTATATGTAAAAA | |
| | ATAAAAGGGAACCCATATGTCATACCATACACACAAAAAAATTCCAGTGAATTATAAGTCTAAATGGAGA | |
| | AGGCAAAACTTTAAATCTTTTAGAAAATAATATAGAAGCATGCCATCAAGACTTCAGTGTAGAGAAAAAT | |
| | TTCTTATGACTCAAAGTCCTAACCACAAAGAAAAGATTGTTAATTAGATTGCATGAATATTAAGACTTAT | |
| | TTTTAAAATTAAAAAACCATTAAGAAAAGTCAGGCCATAGAATGACAGAAAATATTTGCAACACCCCAGT | |
| | AAAGAGAATTGTAATATGCAGATTATAAAAAGAAGTCTTACAAATCAGTAAAAAATAAAACTAGACAAAA | |
| | ATTTGAACAGATGAAAGAGAAACTCTAAATAATCATTACACATGAGAAACTCAATCTCAGAAATCAGAGA | |
| | ACTATCATTGCATATACACTAAATTAGAGAAATATTAAAAGGCTAAGTAACATCTGTGGCTTAATTAAGG | |
| | CGCGCCGGGCCCCTACGTCACCCGCCCCGTTCCCACGCCCCGCGCCACGTCACAAACTCCACCCCCTCAT | |
| | TATCATATTGGCTTCAATCCAAAATAAGGTATATTATTGATGATGGCCGCAGCGGCCCTGGCGTAATAGC | |
| | GAAGAGGCCCGCACCGATCGCCCTTCCCAACAGTTGCGCAGCCTGAATGGCGAATGGGACGCGCCCTGTA | |
| | GCGGCGCATTAAGCGCGGCGGGTGTGGTGGTTACGCGCAGCGTGACCGCTACACTTGCCAGCGCCCTAGC | |
| | GCCCGCTCCTTTCGCTTTCTTCCCTTCCTTTCTCGCCACGTTCGCCGGCTTTCCCCGTCAAGCTCTAAAT | |
| | CGGGGGCTCCCTTTAGGGTTCCGATTTAGTGCTTTACGGCACCTCGACCCCAAAAAACTTGATTAGGGTG | |
| | ATGGTTCACGTAGTGGGCCATCGCCCTGATAGACGGTTTTTCGCCCTTTGACGTTGGAGTCCACGTTCTT | |
| | TAATAGTGGACTCTTGTTCCAAACTGGAACAACACTCAACCCTATCTCGGTCTATTCTTTTGATTTATAA | |
| | GGGATTTTGCCGATTTCGGCCTATTGGTTAAAAAATGAGCTGATTTAACAAAAATTTAACGCGAATTTTA | |
| | ACAAAATATTAACGCTTACAATTTAGGTGGCACTTTTCGGGGAAATGTGCGCGGAACCCCTATTTGTTTA | |
| | TTTTTCTAAATACATTCAAATATGTATCCGCTCATGAGACAATAACCCTGATAAATGCTTCAATAATATT | |
| | GAAAAAGGAAGAGTATGAGTATTCAACATTTCCGTGTCGCCCTTATTCCCTTTTTTGCGGCATTTTGCCT | |
| | TCCTGTTTTTGCTCACCCAGAAACGCTGGTGAAAGTAAAAGATGCTGAAGATCAGTTGGGTGCACGAGTG | |
| | GGTTACATCGAACTGGATCTCAACAGCGGTAAGATCCTTGAGAGTTTTCGCCCCGAAGAACGTTTTCCAA | |
| | TGATGAGCACTTTTAAAGTTCTGCTATGTGGCGCGGTATTATCCCGTATTGACGCCGGGCAAGAGCAACT | |
| | CGGTCGCCGCATACACTATTCTCAGAATGACTTGGTTGAGTACTCACCAGTCACAGAAAAGCATCTTACG | |
| | GATGGCATGACAGTAAGAGAATTATGCAGTGCTGCCATAACCATGAGTGATAACACTGCGGCCAACTTAC | |
| | TTCTGACAACGATCGGAGGACCGAAGGAGCTAACCGCTTTTTTGCACAACATGGGGGATCATGTAACTCG | |
| | CCTTGATCGTTGGGAACCGGAGCTGAATGAAGCCATACCAAACGACGAGCGTGACACCACGATGCCTGTA | |
| | GCAATGGCAACAACGTTGCGCAAACTATTAACTGGCGAACTACTTACTCTAGCTTCCCGGCAACAATTAA | |
| | TAGACTGGATGGAGGCGGATAAAGTTGCAGGACCACTTCTGCGCTCGGCCCTTCCGGCTGGCTGGTTTAT | |
| | TGCTGATAAATCTGGAGCCGGTGAGCGTGGGTCTCGCGGTATCATTGCAGCACTGGGGCCAGATGGTAAG | |
| | CCCTCCCGTATCGTAGTTATCTACACGACGGGGAGTCAGGCAACTATGGATGAACGAAATAGACAGATCG | |
| | CTGAGATAGGTGCCTCACTGATTAAGCATTGGTAACTGTCAGACCAAGTTTACTCATATATACTTTAGAT | |
| | TGATTTAAAACTTCATTTTTAATTTAAAAGGATCTAGGTGAAGATCCTTTTTGATAATCTCATGACCAAA | |
| | ATCCCTTAACGTGAGTTTTCGTTCCACTGAGCGTCAGACCCCGTAGAAAAGATCAAAGGATCTTCTTGAG | |
| | ATCCTTTTTTTCTGCGCGTAATCTGCTGCTTGCAAACAAAAAAACCACCGCTACCAGCGGTGGTTTGTTT | |
| | GCCGGATCAAGAGCTACCAACTCTTTTTCCGAAGGTAACTGGCTTCAGCAGAGCGCAGATACCAAATACT | |
| | GTTCTTCTAGTGTAGCCGTAGTTAGGCCACCACTTCAAGAACTCTGTAGCACCGCCTACATACCTCGCTC | |
| | TGCTAATCCTGTTACCAGTGGCTGCTGCCAGTGGCGATAAGTCGTGTCTTACCGGGTTGGACTCAAGACG | |
| | ATAGTTACCGGATAAGGCGCAGCGGTCGGGCTGAACGGGGGGTTCGTGCACACAGCCCAGCTTGGAGCGA | |
| | ACGACCTACACCGAACTGAGATACCTACAGCGTGAGCTATGAGAAAGCGCCACGCTTCCCGAAGGGAGAA | |
| | AGGCGGACAGGTATCCGGTAAGCGGCAGGGTCGGAACAGGAGAGCGCACGAGGGAGCTTCCAGGGGGAAA | |
| | CGCCTGGTATCTTTATAGTCCTGTCGGGTTTCGCCACCTCTGACTTGAGCGTCGATTTTTGTGATGCTCG | |
| | TCAGGGGGGCGGAGCCTATGGAAAAACGCCAGCAACGCGGCCTTTTTACGGTTCCTGGCCTTTTGCTGGC | |
| | CTTTTGCTCACATGTTCTTTCCTGCGTTATCCCCTGATTCTGTGGATAACCGTATTACCGCCTTTGAGTG | |
| | AGCTGATACCGCTCGCCGCAGCCGAACGACCGAGCGCAGCGAGTCAGTGAGCGAGGAAGCGGAAGAGCGC | |
| | CCAATACGCAAACCGCCTCTCCCCGCGCGTTGGCCGATTCATTAATGCAGGGGCCGCTGCGGCCATCATC | |
| | AATAATATACCTTATTTTGGATTGAAGCCAATA | |
| | Description of the elements: | |
| | (1) . . . (585): encodes the shuttle vector | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2291): encodes transgene (opt OXM 1) from ATG to stop | |
| | codon (TAA) | |
| | (2292) . . . (2319): encodes a multiple cloning site | |
| | (2320) . . . (2550): encodes SV40 poly Adenylation signal | |
| | (2551) . . . (3358): encodes MAR 5′ region from human IFN-beta gene | |
| | (3359) . . . (3366): AscI Restriction enzyme site | |
| | (3367) . . . (6053): encodes the shuttle vector | |
| HDAd-EF1a-Oxy | GGCCGATTCATTAATGCAGGGGCCGCTGCGGCCATCATCAATAATATACCTTATTTTGGATTGAAGCCAA | 59 |
| Approach B | TATGATAATGAGGGGGTGGAGTTTGTGACGTGGCGCGGGGCGTGGGAACGGGGCGGGTGACGTAGTAGTG | |
| Codon | TGGCGGAAGTGTGATGTTGCAAGTGTGGCGGAACACATGTAAGCGACGGATGTGGCAAAAGTGACGTTTT | |
| Optimized | TGGTGTGCGCCGGTGTACACAGGAAGTGACAATTTTCGCGCGGTTTTAGGCGGATGTTGTAGTAAATTTG | |
| Nucleic Acid | GGCGTAACCGAGTAAGATTTGGCCATTTTCGCGGGAAAACTGAATAAGAGGAAGTGAAATCTGAATAATT | |
| Sequence | TTGTGTTACTCATAGCGCGTAATATTTGTCTAGGGCCGCGGGGACTTTGACCGTTTACGTGGAGACTCGC | |
| Encoding Oxy | CCAGGTGTTTTTCTCAGGTGTTTTCCGCGTTCCGGGTCAAAGTTGGCGTTTTATTATTATAGTCAGCTGA | |
| Approach B | CGTGTAGTGTATTTATACCCGGTGAGTTCCTCAAGAGGCCACTCTTGAGTGCCAGCGAGTAGAGTTTTCT | |
| Construct in | CCTCCGAGCCGCTCCGACACCGGGAGGCGCGCCTTAATTAAAATTATCTCTAAGGCATGTGAACTGGCTG | |
| HD-MAR-EF1a | TCTTGGTTTTCATCTGTACTTCATCTGCTACCTCTGTGACCTGAAACATATTTATAATTCCATTAAGCTG | |
| shuttle | TGCATATGATAGATTTATCATATGTATTTTCCTTAAAGGATTTTTGTAAGAACTAATTGAATTGATACCT | |
| vector | GTAAAGTCTTTATCACACTACCCAATAAATAATAAATCTCTTTGTTCAGCTCTCTGTTTCTATAAATATG | |
| | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGAAGTCCA | |
| | TTTACTTCGTGGCAGGTCTCTTTGTGATGTTGGTTCAGGGTAGCTGGCAGCACTCACAGGGCACTTTCAC | |
| | TTCCGATTACTCTAAGTACCTGGACAGCAGGCGGGCCCAGGATTTCGTGCAGTGGCTTATGAACACAAAG | |
| | AGAAACAGAAATAATATTGCCCGGCATGACTTTCCTGAAGAGGTGGCTATCGTTGAGGAGCTGGGAAGAC | |
| | GGCACTCTCAGGGCACCTTTACATCCGACTACTCAAAATACCTGGATTCAAGAAGGGCGCAGGATTTTGT | |
| | GCAGTGGCTTATGAACACAAAACGCAACCGCAATAACATCGCCTAAGGATCCCCGGGAGATATCCTAGGC | |
| | TTGGCCAGACATGATAAGATACATTGATGAGTTTGGACAAACCACAACTAGAATGCAGTGAAAAAAATGC | |
| | TTTATTTGTGAAATTTGTGATGCTATTGCTTTATTTGTAACCATTATAAGCTGCAATAAACAAGTTAACA | |
| | ACAACAATTGCATTCATTTTATGTTTCAGGTTCAGGGGGAGGTGTGGGAGGTTTTTTAAAGCAAGTAAAA | |
| | CCTCTACAAATGTGGTATGGAATTCAGTCAATATGTTCACCCCAAAAAAGCTGTTTGTTAACTTGCCAAC | |
| | CTCATTCTAAAATGTATATAGAAGCCCAAAAGACAATAACAAAAATATTCTTGTAGAACAAAATGGGAAA | |
| | GAATGTTCCACTAAATATCAAGATTTAGAGCAAAGCATGAGATGTGTGGGGATAGACAGTGAGGCTGATA | |
| | AAATAGAGTAGAGCTCAGAAACAGACCCATTGATATATGTAAGTGACCTATGAAAAAAATATGGCATTTT | |
| | ACAATGGGAAAATGATGGTCTTTTTCTTTTTTAGAAAAACAGGGAAATATATTTATATGTAAAAAATAAA | |
| | AGGGAACCCATATGTCATACCATACACACAAAAAAATTCCAGTGAATTATAAGTCTAAATGGAGAAGGCA | |
| | AAACTTTAAATCTTTTAGAAAATAATATAGAAGCATGCCATCAAGACTTCAGTGTAGAGAAAAATTTCTT | |
| | ATGACTCAAAGTCCTAACCACAAAGAAAAGATTGTTAATTAGATTGCATGAATATTAAGACTTATTTTTA | |
| | AAATTAAAAAACCATTAAGAAAAGTCAGGCCATAGAATGACAGAAAATATTTGCAACACCCCAGTAAAGA | |
| | GAATTGTAATATGCAGATTATAAAAAGAAGTCTTACAAATCAGTAAAAAATAAAACTAGACAAAAATTTG | |
| | AACAGATGAAAGAGAAACTCTAAATAATCATTACACATGAGAAACTCAATCTCAGAAATCAGAGAACTAT | |
| | CATTGCATATACACTAAATTAGAGAAATATTAAAAGGCTAAGTAACATCTGTGGCTTAATTAAGGCGCGC | |
| | CGGGCCCCTACGTCACCCGCCCCGTTCCCACGCCCCGCGCCACGTCACAAACTCCACCCCCTCATTATCA | |
| | TATTGGCTTCAATCCAAAATAAGGTATATTATTGATGATGGCCGCAGCGGCCCTGGCGTAATAGCGAAGA | |
| | GGCCCGCACCGATCGCCCTTCCCAACAGTTGCGCAGCCTGAATGGCGAATGGGACGCGCCCTGTAGCGGC | |
| | GCATTAAGCGCGGCGGGTGTGGTGGTTACGCGCAGCGTGACCGCTACACTTGCCAGCGCCCTAGCGCCCG | |
| | CTCCTTTCGCTTTCTTCCCTTCCTTTCTCGCCACGTTCGCCGGCTTTCCCCGTCAAGCTCTAAATCGGGG | |
| | GCTCCCTTTAGGGTTCCGATTTAGTGCTTTACGGCACCTCGACCCCAAAAAACTTGATTAGGGTGATGGT | |
| | TCACGTAGTGGGCCATCGCCCTGATAGACGGTTTTTCGCCCTTTGACGTTGGAGTCCACGTTCTTTAATA | |
| | GTGGACTCTTGTTCCAAACTGGAACAACACTCAACCCTATCTCGGTCTATTCTTTTGATTTATAAGGGAT | |
| | TTTGCCGATTTCGGCCTATTGGTTAAAAAATGAGCTGATTTAACAAAAATTTAACGCGAATTTTAACAAA | |
| | ATATTAACGCTTACAATTTAGGTGGCACTTTTCGGGGAAATGTGCGCGGAACCCCTATTTGTTTATTTTT | |
| | CTAAATACATTCAAATATGTATCCGCTCATGAGACAATAACCCTGATAAATGCTTCAATAATATTGAAAA | |
| | AGGAAGAGTATGAGTATTCAACATTTCCGTGTCGCCCTTATTCCCTTTTTTGCGGCATTTTGCCTTCCTG | |
| | TTTTTGCTCACCCAGAAACGCTGGTGAAAGTAAAAGATGCTGAAGATCAGTTGGGTGCACGAGTGGGTTA | |
| | CATCGAACTGGATCTCAACAGCGGTAAGATCCTTGAGAGTTTTCGCCCCGAAGAACGTTTTCCAATGATG | |
| | AGCACTTTTAAAGTTCTGCTATGTGGCGCGGTATTATCCCGTATTGACGCCGGGCAAGAGCAACTCGGTC | |
| | GCCGCATACACTATTCTCAGAATGACTTGGTTGAGTACTCACCAGTCACAGAAAAGCATCTTACGGATGG | |
| | CATGACAGTAAGAGAATTATGCAGTGCTGCCATAACCATGAGTGATAACACTGCGGCCAACTTACTTCTG | |
| | ACAACGATCGGAGGACCGAAGGAGCTAACCGCTTTTTTGCACAACATGGGGGATCATGTAACTCGCCTTG | |
| | ATCGTTGGGAACCGGAGCTGAATGAAGCCATACCAAACGACGAGCGTGACACCACGATGCCTGTAGCAAT | |
| | GGCAACAACGTTGCGCAAACTATTAACTGGCGAACTACTTACTCTAGCTTCCCGGCAACAATTAATAGAC | |
| | TGGATGGAGGCGGATAAAGTTGCAGGACCACTTCTGCGCTCGGCCCTTCCGGCTGGCTGGTTTATTGCTG | |
| | ATAAATCTGGAGCCGGTGAGCGTGGGTCTCGCGGTATCATTGCAGCACTGGGGCCAGATGGTAAGCCCTC | |
| | CCGTATCGTAGTTATCTACACGACGGGGAGTCAGGCAACTATGGATGAACGAAATAGACAGATCGCTGAG | |
| | ATAGGTGCCTCACTGATTAAGCATTGGTAACTGTCAGACCAAGTTTACTCATATATACTTTAGATTGATT | |
| | TAAAACTTCATTTTTAATTTAAAAGGATCTAGGTGAAGATCCTTTTTGATAATCTCATGACCAAAATCCC | |
| | TTAACGTGAGTTTTCGTTCCACTGAGCGTCAGACCCCGTAGAAAAGATCAAAGGATCTTCTTGAGATCCT | |
| | TTTTTTCTGCGCGTAATCTGCTGCTTGCAAACAAAAAAACCACCGCTACCAGCGGTGGTTTGTTTGCCGG | |
| | ATCAAGAGCTACCAACTCTTTTTCCGAAGGTAACTGGCTTCAGCAGAGCGCAGATACCAAATACTGTTCT | |
| | TCTAGTGTAGCCGTAGTTAGGCCACCACTTCAAGAACTCTGTAGCACCGCCTACATACCTCGCTCTGCTA | |
| | ATCCTGTTACCAGTGGCTGCTGCCAGTGGCGATAAGTCGTGTCTTACCGGGTTGGACTCAAGACGATAGT | |
| | TACCGGATAAGGCGCAGCGGTCGGGCTGAACGGGGGGTTCGTGCACACAGCCCAGCTTGGAGCGAACGAC | |
| | CTACACCGAACTGAGATACCTACAGCGTGAGCTATGAGAAAGCGCCACGCTTCCCGAAGGGAGAAAGGCG | |
| | GACAGGTATCCGGTAAGCGGCAGGGTCGGAACAGGAGAGCGCACGAGGGAGCTTCCAGGGGGAAACGCCT | |
| | GGTATCTTTATAGTCCTGTCGGGTTTCGCCACCTCTGACTTGAGCGTCGATTTTTGTGATGCTCGTCAGG | |
| | GGGGCGGAGCCTATGGAAAAACGCCAGCAACGCGGCCTTTTTACGGTTCCTGGCCTTTTGCTGGCCTTTT | |
| | GCTCACATGTTCTTTCCTGCGTTATCCCCTGATTCTGTGGATAACCGTATTACCGCCTTTGAGTGAGCTG | |
| | ATACCGCTCGCCGCAGCCGAACGACCGAGCGCAGCGAGTCAGTGAGCGAGGAAGCGGAAGAGCGCCCAAT | |
| | ACGCAAACCGCCTCTCCCCGCGCGTTGGCCGATTCATTAATGCAGGGGCCGCTGCGGCCATCATCAATAA | |
| | TATACCTTATTTTGGATTGAAGCCAATA | |
| | Description of the elements: | |
| | (1) . . . (585): encodes the shuttle vector | |
| | (586) . . . (593): AscI Restriction enzyme site | |
| | (594) . . . (1020): encodes CpG free MAR from human beta-globin gene | |
| | (1021) . . . (1443): encodes CMV Enhancer | |
| | (1444) . . . (1698): encodes human EF1-alpha promotor | |
| | (1699) . . . (1826): encodes synthetic intron | |
| | (1827) . . . (1880): encodes a multiple cloning site | |
| | (1881) . . . (2216): encodes transgene (opt OXM ver B) from ATG to | |
| | stop codon (TAA) | |
| | (2217) . . . (2244): encodes a multiple cloning site | |
| | (2245) . . . (2475): encodes SV40 poly Adenylation signal | |
| | (2476) . . . (3283): encodes MAR 5′ region from human IFN-beta gene | |
| | (3284) . . . (3291): AscI Restriction enzyme site | |
| | (3292) . . . (5978): encodes the shuttle vector | |
| nucleic acid | ATTAAACCTGAGGCGCCAGGGGAGGATGCAAGCCCCGAGGAACTGAACCGCTATTATGCCAGTCTGCGGC | 60 |
| sequence | ATTATTTGAACCTGGTGACCAGACAGCGCTAC | |
| of PYY | | |
| (3-36) | | |
| oxyX4 AA | MGVHECPAWLWLLLSLLSLPLGLPVLGRHSQGTFTSDYSKYLDSRRAQDFVQWLMNTKRNRNNIARHHSQ | 61 |
| sequence | GTFTSDYSKYLDSRRAQDFVQWLMNTKRNRNNIARHHSQGTFTSDYSKYLDSRRAQDFVQWLMNTKRNRN | |
| | NIARHHSQGTFTSDYSKYLDSRRAQDFVQWLMNTKRNRNNIA | |
| | (1) . . . (27) : EPO signal sequence | |
| | (29) . . . (65): Oxyntomodulin sequence | |
| optimized | GGCCGATTCATTAATGCAGGGGCCGCTGCGGCCATCATCAATAATATACCTTATTTTGGATTGAAGCCAA | 62 |
| nucleotide | TATGATAATGAGGGGGTGGAGTTTGTGACGTGGCGCGGGGCGTGGGAACGGGGCGGGTGACGTAGTAGTG | |
| sequence in | TGGCGGAAGTGTGATGTTGCAAGTGTGGCGGAACACATGTAAGCGACGGATGTGGCAAAAGTGACGTTTT | |
| EF1a | TGGTGTGCGCCGGTGTACACAGGAAGTGACAATTTTCGCGCGGTTTTAGGCGGATGTTGTAGTAAATTTG | |
| expression | GGCGTAACCGAGTAAGATTTGGCCATTTTCGCGGGAAAACTGAATAAGAGGAAGTGAAATCTGAATAATT | |
| cassette: | TTGTGTTACTCATAGCGCGTAATATTTGTCTAGGGCCGCGGGGACTTTGACCGTTTACGTGGAGACTCGC | |
| pAd-MAR- | CCAGGTGTTTTTCTCAGGTGTTTTCCGCGTTCCGGGTCAAAGTTGGCGTTTTATTATTATAGTCAGCTGA | |
| EF1a-opt | CGTGTAGTGTATTTATACCCGGTGAGTTCCTCAAGAGGCCACTCTTGAGTGCCAGCGAGTAGAGTTTTCT | |
| Oxy X4 | CCTCCGAGCCGCTCCGACACCGGGAGGCGCGCCTTAATTAAAATTATCTCTAAGGCATGTGAACTGGCTG | |
| | TCTTGGTTTTCATCTGTACTTCATCTGCTACCTCTGTGACCTGAAACATATTTATAATTCCATTAAGCTG | |
| | TGCATATGATAGATTTATCATATGTATTTTCCTTAAAGGATTTTTGTAAGAACTAATTGAATTGATACCT | |
| | GTAAAGTCTTTATCACACTACCCAATAAATAATAAATCTCTTTGTTCAGCTCTCTGTTTCTATAAATATG | |
| | TACCAGTTTTATTGTTTTTAGTGGTAGTGATTTTATTCTCTTTCTATATATATACACACACATGTGTGCA | |
| | TTCATAAATATATACAATTTTTATGAATAAAAAATTATTAGCAATCAATATTGAAAACCACTGATTTTTG | |
| | TTTATGTGAGCAAACAGCAGATTAAAAGGCTAGCCTGCAGGAGTCAATGGGAAAAACCCATTGGAGCCAA | |
| | GTACACTGACTCAATAGGGACTTTCCATTGGGTTTTGCCCAGTACATAAGGTCAATAGGGGGTGAGTCAA | |
| | CAGGAAAGTCCCATTGGAGCCAAGTACATTGAGTCAATAGGGACTTTCCAATGGGTTTTGCCCAGTACAT | |
| | AAGGTCAATGGGAGGTAAGCCAATGGGTTTTTCCCATTACTGACATGTATACTGAGTCATTAGGGACTTT | |
| | CCAATGGGTTTTGCCCAGTACATAAGGTCAATAGGGGTGAATCAACAGGAAAGTCCCATTGGAGCCAAGT | |
| | ACACTGAGTCAATAGGGACTTTCCATTGGGTTTTGCCCAGTACAAAAGGTCAATAGGGGGTGAGTCAATG | |
| | GGTTTTTCCCATTATTGGCACATACATAAGGTCAATAGGGGTGACTAGTGGAGAAGAGCATGCTTGAGGG | |
| | CTGAGTGCCCCTCAGTGGGCAGAGAGCACATGGCCCACAGTCCCTGAGAAGTTGGGGGGAGGGGTGGGCA | |
| | ATTGAACTGGTGCCTAGAGAAGGTGGGGCTTGGGTAAACTGGGAAAGTGATGTGGTGTACTGGCTCCACC | |
| | TTTTTCCCCAGGGTGGGGGAGAACCATATATAAGTGCAGTAGTCTCTGTGAACATTCAAGCATCTGCCTT | |
| | CTCCCTCCTGTGAGTTTGgtaagtcactgactgtctatgcctgggaaagggtgggcaggaggtggggcag | |
| | tgcaggaaaagtggcactgtgaaccctgcagccctagacaattgtactaaccttcttctctttcctctcc | |
| | tgacagGTTGGTGTACAGTAGTAGCAAGCTTGCATGCCTGCAGGTCGACTCTAGACTGCCATGGGCGTCC | |
| | ACGAATGTCCCGCATGGCTGTGGCTTCTCCTCAGCCTCCTGAGCCTGCCGCTGGGTTTGCCTGTCTTGGG | |
| | ACGACATTCACAAGGCACATTCACCAGCGACTACAGTAAGTACCTCGATAGCCGCAGAGCCCAGGATTTT | |
| | GTGCAATGGTTGATGAACACAAAGCGGAACAGGAACAACATAGCCAGACACCACTCTCAAGGGACCTTCA | |
| | CATCTGACTATTCTAAATACCTTGACAGCCGCCGGGCTCAAGACTTCGTTCAGTGGTTGATGAATACCAA | |
| | ACGCAATAGAAACAATATTGCTAGGCATCACTCACAGGGGACTTTCACTAGTGACTATTCAAAATACCTG | |
| | GACTCCCGGAGAGCACAGGATTTTGTGCAGTGGCTGATGAACACAAAGCGCAATCGAAACAACATCGCCA | |
| | GACACCATTCCCAGGGCACATTTACCAGCGATTACTCCAAGTATCTCGACAGTCGACGGGCCCAAGATTT | |
| | TGTCCAGTGGCTTATGAATACAAAGCGGAACCGGAATAATATCGCATGAGGATCCCCGGGAGATATCCTA | |
| | GGCTTGGCCAGACATGATAAGATACATTGATGAGTTTGGACAAACCACAACTAGAATGCAGTGAAAAAAA | |
| | TGCTTTATTTGTGAAATTTGTGATGCTATTGCTTTATTTGTAACCATTATAAGCTGCAATAAACAAGTTA | |
| | ACAACAACAATTGCATTCATTTTATGTTTCAGGTTCAGGGGGAGGTGTGGGAGGTTTTTTAAAGCAAGTA | |
| | AAACCTCTACAAATGTGGTATGGAATTCAGTCAATATGTTCACCCCAAAAAAGCTGTTTGTTAACTTGCC | |
| | AACCTCATTCTAAAATGTATATAGAAGCCCAAAAGACAATAACAAAAATATTCTTGTAGAACAAAATGGG | |
| | AAAGAATGTTCCACTAAATATCAAGATTTAGAGCAAAGCATGAGATGTGTGGGGATAGACAGTGAGGCTG | |
| | ATAAAATAGAGTAGAGCTCAGAAACAGACCCATTGATATATGTAAGTGACCTATGAAAAAAATATGGCAT | |
| | TTTACAATGGGAAAATGATGGTCTTTTTCTTTTTTAGAAAAACAGGGAAATATATTTATATGTAAAAAAT | |
| | AAAAGGGAACCCATATGTCATACCATACACACAAAAAAATTCCAGTGAATTATAAGTCTAAATGGAGAAG | |
| | GCAAAACTTTAAATCTTTTAGAAAATAATATAGAAGCATGCCATCAAGACTTCAGTGTAGAGAAAAATTT | |
| | CTTATGACTCAAAGTCCTAACCACAAAGAAAAGATTGTTAATTAGATTGCATGAATATTAAGACTTATTT | |
| | TTAAAATTAAAAAACCATTAAGAAAAGTCAGGCCATAGAATGACAGAAAATATTTGCAACACCCCAGTAA | |
| | AGAGAATTGTAATATGCAGATTATAAAAAGAAGTCTTACAAATCAGTAAAAAATAAAACTAGACAAAAAT | |
| | TTGAACAGATGAAAGAGAAACTCTAAATAATCATTACACATGAGAAACTCAATCTCAGAAATCAGAGAAC | |
| | TATCATTGCATATACACTAAATTAGAGAAATATTAAAAGGCTAAGTAACATCTGTGGCTTAATTAAGGCG | |
| | CGCCGGGCCCCTACGTCACCCGCCCCGTTCCCACGCCCCGCGCCACGTCACAAACTCCACCCCCTCATTA | |
| | TCATATTGGCTTCAATCCAAAATAAGGTATATTATTGATGATGGCCGCAGCGGCCCTGGCGTAATAGCGA | |
| | AGAGGCCCGCACCGATCGCCCTTCCCAACAGTTGCGCAGCCTGAATGGCGAATGGGACGCGCCCTGTAGC | |
| | GGCGCATTAAGCGCGGCGGGTGTGGTGGTTACGCGCAGCGTGACCGCTACACTTGCCAGCGCCCTAGCGC | |
| | CCGCTCCTTTCGCTTTCTTCCCTTCCTTTCTCGCCACGTTCGCCGGCTTTCCCCGTCAAGCTCTAAATCG | |
| | GGGGCTCCCTTTAGGGTTCCGATTTAGTGCTTTACGGCACCTCGACCCCAAAAAACTTGATTAGGGTGAT | |
| | GGTTCACGTAGTGGGCCATCGCCCTGATAGACGGTTTTTCGCCCTTTGACGTTGGAGTCCACGTTCTTTA | |
| | ATAGTGGACTCTTGTTCCAAACTGGAACAACACTCAACCCTATCTCGGTCTATTCTTTTGATTTATAAGG | |
| | GATTTTGCCGATTTCGGCCTATTGGTTAAAAAATGAGCTGATTTAACAAAAATTTAACGCGAATTTTAAC | |
| | AAAATATTAACGCTTACAATTTAGGTGGCACTTTTCGGGGAAATGTGCGCGGAACCCCTATTTGTTTATT | |
| | TTTCTAAATACATTCAAATATGTATCCGCTCATGAGACAATAACCCTGATAAATGCTTCAATAATATTGA | |
| | AAAAGGAAGAGTATGAGTATTCAACATTTCCGTGTCGCCCTTATTCCCTTTTTTGCGGCATTTTGCCTTC | |
| | CTGTTTTTGCTCACCCAGAAACGCTGGTGAAAGTAAAAGATGCTGAAGATCAGTTGGGTGCACGAGTGGG | |
| | TTACATCGAACTGGATCTCAACAGCGGTAAGATCCTTGAGAGTTTTCGCCCCGAAGAACGTTTTCCAATG | |
| | ATGAGCACTTTTAAAGTTCTGCTATGTGGCGCGGTATTATCCCGTATTGACGCCGGGCAAGAGCAACTCG | |
| | GTCGCCGCATACACTATTCTCAGAATGACTTGGTTGAGTACTCACCAGTCACAGAAAAGCATCTTACGGA | |
| | TGGCATGACAGTAAGAGAATTATGCAGTGCTGCCATAACCATGAGTGATAACACTGCGGCCAACTTACTT | |
| | CTGACAACGATCGGAGGACCGAAGGAGCTAACCGCTTTTTTGCACAACATGGGGGATCATGTAACTCGCC | |
| | TTGATCGTTGGGAACCGGAGCTGAATGAAGCCATACCAAACGACGAGCGTGACACCACGATGCCTGTAGC | |
| | AATGGCAACAACGTTGCGCAAACTATTAACTGGCGAACTACTTACTCTAGCTTCCCGGCAACAATTAATA | |
| | GACTGGATGGAGGCGGATAAAGTTGCAGGACCACTTCTGCGCTCGGCCCTTCCGGCTGGCTGGTTTATTG | |
| | CTGATAAATCTGGAGCCGGTGAGCGTGGGTCTCGCGGTATCATTGCAGCACTGGGGCCAGATGGTAAGCC | |
| | CTCCCGTATCGTAGTTATCTACACGACGGGGAGTCAGGCAACTATGGATGAACGAAATAGACAGATCGCT | |
| | GAGATAGGTGCCTCACTGATTAAGCATTGGTAACTGTCAGACCAAGTTTACTCATATATACTTTAGATTG | |
| | ATTTAAAACTTCATTTTTAATTTAAAAGGATCTAGGTGAAGATCCTTTTTGATAATCTCATGACCAAAAT | |
| | CCCTTAACGTGAGTTTTCGTTCCACTGAGCGTCAGACCCCGTAGAAAAGATCAAAGGATCTTCTTGAGAT | |
| | CCTTTTTTTCTGCGCGTAATCTGCTGCTTGCAAACAAAAAAACCACCGCTACCAGCGGTGGTTTGTTTGC | |
| | CGGATCAAGAGCTACCAACTCTTTTTCCGAAGGTAACTGGCTTCAGCAGAGCGCAGATACCAAATACTGT | |
| | TCTTCTAGTGTAGCCGTAGTTAGGCCACCACTTCAAGAACTCTGTAGCACCGCCTACATACCTCGCTCTG | |
| | CTAATCCTGTTACCAGTGGCTGCTGCCAGTGGCGATAAGTCGTGTCTTACCGGGTTGGACTCAAGACGAT | |
| | AGTTACCGGATAAGGCGCAGCGGTCGGGCTGAACGGGGGGTTCGTGCACACAGCCCAGCTTGGAGCGAAC | |
| | GACCTACACCGAACTGAGATACCTACAGCGTGAGCTATGAGAAAGCGCCACGCTTCCCGAAGGGAGAAAG | |
| | GCGGACAGGTATCCGGTAAGCGGCAGGGTCGGAACAGGAGAGCGCACGAGGGAGCTTCCAGGGGGAAACG | |
| | CCTGGTATCTTTATAGTCCTGTCGGGTTTCGCCACCTCTGACTTGAGCGTCGATTTTTGTGATGCTCGTC | |
| | AGGGGGGCGGAGCCTATGGAAAAACGCCAGCAACGCGGCCTTTTTACGGTTCCTGGCCTTTTGCTGGCCT | |
| | TTTGCTCACATGTTCTTTCCTGCGTTATCCCCTGATTCTGTGGATAACCGTATTACCGCCTTTGAGTGAG | |
| | CTGATACCGCTCGCCGCAGCCGAACGACCGAGCGCAGCGAGTCAGTGAGCGAGGAAGCGGAAGAGCGCCC | |
| | AATACGCAAACCGCCTCTCCCCGCGCGTTGGCCGATTCATTAATGCAGGGGCCGCTGCGGCCATCATCAA | |
| | TAATATACCTTATTTTGGATTGAAGCCAATA | |

What is claimed is:

1. A genetically-modified micro-organ that provides a sustained delivery of at least one therapeutic peptide when implanted in a human subject in vivo comprising a viral vector comprising at least two nucleic acid sequences encoding at least one therapeutic peptide separated by at least one cleavable linker, wherein the genetically-modified micro-organ provides the peptide as a monomer for a sustained period of at least three months as measured in vitro or in vivo.

2. A genetically-modified micro-organ that provides a sustained delivery of at least one therapeutic peptide when implanted in a human subject in vivo, comprising a nucleic acid expression cassette comprising at least two nucleic acid sequences encoding at least one therapeutic peptide separated by at least one cleavable linker, wherein the genetically-modified micro-organ provides the peptide as a monomer for a sustained period of at least three months as measured in vitro or in vivo.

3. The genetically-modified micro-organ of claim 2, wherein there are at least two copies of the therapeutic peptide, and wherein the peptide is expressed first as a polypeptide, and wherein the polypeptide is cleaved by an endogenous protease in vivo to produce at least two peptide monomers.

4. The genetically-modified micro-organ of claim 2, wherein the sustained period of at least three months is measured in vivo in serum.

5. The genetically-modified micro-organ of claim 2, wherein the therapeutic peptide is GLP-2 or a variant of GLP-2 that retains GLP-2 functional activity.

6. The genetically-modified micro-organ of claim 2, wherein the therapeutic peptide is oxyntomodulin or a variant of oxyntomodulin that retains oxyntomodulin functional activity.

7. The genetically-modified micro-organ of any one of claim 2, wherein the therapeutic peptide is peptide YY (PYY) or a variant of PYY that retains PYY functional activity.

8. The genetically-modified micro-organ of claim 2, wherein the therapeutic peptide comprises or consists of peptide YY (PYY) or a variant of PYY that retains PYY functional activity, and oxyntomodulin or a variant of oxyntomodulin that retains oxyntomoulin functional activity.

9. The genetically-modified micro-organ of claim 1, wherein said vector is a helper-dependent adenoviral vector or an adeno-associated viral vector.

10. The genetically-modified micro-organ of claim 2, wherein the nucleic acid encoding the therapeutic peptide is operably-linked to an upstream regulatory sequence.

11. The genetically-modified micro-organ of claim 10, wherein the upstream regulatory sequence is chosen from a MAR sequence, a CAG promoter sequence, an EF1α promoter sequence and a WPRE sequence.

12. The genetically-modified micro-organ of claim 11, wherein the nucleic acid further encodes a downstream regulatory sequence chosen from a MAR sequence, a CAG promoter sequence, an EF1α promoter sequence and a WPRE sequence.

13. The genetically-modified micro-organ of claim 2, wherein the nucleic acids are CpG-free.

14. The genetically-modified micro-organ of claim 2, wherein said at least one genetically modified micro-organ is a genetically modified dermal micro-organ.

15. The genetically-modified micro-organ of claim 2, wherein the genetically-modified micro-organ expresses said therapeutic peptide for a sustained period of at least three, four, five, or six months as measured in vitro or in vivo.

16. The genetically-modified micro-organ of claim 2, wherein the vector comprises nucleic acids encoding a signal peptide.

17. The genetically-modified micro-organ of claim 16, wherein the signal peptide comprises SEQ ID NO: 9, 18, 19, 20, or 29.

18. The genetically-modified micro-organ of claim 17, wherein the signaling peptide is chosen from a proglucagon signaling peptide, an EPO signaling peptide, a PYY signaling peptide, a glucagon signaling peptide, and a tripsinogen-2 signaling peptide.

19. The genetically-modified micro-organ of claim 16, wherein the signaling peptide is encoded by nucleic acids comprising SEQ ID NO: 8, 28, or 30.

20. The genetically-modified micro-organ of claim 2, wherein at least one therapeutic peptide comprises or consists of SEQ ID NO: 1.

21. The genetically-modified micro-organ of claim 2, wherein at least one therapeutic peptide is encoded by nucleic acids comprising of or consisting of one of the following sequences: SEQ ID NO: 2, SEQ ID NO: 3, SEQ ID NO: 10 or SEQ ID NO: 11.

22. (canceled)

23. (canceled)

24. The genetically-modified micro-organ of claim 2, wherein at least one linker comprises any one of SEQ ID NO: 13-17, 24, or 32-33.

25. The genetically-modified micro-organ of claim 24, wherein at least one linker is encoded by nucleic acids comprising SEQ ID NO: 12.

26. The genetically-modified micro-organ of claim 2, further comprising a furin or convertase cleavage site.

27. The genetically-modified micro-organ of claim 26, wherein the furin or convertase cleavage site is non-native to the linker and/or signaling peptide sequence and is immediately upstream and/or downstream of the linker and/or signaling peptide.

28. The genetically-modified micro-organ of claim 2, wherein the expression cassette comprises nucleic acids encoding at least three copies of the same therapeutic peptide.

29. The genetically-modified micro-organ of claim 28, wherein the at least three nucleic acids encoding the therapeutic peptide are separated by cleavable linkers in the following pattern: therapeutic peptide-linker-therapeutic peptide-linker-therapeutic peptide.

30. The genetically-modified micro-organ of claim 29, wherein the linkers are the same or different.

31. (canceled)

32. The genetically-modified micro-organ of claim 29, further comprising a furin or convertase cleavage site.

33. The genetically-modified micro-organ of claim 32, wherein the furin or convertase cleave site is non-native to the linker and signaling peptide and is immediately upstream and/or downstream of the linker and/or signaling peptide.

34. The genetically-modified micro-organ of claim 2, wherein the nucleic acid sequences encoding the therapeutic peptide are the same or different.

35. (canceled)

36. The genetically-modified micro-organ of claim 2, wherein the vector or expression cassette comprises nucleic acids encoding a signaling peptide at the N-terminus of the expressed polypeptide.

37. A genetically-modified micro-organ comprising i) the nucleic acids of SEQ ID NO: 7 or SEQ ID NO: 5, or comprising nucleic acids having 85%, 90%, or 95% identity to SEQ ID NO: 7 or SEQ ID NO: 5; or ii) a viral vector comprising the nucleic acids of SEQ ID NO: 21, or comprising nucleic acids having 85%, 90%, or 95% identity to SEQ ID NO: 21.

38.-43. (canceled)

44. A method of treating or preventing a disease or disorder in a human subject that can be treated or prevented by administration of a therapeutic peptide over a sustained time period comprising:
a. providing at least one genetically-modified micro-organ or composition according to claim 2 that provides a sustained delivery of a therapeutic peptide;
b. determining the therapeutic peptide secretion levels of the at least one genetically-modified micro-organ in vitro;
c. implanting the at least one genetically-modified micro-organ in the human subject; and
d. measuring therapeutic peptide levels in the blood serum of said subject; wherein implantation of said at least one genetically-modified micro-organ or composition increases the in vivo serum peptide levels over basal levels for at least three months, thereby treating or preventing the disease or disorder.

45. The method of claim 44, wherein the therapeutic peptide is GLP-2 or a GLP-2 variant that retains at least one GLP-2 functional activity, oxyntomodulin or a oxyntomodulin variant that retains at least one oxyntomodulin functional activity, or PYY or a PYY variant that retains at least one PYY functional activity.

46. (canceled)

47. (canceled)

48. (canceled)

49. The method of claim 44, wherein the therapeutic peptide is oxyntomodulin and PYY, wherein oxyntomodulin and PYY may be variants that retain at least one of their functional activities.

50. The method of claim 44, wherein the disease or disorder is chosen from short bowel syndrome (SBS), Crohn's disease, ulcerative colitis, inflammatory bowel disease (IBD), or osteoporosis.

51. The method of any one of claim 44, wherein the disease or disorder is chosen from Prader-Willi Syndrome, hypothalamic hyperphagia, binge eating disorder, over-eating, obesity, and diabetes.

52. The method of claim 44, wherein the therapeutic peptide provides adjuvant therapy during cancer chemotherapy.

53. The method of claim 44, wherein the patient has SBS and the patient is dependent on parenteral support.

54. (canceled)


SEQUENCE LISTING (62 sequences)

SEQ ID NO: 1 - protein, 33 aa, Homo sapiens
  misc_feature (1)..(33): Human GLP-2 Wild Type Amino Acid Sequence
HADGSFSDEMNTILDNLAARDFINWLIQTKITD

SEQ ID NO: 2 - DNA, 99 bp, Homo sapiens
  misc_feature (1)..(99): Human GLP-2 Wild Type Nucleic Acid Sequence
catgctgatggttctttctctgatgagatgaacaccattcttgataatcttgccgccagggactttataaactggttgattcagaccaaaatcactgac

SEQ ID NO: 3 - protein, 33 aa, Artificial Sequence
  Synthetic  GLP-2 Variant Amino Acid Sequence (point mutation in second amino acid)
HGDGSFSDEMNTILDNLAARDFINWLIQTKITD

SEQ ID NO: 4 - protein, 103 aa, Artificial Sequence
  Synthetic  GLP-2 Wild Type Amino Acid Sequence in Approach B Construct(signal peptide-GLP-2 wt-linker-GLP-2 wt)
MKSIYFVAGLFVMLVQGSWQHADGSFSDEMNTILDNLAARDFINWLIQTKITDRHDFPEEVAIVEELGRRHADGSFSDEMNTILDNLAARDFINWLIQTKITD

SEQ ID NO: 5 - DNA, 309 bp, Artificial Sequence
  Synthetic  Codon Optimized Nucleic Acid Sequence Encoding Wild Type GLP-2 in Approach B Construct Encoding SEQ ID NO 4
atgaagtccatttactttgtggctggactgtttgtcatgctcgtgcagggaagctggcagcatgctgacgggagtttctccgacgagatgaacaccatcctggataatctggcagcccgggatttcatcaattggctcatacaaacaaagattacagatcggcacgacttcccagaagaggttgccatagttgaagagctgggacggcggcatgcagacggctccttctccgatgagatgaataccattctggataaccttgccgcaagagacttcatcaactggctgattcaaaccaagatcaccgac

SEQ ID NO: 6 - protein, 101 aa, Artificial Sequence
  Synthetic  GLP-2 Variant Amino Acid Sequence in Approach B Construct(signal peptide-GLP-2 variant-linker-GLP-2 variant)
MKSIYFVAGLFVMLVQGSWQHGDGSFSDEMNTILDNLAARDFINWLIQTKITDDFPEEVAIVEELGRRHGDGSFSDEMNTILDNLAARDFINWLIQTKITD

SEQ ID NO: 7 - DNA, 306 bp, Artificial Sequence
  Synthetic  Codon Optimized GLP-2 Variant Nucleic Acid Sequence in Approach B Construct Encoding SEQ ID NO 6
atgaaatctatatacttcgtggccggcctctttgtgatgctggtgcaagggtcttggcagcacggggatggctccttcagtgacgagatgaacactatactggataacctggcggcacgggatttcatcaattggctcatccagactaagattactgatgactttcctgaggaggtggcgattgtggaggaactcgggaggcgccatggcgacggctcattttccgacgagatgaacacgatactggacaatctggccgcccgggactttataaattggctcatccagacaaagattactgactaa

SEQ ID NO: 8 - DNA, 60 bp, Artificial Sequence
  Synthetic  Signaling Peptide Nucleic Acid Encoding SEQ ID NO 9
atgaaatctatatacttcgtggccggcctctttgtgatgctggtgcaagggtcttggcag

SEQ ID NO: 9 - protein, 20 aa, Homo sapiens
  misc_feature (1)..(20): Human Proglucagon Signaling Peptide Amino Acid
MKSIYFVAGLFVMLVQGSWQ

SEQ ID NO: 10 - DNA, 99 bp, Artificial Sequence
  Synthetic  A First Codon Optimized Nucleic Acid Sequence Encoding GLP-2 Variant SEQ ID NO 3
cacggggatggctccttcagtgacgagatgaacactatactggataacctggcggcacgggatttcatcaattggctcatccagactaagattactgat

SEQ ID NO: 11 - DNA, 99 bp, Artificial Sequence
  Synthetic  A Second Codon Optimized Nucleic Acid Sequence Encoding GLP-2 Variant SEQ ID NO 3
catggcgacggctcattttccgacgagatgaacacgatactggacaatctggccgcccgggactttataaattggctcatccagacaaagattactgac

SEQ ID NO: 12 - DNA, 45 bp, Artificial Sequence
  Synthetic  IP-2 Linker Nucleic Acid Sequence Encoding SEQ ID NO 13
gactttcctgaggaggtggcgattgtggaggaactcgggaggcgc

SEQ ID NO: 13 - protein, 15 aa, Artificial Sequence
  Synthetic  IP-2 Linker Amino Acid Sequence
DFPEEVAIVEELGRR

SEQ ID NO: 14 - protein, 17 aa, Artificial Sequence
  Synthetic  PCK1/2 linker, IP-2 Linker Amino Acid Sequence with RH cleavage site added
RHDFPEEVAIVEELGRR

SEQ ID NO: 15 - protein, 6 aa, Artificial Sequence
  Synthetic  IP-1 Linker Sequence
NRNNIA

SEQ ID NO: 16 - protein, 4 aa, Artificial Sequence
  Synthetic  GS Linker Sequence 1
GGGS

SEQ ID NO: 17 - protein, 6 aa, Artificial Sequence
  Synthetic  GS Linker Sequence 2
GSGGGS

SEQ ID NO: 18 - protein, 27 aa, Homo sapiens
  misc_feature (1)..(27): EPO Signal Peptide Sequence
MGVHECPAWLWLLLSLLSLPLGLPVLG

SEQ ID NO: 19 - protein, 15 aa, Homo sapiens
  misc_feature (1)..(15): Trypsin 1 Signal Peptide Sequence
MNPLLILTFVAAALA

SEQ ID NO: 20 - protein, 15 aa, Homo sapiens
  misc_feature (1)..(15): Trypsin 2 Signal Peptide Sequence
MNLLLILTFVAAAVA

SEQ ID NO: 21 - DNA, 5939 bp, Artificial Sequence
  Synthetic  HDAd-EF1a-GLP-2 variant ver B
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaaatctatatacttcgtggccggcctctttgtgatgctggtgcaagggtcttggcagcacggggatggctccttcagtgacgagatgaacactatactggataacctggcggcacgggatttcatcaattggctcatccagactaagattactgatgactttcctgaggaggtggcgattgtggaggaactcgggaggcgccatggcgacggctcattttccgacgagatgaacacgatactggacaatctggccgcccgggactttataaattggctcatccagacaaagattactgactaaggatccccatccagcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 22 - protein, 37 aa, Homo sapiens
  misc_feature (1)..(37): Amino acid sequence of oxyntomodulin
HSQGTFTSDYSKYLDSRRAQDFVQWLMNTKRNRNNIA

SEQ ID NO: 23 - DNA, 111 bp, Homo sapiens
  misc_feature (1)..(111): Nucleic acid sequence of oxyntomodulin
cactcacagggcactttcacttccgattactctaagtacctggacagcaggcgggcccaggatttcgtgcagtggcttatgaacacaaagagaaacagaaataatattgcc

SEQ ID NO: 24 - protein, 4 aa, Homo sapiens
  misc_feature (1)..(4): Amino acid sequence of furin linker
RAKR

SEQ ID NO: 25 - protein, 36 aa, Homo sapiens
  misc_feature (1)..(36): Amino acid sequence of protein YY (PYY)
YPIKPEAPGEDASPEELNRYYASLRHYLNLVTRQRY

SEQ ID NO: 26 - DNA, 108 bp, Homo sapiens
  misc_feature (1)..(108): Nucleic acid sequence of PYY
tatcctattaaacctgaggcgccaggggaggatgcaagccccgaggaactgaaccgctattatgccagtctgcggcattatttgaacctggtgaccagacagcgctac

SEQ ID NO: 27 - protein, 20 aa, Homo sapiens
  misc_feature (1)..(20): Amino acid sequence of proglucagon signaling peptide
MKSIYFVAGLFVMLVQGSWQ

SEQ ID NO: 28 - DNA, 60 bp, Homo sapiens
  misc_feature (1)..(60): Nucleic acid sequence of proglucagon signaling peptide
atgaagtctatctacttcgtggccggattgtttgttatgctggtgcagggttcctggcag

SEQ ID NO: 29 - protein, 28 aa, Homo sapiens
  misc_feature (1)..(28): Amino acid sequence of PYY signaling peptide
MVFVRRPWPALTTVLLALLVCLGALVDA

SEQ ID NO: 30 - DNA, 84 bp, Homo sapiens
  misc_feature (1)..(84): WT Nucleic acid sequence of PYY signaling peptide
atggtgttcgtgcgcaggccgtggcccgccttgaccacagtgcttctggccctgctcgtctgcctaggggcgctggtcgacgcc

SEQ ID NO: 31 - protein, 34 aa, Homo sapiens
  misc_feature (1)..(34): Amino acid sequence of PYY (3-36)
IKPEAPGEDASPEELNRYYASLRHYLNLVTRQRY

SEQ ID NO: 32 - protein, 15 aa, Homo sapiens
  misc_feature (1)..(15): Peptide sequence of propeptide (PP) linker
DFPEEVAIVEELGRR

SEQ ID NO: 33 - protein, 24 aa, Artificial Sequence
  Synthetic  Amino acid sequence of 2A linker
APVKQTLNFDLLKLAGDVESNPGP

SEQ ID NO: 34 - protein, 113 aa, Artificial Sequence
  Synthetic  PYY (3-36) Amino Acid Sequence in Approach 1 Construct, (PYY signal peptide-PYY-PCK1-Propeptide- PCK1-PYY)
MVFVRRPWPALTTVLLALLVCLGALVDAIKPEAPGEDASPEELNRYYASLRHYLNLVTRQRYRHDFPEEVAIVEELGRRIKPEAPGEDASPEELNRYYASLRHYLNLVTRQRY

SEQ ID NO: 35 - DNA, 339 bp, Artificial Sequence
  Synthetic  Codon Optimized Nucleic Acid Sequence Encoding PYY (3-36) Approach 1 Construct
atggtatttgtcagacgaccttggcctgccctcaccacggtgttgctcgccttgctcgtttgcttgggggccttggtggacgctatcaagcctgaggcacccggagaagacgcatcccccgaggaattgaaccgctattacgcatccttgcggcactacctgaacctcgtgaccaggcaacgctatcggcacgacttccctgaagaagtcgcaatcgtcgaagagctcggcaggagaatcaaacctgaggctcctggtgaggacgccagtccggaggaactgaatcgctactacgcgtcacttaggcactacctgaacctggtcactcgccagagatac

SEQ ID NO: 36 - protein, 99 aa, Artificial Sequence
  Synthetic  PYY (3-36) Amino Acid Sequence in Approach 2 Construct, (PYY signal peptide-PYY-PAM-PCK1-PYY)
MVFVRRPWPALTTVLLALLVCLGALVDAIKPEAPGEDASPEELNRYYASLRHYLNLVTRQRYGKRIKPEAPGEDASPEELNRYYASLRHYLNLVTRQRY

SEQ ID NO: 37 - DNA, 297 bp, Artificial Sequence
  Synthetic  Codon Optimized Nucleic Acid Sequence Encoding PYY (3-36) Approach 2 Construct
atggtctttgttagacgaccgtggccggctctgacaactgtgctgctcgcccttctcgtgtgcctgggagctcttgttgatgccatcaagcctgaggcacctggagaagacgcatcccctgaggaactgaaccgctattacgcatcattgagacactaccttaacctcgtgacaagacagcggtacggtaagagaatcaagccagaggcccccggggaggatgcttccccagaggagttgaataggtattacgctagcctgagacattacttgaaccttgtgacacgacagcgatat

SEQ ID NO: 38 - protein, 117 aa, Artificial Sequence
  Synthetic  PYY (3-36) Amino Acid Sequence in Approach 3 Construct, (PYY signal peptide-PYY-PAM-Propeptide- PAM-PYY)
MVFVRRPWPALTTVLLALLVCLGALVDAIKPEAPGEDASPEELNRYYASLRHYLNLVTRQRYGKRDFPEEVAIVEELGRRGKRIKPEAPGEDASPEELNRYYASLRHYLNLVTRQRY

SEQ ID NO: 39 - DNA, 351 bp, Artificial Sequence
  Synthetic  Codon Optimized Nucleic Acid Sequence Encoding PYY (3-36) Approach 3 Construct
atggtatttgtgagacgcccctggcccgcgctgacaaccgtgttgctggcacttcttgtgtgccttggagctctcgttgatgccatcaagcctgaagcaccgggggaagacgcttcccccgaggagctcaatcgatactatgctagtctgcgacattatctgaaccttgtgaccaggcagcggtacggcaaacgggattttccagaggaagtggcgatcgtcgaggagctcggacggcggggaaaacgcatcaaacccgaggccccaggcgaggatgcttcacctgaggaacttaaccgctactacgcaagcctgcggcattaccttaacctcgtgaccagacagcgctac

SEQ ID NO: 40 - protein, 116 aa, Artificial Sequence
  Synthetic  PYY (3-36) Amino Acid Sequence in Approach 6 Construct, (proGlucagon signal peptide-PYY- Furin-2A-PYY)
MKSIYFVAGLFVMLVQGSWQIKPEAPGEDASPEELNRYYASLRHYLNLVTRQRYRAKRAPVKQTLNFDLLKLAGDVESNPGPIKPEAPGEDASPEELNRYYASLRHYLNLVTRQRY

SEQ ID NO: 41 - DNA, 348 bp, Artificial Sequence
  Synthetic  Codon Optimized Nucleic Acid Sequence Encoding PYY (3-36) Approach 6 Construct
atgaagtctatctacttcgtggccggattgtttgttatgctggtgcagggttcctggcagattaagcctgaggctcccggagaagacgccagccccgaagagctcaaccgctactacgctagtctgcggcactaccttaacctcgtaacaagacagcggtatcgggccaagagggcacccgtgaaacagacattgaatttcgacctgctgaaactcgcaggagacgtcgagtctaatcctggaccgatcaaacctgaagcccccggtgaagacgcatcaccagaagagttgaaccgctattacgcttctcttagacactacttgaacttggtgacacggcaacggtac

SEQ ID NO: 42 - protein, 30 aa, Artificial Sequence
  Synthetic  Amino acid sequence of peptide C
DGPDRLLSKTFFPDGEDRPVRSRSEGPDLW

SEQ ID NO: 43 - DNA, 30669 bp, Artificial Sequence
  Synthetic  HDAd-EF1a-PYY Approach 1 Codon Optimized Nucleic Acid Sequence Encoding PYY (3-36) Approach 1 Construct in HDdelta28E4-MAR-EF1a shuttle vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagaatgcgccacttcactccagcctgggaaacagagcaagactctgtctcaaaaaaaacaaaacaaacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactggggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtggctcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactccgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagcttatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagattgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgagtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggtatttgtcagacgaccttggcctgccctcaccacggtgttgctcgccttgctcgtttgcttgggggccttggtggacgctatcaagcctgaggcacccggagaagacgcatcccccgaggaattgaaccgctattacgcatccttgcggcactacctgaacctcgtgaccaggcaacgctatcggcacgacttccctgaagaagtcgcaatcgtcgaagagctcggcaggagaatcaaacctgaggctcctggtgaggacgccagtccggaggaactgaatcgctactacgcgtcacttaggcactacctgaacctggtcactcgccagagatactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatga

SEQ ID NO: 44 - DNA, 30627 bp, Artificial Sequence
  Synthetic  HDAd-EF1a-PYY Approach 2 Codon Optimized Nucleic Acid Sequence Encoding PYY (3-36) Approach 2 Construct in HDdelta28E4-MAR-EF1a shuttle vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagaatgcgccacttcactccagcctgggaaacagagcaagactctgtctcaaaaaaaacaaaacaaacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactggggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtggctcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactccgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagcttatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagattgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgagtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggtctttgttagacgaccgtggccggctctgacaactgtgctgctcgcccttctcgtgtgcctgggagctcttgttgatgccatcaagcctgaggcacctggagaagacgcatcccctgaggaactgaaccgctattacgcatcattgagacactaccttaacctcgtgacaagacagcggtacggtaagagaatcaagccagaggcccccggggaggatgcttccccagaggagttgaataggtattacgctagcctgagacattacttgaaccttgtgacacgacagcgatattgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatga

SEQ ID NO: 45 - DNA, 30681 bp, Artificial Sequence
  Synthetic  HDAd-EF1a-PYY Approach 3 Codon Optimized Nucleic Acid Sequence Encoding PYY (3-36) Approach 3 Construct in HDdelta28E4-MAR-EF1a shuttle vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagaatgcgccacttcactccagcctgggaaacagagcaagactctgtctcaaaaaaaacaaaacaaacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactggggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtggctcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactccgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagcttatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagattgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgagtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggtatttgtgagacgcccctggcccgcgctgacaaccgtgttgctggcacttcttgtgtgccttggagctctcgttgatgccatcaagcctgaagcaccgggggaagacgcttcccccgaggagctcaatcgatactatgctagtctgcgacattatctgaaccttgtgaccaggcagcggtacggcaaacgggattttccagaggaagtggcgatcgtcgaggagctcggacggcggggaaaacgcatcaaacccgaggccccaggcgaggatgcttcacctgaggaacttaaccgctactacgcaagcctgcggcattaccttaacctcgtgaccagacagcgctactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatga

SEQ ID NO: 46 - DNA, 30678 bp, Artificial Sequence
  Synthetic  HDAd-EF1a-PYY Approach 6 Codon Optimized Nucleic Acid Sequence Encoding PYY (3-36)Approach 6 in HDdelta28E4-MAR-EF1a shuttle vector
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttgatatcaagcttatcgataccgtaaacaagtctttaattcaagcaagactttaacaagttaaaaggagcttatgggtaggaagtagtgttatgatgtatgggcataaagggttttaatgggatagtgaaaatgtctataataatacttaaatggctgcccaatcacctacaggattgatgtaaacatggaaaaggtcaaaaacttgggtcactaaaatagatgattaatggagaggatgaggttgatagttaaatgtagataagtggtcttattctcaataaaaatgtgaacataaggcgagtttctacaaagatggacaggactcattcatgaaacagcaaaaactggacatttgttctaatctttgaagagtatgaaaaattcctattttaaaggaaaacagtaactcacaggaaataccaacccaacataaaatcagaaacaatagtctaaagtaataaaaatcaaacgtttgcacgatcaaattatgaatgaaattcactactaaaattcacactgattttgtttcatccacagtgtcaatgttgtgatgcatttcaattgtgtgacacaggcagactgtggatcaaaagtggtttctggtgcgacttactctcttgagtatacctgcagtcccctttcttaagtgtgttaaaaaaaaagggggatttcttcaattcgccaatactctagctctccatgtgctttctaggaaacaagtgttaacccaccttatttgtcaaacctagctccaaaggacttttgactccccacaaaccgatgtagctcaagagagggtatctgtcaccagtatgtatagtgaaaaaagtatcccaagtcccaacagcaattcctaaaaggagtttatttaaaaaaccacacacacctgtaaaataagtatatatcctccaaggtgactagttttaaaaaaacagtattggctttgatgtaaagtactagtgaatatgttagaaaaatctcactgtaaccaagtgaaatgaaagcaagtatggtttgcagagattcaaagaaaatataagaaaacctactgttgccactaaaaagaatcatatattaaatatactcacacaatagctcttcagtctgataaaatctacagtcataggaatggatctatcactatttctattcagtgctttgatgtaatccagcaggtcagcaaagaatttatagccccccttgagcacacagagggctacaatgtgatggcctcccatctccttcatcacatctcgagcaagacgttcagtcctacagaaataaaatcaggaatttaatagaaagtttcatacattaaactttataacaaacacctcttagtcattaaacttccacaccaacctgggcaatatagtgagaccccatgcctgcaaaaaaaaaaaaattagccaggcatggtagcatgtacctgtagtcccagctacttgagaggtgaggtgggaaaatcactttagtgcaggatgttgaggctggagtgaactgtgattgtgccactgcactccagcctggacaatagagcaagaccttgtctcaaaaaaatgcattaaaaattttttttaaatcttccacgtaacacatcctttgccctcatgtttcataaggtaaaaaatttgataccttcaaaaaaaccaagcataccactatcataattttttttaaatgcaaataaaaacaagataccattttcacctatcagactggcaggttctgattaaatgaaatttcttggataatatacaatattaagagagactgtagaaactgggccagtggctcatgcctgtaatcccagcactttgggaggctgggtaacatggcgaaccctgtttctacaaaataaaaatattagctgggagtggtggcgcacacctatagtcccagctactcaggaggctgaggtggaaggatcgcttgaacccaggaggttgagactgcagtgaactgtgatcattctgctgcactgcaccccagcctgggcaacagagaccttgtctcaaaaaaaaaaaaaaaagagacaaattgtgaagagaaaggtactctcatataacatcaggagtataaaatgattcaacttcttagaggaaaatttggcaataccaaaatattcaataaactctttccccttgacccagaaattccacttgaataaagctgaacaagtaccaaacatgtaaaagaatgtttcttctagtacagtcggtaagaacaaaatagtgtctatcaatagtggactggttaaatcagttatggtatctccataagacagaatgctatgcaacctttaaaatatattagatagctctagacacactaatattaaaagtgtccaataacatttaaaactatactcatacgttaaaatataaatgtatatatgtacttttgcatatagtatacatgcatagccagtgcttgagaagaaatgtgtacagaaggctgaaaggagagaactttagtcttcttgtttatggcctccatagttagaatattttataacacaaatattttgatattataattttaaaataaaaacacagaatagccagacatacaatgcaagcattcaataccaggtaaggtttttcactgtaattgacttaacagaaaattttcaagctagatgtgcataataataaaaatctgaccttgccttcatgtgattcagccccagtccattaccctgtttaggactgagaaatgcaagactctggctagagttccttcttccatctcccttcaatgtttactttgttctggtccctacagagtcccactataccacaactgatactaagtaattagtaaggccctcctcttttatttttaataaagaagattttagaaagcatcagttatttaataagttggcctagtttatgttcaaatagcaagtactcagaacagctgctgatgtttgaaattaacacaagaaaaagtaaaaaacctcattttaagatcttacttacctgtccataattagtccatgaggaataaacaccctttccaaatcctcagcataatgattaggtatgcaaaataaatcaaggtcataacctggttcatcatcactaatctgaaaaagaaatatagctgtttcaatgagagcattacaggatacaaacatttgattggattaagatgttaaaaaataaccttagtctatcagagaaatttaggtgtaagatgatattagtaactgttaactttgtaggtatgataatgaattatgtaagaaaacaacaggccgggcgggttggttcacacgtgtaatcccagcactttgggaggctgaggcaggcagactgcctgagctcaggagttcgagaccagcctgggcaacacggtgaaatcccgtctctactaaaaatacaaaaaaattagccgggtgtggtgacacatgcctgtagtcccagctacttgggaggctgaggcaggagaatcacttgaacctgggaggtgaaggttgcagtgagccaagaatgcgccacttcactccagcctgggaaacagagcaagactctgtctcaaaaaaaacaaaacaaacaaacaaaaaaacaggctgggcgcggtggctcacgcctgtaatcccagcactttgggaggccgaggcgggtggatcacctgaggtcaggagttccagaccagccttgtcaacatggtgaaacctccccccgccgtctctactaaaaatacaaaaattagccaggcgtggtggcaggagcctgtaatcccagctacttgggaggctgaggcaggagaatcgcttgtacccagaaggcagaggttgcactgagctgagatggcaccattgcactccagcctgggggacaagagcgagatttcgtctttaaaaaacaaaaaacaaaacaaaaaaccatgtaactatatgtcttagtcatcttagtcaagaatgtagaagtaaagtgataagatatggaatttcctttaggtcacaaagagaaaaagaaaaattttaaagagctaagacaaacgcagcaaaatctttatatttaataatattctaaacatgggtgatgaacatacgggtattcattatactattctctccacttttgagtatgtttgaaaatttagtaaaacaagttttaacacactgtagtctaacaagataaaatatcacactgaacaggaaaaactggcatggtgtggtggctcacacttgtaatcccagtgctttgggaggctgagacaggagagttgcttgaggccaggagttcaagaccgacatggggaatgtagcaagaccccgtccctacaaaaaactttgtaaaaatttgccaggtatggtggtgcatacctgtagtcccagctactcgggaggcggaggcagaaggaatcacttgagcccaggagtttgaggctgcagtgagctacgatcataccacagcactccagcgtggacaacagagtaagaccctatctcaaaaacaaaacaaaacaaaacaaacaaaaaaaaccacaagaaaaactgctggctgatgcagcggctcatgcctgtaatcccagtattttgggaggcccaggtgggcgtatcacctgaggtcaggagttagagaccagcctggccaacatggtgaaaccccatctctactaaaaatacaaaattagccaggcatgtggcacgcgcctgtagtcccagttactggggaggctgaagcaggaggatcacctgagcccgggaggtggaggttgcagtgagccgagatcacaccactgcactccagcctgggtgacacagcaataccctacctcaaaataaaaaagaaaaagaaaagaaaagttgctgtccccgctaccccaatcccaaatccaaacagcctctctcatctcacagtaagggggaaaaatcacccaaaaaagctaagtgatcttttgaaaacccaaactcttagaagtctaagattattatagtcaactcatgaagtgtcatcataaaagatactctaatattatttaagtagaaccacatattggttgtcttggtatgtctagcccctggcatacaaaatatttaataacactgatatggtacctgtgatgtgaaaatgtactatgagtacagctttataaatactatatatgtacctatatacagaaaaaaatacaacaaaatcataaaagcacttatctttgaaagaggagttacagcaattttatttagttctttattgctttgctatatattctaaatttttttcaatgaatatatatcacttttaaaaaaattcaatggtctttcttataaattatctttggcagcatgcgtttttatatatacatataaaatgtatgggaaatttttaaaggatacattaaattaaagcaaaatatacaaacaaaaaatcagaatacaaaaagataaaaagattgggaagggagggagggagtaaggaggaagggtgggtgggtatagagaaatataccaaataatggtaagaagtggggtcttgacactttctacactttttttaaataaaaaaaatttttttctctctcttttttttttttagagacgaagtctcgctatgttgcccaggctggtcttgaactcctgggatcaagagatcctcctgcctcagcctcccaaggtgcttggattacaggtgtgagccaccacgcctggtcactttctacactttaatatatatattttttcattttcaatgtcatttttattagttaatttataatacccattcaccattatattcaaagtctatttgaagaaataaaccagaaagaatgaaatactctagctcacatgctattcaatactaaattacctttcaaatcacattcaagaagctgatgatttaagctttggcggtttccaataaatattggtcaaaccataattaaatctcaatatatcagttagtacctattgagcatctccttttacaacctaagcattgtattaggtgcttaaatacaagcagcttgacttttaatacatttaaaaatacatatttaagacttaaaatcttatttatggaattcagttatattttgaggtttccagtgctgagaaatttgaggtttgtgctgtctttcagtccccaaagctcagttctgagttctcagactttggtggaacttcatgtattgtcaggttggcccgtaatacctgtgggacaacttcagcccctgtgcacatggccaggaggctggttgcaaacattttcaggtaggtggaccaggacatgcccctggtcatggccaggtggaggcatagtgctatacagcaggcagaagtcaatattgatttgtttttaaagaaacatgtactactttcataagcagaaaaaatttctattcttgggggaaaagattatgccagatcctctaggattaaatgctgatgcatctgctaaaccttcacatatcagaacatatttactatagaaagaatgaaaatgggacatttgtgtgtcacctatgtgaacattccaaaaatattttacaacaactaagtattttataaattttatgaactgaaatttagttcaagttctaggaaaatacaaaccttgctagatattataaaaatgatacaatatatattcatttcaggctcatcagaatatatctgttatcacttgacaagaatgaaaatgcaccattttgtagtgctttaaaatcaggaagatccagagtactaaaaatgacttcttccttgaagcttactcaccaacttcctcccagttactcactgcttctgccacaagcataaactaggacccagccagaactccttgaaatatacacttgcaacgattactgcatctatcaaaatggttcagtgcctggctacaggttctgcagatcgactaagaatttgaaaagtcttgtttatttcaaaggaagcccatgtgaattctgcccagagttcatcccagatatgcagtctaagaatacagacagatcagcagagatgtattctaaaacaggaattctggcaatataacaaattgatttccaatcaaaacagatttacataccatacttatgtcaagaagttgttttgttttattgcatcctagattttatttttttgatttatggtttactttaagcataaaaaatttgtcaatacaactcttcccaaaaggcataaacaaaaattcataaaacttgcatcacttgagatacttcaggtatgaattcacaactttgttacaacttactatatatatgcacacatatatatatatttgggtatattgggggggttctaatttaagaaatgcataattggctatagacagacagttgtcagaacttggcaatgggtacgtgcaggttcattataccaagtctacttgtagttgttcaaaatgtatcataatacaaggccgggcgaggtggctcacgcctgtaatcccagcattttgggaggctaaggcaggaggattgcttgaggtcaggagtttgtgaccagcctgggcaacagagcaagaccctgtctccaaaaagaaaaaaaataattttttacaaaataaaaacaaaatgtatcatcagacgaaattaaataagaggcaattcatttaaatgacaacttttcccagcttgacatttaacaaaaagtctaagtcctcttaattcatatttaatgatcaaatatcaaatactaattttttttttttttttttttttgagacggagtctcgctctgtcgcccaggctggagtgcagtggcgcgatcctggctcactgcaagctccgcctcccgggttcacgccattctcctgcctcagcctcccgagtagctgggattacagacatgcgccaccacgcccggctaattttgtatttttagtagagatggggtttctccatgttggtcaggctggtcttgaatttcccacctcaggtgatctgcctgcctcagcctcacaaagcagtagctgggactacaggcacccaccaccacacttggttaattcttttgtattttttttgtaaagacgggatttcaccatgttagccaggatggtctcgatctcctgatctcatgatccgcccgcctcagcctcccaaagtgctgggattacaggcgtgagccaccccgcccggccatcaaatactaattcttaaatggtaaggacccactattcagaacctgtatccttatcactaatatgcaaatatttattgaatacttactatgtcatgcatactagagagagttagataaatttgatacagctaccctcacagaacttacagtgtaatagatggcatgacatgtacatgagtaactgtgaacagtgttaaattgctatttaaaaaaaaagacggctgggcgctgtggctcatgcctgtaatcccagcactttgggaggccaaggcaagttgatcgctcgaggtcaagagttcgagaccagcctggccaacgtggtaaaaccccgtctctactaaaaatacaaaaaaaaaattagccaggcatggtggcacaggcctgtaatcccagctactagggaggctgagacatggagaactgcttgaatccaggaggcagaggttacagtgagccgagatcataccactacactccagcctgagtgacagagcgagactccgtctaaaaaaaaaaaaaaaaaaaaagatacaggttaagtgttatggtagttgaagagagaactcaaactctgtctcagaagcctcacttgcatgtggaccactgatatgaaataatataaataggtataattcaataaataggaacttcagttttaatcatcccaaacaccaaaacttcctatcaaacaggtccaataaactcaatctctataagagctagacagaaatctacttggtggcctataatcttattagcccttacttgtcccatctgatattaattaaccccatctaatatggattagttaacaatccagtggctgctttgacaggaacagttggagagagttggggattgcaacatattcaattatacaaaaatgcattcagcatctaccttgattaaggcagtgtgcaacagaatttgcaggagagtaaaagaatgattataaatttacaacccttaaagagcttatagctgggcgtggtggctcatgcctgtaaatcccagcactttgggaggctgaggcgggtggatcacctgaggccagaagttcaagaccagcctagccaacatggcgaaaccctgtctctacaaaaaatacaaaaattagccgggtgtggtggcacgtgcctgtagtcccagttacttgggaggccgaggcaggagaatcgcttgaacctaggaggtggaggctgcagtgagccgagattgtgccactgcactccacttcagcctgggcgacaagagcaagactccgtcacaaaaaaaaaaaaaaaaaaaaagcttaaaatctagtgggaaaggcatatatacatacaactaactgtatagcataataaagctcataatctgtaacaaaatctaattcgacaagcccagaaacttgtgatttaccaaaaacagttatatatacacaaaaagtaaacctagaacccaaagttacccagcaccaatgattctctccctaagcagtatcaagtttaaagcagtgattacattctactgcctagattgtaaactgagtaaaggagaccagcacctttctgctactgaactagcacagccgtgtaaaccaacaaggcaatggcagtgcccaactttctgtatgaatataagttacatctgttttattatttgtgacttggtgttgcatgtggttattatcaacaccttctgaaagaacaactacctgctcaggctgccataacaaaataccacagactgagtgacttaacagaaacttatttctcacagttttggaggctgggaagtccaaaattaaggtacctgcaaggtaggtttcaatctcaggcctcttctttggcttgaaggtcttctaactgtgtgctcacatgacctcttctaacaagctctctggtgtctctttttttttttttttcttttttgagacagagtctcactctgtcacccaggctggagtacagtggcacaatctgggctcactgcaacctccaactcccgggttcaagtgattctcatgcctcaccctcccgagtagcttggatgacaggagcccgctaccacacccagctaatttttgtatttttagtagagatggtgtttcactacattggccaggctggtctcaaactcctgacctcgtgatccacccaccttggcctcccaaagtgctgggattacaggtgtgagccactgcgcccgtcctggtgtcttttcatataagggcactaatccaatcagacctgggcccaaccctcccgacttcttctaactgtaattaccttccaaaggccctgtctccaaataccatcacactgggggttaggacttcaaaaaaggtatggggggggtgtgggaggacataaatgctcagtccataacaagcacccaacataaaaatggctagaacagatcacaaaaaaaaggtcctgtatggctttggggaagggctcaaccccaaaatatctgagagctctggaggggcctagaagtggtaaatgaatgaaaacgtggttactctccagatctgcctttcccaaatatggccattcttggctgaatcagaaatcaaaggacaggttattaattactagctctaagttacttaccatttgctgagacagttcagaaatctgactgcatctcctcagagatctagaacacagttctcaaattctaacttacttgtgatatacttgtgaatgataaaaatcgctacaggtacttttattaatctgaaagagtattgagaaattacctttcattctgacttttgtctggaatgaaaatcaatacttttgctataatcgattactgaaataattttactttccagtaaaactggcattataattttttttaatttttaaaacttcataattttttgccagactgacccatgtaaacatacaaattactaataattatgcacgtcacatctgtaataatggccttcatgtaaacatttttgtggtttacacataaaatctctaattacaaagctatattatctaaaattacagtaagcaagaaaattaatccaagctaagacaatacttgcaacatcaattcatcatctgtgacaaggactgcttaagtctctttgtggttaaaaaggaaaaaaaaaaaaaagacatgttggccagatgcggtggctcacacctgtaatcccagcactttgggaggctgaggtgggcggatcacccctggcctgcccaacatggtgaaaccccgtctctactaaaaacacaaaaattagctgggcgtggtggcgggcgcctgtaattccagctactcgggaggctgaggcaggagaattgctagaacccaggaggcagagattgcagtgagctgagattgcaccattgcactacagtctgggcaacaaaagtgaaactccatcttaaaaaaaaaaagacaatgttcgtgggtccaaacaagacttaatggaagtgagtctaaaaatgagctatgtgggccaggcgtagtggctcccacctgtaatcccagcactttgggaggccgaagcaggcagatcatgaggtcaggagatggagaccatcctggccaacacggtgaaatcctgtctctacaaaaattagctgggcgtggtggtgcctgcctgtaatcccagctactcagaaggctcaggcaggagaatcgcttgaaccagggagtcggtggctagagtgagccgagattgcatcactgcactcctgcctggtgacagagcaagactccatctcaaaaaaaacaaacaaaaataaaagataaaaatgagctatgtgaattaaaagaggtataacaatagataaaccatattttatttaattcctagtaatgagtaatatttccaaacttctggaatgggcagaaattgctagttggcatatttttaccttttatattcagatacattaaaattctcaaaaaaaaacacctcaaagcagatgatccgccatctccttggataatttgtgttaactcaggataacagaaaaccaaaattatgagttactgatgcaatattcctaaatgtaaaaataattaaagctaatagtagattcatcttccaatttcatatcagtcttacaaataaactacatatataacttgcttgccttcccttctgagggataaagctgttagaagaattaaaatcagcattcttgactattcaaccaagggagggataaattattactcattctagggacatgggctcataactactacatgtgtaaggacatgaatttacccaatattacaatttttccttttattagtgtgtacagtggaagaatagacatgttcactctggacaaaaaaaaaattatacttatcagttatcagaagcacaatgctgaagacagtagttccataacaatttgaagtatgtgatcgaactagtagattatcttagtagtagtgaattattgtaaatgttagtaatttggcagccactgggcagaaaaataagaattgaggctcaatattgatattaatggtggtgattgacacataaattttatcaagtctacacaatataaaattacagaaaggtagaagagtataccagtacaacttcaacatatcttcactacaagggagtaaaatgacatggcctagttactatctaatgaactgcagaaaactaaaagaaaactccaaggcaactcttctctgctgatctggttggtccttttcctaccttttgcaatacccagatacaaacaatggatagaaaacaaagtagacttgtagtatgcaggtcacagtgctaaattcacagaaagaaacccctgaactgaactgctctatttcctggtggtcacaaagagtaattctggtttacacctacagattgatgtcaatctacaccctgttgataacagtgtggccaaggacaaaaaaaaggtgctccgttttaccaattctgtaaaaaattattggcagggtaagctcggctagggcaggattacatttctaggactaccatccccgaaatttagaagatattatatccacataaagcatatctttcacattaatttgcaaaaatctaaaagctttttcttagctcaagtgtgtccaagtttaccctggcagtttaaaacgatagttacaagcagcatgggttgtatcagacacatttgagggccaatttcatgtaagtgatattgggcaagttacttcaactatctgtgcctccaaggtcatactagtgtttatttacctaaagggtacctgttatgtaactttagggtgtttacattagataatgcctgcaaaatatttacttcaacgcctaaaacatagttaagtattcaataaatacctactattgtcactactaacttaaaagtttagagattaagagcagaatctggggtgagacaaacttaggttcaaatcctagtattgttgggtaatcttgggcaagttacttaacctctctgatttgtgtaatttaaaaaattagttaatatacataacagggcttagaagagtatctagcacatagcaccatttaagcatttgttattgctaacatgcaaacaatttaagggaaagaaattttttaaaaaggaagagggatttgcaaactaaaaacaatgagtatcttatgttcaaagaaaactaacaaacagccagctctagcaataattaaattcactatatactggggcaggcatcacaccccaaagctaaaagcgtctacctaggccaggcacggtggctcatgcctgtaatcccagcactttgggaagcagaggcgggcagatcgcttgagctcaggagttcaagaccagcctggacaacatggcaaaacaccatctctacaaaaaatacaaatattaggccgggcgcagtggctcacgcctgtaatcccagcactttgggaggccaaggcgggtggatcacctgagatcaggagttcgagagtagcctggccaacatggtgaaacctcgtctctattaaaaatacaaaaaattagccaggcatggtggcaggcgcctgtaatcccagctactcagggggatgaggtaggagaatcgcttgaacccgggaggcagaggttgcactgagccgagatcatgccactgtactccagcccgggcaacaagagcgaaactccatctcaaaaaataaataaataaataaataaaataaagtacaaatattagccagggatggtggtgcgcacctgtagtcccagctacttgggaggctgaagtgggagaatcccctgagcctggggagaatcacccgagcccgggaagtcgaggctgcagtgagcagtgattgtgccactgcactccatcctaggtgacagagtgagaccctgtctcaaaaaaaagaaattggcagaattaagtaagttgatgtttagagatgaaaaatcaacattttttcctcagcaactgaataaaaacaacagccactaccatttttttgagtacctatttgtagcctattttttaactggtattactcgagagagagagagctaggttcgagacagagctccttctcttaataactgtatgacctagggtatgtctgttagcctctctgaggcttcaaaggttcctcatctgtaaaatggtaataatcataccattgctacagggctgttttgaagactaattaggactatgtaagtaaacatgatgatggctattattactgttccccgccaggggccatgcaagggttgctgattcacatagactgtcttataatcctctcaataactccaagaggtagccagcacctcagatatacataaaatgacttaagcccagagaggtgaagtaagttgcccacagccacacaactagtaaatagcccaaacaagctggattcccagttagactccgttaatagcactgctctttaccttaagtcattacaatgcctaatatgaaatagaatcgcttctttcttagggttcaagtggttaattatttaatgtattcattcaacaaaccatcatcgaggacctcttacaagccaagtactgtgctaagtgctagagttacggcggtgattcctgcccttaaaaagttttagtgggagaaacaacaggtaaccaggtcattgccaaaacaacaaaaataatcataataaagcaggctaaagcatatttaactggccggggttttgactattttagcaagcatgatcagaacggttgaggagggaggccagcagcttggccggttcaacaaacaagaaaaaaccagtgagggtggagctaagataccagaggctgattacggttaagaatgttcttgaaggtaaggaccagattctcattttctatatcctggggcatcggtcagcatggaatctggattctagcacatgtgaatttcggcttgaaatgacctaatgccttttccctagttccttcgtgtgtcaaatacgcatggttaccgctaccagagctgtagtggggcttcaatgaggccatgagcatctccataaagatgaactacagtgtgtgcaaaactaaaggcaaaacctggtccccacacgccctcccaggtggtcgctttccgtgccgaggcccctccagaggtgccccgagaacctcaccatcgcaccccaaacttccagggaagggcctctcccgagaaagcccccacgcccccaccccgcgccatcattcccgaatctgccctcggcccctccccgcagcacgctcgcaggcggcacatgtcaaccaaaacgccatttccaccttctcttcccacacgcagtcctcttttcccagggctcccccgaggagggacccaccccaaaccccgccattccgtcctccctgccgccctcgcgtgacgtaaagccgaacccgggaaactggccgcccccgcctgcggggttccctgggcccggccgctctagaactagtggatcccaattgaaggcctggtctaaatgactccaaaatcaccacttaattcaagagactgatttccctgagtcaggccccttaaagcagctatttcaatgggacagggaaacaaccctaggatctggattagaatcacttgggggctgccacacccccagggctctgatcctgcccttctcccacacgcacattcacatactgctgcagtgaccttccatttctaatgggttcctgggccatctgtcaggtatagggaatggaaaaggggttggggaggctctgcttcagaaagtttgtgtcaggggctcccagagcctccacagatagatagcaggggtccccaccctaccatggcagctataaatgtgatcaacatttattggcctaggatacagcagttagcaaaatgcctgatgtagttcccactccgtggaggttgcaggctagccaagaagtcatgagttcagcaacccttacgcaccagtgggatgagattggaccaggccgagggtagtcttgggaacactcagcatttgtctgagggccagaagaggctgcttgccctcagacaggaggtcagcatctttattgtagcccatgacacctctacaccattgctcttctggtcttatggaagacatctttgggcctgataacagcggagtctgtgtcccacttgtccaggctggagtgccacatcaggcacactccagttgcagggacagcacagacaagtttcaggaaggctggtggcctccaggaggttaaccttataaggccagattgtaacctagttgaaaaacatacacatgccatgataataaaagaacctaggcaccattacaagagaaaaaatcatttttgtagatacgagcatggattcttgggtgggtcagacacactgggcttgtgctctgactgcactgtctcccctacctgaccttgggtaaaccataagactgctgcatgactcagtgtccaccccaaaaaagtaccggtagatattggccacagtagatatcagctagagtggactctcatgacaatgaggggagatgtattccccatcttaggcacctgggactctaccttccatcttctgctccgtgtctctccatccccaggctcttcagaactcagggagtccagaatgtcagctcccagatttcagccttcagaaaggaaacccattaccgttcagttgaacaaatgttgtctgagccccagatctgggctcagaggccatctaggctatgagacaagaggggaacaaagcaccgtctgcactcactcaccacactcacttgctgtcccaggtcacatccatcgggtagagaatctaagaggctgagctagctcccgccaccagcccagcccaccccacctggccccttccttccttctacaaaatatgcaccacctgtcaaagggtgggcagtgccaggcctgcatacagagcactgagtgtaaaagcagacatggaccctgacctccaggagcttccaattttcttgaagagacaaatcagctggcatttcagtccagtgtgatctgctcttggtgagcacagacctagggagttggggcagcttcccagaagaactgcagtccaggctgagggcagagaaatgaggggaatggcgaggaattggggagcaggggggagctcagtagagagccaagggcgggaggtgagaagtccgtgttgggccaggagctaccctccggtggccacagccgaagtcgaggatgcctttggaactcatccccacttctctctttctgtatgtagccgtccaagaacaagtcacctccaagtgtagccggatcaaggcaagccccccatctagcaagcacttgatgccacccagaactgggcttcttcagaacaatctgagtccaggaatgatcccactcaccaggcaccagagctgcgagggcatgggagtgatctcaccaactctggggaagcggcaaggaattttcacctccagcccccagtgtcccatcctctcacactcaggccagactcccctgggcagacttgactctgtctgccagcatatgcagagccccaaggccaccccaccagaagtgcccctgcctgggttctgtcccagctccctgggcacccagtccttgagtccccaccagctcagacggcctagtgtgccaagaatgcccactgcgttcaacaatgctgcatgggtcacagcggcagcagctgtgaccacagcagtttcggggaaaacacccctcagccaagtggataatagcgttcagcagcactcaccttctggccaggcctgccttcagaggccatctgattgggaggcacaagtgcccgctgcgatgggaacacaagtgcccctggccaacaaccccagcttcagcctgctgggcagccagagcctcaggcagagcccggtacagggcccggtgcctgtagcaaacaccaccaagttcctccagcagggtatggccagctttagtcccctgagccccatacagggcatcgagccaccaagctatgtggctgctgctgccaccgctgctgctgcttctgccgttgctgccagccagttcccaggtccgttcgacagaacggatattccccctgagctgccacctgccgactttttgcgccagccccaacccccactaaatgatctgatttcgtcacctgactgcaatgaggtagatttcattgaagctctcttgaaaggctcctgtgtgagcccagatgaagactgggtgtgcaacttgaggctgatcgacgacattttggaacagcatgctgctgctcaaaatgccacagcccagaattctgggcaagtcacccaggatgctggggcactttaaatctgagcaggatgcccatagaaacccccatggtgacatcactctaggaagtggtgtcgatccatacccgcagttgtctcccgttacaatttgagtggtgttgtcagcccatgcttatccctctctctacctgtgacaaaatggaaagctggtgatttttcaagctacgtgtacatatttgaaaattttgtaaatggttttcctaaacattaatgacagaagtatttatacttcattttgtgactttgtaaataaagcgacggcttttgtttcagtagagttgtgtttactatgcattgttttgtgtttattatacaatgttacaaatatgcagaccgtgttgtttgctccagtgataccttgttaagctaggtggctgagtcgcttatggttttaatgcaatgagcaatgtggatatgaccaagagttgttgtgcaagttgacaaatgccaaatagaaaaccacttggccatttatttctatgttcactaaaaatcctattgccttgtgtgattcttaatctcttttgcgaacctttcagtctccgctagctctttcctaatgagctttacagcagaagctgttttatcgttaagtgccccacagagacactttaccaggaggctgggagagttctccagatttgggagaggcgcagagacagtgtgtgagccgagccctgtctcagcaatccacctggaggagctagagtatcctcctccctttaccattcagaccgagagaaaaagcccagcttgtgtgcaccctcgtggggttaaggcgagctgttcctggtttaaagcctttcagtatttgttttgatgtaaggctctgtggtttgggggggaacatctgtaaacattattagttgatttggggtttgtctttgatggtttctatctgcaattatcgtcatgtatatttaagtgtctgttatagaaaacccacacccactgtcctgtaaacttttctcagtgtccagactttctgtaatcacattttaattgccacctcgtatttcacctctacatttgaaatctggcgtctgtttcaagccagtgtgttttttcttcgttctgtaataaacagccaggagaaaagtgcctctatgtttttatttttcaagggagtattcagtacctacaaacccaagtcaggaagcctgctagtggctttggttctttcagaggctgctcgatgccttgtgtgtcagaaagaaagattcagcagttttgcatcatggcaaagaagcctgttattttggggctcagcccctcattttatagaggatgaaacagagggggatgggaggtcacaaagacaactgccccgggagcaggtgtgggggagacttgccctgagggtctagacgctctgcaccaccgtcctgtctcccttgctgaagaccacacatgcccttctttgaccagaccctgccacctgataggccaggacctggtaggcgggtacccaggtttcatggatggaaccacatctccccaaaagtggggaggtagctactgggatgcacgcctcccgccatgtgctataggagagcagctgaagcaacagttgggatcagatgtagtcacaattgaatgcatcatcacatttatccctctaagtggctgggagagttgatatcctcatccctaaggtacaaaatgttccaatttgatcagtggctttcaggagctgagaaaggcatgtgctctgaggcagagctgttatgtcccgcagagcctaaaaatgctctaagaacatgctccctgccaaaattctcaatggctgtgacaagggacaacgatcgaccaatgggggtggaagcagacctccgcagtccaggggccagagctaggacagaggggtcggagaaagagtcattttcccaacactccagctcttggccagtcctcacacagtcccctcctgcttcctgctgagagagatatcctcataggtctgggtaaagtccttcagtcagctttcattccctgtcaccaactttgtctctgttctccctgcccgtctcaggcagcactcctcaggaaacctctccaagagccagcctcactgcagcgcccactattgtccctctgcctcaagtgtcccatccatgccaggccccaggcaggctgcagctttccctcagggccacaccaaagcacttgggctcagctgtgctgtccccctccatcactgagctcaggggcagcaggggtggggtgccaggaggcccattcacccttctctggctctgtgttggacccacctgcccagccactgctgcttagaacctacccgctgggaaaatgaagccctcccggaggggccacctcaacctgagagcctcacggatcacagttgtccccactcagctctgccagccctcagagacccatagataaaagctgagcttggctcgcagagctggttccatcttccattcccagagggttcaacttcctaccccaaccacacagggaacctcaaggctgagccagtgtgggctgcagtgcagaccagcttcctggacacgtcctgccacctgaccccaggctggcctcactgcccctggcactcctgaccctatcctcattcctcctggcagtgcgtgttctgccattccgctttcccttagctgtcctctcactgtactgtcagcttctccttttccaggtgccccccaggggctttccacatgaccctgtcaccccacagcccatccagcaccaattccagctctctgccacccttcaaaggagtgacagtgccctgcttcacctcccactcacccctcaacccagagcaatctggctccagtcttgcctccttccccctaagtactctagtcacagttccaaattcctcctggtcataaagccaaatgaagcttcctggtcctcagcggacttgccacttcagcagtactggactctctcctcccagaaacctgtttccccttggctcctggagcccacactctgctggaatccttctgcctctctggcctgtagcctggccctctctcccaacctgaggtccattctctcctgctcctccacaagatgttgctccttccattacttcctccctctcaaccaaagctccttcattagctctttatcttctggtttcttcccctgggcagacgaatggattcaagagcctgtggcccagcagcccagcactccaggatctcagcacttcagcatcccagtaccctagcatctcaataccccagcaccccagcaccatagtattccagcaccccattgtccaagcatctcagcactccagcatcccagcaccccaacactccagcagcccagaatctcagcaccctagcactgcagcatctcaggaccccagcacttcagcatcccagcacactagtactccagcatctcggcaccccagcacctaggcatcccaacacccagcaccccagcacttaagcatcccaccactacagtatctcaacactccagcaccccagcaccatagtgttccagcaccccagcatcccaacaccccagcacttaagcatcccaacacctcggcatcccaacaccccagcactgcagcatctcagcaccttagcatcccagtgccctagcatctcaatgctccagcacaccagtactacagtattccagcaccccagcactccagcatctcagcactgcagcactgcagcactccagcatcccaaaatcccagcatcccaacaccccagcagaccagcagaccagcatctcagcaccgcagcatccaaggactatcccagcatcccagcaacccagcacctcagcatcccaacaccccagcatttcagcatggcaacaccccagtaccccagcacttcagcaccccagtatcccagcatctcagcgacccagtatcacaaaacctcagcatcctagcaccccagcaccccagcaccttagcaccttagcatcccagcatctcagcgcctcagcatcttgatattctggctgaggtcagcgtggtgtatctagtcagggtcctaactttcacttcgcagggaaatgctgctggactgggtctcatgttgggctgaagctctctagaccccttgaagacagcataaaagagcttggagacgctgggtgtcccccatggaagagttcactctcatcctgctttgacaacagccttctctggggtccctcacgggcccctctttcttactgcaagtttgtctctgagaagactgtgatgcagaagtcactcagctgcctgtggctcctgaagagctgaaggtggaggcctgtaggcctccctatgagaggcgcagaaaaaaccatgattgctagtggggaggtgctccctctacaacccactccataatctgcccccgcccagctctgaggccagccccaggggaaaatgccagatccccagggaggtgtgtgagacctcaggggctccctcctcccttacagcaggctcaggcccctgggggcctcagggccaaggtctgtgggtaagctactatctctcacttgtcctctagccacaaaagccagggagatctggcaatggacatgaggttctgaagaagcacatatgactggcttcctaatgcgtggttgttcagtgattcaataaacacgcatgggccaggcatggggaaatagacaaacatgatccccaacctctcccagagtgaactgggagggaggagtgttcatccctcaggattacaccagagaaacaaaccagcaggagatatatatggttttggggggtcaagaaagaggaaaaacctggcaaggcaagtccaaaatcataggacaggctgtcaggaagggcagcctggaacctctcaagcaggagctgatgctgcagtccacaggcagaatttcttcttcctcggggaaatctcagctttgttcttaaggcctttcaactgattggctgaggtctgccccttcccccacattctccaggataatcttccttacttaaagtcaactattaatcacagctacaaaatcccttcacagctacacatagatcagtgtttgattgacgaacagcccctacagcctagccaagttgacacataaaactaaccatcacagggggacaaatgatgtaaacacatcaacaaataaaacagtaacaagttaaggtctatggaaaaaacacagaaggggcagagagaaagaaagcaagaaggagagtcccagtttgctagggcttgtgggaagtggggagcagttctctttagctaggatatttgggaaaggcatatctgaaggagtgatatttgagcttagattaaaagatgggaaggagcaagccatgcaaagagctaggatgttccaagcagagacggaacagcaagtgcaaatgtcaggaggaatagaaggaggctggtgggtggggtccagtgagcaagaggagggcaggcaggagaggggatggggaggtgggcaggcccagaccacccagggccctggagactatcctgatccaacaagggaagccttgagtcacttcagtgtccatgtggagaatggacctcagactgaatgagggaggcagtaaggagggcctctacctccagggcttcgccctgtggactgcgcatagacatctccaactcagaaagtctgaaccaaactttccatagttcccccaagtctgggcatcctcctactcagtgaaaggcagccatcacacctccctgccctgctcccggatgccccaaatcctcttggtctccaagtccagaacctgagacttgtccttgatgtttgtctttccctcaccctttctgtattctgggaagatgggtttttttcccccagatgaatctgtaaaacttctgtgatcacaataaaaattctggcagtattattttctggaacatgacaaagtgattcaaaattatttatctggaagactacaaaacaagaatagccaggaaatttctaaaaagaaagaagaaggaggaggagaaagaaggaggaggaaaaggaggagaagaagaaaagaaaaagaaccaagaaagggttctagctctaccaaatattaaaacatatcatgaagctatttaaaacaatatggttgtggatactgaaaaagatgtgaataaagtggaaggaaaataaatagaaatgcacatggggattgagactgtgaaaaaggcagcatctcacatcagtgagggatgttcaacacctggtgttgggaaaactggctagtcatttaaaccaaacaactgggtcctctacctcactcctgacattaagatacatttagatgattcaaagagtaagacagaaaaaataacacgtgaaaacactatcagaaaacaacgtgggccaggtgtggtgggtcacgcctgtaatcccagcactttgggaggccgaggcagacagatcacctgaggtggggagttcaagaccagcctgaccaacatggtgaaatcctgtctctactaaaaatacaaaattagctgagcgtggtggcgcatgcctgtaatcccagctactcaggaggccgaggcaggagaatcacttgaacctgggaggcagaggttgtggtgagccgagatcacgccattgcactccagcctgggcaacaagagtgaaaatccatctaaaaaaaaaaaaaaaagccaaggtggatatttttatagtatcagggtagatcaagcttctccaatcatgacatgaaacccagaaaccataaaagaaaagaatgataaaattgcccacgtaaagtaaaaagcttgcacacagaaaaacaccatacaggttacaagatgagcagcaaaatcagagaaaaaacattgcaattcaggacacacagaggctattgttcctaatatttaaaaataaaagtagtggattgtctacaaaaagatgaagacaagaatttcagaaaaccaaatactgcatgttttcacttacaagtggaagctaaacactgagtacacgtgtacacaaagaatggaaccataggccaggcaccgtggctcacgcctgtaatcccagtactttgcgaggccgaagcgggcggatcacctgaggtgaggagttcgagaccatcctggccaacatggtgaaacccagtctctactaaaaatacaaaaattagccgggcgtggtggtgggtgcctgtaatcccagctactcgggaggctgcggcagtagaatcgcttgaaccctggaggtggaccttgcagtgagccgagatcgcaccactgcactccagcctgggcaacagagtgagactccatctcaaaaaaaaaaaaaaggaatagaacaatagacactggggcctacttgagggaggagggtgaggatcaaaaacctgcctatcaggtactatgcttattacctgggtggtgaaataatctgtacaccaaaccccagtgacatgcaatttaccgatgtaacaaacctgcccatgtacccgctgaacctaaaataaaagttggaaaaaaatatagaaattttctttgtaatagccaaaaactgcaaacagcccaggtgtctattagtagaatgcataaacaaactcgggcatgttcatacaatgtaaaactactcatcaataaaaagtgatacttctcagcaatgaaaagaaactagctactgataccagctacaacatggatggatttcaagtgctttatgatgagagcaagaagccagacacaaaagtgtctatatatatatacagtatatatacgtatatatacacatatatacagtatatatatacatatacatgtatatatatactgtatatatactgtatatatatacacagtatatatatacatatatacagtgtatatatactgtgtatatatacatgtatatatactgagtatatatacatgtatatatatgtatactgtatatatactgtatatatatatacacatatatacagtatatatatacagtatatactgtatatatacagtatatacgtgtatatatacatatatacagtatatatgtaaatatacatatatacagtatatatgtaaatatacatatatacatgtatatatatacactatatatatacatatatagtgtatatatacatatatacatgtatatatttactatatgattccatttatataaagtgccaaaacagtcaaaaataatctatgtggaaaaaatcaacaaagggatcccccgggctgcaggaattcgatggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaagtctatctacttcgtggccggattgtttgttatgctggtgcagggttcctggcagattaagcctgaggctcccggagaagacgccagccccgaagagctcaaccgctactacgctagtctgcggcactaccttaacctcgtaacaagacagcggtatcgggccaagagggcacccgtgaaacagacattgaatttcgacctgctgaaactcgcaggagacgtcgagtctaatcctggaccgatcaaacctgaagcccccggtgaagacgcatcaccagaagagttgaaccgctattacgcttctcttagacactacttgaacttggtgacacggcaacggtactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgcctaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatga

SEQ ID NO: 47 - DNA, 5984 bp, Artificial Sequence
  Synthetic  HDAd-EF1a-PYY Approach 1 Codon Optimized Nucleic Acid Sequence Encoding PYY (3-36) Approach 1 Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggtatttgtcagacgaccttggcctgccctcaccacggtgttgctcgccttgctcgtttgcttgggggccttggtggacgctatcaagcctgaggcacccggagaagacgcatcccccgaggaattgaaccgctattacgcatccttgcggcactacctgaacctcgtgaccaggcaacgctatcggcacgacttccctgaagaagtcgcaatcgtcgaagagctcggcaggagaatcaaacctgaggctcctggtgaggacgccagtccggaggaactgaatcgctactacgcgtcacttaggcactacctgaacctggtcactcgccagagatactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 48 - DNA, 5942 bp, Artificial Sequence
  Synthetic  HDAd-EF1a-PYY Approach 2 Codon Optimized Nucleic Acid Sequence Encoding PYY (3-36) Approach 2 Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggtctttgttagacgaccgtggccggctctgacaactgtgctgctcgcccttctcgtgtgcctgggagctcttgttgatgccatcaagcctgaggcacctggagaagacgcatcccctgaggaactgaaccgctattacgcatcattgagacactaccttaacctcgtgacaagacagcggtacggtaagagaatcaagccagaggcccccggggaggatgcttccccagaggagttgaataggtattacgctagcctgagacattacttgaaccttgtgacacgacagcgatattgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 49 - DNA, 5996 bp, Artificial Sequence
  Synthetic  HDAd-EF1a-PYY Approach 3 Codon Optimized Nucleic Acid Sequence Encoding PYY (3-36) Approach 3 Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatggtatttgtgagacgcccctggcccgcgctgacaaccgtgttgctggcacttcttgtgtgccttggagctctcgttgatgccatcaagcctgaagcaccgggggaagacgcttcccccgaggagctcaatcgatactatgctagtctgcgacattatctgaaccttgtgaccaggcagcggtacggcaaacgggattttccagaggaagtggcgatcgtcgaggagctcggacggcggggaaaacgcatcaaacccgaggccccaggcgaggatgcttcacctgaggaacttaaccgctactacgcaagcctgcggcattaccttaacctcgtgaccagacagcgctactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 50 - DNA, 6053 bp, Artificial Sequence
  Synthetic  HDAd-EF1a-PYY Approach 4 Codon Optimized Nucleic Acid Sequence Encoding PYY (3-36) Approach 4 Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaagtccatctacttcgtggctggcctgtttgtgatgctggtgcagggtagctggcagatcaagccagaggcacctggggaggacgcatcaccagaagaattgaaccggtattacgcgtcactccgccactatcttaacctggtcacgaggcagagataccgcgcaaagagagctcctgtcaagcagacactcaactttgacctgctgaagctggcaggagacgtagagtccaatcccggtcctatgaagagcatctactttgtggctgggttgtttgtgatgctggtccaagggtcctggcagatcaagcccgaggcccccggggaggatgccagtccagaggagctgaatcgatattacgccagcctgcggcactacctcaaccttgtaacacgacagagatactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 51 - DNA, 6098 bp, Artificial Sequence
  Synthetic  HDAd-EF1a-PYY Approach 5 Codon Optimized Nucleic Acid Sequence Encoding PYY (3-36) Approach 5 Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaagtcaatctatttcgtggcgggcctgttcgtaatgctggtgcagggctcttggcagatcaagcctgaagcgcctggagaggacgcatcccctgaagaactgaatcgctactatgccagcttgcgccattatctgaacttggtgactagacagcgctatagagccaagagggctcccgtgaaacaaacactgaattttgatttgcttaagctcgccggggacgtggaaagcaatcctggccccgactttcctgaggaagtagctatcgtggaagagctcggaaggcgaatgaaatccatttattttgtcgctggcctgtttgtgatgctggttcaggggagttggcaaatcaagcccgaggcgcccggcgaagacgcgtcccctgaagagttgaacaggtactatgccagcctgaggcactatcttaacctggtgacgaggcaaagatactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 52 - DNA, 5993 bp, Artificial Sequence
  Synthetic  HDAd-EF1a-PYY Approach 6 Codon Optimized Nucleic Acid Sequence Encoding PYY (3-36) Approach 6 Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaagtctatctacttcgtggccggattgtttgttatgctggtgcagggttcctggcagattaagcctgaggctcccggagaagacgccagccccgaagagctcaaccgctactacgctagtctgcggcactaccttaacctcgtaacaagacagcggtatcgggccaagagggcacccgtgaaacagacattgaatttcgacctgctgaaactcgcaggagacgtcgagtctaatcctggaccgatcaaacctgaagcccccggtgaagacgcatcaccagaagagttgaaccgctattacgcttctcttagacactacttgaacttggtgacacggcaacggtactaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 53 - DNA, 6038 bp, Artificial Sequence
  Synthetic  HDAd-EF1a-PYY Approach 7 Codon Optimized Nucleic Acid Sequence Encoding PYY (3-36) Approach 7 Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaaatctatatactttgtcgctggcctctttgtgatgctcgttcagggctcatggcagatcaagcccgaggctcctggagaagatgcatctccagaggaactcaatagatattatgcctcactgcgccattacctgaacctggtcacacgccagcggtatcgggccaagcgggctcccgtaaagcaaaccctgaacttcgatctgctgaaactggccggagacgtagagtccaatcccggccccgatttccctgaggaggtcgctatcgtcgaagagttgggcaggaggatcaagcccgaagcaccgggggaggacgctagccccgaggaacttaaccgatactacgctagcctgagacattatctcaatctggtgacacgccaacggtattaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 54 - protein, 136 aa, Artificial Sequence
  Synthetic  Oxyntomodulin Amino Acid Sequence in Approach A Construct, (Proglucagon signal peptide-Oxy- PCK1/2-Oxy-PCK1/2-Oxy)
MKSIYFVAGLFVMLVQGSWQRHSQGTFTSDYSKYLDSRRAQDFVQWLMNTKRNRNNIARHHSQGTFTSDYSKYLDSRRAQDFVQWLMNTKRNRNNIARHHSQGTFTSDYSKYLDSRRAQDFVQWLMNTKRNRNNIA

SEQ ID NO: 55 - DNA, 411 bp, Artificial Sequence
  Synthetic  Codon Optimized Nucleic Acid Sequence Encoding Oxyntomodulin Approach A Construct
atgaaatctatctactttgtcgctggtctctttgtgatgctcgtccaagggtcctggcagagacacagtcaggggacttttacatcagattatagtaagtatctcgacagcagacgcgcgcaggactttgtgcagtggctgatgaatactaaacgaaaccgaaataacatcgccagacatcacagccagggcacttttacaagcgactatagcaaatatctggacagtaggcgagcccaagatttcgtgcaatggctgatgaatacaaagagaaaccgcaataacatagcgcgacatcactctcagggcaccttcaccagtgattactccaagtacctggattctcggagagcgcaagatttcgtgcagtggctgatgaatactaagagaaatcgcaacaacatcgcatga

SEQ ID NO: 56 - protein, 111 aa, Artificial Sequence
  Synthetic  Oxyntomodulin Amino Acid Sequence in Approach B Construct, (Proglucagon signal peptide-Oxy-IP-2-Oxy)
MKSIYFVAGLFVMLVQGSWQHSQGTFTSDYSKYLDSRRAQDFVQWLMNTKRNRNNIARHDFPEEVAIVEELGRRHSQGTFTSDYSKYLDSRRAQDFVQWLMNTKRNRNNIA

SEQ ID NO: 57 - DNA, 399 bp, Artificial Sequence
  Synthetic  Codon Optimized Nucleic Acid Sequence Encoding Oxyntomodulin Approach B Construct
aagcttgcatgcctgcaggtcgactctagactgccatgaagtccatttacttcgtggcaggtctctttgtgatgttggttcagggtagctggcagcactcacagggcactttcacttccgattactctaagtacctggacagcaggcgggcccaggatttcgtgcagtggcttatgaacacaaagagaaacagaaataatattgcccggcatgactttcctgaagaggtggctatcgttgaggagctgggaagacggcactctcagggcacctttacatccgactactcaaaatacctggattcaagaagggcgcaggattttgtgcagtggcttatgaacacaaaacgcaaccgcaataacatcgcctaaggatccccgggagatatcctaggcttgg

SEQ ID NO: 58 - DNA, 6053 bp, Artificial Sequence
  Synthetic  HDAd-EF1a-Oxy Approach A Codon Optimized Nucleic Acid Sequence Encoding Oxy Approach A Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaaatctatctactttgtcgctggtctctttgtgatgctcgtccaagggtcctggcagagacacagtcaggggacttttacatcagattatagtaagtatctcgacagcagacgcgcgcaggactttgtgcagtggctgatgaatactaaacgaaaccgaaataacatcgccagacatcacagccagggcacttttacaagcgactatagcaaatatctggacagtaggcgagcccaagatttcgtgcaatggctgatgaatacaaagagaaaccgcaataacatagcgcgacatcactctcagggcaccttcaccagtgattactccaagtacctggattctcggagagcgcaagatttcgtgcagtggctgatgaatactaagagaaatcgcaacaacatcgcatgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 59 - DNA, 5978 bp, Artificial Sequence
  Synthetic  HDAd-EF1a-Oxy Approach B Codon Optimized Nucleic Acid Sequence Encoding Oxy Approach B Construct in HD-MAR-EF1a shuttle vector
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgaagtccatttacttcgtggcaggtctctttgtgatgttggttcagggtagctggcagcactcacagggcactttcacttccgattactctaagtacctggacagcaggcgggcccaggatttcgtgcagtggcttatgaacacaaagagaaacagaaataatattgcccggcatgactttcctgaagaggtggctatcgttgaggagctgggaagacggcactctcagggcacctttacatccgactactcaaaatacctggattcaagaagggcgcaggattttgtgcagtggcttatgaacacaaaacgcaaccgcaataacatcgcctaaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata

SEQ ID NO: 60 - DNA, 102 bp, Artificial Sequence
  Synthetic nucleic acid sequence of PYY(3-36)
attaaacctgaggcgccaggggaggatgcaagccccgaggaactgaaccgctattatgccagtctgcggcattatttgaacctggtgaccagacagcgctac

SEQ ID NO: 61 - protein, 182 aa, Artificial Sequence
  Synthetic oxyX4 AA sequence
MGVHECPAWLWLLLSLLSLPLGLPVLGRHSQGTFTSDYSKYLDSRRAQDFVQWLMNTKRNRNNIARHHSQGTFTSDYSKYLDSRRAQDFVQWLMNTKRNRNNIARHHSQGTFTSDYSKYLDSRRAQDFVQWLMNTKRNRNNIARHHSQGTFTSDYSKYLDSRRAQDFVQWLMNTKRNRNNIA

SEQ ID NO: 62 - DNA, 6191 bp, Artificial Sequence
  Synthetic optimized nucleotide sequence in EF1a expression cassette pAd-MAR-EF1a-opt Oxy X4
ggccgattcattaatgcaggggccgctgcggccatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggctagcctgcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcatctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagtagcaagcttgcatgcctgcaggtcgactctagactgccatgggcgtccacgaatgtcccgcatggctgtggcttctcctcagcctcctgagcctgccgctgggtttgcctgtcttgggacgacattcacaaggcacattcaccagcgactacagtaagtacctcgatagccgcagagcccaggattttgtgcaatggttgatgaacacaaagcggaacaggaacaacatagccagacaccactctcaagggaccttcacatctgactattctaaataccttgacagccgccgggctcaagacttcgttcagtggttgatgaataccaaacgcaatagaaacaatattgctaggcatcactcacaggggactttcactagtgactattcaaaatacctggactcccggagagcacaggattttgtgcagtggctgatgaacacaaagcgcaatcgaaacaacatcgccagacaccattcccagggcacatttaccagcgattactccaagtatctcgacagtcgacgggcccaagattttgtccagtggcttatgaatacaaagcggaaccggaataatatcgcatgaggatccccgggagatatcctaggcttggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattcagtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggcttaattaaggcgcgccgggcccctacgtcacccgccccgttcccacgccccgcgccacgtcacaaactccaccccctcattatcatattggcttcaatccaaaataaggtatattattgatgatggccgcagcggccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggccatcatcaataatataccttattttggattgaagccaata